(19) United States
(12) Patent Application Publication
(10) Pub. No.: US 2025/0304955 A1
(43) Pub. Date: Oct. 2, 2025

(54) COMPOSITIONS AND METHODS FOR MYOSIN HEAVY CHAIN BASE EDITING

(71) Applicant: THE BOARD OF REGENTS OF THE UNIVERSITY OF TEXAS SYSTEM, Austin, TX (US)

(72) Inventors: Eric N. OLSON, Dallas, TX (US); Rhonda BASSEL-DUBY, Dallas, TX (US); Andreas CHAI, Dallas, TX (US)

(73) Assignee: THE BOARD OF REGENTS OF THE UNIVERSITY OF TEXAS SYSTEM, Austin, TX (US)

(21) Appl. No.: 18/573,776

(22) PCT Filed: Jul. 1, 2022
(86) PCT No.: PCT/US2022/073386

§ 371 Date: Dec. 22, 2023

Related U.S. Application Data
(60) Provisional application No. 63/217,618, filed on Jul. 1, 2021.
(60) Provisional application No. 63/218,221, filed on Jul. 2, 2021.

(51) Int. Cl. C12N 15/113; A01K 67/0276; A61K 31/7088; A61K 48/00; A61P 9/00; C12N 9/22; C12N 9/78; C12N 15/62; C12N 15/86

(52) U.S. Cl. CPC C12N 15/113; A01K 67/0276; A61K 31/7088; A61K 48/005; A61P 9/00; C12N 9/226; C12N 9/78; C12N 15/625; C12N 15/86; C12Y 305/04; A01K 2207/15; A01K 2227/105

(57) ABSTRACT

Disclosures herein are directed to compositions comprising single guide RNA (sgRNA) and fusion proteins comprising a Cas9 nickase and deaminase designed for a CRISPR-Cas9 system and method of using thereof for preventing, ameliorating or treating one or more cardiomyopathies.

### CROSS-REFERENCE TO RELATED APPLICATION

This application claims the benefit of U.S. Provisional Application No. 63/217,618, filed Jul. 1, 2021 and U.S. Provisional Application No. 63/218,221 filed Jul. 2, 2021, the disclosures of which are hereby incorporated by reference in its entirety.

### INCORPORATION BY REFERENCE OF SEQUENCE LISTING

This application contains a Sequence listing that has been submitted via Patent Center in a computer readable format and is hereby incorporated by reference in its entirety. The computer readable file, created on Jul. 1, 2022, is named UTSW-3923-PCT (106546-728561).xml and is about 368,000 bytes in size.

### BACKGROUND

### 1. Field

The present inventive concept is directed to compositions comprising single guide RNA (sgRNA) and fusion proteins comprising a deaminase and an Cas9 nickase or deactivated Cas9 endonuclease and method of using thereof for preventing, ameliorating or treating one or more cardiomyopathies.

### 2. Discussion of Related Art

Cardiomyopathy is a disease of the heart muscle that causes the heart muscle to become enlarged, thick, and/or rigid. As cardiomyopathy progresses, the heart becomes weaker and can lead to heart failure or irregular heartbeats (i.e., arrhythmias). Hypertrophic cardiomyopathy (HCM) is a principal types of cardiomyopathies that often arises from genetic mutations in sarcomeric, cytoskeletal, and/or desmosomal genes. Currently, there is no cure for these cardiomyopathies aside from transplant. As such, there is a need in the medical field for treatment of these cardiac diseases.

### SUMMARY

The present disclosure is based, at least in part, on the discovery of guide RNAs (gRNAs) for use with Clustered Regularly Interspaced Short Palindromic Repeats (CRISPR)-CRISPR associate protein 9 (Cas9) systems that successfully reverse phenotypes associated with familial cardiomyopathies such as HCM by correcting genetic mutations through base-pair editing.

Aspects of the present disclosure provide a gRNA comprising a spacer sequence corresponding to a DNA nucleotide sequence of SEQ ID NO: 1 or 2. In some aspects, the gRNA comprises a spacer sequence having at least 85%, at least 90%, at least 95%, at least 96%, at least 97%, at least 98%, at least 99% or 100% sequence identity to SEQ ID NO: 5 or 6. For instance, in some aspects the gRNA may comprise a spacer sequence comprising or consisting of SEQ ID NO: 5 or 6.

Other aspects of the present disclosure provide a fusion protein comprising a deaminase covalently linked to a Cas9 nickase or deactivated Cas9 endonuclease.

In various aspects, the deaminase may be selected from the group consisting of ABEmax, ABE8e, ABE7.10 and any functional variant thereof. In various instances, the deaminase may comprise an amino acid sequence having at least 85%, at least 90%, at least 95%, at least 96%, at least 97%, at least 98%, at least 99% sequence homology to any one of SEQ ID NOs: 7, 9 and 11. For example, the deaminase may comprise an amino acid sequence comprising SEQ ID NO: 7, 9 and 11. In some embodiments, the deaminase comprises an amino acid sequence comprising SEQ ID NO: 7.

In various aspects of the present disclosure the Cas9 nickase or deactivated Cas9 endonuclease is selected from SpRY, SpG, SpCas9-NG, SpCas9-VRQR or a variant thereof. In some aspects, the Cas9 nickase or deactivated Cas9 endonuclease comprises an amino acid sequence having at least 85%, at least 90%, at least 95%, at least 96%, at least 97%, at least 98%, at least 99% sequence homology with any one of SEQ ID NOs: 15, 17, 19, and 21). For instance, the Cas9 nickase or deactivated Cas9 endonuclease may comprise an amino acid sequence comprising any one of SEQ ID NOs: 15, 17, 19, and 21 (SpRY, SpG, SpCas9-NG, SpCas9-VRQR). In some aspects, the Cas9 nickase or deactivated Cas9 endonuclease comprises an amino acid sequence comprising SEQ ID NO: 15.

In any of the aspects of the present disclosure, the deaminase may be covalently linked to the Cas9 nickase or deactivated Cas9 endonuclease via a peptide linker. In some aspects, the peptide linker comprises an amino acid sequence comprising SEQ ID NO: 27.

In any of the fusion proteins described herein, the deaminase and/or Cas9 nickase or deactivated Cas9 endonuclease further comprises a nuclear localization signal (NLS) peptide. In various aspects, the nuclear localization signal (NLS) peptide may be selected from any one of SEQ ID NOs 31-42. In some aspects, the nuclear localization signal (NLS) peptide can comprise SEQ ID NO: 31 or SEQ ID NO: 32.

In any of the aspects of the present disclosure, a fusion protein is provided comprising an amino acid sequence having at least 85%, at least 90%, at least 95%, at least 96%, at least 97%, at least 98%, or at least 99% sequence homology to any one of SEQ ID NOs: 45-60. In some aspects, the amino acid sequence of the fusion protein comprises or consists of any one of SEQ ID NOs: 45 to 60. In some aspects, the amino acid sequence of the fusion protein comprises or consists of SEQ ID NO: 45 or 46 (ABEmax-SpCas9_VRQR).

Further aspects of the present disclosure provide isolated nucleic acids encoding any gRNA described herein. Other aspects provide isolated nucleic acids encoding the fusion protein provided herein. Also provided are viral vectors comprising one or more of the nucleic acids encoding the gRNA and/or the fusion protein or a fragment thereof. In some aspects a pair of viral vectors are provided comprising (a) a first viral vector comprising a nucleic acid encoding a first fragment of the fusion protein of any one of claims 4 to 20 and (b) a second viral vector encoding a second fragment of the fusion protein, wherein the first fragment and the second fragment of the fusion protein can undergo protein trans-splicing to form the fusion protein. In any aspect the first and/or second viral vector may further comprise a nucleic acid encoding a gRNA targeting SEQ ID NO: 1 or 2.

Further aspects of the present disclosure provide a pharmaceutical composition comprising any isolated nucleic acid encoding a gRNA or fusion protein (or fragment thereof) as provided herein, the viral vector, and/or the pair of viral vectors as provided herein and a pharmaceutically acceptable carrier, diluent and/or excipient. In some aspects, the pharmaceutical composition may further comprise a liposome.

Further aspects of the present disclosure provide a method of correcting a mutation in an MYH7 gene in a cell, the method comprising delivering to the cell: a Cas9 nickase or deactivated Cas9 endonuclease, a deaminase, and a gRNA targeting a DNA nucleotide sequence selected from any one of SEQ ID NOs. 1 or 2, or one or more nucleic acids encoding the Cas9 nickase or deactivated Cas9 endonuclease, deaminase and/or gRNA, to effect one or more single-strand breaks (SSBs) within or near the MYH7 gene that results in one or more mutations of at least one nucleotide within or near the MYH7 gene, thereby correcting the mutation in the MYH7 gene. In some aspects, the method comprises delivering to the cell a nucleic acid, viral vector or pair of viral vectors described herein.

Further aspects of the present disclosure a method of treating a cardiomyopathy caused by a mutation in an MYH7 gene in a subject in need thereof, the method comprising delivering to at least one cell in the subject expressing the MYH7 gene: an RNA-guided DNA-nickase, a deaminase, and a gRNA targeting a DNA nucleotide sequence selected from any one of SEQ ID NOs. 1 or 2, or one or more nucleic acids encoding the RNA guided nickase, deaminase and/or gRNA, a to effect one or more single-strand breaks (SSBs) within or near the MYH7 gene that results in one or more mutations of at least one nucleotide within or near the MYH7 gene, thereby correcting the mutation in the MYH7 gene in at least one cell of the subject. In some aspects, the method comprises administering a pharmaceutical composition comprising a nucleic acid or viral vector comprising the nucleic acid encoding one or more of the gRNA and/or fusion protein provided herein to the subject. In various aspects, the mutation in the MYH7 gene comprises one or more single nucleotide polymorphisms that result in a single amino acid substitution in a protein product encoded by the mutated MYH7 gene. In various aspects, the protein product may be a myosin protein or peptide and the single amino substitution comprises R403Q according to SEQ ID NO: 96.

Further aspects of the present disclosure are directed to a gene edited mouse comprising a human nucleic acid comprising a MYH7 c.1208 G>A (p.R403Q) human missense mutation inserted within an endogenous murine Myh6 gene to form a humanized mutant Myh6 allele. In some aspects, the human nucleic acid further comprises a first polynucleotide adjacent to and upstream of the missense mutation and a second polynucleotide adjacent to and downstream of the missense mutation. In various aspects, the first polynucleotide comprises about 30 to 75 nucleotides, about 35 to about 70 nucleotides, about 40 to about 65 nucleotides, or about 45 to about 60 nucleotides. In some aspects, the first polynucleotide comprises or consists of 55 nucleotides. In some aspects, the second polynucleotide comprises about 10 to 30 nucleotides, about 15 to 25 nucleotides, or about 20 to 25 nucleotides. In further aspects, the second polynucleotide comprises or consists of 21 nucleotides. In various aspects, the human nucleic acid comprises a nucleotide sequence of SEQ ID NO: 97. In any of the aspects herein, at least one cell of the mouse expresses a mutant myosin protein comprising a R404Q substitution relative to a wildtype myosin protein comprising SEQ ID NO: 94. In further aspects, the mouse may also comprise a wildtype Myh6 allele, and the mouse is heterozygous for the humanized mutant Myh6 allele.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein. Embodiments of the present inventive concept are illustrated by way of example in which like reference numerals indicate similar elements and in which:

FIGS. 1A-1C depict representative schematic diagrams and a graph illustrating an exemplary CRISPR-Cas9 system used for correction of a MYH7 mutation in human cell according to various aspects of the disclosure. FIG. 1A shows a schematic illustrating an exemplary overview of gRNA design. FIG. 1B shows a schematic illustrating an exemplary overview of a CRISPR-Cas9 system transfection into human iPSC cells. FIG. 1C shows a graph illustrating editing efficiency of an exemplary CRISPR-Cas9 system for correcting a MYH7 R403Q mutation.

FIGS. 2A and 2B depict a representative schematic diagram and a graph illustrating an exemplary CRISPR-Cas9 system used for correction of a MYH7 mutation in human cell according to various aspects of the disclosure. FIG. 2A shows a schematic illustrating an exemplary overview of differentiation of human iPSC cells after administration of a CRISPR-Cas9 system correcting a MYH7 R403Q mutation. FIG. 2B shows a graph depicting decreased hypercontractility in human iPSC cells differentiated into cardiomyocytes after administration of a CRISPR-Cas9 system correcting a MYH7 R403Q mutation.

FIGS. 3A and 3B depict representative schematic diagrams illustrating a genetically modified mouse line generated to model the human MYH7 p.R403Q mutation (FIG. 3A) targeting the same human disease-causing mutation within the mouse myosin heavy chain 6 (Myh6) gene (FIG. 3B) according to various aspects of the disclosure.

FIGS. 4A-4E depict representative images illustrating development of cardiac phenotypes in wild-type (WT; FIG. 4A), 403/+ (FIG. 4B), and 403/403 mice (FIG. 4C) mice at stage P8 of development and cardiac fibrosis in wild-type (WT; FIG. 4D) and 403/+ (FIG. 4E) mice 6 months after birth according to various aspects of the disclosure.

FIG. 5 depicts a representative schematic diagram illustrating a CRISPR-Cas9 system for correction of the Myh6.R403Q mutation in the mouse model of the human MYH7 p.R403Q mutation according to various aspects of the disclosure.

FIG. 6A depicts a representative schematic diagram for generating isogenic HD^{403/+} and HD^{403/403} iPSCs by homology-directed repair. Using iPSCs derived from a healthy donor (HD^{WT}), the MYH7 p.R403Q (c.1208G>A) mutation was introduced by CRISPR-Cas9-based homology-directed repair using SpCas9, a sgRNA (spacer sequence colored in green, PAM sequence colored in gold), and a single-stranded oligodeoxynucleotide (ssODN) donor template containing the mutation. A heterozygous genotype (HD^{403/+}) and homozygous genotype (HD^{403/403}) were isolated. Chromatograms highlighting mutational insertion and corresponding amino acid changes are shown for indicated genotypes. Red arrows indicate coding nucleotide 1208 in amino acid 403.

FIG. 6B depicts a Sanger sequencing chromatogram showing no mutational insertion on the highly homologous MYH6 gene. Red arrow indicates coding nucleotide 1211 and amino acid 404.

FIG. 6C depicts representative images of cardiomyocytes derived from iPSCs generated in FIGS. 6A-6B. (Alpha-actinin is colored in green; nuclei are marked by DAPI (4′,6-diamidino-2-phenylindole) in blue. Scale bar, 25 m.

FIG. 7A depicts a schematic depicting how an illustrative sgRNA, h403_sgRNA, can be used in a method of base editing to correct a MYH7 c.1208G>A (p.R403Q) missense mutation. Specifically, base editing could convert the mutant neutrally charged glutamine back to a positively charged arginine, restoring proper function of the myosin head.

FIG. 7B depicts a schematic illustrating how in some exemplary methods, eight candidate base editor variants were screened for their efficiencies in correcting the pathogenic adenine to a guanine using the candidate h403_sgRNA within a homozygous MYH7 c.1208G>A iPSC line (HD^{403/403}).

FIG. 7C depicts a representative bar graph depicting DNA editing efficiency of all adenines within a target protospacer in HD^{403/403} iPSCs 72 h post-transfection with candidate base editors. Data are means±s.d. across three technical replicates. Numbering is with the first base 5′ of the PAM as 1; target mutant adenine is position A16.

FIG. 8A depicts a workflow for reprogramming iPSCs from a healthy donor (HD) and two HCM patients (HCM1 and HCM2) followed by mutation knock-in for the HD line, and base editing correction for the HDMI and HCM2 lines. Isogenic clonal lines were isolated and differentiated into CMs for downstream analysis of iPSC-CM function.

FIG. 8B depicts results from a deep sequencing experiment to measure editing of all adenine residues within an on-target protospacer, h403_sgRNA. Target pathogenic adenine is A16. Deep sequencing was performed for ABE-treated MYH7^{403/+} HCM1, and MYH7^{403/+} HCM2 iPSCs.

FIG. 8C depicts peak systolic force of MYH7^{403/+} and MYH7^{WT} iPSC-CMs from HD, HCM1, and HCM2 patients. **P<0.01, ****P<0.0001 by Student's unpaired two-sided t-test.

FIG. 8D depicts oxygen consumption rate (OCR) as a function of time in indicated cell lines following exposure to the electron transport chain complex inhibitors, oligomycin, carbonyl cyanide m-chlorophenyl hydrazone (CCCP), and Antimycin A (AntA) (top), and mean and distribution of values across four timepoints for basal OCR (bottom left) and maximal OCR (bottom right) for indicated cell lines. ***P<0.001, ****P<0.0001 by Student's unpaired two-sided t-test.

FIG. 9 depicts results from a deep sequencing analysis to measure editing for 58 adenines within protospacers of top 8 CRISPOR-identified candidate off-target loci.

FIG. 10 depicts a homology comparison for mouse α-myosin heavy chain (Myh6) and human β-myosin heavy chain (MYH7) at the amino acid level (top) and DNA sequence level (bottom) around glutamine 403. The h403_sgRNA is illustrated in green and the PAM sequence is illustrated in yellow. The pathogenic c.1208 G>A nucleotide is within the canonical base editing window of positions 14-17, counting the adenine nucleotide immediately 5′ of the PAM as position 1.

FIG. 11A depicts how a humanized HCM mouse model was generated by replacing part of the native murine Myh6 genomic sequence with the human MYH7 sequence containing the p.R403Q mutation. Sanger sequencing chromatograms show the native Myh6^{WT} sequence (top), the humanized Myh6^{h403/+} mouse model sequence (middle), and a patient-derived iPSC line sequence (bottom). Yellow squares indicate knocked-in human nucleotides.

FIG. 11B depicts gross histology (top), and Masson's trichrome staining of coronal (4-chamber) (middle) and transverse (bottom) sections of the humanized mouse model for the wildtype (left), heterozygous (middle), and homozygous (right) genotypes at postnatal day 8. Scale bar, 1 mm

FIG. 11C depicts Masson's trichrome, Picrosirius red, and hematoxylin & eosin staining of heart sections of the humanized mouse model for the wildtype (left) and heterozygous (right) genotypes at 9 months of age. Scale bar, 1 mm for 10× images top, 100 μm for 10× images middle, 25 m for 40× images bottom.

FIG. 12A, depicts a schematic of a dual AAV9 ABE system encoding ABEmax-VRQR base editor halves and h403_sgRNA to target the human MYH7 p.R403Q mutation and.

FIG. 12B depicts an experimental outline for intrathoracic injection of Myh6^{h403/+} or Myh6^{h403/+} mice with saline or dual AAV9 ABE at P0 followed by serial echocardiograms. Chow diet supplemented with 0.1% Cyclosporine A was given at 5 weeks of age for 11 weeks.

FIG. 12C-12H depicts left ventricular anterior wall thickness at diastole (C), left ventricular posterior wall thickness at diastole (D), left ventricular internal diameter at diastole (E) and systole (F), ejection fraction (G), and fractional shortening (H), of Myh6^{WT} mice, Myh6^{h403/+} mice, or ABE-treated Myh6^{h403/+} mice from 8-16 weeks of age. n=5 for each group.

FIG. 12I depicts representative Masson's trichrome staining of serial (500 m interval) transverse sections for Myh6^{WT} mice, Myh6^{h403/+} mice, or ABE-treated Myh6^{h403/+} mice. Scale bar, 1 mm.

FIG. 12J-M depicts ventricular cross-sectional area (12J), average wall thickness (12K), heart weight (HW) to tibia length (TL) (12L), percentage of collagen area (12M) from n=3-5 mice for each experimental group in 12I. Data are mean±s.d. *P<0.05, **P<0.01 by Student's unpaired two-sided t-test.

FIG. 13A depicts injection details for treating Myh6^{h403/h1403} mice with ABE-AAV9 or saline.

FIG. 13B is a representative Kaplan-Meier curve for Myh6^{WT} mice (n=7), Myh6^{h403/+} mice (n=8), Myh6^{h403/h403} mice (n=6), and ABE-treated Myh6^{h403/h403} mice at a low (AAV LOW, n=3) or high dose (AAV HIGH, n=5). Median lifespans: Myh6^{WT} and Myh6^{h403/+} mice, >40 days; Myh6^{h403/h1403} mice, 7 days; AAV LOW Myh6^{h403/h1403} mice, 9 days (1.3-fold longer, P<0.05); AAV HIGH Myh6^{h403/h1403} mice, 15 days (2.1-fold longer, P<0.01). *P<0.05, **P<0.01 by Mantel-Cox test.

FIG. 13C depicts Sanger sequencing chromatograms for a Myh6^{h403/h403} mouse and a AAV HIGH Myh6^{h403/h1403} mouse showing 35% on-target editing of the target pathogenic adenine at the cDNA level.

FIG. 13D depicts Four-chamber sectioning and Masson's trichrome staining of a AAV HIGH Myh6^{h403/h403} mouse at 15 days old.

FIG. 14A depicts a schematic for measuring genomic and transcriptomic changes following dual AAV9 ABE injection in mice. Cardiomyocyte nuclei were isolated from 18 weeks old Myh6^{WT} mice, Myh6^{h403/+} mice, or ABE-treated Myh6^{h403/+} mice to assess genomic correction and transcriptomic changes.

FIG. 14B depicts DNA-editing efficiency for correcting the pathogenic adenine nucleotide following dual AAV9 ABE treatment. Data are mean±s.d.

FIG. 14C depicts a percentage of expressed mutant transcripts in ABE-treated Myh6^{h403/+} mice compared to Myh6^{h403/+} mice. Data are mean±s.d. *P<0.05 by Student's unpaired two-sided t-test, n=3 biological replicates for each group.

FIG. 14D depicts Bystander editing in ABE-treated Myh6^{h403/+} mice compared to saline-treated mice. Data are mean±s.d. *P<0.05 by Student's unpaired two-sided t-test, n=3 biological replicates for each group.

FIG. 14E depicts transcriptome-wide nuclear levels of A-to-I RNA editing in Myh6^{WT} mice, Myh6^{h403/+} mice, and ABE-treated Myh6^{h403+} mice. Data are mean±s.d.

FIG. 14F depicts a heat map of 257 differentially expressed genes amongst Myh6^{WT} or Myh6^{h403/+} mice and ABE-treated Myh6^{h403/+} mice. Samples and genes are ordered by hierarchical clustering. Data was scaled by the sum of each row and are displayed as row min and row max. ABE-treated Myh6^{h403/+} mice cluster with Myh6^{WT} mice.

FIG. 14G depicts fold change expression of Nppa mRNA expression for Myh6^{h403/+} mice and ABE-treated Myh6^{h403/+} mice normalized to Myh6^{WT} mice. Data from RNA-seq and qPCR. Data are mean±s.d. *P<0.05 by Student's unpaired two-sided t-test, n=3 biological replicates for each group.

FIG. 15A depicts representative M-mode images for Myh6^{WT} mice, Myh6^{h403/+} mice, or ABE-treated Myh6^{h403/+} mice at 16 weeks of age.

FIGS. 15B-15D depicts representative volcano plots showing fold-change and p-value of genes up-regulated (red) and down-regulated (blue) in Myh6^{h403/+} mice compared to Myh6^{WT} mice (FIG. 15B), ABE-treated Myh6^{h403/+} mice compared to Myh6^{h403/+} mice (FIG. 15C), and ABE-treated Myh6^{h403/+} mice compared to Myh6^{WT} mice (FIG. 15D).

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part, on the discovery of guide RNAs (gRNAs) for use with Clustered Regularly Interspaced Short Palindromic Repeats (CRISPR)-CRISPR associate protein 9 (Cas9) systems that successfully reverse phenotypes associated with familial cardiomyopathies HCM by correcting genetic mutations through base-pair editing. In various aspects, the present disclosure also provides novel fusion proteins that combine a deaminase and a Cas9-related nickase (e.g., an endonuclease that generates single stranded cuts) to perform base-pair editing to correct these genetic mutations. Accordingly, provided herein are compositions comprising single guide RNA (sgRNA) designed for a CRISPR-Cas9 system and method of using thereof for preventing, ameliorating or treating one or more cardiomyopathies. Also provided are mouse models comprising mutations associated with HCM that may be used to test the compositions and methods provided herein.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. Compositions

The present disclosure provides for compositions for preventing, ameliorating or treating one or more cardiomyopathies. In some embodiments, compositions herein can include a guide RNA (gRNA). In some embodiments, compositions herein can comprise a fusion protein comprising a deaminase covalently linked to an RNA-guided endonuclease. In some embodiments, compositions herein can include a Clustered Regularly Interspaced Short Palindromic Repeats (CRISPR)-CRISPR associate protein 9 (Cas9) system. In some embodiments, compositions herein can include AAV vectors, AAV viral particles, or a combination thereof for delivery of gRNA and/or CRISPR-Cas9 systems disclosed herein. In some embodiments, compositions herein can be formulated to form one or more pharmaceutical compositions.

(a) gRNA

In general, a guide polynucleotide can complex with a compatible nucleic acid-guided nuclease and can hybridize with a target sequence, thereby directing the nuclease to the target sequence. A subject nucleic acid-guided nuclease capable of complexing with a guide polynucleotide can be referred to as a nucleic acid-guided nuclease that is compatible with the guide polynucleotide. In addition, a guide polynucleotide capable of complexing with a nucleic acid-guided nuclease can be referred to as a guide polynucleotide or a guide nucleic acid that is compatible with the nucleic acid-guided nucleases.

In some embodiments, an engineered polynucleotide (gRNA) disclosed herein can be split into fragments encompassing a synthetic tracrRNA and crRNA. In some aspects, a gRNA herein can comprise a nucleic acid sequence having at least 85% sequence identity (e.g., about 85%, 90%, 95%, 99%, 100%) with the nucleotide sequence of 5′-CCT CAG GTG AAA GTG GGC AA-3′ (SEQ ID NO: 1). In some aspects, a gRNA herein can comprise a nucleic acid sequence having at least 85% sequence identity (e.g., about 85%, 90%, 95%, 99%, 100%) with the nucleotide sequence of 5′-CCT CAG GTG AAG GTG GGG AA-3′ (SEQ ID NO: 2). In some aspects, a gRNA herein can comprise an nucleic acid sequence having at least 85% sequence identity (e.g., about 85%, 90%, 95%, 99%, 100%) with the nucleotide sequence of 5′-CCU CAG GUG AAA GUG GGC AA-3′ (SEQ ID NO: 5). In some aspects, a gRNA herein can comprise a nucleic acid sequence having at least 85% sequence identity (e.g., about 85%, 90%, 95%, 99%, 100%) with the nucleotide sequence of 5′-CCU CAG GUG AAG GUG GGG AA-3′ (SEQ ID NO: 6). In some aspects, a gRNA herein can comprise a nucleic acid sequence of 5′-CCT CAG GTG AAA GTG GGC AA-3′ (SEQ ID NO: 1). In some aspects, a gRNA herein can comprise the nucleotide sequence of 5′-CCT CAG GTG AAG GTG GGG AA-3′ (SEQ ID NO: 2). In some aspects, a gRNA herein can comprise the nucleotide sequence of CCU CAG GUG AAA GUG GGC AA-3′ (SEQ ID NO: 5). In some aspects, a gRNA herein can comprise the nucleotide sequence of 5′-CCU CAG GUG AAG GUG GGG AA-3′ (SEQ ID NO: 6).

In some embodiments, a gRNA herein can include modified or non-naturally occurring nucleotides. In some embodiments a gRNA can be encoded by a DNA sequence on a polynucleotide molecule such as a plasmid, linear construct, or editing cassette as disclosed herein. In some aspects, the gRNA can be encoded by a DNA sequence comprising SEQ ID NO: 1. In some aspects, the RNA guide polynucleotide can be encoded by a DNA sequence comprising SEQ ID NO: 2.

In some embodiments, a guide polynucleotide (e.g., gRNA) herein can comprise a spacer sequence. A spacer sequence is a polynucleotide sequence having sufficient complementarity with a target polynucleotide sequence to hybridize with the target sequence and direct sequence-specific binding of a complexed nucleic acid-guided nuclease to the target sequence. In other words, a spacer sequence of a gRNA molecule is understood to “target” a DNA sequence or “correspond to” a DNA sequence. The degree of complementarity between a guide sequence and its corresponding target sequence, when optimally aligned using a suitable alignment algorithm, may be about or more than about 50%, 60%, 75%, 80%, 85%, 90%, 95%, 97.5%, 99%, or more. Optimal alignment can be determined with the use of any suitable algorithm for aligning sequences. In some embodiments, a guide sequence herein can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In other embodiments, a spacer sequence herein can be less than about 75, 50, 45, 40, 35, 30, 25, 20 nucleotides in length. Preferably the spacer sequence is 10-30 nucleotides long. In some aspects, a spacer sequence herein can be 15-20 nucleotides in length.

In some embodiments, a guide polynucleotide (e.g., gRNA) herein can include a scaffold sequence. In general, a “scaffold sequence” can include any sequence that has sufficient sequence to promote formation of a targetable nuclease complex (e.g., a CRISPR-Cas9 system), wherein the targetable nuclease complex includes, but is not limited to, a nucleic acid-guided nuclease and a guide polynucleotide can include a scaffold sequence and a guide sequence. Sufficient sequence within the scaffold sequence to promote formation of a targetable nuclease complex can include a degree of complementarity along the length of two sequence regions within the scaffold sequence, such as one or two sequence regions involved in forming a secondary structure. In some aspects, the one or two sequence regions may be included or encoded on the same polynucleotide. In some aspects, the one or two sequence regions may be included or encoded on separate polynucleotides. Optimal alignment can be determined by any suitable alignment algorithm, and can further account for secondary structures, such as self-complementarity within either the one or two sequence regions. In some embodiments, the degree of complementarity between the one or two sequence regions along the length of the shorter of the two when optimally aligned can be about or more than about 25%, 30%, 40%, 50%, 60%, 70%, 80%, 90%, 95%, 97.5%, 99%, or higher. In some embodiments, at least one of the two sequence regions can be about or more than about 5, 6, 7, 8, 9, 10, 11, 12, 13, 14, 15, 16, 17, 18, 19, 20, 25, 30, 40, 50, or more nucleotides in length.

In some embodiments, a scaffold sequence of a subject guide polynucleotide herein can comprise a secondary structure. In some embodiments, a secondary structure can comprise a pseudoknot region. In some embodiments, binding kinetics of a guide polynucleotide herein to a nucleic acid-guided nuclease is determined in part by secondary structures within the scaffold sequence. In some embodiments, binding kinetics of a guide polynucleotide herein to a nucleic acid-guided nuclease is determined in part by nucleic acid sequence with the scaffold sequence.

In certain embodiments, spacer mutations can be introduced to a plasmid to test when a substitution gRNA sequence is created or a deletion or insertion mutant is created. Each of these plasmid constructs can be used to test genome editing accuracy and efficiency, for example, having a deletion, substitution or insertion. Alternatively, in some embodiments, gRNA constructs created by compositions and methods disclosed herein can be tested for optimal genome editing time on a select target by observing editing efficiencies over pre-determined time periods. In accordance with these embodiments, gRNA constructs created by compositions and methods disclosed herein can be tested for optimal genome editing windows to optimize editing efficiency and accuracy.

Examples of target polynucleotides for use of engineered gRNA disclosed herein can include a sequence/gene or gene segment associated with a signaling biochemical pathway, e.g., a signaling biochemical pathway-associated gene or polynucleotide. Other embodiments contemplated herein concern examples of target polynucleotides for use of engineered gRNA disclosed herein can include those related to a disease-associated gene or polynucleotide.

A “disease-associated” or “disorder-associated” gene or polynucleotide can refer to any gene or polynucleotide which results in a transcription or translation product at an abnormal level compared to a control or results in an abnormal form in cells derived from disease-affected tissues compared with tissues or cells of a non-disease control. It can be a gene that becomes expressed at an abnormally high level; it can be a gene that becomes expressed at an abnormally low level, or where the gene contains one or more mutations and where altered expression or expression of the mutated gene directly correlates with the occurrence and/or progression of a health condition or disorder. A disease or disorder-associated gene can refer to a gene possessing mutation(s) or genetic variation that are directly responsible or is in linkage disequilibrium with a gene(s) that is responsible for the cause or progression of a disease or disorder. The transcribed or translated products can be known or unknown, and can be at a normal or abnormal level.

In some embodiments, a gRNA disclosed herein may target polynucleotides related to a cardiomyopathy-associated gene or polynucleotide. In some aspects, a cardiomyopathy-associated gene or polynucleotide may be a HCM-associated gene or polynucleotide. In some embodiments, a gRNA disclosed herein may target polynucleotides related to a cardiomyopathy-associated gene such as but not limited to TTN, MYH7, MYH6, MYPN, TNNT2, TPM1, or any combination thereof. In some aspects, gRNA disclosed herein may target polynucleotides related to one or more cardiomyopathy-associated genes such as MYH7, MYBPC3, TNNC1, or a combination thereof.

In some embodiments, a gRNA disclosed herein may target polynucleotides related to a cardiomyopathy-associated gene or polynucleotide possessing one or more mutation(s). In some embodiments, a gRNA disclosed herein may target polynucleotides related to a cardiomyopathy-associated gene possessing one or more mutation(s) wherein the cardiomyopathy-associated gene can be TTN, MYH7, MYH6, MYPN, TNNT2, TPM1, or any combination thereof. In some aspects, a gRNA disclosed herein may target polynucleotides related to a cardiomyopathy-associated gene possessing one or more mutation(s) wherein the cardiomyopathy-associated gene can be MYH7 or a combination thereof. In some examples, a gRNA disclosed herein may target polynucleotides related to a R403Q mutation in a MYH7 gene or its mammalian equivalent thereof.

### (b) Base Editor

Base editing has emerged as an attractive method to correct and potentially cure genetically based diseases. Base editors are fusion proteins of Cas9 nickase or deactivated Cas9 and a deaminase protein, which allow base pair edits without double-strand breaks within a defined editing window in relation to the protospacer adjacent motif (PAM) site of a single-guide RNA (sgRNA). Adenine base editors (ABEs) use deoxyadenosine deaminase to convert DNA A•T base pairs to G•C base pairs via an inosine intermediate and have been previously shown to function in many post-mitotic cells in vivo and in vitro.

Accordingly, in some embodiments, compositions herein further comprise a fusion protein comprising a deaminase and a Cas9 nickase or deactivated Cas9 endonuclease. Suitable deaminases and a Cas9 nickase or deactivated Cas9 endonuclease are described in more detail below. In some aspects, the fusion protein may further comprise a flexible peptide linker connecting the deaminase and the RNA-guided endonuclease. In still other aspects, other secondary components (e.g., nuclear localization sequences) may also be included in the fusion protein.

In some embodiments, the base editors provided herein can be made as a recombinant fusion protein comprising one or more protein domains, thereby generating a base editor. In certain embodiments, the base editors provided herein comprise one or more features that improve the base editing activity (e.g., efficiency, selectivity, and/or specificity) of the base editor proteins. For example, the base editor proteins provided herein may comprise a Cas9 domain that has reduced nuclease activity. In some embodiments, the base editor proteins provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand.

### (i) Deaminases

In various aspects, the fusion protein comprises a deaminase as an adenine base editor (ABE). Suitable deaminases that can be used in the complex are ABE-max, ABE8e or ABE7.10. For ease of reference, amino acid sequences and nucleic acid sequences encoding these exemplary deaminases are provided in the Table 1 and 2. Also included are sequences of exemplary deaminases that include nuclear localization signals (NLS) (underlined and bolded in each table), discussed in more detail below.

In various aspects, the deaminase comprises an amino acid sequence having at least 85%, at least 90%, at least 95%, at least 96%, at least 97%, at least 98%, at least 99% sequence homology to any one of SEQ ID NOs: 7, 9 and 11. In various aspects, the deaminase comprises an amino acid sequence of any one of SEQ ID NOs: 7, 9 and 11. In some aspects, the deaminase comprises an amino acid sequence of SEQ ID NO: 7. In some aspects, the deaminase comprises an amino acid sequence of SEQ ID NO: 9. In some aspects, the deaminase comprises an amino acid sequence of SEQ ID NO: 11.

In various aspects, the deaminase further comprises a nuclear localization signal (NLS). Suitable nuclear localization signals are described below. In some aspects, the nuclear localization signal comprises MKRTADGSEFESPKKKRKV (SEQ ID NO: 31). In some aspects, the deaminase further comprising a NLS comprises an amino acid sequence having at least 85%, at least 90%, at least 95%, at least 96%, at least 97%, at least 98%, or at least 99% sequence homology to any one of SEQ ID NOs: 8 or 10. In various aspects, the deaminase further comprising an NLS comprises an amino acid sequence of SEQ ID NO: 8 or 10. In various aspects, the deaminase further comprising an NLS comprises an amino acid sequence of SEQ ID NO: 8. In various aspects, the deaminase further comprising an NLS comprises an amino acid sequence of SEQ ID NO: 10.

TABLE 1
Exemplary Deaminase (Amino Acid)

| | | SEQ |
|---|---|---|
| | | ID |
| Deaminase | Amino Acid Sequence | NO: |
| ABEmax | SEVEFSHEYWMRHALTLAKRAWDEREVPVG | 7 |
| | AVLVHNNRVIGEGWNRPIGRHDPTAHAEIM | |
| | ALRQGGLVMQNYRLIDATLYVTLEPCVMCA | |
| | GAMIHSRIGRVVFGARDAKTGAAGSLMDVL | |
| | HHPGMNHRVEITEGILADECAALLSDFFRM | |
| | RRQEIKAQKKAQSSTDSGGSSGGSSGSETP | |
| | GTSESATPESSGGSSGGSSEVEFSHEYWMR | |
| | HALTLAKRARDEREVPVGAVLVLNNRVIGE | |
| | GWNRAIGLHDPTAHAEIMALRQGGLVMQNY | |
| | RLIDATLYVTFEPCVMCAGAMIHSRIGRVV | |
| | FGVRNAKTGAAGSLMDVLHYPGMNHRVEIT | |
| | EGILADECAALLCYFFRMPRQVFNAQKKAQ | |
| | SSTD | |
| ABEmax | MKRTADGSEFESPKKKRKVSEVEFSHEYWM | 8 |
| with | RHALTLAKRAWDEREVPVGAVLVHNNRVIG | |
| NLS | EGWNRPIGRHDPTAHAEIMALRQGGLVMQN | |
| | YRLIDATLYVTLEPCVMCAGAMIHSRIGRV | |
| | VFGARDAKTGAAGSLMDVLHHPGMNHRVEI | |
| | TEGILADECAALLSDFFRMRRQEIKAQKKA | |
| | QSSTDSGGSSGGSSGSETPGTSESATPESS | |
| | GGSSGGSSEVEFSHEYWMRHALTLAKRARD | |
| | EREVPVGAVLVLNNRVIGEGWNRAIGLHDP | |
| | TAHAEIMALRQGGLVMQNYRLIDATLYVTF | |
| | EPCVMCAGAMIHSRIGRVVFGVRNAKTGAA | |
| | GSLMDVLHYPGMNHRVEITEGILADECAAL | |
| | LCYFFRMPRQVFNAQKKAQSSTD | |
| ABE8e | SEVEFSHEYWMRHALTLAKRARDEREVPVG | 9 |
| | AVLVLNNRVIGEGWNRAIGLHDPTAHAEIM | |
| | ALRQGGLVMQNYRLIDATLYVTFEPCVMCA | |
| | GAMIHSRIGRVVFGVRNSKRGAAGSLMNVL | |
| | NYPGMNHRVEITEGILADECAALLCDFYRM | |
| | PRQVFNAQKKAQSSIN | |
| ABE8e | MKRTADGSEFESPKKKRKVSEVEFSHEYWM | 10 |
| w/NLS | RHALTLAKRARDEREVPVGAVLVLNNRVIG | |
| | EGWNRAIGLHDPTAHAEIMALRQGGLVMQN | |
| | YRLIDATLYVTFEPCVMCAGAMIHSRIGRV | |
| | VFGVRNSKRGAAGSLMNVLNYPGMNHRVEI | |
| | TEGILADECAALLCDFYRMPRQVFNAQKKA | |
| | QSSIN | |
| ABE7.10 | MSEVEFSHEYWMRHALTLAKRAWDEREVPV | 11 |
| | GAVLVHNNRVIGEGWNRPIGRHDPTAHAEI | |
| | MALRQGGLVMQNYRLIDATLYVTLEPCVMC | |
| | AGAMIHSRIGRVVFGARDAKTGAAGSLMDV | |
| | LHHPGMNHRVEITEGILADECAALLSDFFR | |
| | MRRQEIKAQKKAQSSTDSGGSSGGSSGSET | |
| | PGTSESATPESSGGSSGGSSEVEFSHEYWM | |
| | RHALTLAKRARDEREVPVGAVLVLNNRVIG | |
| | EGWNRAIGLHDPTAHAEIMALRQGGLVMQN | |
| | YRLIDATLYVTFEPCVMCAGAMIHSRIGRV | |
| | VFGVRNAKTGAAGSLMDVLHYPGMNHRVEI | |
| | TEGILADECAALLCYFFRMPRQVFNAQKKA | |
| | QSSTD | |

In various aspects, the deaminase is encoded by a nucleic acid comprising any one of SEQ ID NOs: 12, 13, 14, 28, 74 and 75. As shown in Table 2, below, SEQ ID NOs: 12, 13 and 28 correspond to ABEmax and ABE8e further including a nuclear localization signal (NLS), where the sequence encoding the NLS is bolded and underlined in the table below. SEQ ID NOs: 74, 75 and 14 correspond to ABEmax, ABE8e and ABE7.10 without a nuclear localization signal, respectively. In some aspects, the deaminase in the fusion protein provided herein is encoded by a nucleic acid comprising SEQ ID NO: 12 or 74. In some aspects, the deaminase in the fusion protein provided herein is encoded by a nucleic acid comprising SEQ ID NO: 13 or 75. In some aspects, the deaminase in the fusion protein provided herein is encoded by a nucleic acid comprising SEQ ID NO: 14 or 28.

TABLE 2
Exemplary Deaminase (Nucleic Acid)

| | | SEQ |
|---|---|---|
| | | ID |
| Deaminase | Nucleic Acid Sequence | NO: |
| ABEmax | tctgaagtcgagtttagccacgagtattgg | 74 |
| | atgaggcacgcactgaccctggcaaagcga | |
| | gcatgggatgaaagagaagtccccgtgggc | |
| | gccgtgctggtgcacaacaatagagtgatc | |
| | ggagagggatggaacaggccaatcggccgc | |
| | cacgaccctaccgcacacgcagagatcatg | |
| | gcactgaggcagggaggcctggtcatgcag | |
| | aattaccgcctgatcgatgccaccctgtat | |
| | gtgacactggagccatgcgtgatgtgcgca | |
| | ggagcaatgatccacagcaggatcggaaga | |
| | gtggtgttcggagcacgggacgccaagacc | |
| | ggcgcagcaggctccctgatggatgtgctg | |
| | caccaccccggcatgaaccaccgggtggag | |
| | atcacagagggaatcctggcagacgagtgc | |
| | gccgccctgctgagcgatttctttagaatg | |
| | cggagacaggagatcaaggcccagaagaag | |
| | gcacagagctccaccgactctggaggatct | |
| | agcggaggatcctctggaagcgagacacca | |
| | ggcacaagcgagtccgccacaccagagagc | |
| | tccggcggctcctccggaggatcctctgag | |
| | gtggagttttcccacgagtactggatgaga | |
| | catgccctgaccctggccaagagggcacgc | |
| | gatgagagggaggtgcctgtgggagccgtg | |
| | ctggtgctgaacaatagagtgatcggcgag | |
| | ggctggaacagagccatcggcctgcacgac | |
| | ccaacagcccatgccgaaattatggccctg | |
| | agacagggcggcctggtcatgcagaactac | |
| | agactgattgacgccaccctgtacgtgaca | |
| | ttcgagccttgcgtgatgtgcgccggcgcc | |
| | atgatccactctaggatcggccgcgtggtg | |
| | tttggcgtgaggaacgcaaaaaccggcgcc | |
| | gcaggctccctgatggacgtgctgcactac | |
| | cccggcatgaatcaccgcgtcgaaattacc | |
| | gagggaatcctggcagatgaatgtgccgcc | |
| | ctgctgtgctatttctttcggatgcctaga | |
| | caggtgttcaatgctcagaagaaggcccag | |
| | agctccaccgac | |
| ABEmax w. | Atgaaacggacagccgacggaagcgagttc | 12 |
| NLS | gagtcaccaaagaagaagcggaaagtctct | |
| (bolded | gaagtcgagtttagccacgagtattggatg | |
| and | aggcacgcactgaccctggcaaagcgagca | |
| underlined) | tgggatgaaagagaagtccccgtgggcgcc | |
| | gtgctggtgcacaacaatagagtgatcgga | |
| | gagggatggaacaggccaatcggccgccac | |
| | gaccctaccgcacacgcagagatcatggca | |
| | ctgaggcagggaggcctggtcatgcagaat | |
| | taccgcctgatcgatgccaccctgtatgtg | |
| | acactggagccatgcgtgatgtgcgcagga | |
| | gcaatgatccacagcaggatcggaagagtg | |
| | gtgttcggagcacgggacgccaagaccggc | |
| | gcagcaggctccctgatggatgtgctgcac | |
| | caccccggcatgaaccaccgggtggagatc | |
| | acagagggaatcctggcagacgagtgcgcc | |
| | gccctgctgagcgatttctttagaatgcgg | |
| | agacaggagatcaaggcccagaagaaggca | |
| | cagagctccaccgactctggaggatctagc | |
| | ggaggatcctctggaagcgagacaccaggc | |
| | acaagcgagtccgccacaccagagagctcc | |
| | ggcggctcctccggaggatcctctgaggtg | |
| | gagttttcccacgagtactggatgagacat | |
| | gccctgaccctggccaagagggcacgcgat | |
| | gagagggaggtgcctgtgggagccgtgctg | |
| | gtgctgaacaatagagtgatcggcgagggc | |
| | tggaacagagccatcggcctgcacgaccca | |
| | acagcccatgccgaaattatggccctgaga | |
| | cagggggcctggtcatgcagaactacagac | |
| | tgattgacgccaccctgtacgtgacattcg | |
| | agccttgcgtgatgtgcgccggcgccatga | |
| | tccactctaggatcggccgcgtggtgtttg | |
| | gcgtgaggaacgcaaaaaccggcgccgcag | |
| | gctccctgatggacgtgctgcactaccccg | |
| | gcatgaatcaccgcgtcgaaattaccgagg | |
| | gaatcctggcagatgaatgtgccgccctgc | |
| | tgtgctatttctttcggatgcctagacagg | |
| | tgttcaatgctcagaagaaggcccagagct | |
| | ccaccgac | |
| ABE8e | tctgaggtggagttttcccacgagtactgg | 75 |
| | atgagacatgccctgaccctggccaagagg | |
| | gcacgggatgagagggaggtgcctgtggga | |
| | gccgtgctggtgctgaacaatagagtgatc | |
| | ggcgagggctggaacagagccatcggcctg | |
| | cacgacccaacagcccatgccgaaattatg | |
| | gccctgagacagggcggcctggtcatgcag | |
| | aactacagactgattgacgccaccctgtac | |
| | gtgacattcgagccttgcgtgatgtgcgcc | |
| | ggcgccatgatccactctaggatcggccgc | |
| | gtggtgtttggcgtgaggaactcaaaaaga | |
| | ggcgccgcaggctccctgatgaacgtgctg | |
| | aactaccccggcatgaatcaccgcgtcgaa | |
| | attaccgagggaatcctggcagatgaatgt | |
| | gccgccctgctgtgcgatttctatcggatg | |
| | cctagacaggtgttcaatgctcagaagaag | |
| | gcccagagctccatcaac | |
| ABE8e w. | Atgaaacggacagccgacggaagcgagttc | 13 |
| NLS | gagtcaccaaagaagaagcggaaagtctct | |
| | gaggtggagttttcccacgagtactggatg | |
| | agacatgccctgaccctggccaagagggca | |
| | cgggatgagagggaggtgcctgtgggagcc | |
| | gtgctggtgctgaacaatagagtgatcggc | |
| | gagggctggaacagagccatcggcctgcac | |
| | gacccaacagcccatgccgaaattatggcc | |
| | ctgagacagggcggcctggtcatgcagaac | |
| | tacagactgattgacgccaccctgtacgtg | |
| | acattcgagccttgcgtgatgtgcgccggc | |
| | gccatgatccactctaggatcggccgcgtg | |
| | gtgtttggcgtgaggaactcaaaaagaggc | |
| | gccgcaggctccctgatgaacgtgctgaac | |
| | taccccggcatgaatcaccgcgtcgaaatt | |
| | accgagggaatcctggcagatgaatgtgcc | |
| | gccctgctgtgcgatttctatcggatgcct | |
| | agacaggtgttcaatgctcagaagaaggcc | |
| | cagagctccatcaac | |
| ABE7.10 | Atgtccgaagtcgagttttcccatgagtac | 14 |
| | tggatgagacacgcattgactctcgcaaag | |
| | agggcttgggatgaacgcgaggtgcccgtg | |
| | ggggcagtactcgtgcataacaatcgcgta | |
| | atcggcgaaggttggaataggccgatcgga | |
| | cgccacgaccccactgcacatgcggaaatc | |
| | atggcccttcgacagggagggcttgtgatg | |
| | cagaattatcgacttatcgatgcgacgctg | |
| | tacgtcacgcttgaaccttgcgtaatgtgc | |
| | gcgggagctatgattcactcccgcattgga | |
| | cgagttgtattcggtgcccgcgacgccaag | |
| | acgggtgccgcaggttcactgatggacgtg | |
| | ctgcatcacccaggcatgaaccaccgggta | |
| | gaaatcacagaaggcatattggcggacgaa | |
| | tgtgcggcgctgttgtccgacttttttcgc | |
| | atgcggaggcaggagatcaaggcccagaaa | |
| | aaagcacaatcctctactgactctggtggt | |
| | tcttctggtggttctagcggcagcgagact | |
| | cccgggacctcagagtccgccacacccgaa | |
| | agttctggtggttcttctggtggttcttcc | |
| | gaagtcgagttttcccatgagtactggatg | |
| | agacacgcattgactctcgcaaagagggct | |
| | cgagatgaacgcgaggtgcccgtgggggca | |
| | gtactcgtgctcaacaatcgcgtaatcggc | |
| | gaaggttggaatagggcaatcggactccac | |
| | gaccccactgcacatgcggaaatcatggcc | |
| | cttcgacagggagggcttgtgatgcagaat | |
| | tatcgacttatcgatgcgacgctgtacgtc | |
| | acgtttgaaccttgcgtaatgtgcgcggga | |
| | gctatgattcactcccgcattggacgagtt | |
| | gtattcggtgttcgcaacgccaagacgggt | |
| | gccgcaggttcactgatggacgtgctgcat | |
| | tacccaggcatgaaccaccgggtagaaatc | |
| | acagaaggcatattggcggacgaatgtgcg | |
| | gcgctgttgtgttacttttttcgcatgccc | |
| | aggcaggtctttaacgcccagaaaaaagca | |
| | caatcctctactgac | |
| ABE7.10 | Atgaaacggacagccgacggaagcgagttc | 28 |
| with NLS | gagtcaccaaagaagaagcggaaagtctcc | |
| | gaagtcgagttttcccatgagtactggatg | |
| | agacacgcattgactctcgcaaagagggct | |
| | tgggatgaacgcgaggtgcccgtgggggca | |
| | gtactcgtgcataacaatcgcgtaatcggc | |
| | gaaggttggaataggccgatcggacgccac | |
| | gaccccactgcacatgcggaaatcatggcc | |
| | cttcgacagggagggcttgtgatgcagaat | |
| | tatcgacttatcgatgcgacgctgtacgtc | |
| | acgcttgaaccttgcgtaatgtgcgcggga | |
| | gctatgattcactcccgcattggacgagtt | |
| | gtattcggtgcccgcgacgccaagacgggt | |
| | gccgcaggttcactgatggacgtgctgcat | |
| | cacccaggcatgaaccaccgggtagaaatc | |
| | acagaaggcatattggcggacgaatgtgcg | |
| | gcgctgttgtccgacttttttcgcatgcgg | |
| | aggcaggagatcaaggcccagaaaaaagca | |
| | caatcctctactgactctggtggttcttct | |
| | ggtggttctagcggcagcgagactcccggg | |
| | acctcagagtccgccacacccgaaagttct | |
| | ggtggttcttctggtggttcttccgaagtc | |
| | gagttttcccatgagtactggatgagacac | |
| | gcattgactctcgcaaagagggctcgagat | |
| | gaacgcgaggtgcccgtgggggcagtactc | |
| | gtgctcaacaatcgcgtaatcggcgaaggt | |
| | tggaatagggcaatcggactccacgacccc | |
| | actgcacatgcggaaatcatggcccttcga | |
| | cagggagggcttgtgatgcagaattatcga | |
| | cttatcgatgcgacgctgtacgtcacgttt | |
| | gaaccttgcgtaatgtgcgcgggagctatg | |
| | attcactcccgcattggacgagttgtattc | |
| | ggtgttcgcaacgccaagacgggtgccgca | |
| | ggttcactgatggacgtgctgcattaccca | |
| | ggcatgaaccaccgggtagaaatcacagaa | |
| | ggcatattggcggacgaatgtgcggcgctg | |
| | ttgtgttacttttttcgcatgcccaggcag | |
| | gtctttaacgcccagaaaaaagcacaatcc | |
| | tctactgac | |

### (ii) Cas9 Nickase or Deactivated Cas9 Endonuclease

In various aspects, the fusion protein (e.g., base editor) used herein comprises a Cas9 nickase or deactivated Cas9 endonuclease. These proteins are derived from CRISPR-Cas9 systems which are naturally-occurring defense mechanisms in prokaryotes that have been repurposed as an RNA-guided DNA-targeting platform used for gene editing. CRISPR-Cas9 systems relies on the DNA nuclease Cas9, and two noncoding RNAs, crisprRNA (crRNA) and trans-activating RNA (tracrRNA) (i.e., gRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR-Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM). TracrRNA hybridizes with the 3′ end of crRNA to form an RNA-duplex structure that is bound by the Cas9 endonuclease to form the catalytically active CRISPR-Cas9 complex, which can then cleave the target DNA. Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

In some embodiments, the Cas9 (CRISPR associated protein 9) endonuclease can be used in a CRISPR method herein for preventing, ameliorating or treating one or more cardiomyopathies as described herein. A “Cas9 molecule,” as used herein, refers to a molecule that can interact with a gRNA molecule and, in concert with the gRNA molecule, localize (e.g., target or home) to a site which comprises a target sequence and PAM sequence. Cas9 proteins are known to exist in many CRISPR systems including, but not limited to: Methanococcus maripaludis; Corynebacterium diphtheriae; Corynebacterium efficiens; Corynebacterium glutamicum; Corynebacterium kroppenstedtii; Mycobacterium abscessus; Nocardia farcinica; Rhodococcus erythropolis; Rhodococcus jostii; Rhodococcus opacus; Acidothermus cellulolyticus; Arthrobacter chlorophenolicus; Kribbella flavida; Thermomonospora curvata; Bifidobacterium dentium; Bifidobacterium longum; Slackia heliotrinireducens; Persephonella marina; Bacteroides fragilis; Capnocytophaga ochracea; Flavobacterium psychrophilum; Akkermansia muciniphila; Roseiflexus castenholzii; Roseiflexus; Synechocystis; Elusimicrobium minutum; Fibrobacter succinogenes; Bacillus cereus; Listeria innocua; Lactobacillus casei; Lactobacillus rhamnosus; Lactobacillus salivarius; Streptococcus agalactiae; Streptococcus dysgalactiae equisimilis; Streptococcus equi zooepidemicus; Streptococcus gallolyticus; Streptococcus gordonii; Streptococcus mutans; Streptococcus pyogenes; Streptococcus pyogenes M1 GAS; Streptococcus pyogenes MGAS5005; Streptococcus pyogenes MGAS2096; Streptococcus pyogenes MGAS9429; Streptococcus pyogenes MGAS 10270; Streptococcus pyogenes MGAS6180; Streptococcus pyogenes MGAS315; Streptococcus pyogenes SSI-1; Streptococcus pyogenes MGAS 10750; Streptococcus pyogenes NZ131; Streptococcus thermophiles CNRZ1066; Streptococcus thermophiles LMD-9; Streptococcus thermophiles LMG 18311; Staphylococcus aureus; Staphylococcus auricularis; Staphylococcus lutrae; Staphylococcus lugdunensis; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi 1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter Ian RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis; Francisella tularensis WY96-3418; and Treponema denticola ATCC 35405, and the like.

In various embodiments, the improved base editors may comprise a nuclease-inactivated Cas protein may interchangeably be referred to as a “dCas” or “dCas9” protein (for nuclease-“dead” Cas9). Alternatively, as used herein, a nuclease inactivated Cas9 protein may be referred to as a “deactivated Cas9”. Methods for generating a Cas9 protein (or a fragment thereof) having an inactive DNA cleavage domain are known (See, e.g., Jinek et al, Science. 337:816-821(2012); Qi et al, “Repurposing CRISPR as an RNA-Guided Platform for Sequence-Specific Control of Gene Expression” (2013) Cell. 28; 152(5): 1173-83, the entire contents of each of which are incorporated herein by reference). For example, the DNA cleavage domain of Cas9 is known to include two subdomains, the HNH nuclease subdomain and the RuvCl subdomain. The HNH subdomain cleaves the strand complementary to the gRNA, whereas the RuvCl subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al, Science. 337:816-821(2012); Qi et al, Cell. 28; 152(5): 1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9.

In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild-type Cas9.

In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, wild-type Cas9 corresponds to Cas9 from Streptococcus pyogenes (NCBI Reference Sequence: NC_0I7053.1). In other embodiments, wild type Cas9 corresponds to Cas9 from Streptococcus pyogenes (NCBI Reference Sequence: NC_002737.2). In still other embodiments, Cas9 corresponds to, or comprises in part or in whole, a Cas9 amino acid sequence having one or more mutations that inactivate the Cas9 nuclease activity.

In some embodiments, the Cas9 domain comprises a D10A mutation, while the residue at position 840 relative to a wild type sequence such as Cas9 from Streptococcus pyogenes (NCBI Reference Sequence: NC_0I7053.1). Without wishing to be bound by any particular theory, the presence of the catalytic residue H840 restores the activity of the Cas9 to cleave the non-edited (e.g., non-deaminated) strand containing a G opposite the targeted C. Restoration of H840 (e.g., from A840) does not result in the cleavage of the target strand containing the C. Such Cas9 variants are able to generate a single-strand DNA break (nick) at a specific location based on the gRNA-defined target sequence, leading to repair of the non-edited strand. In the context of an adenosine base editor, an adenosine (A) is deaminated to an inosine (I) and the non-edited strand (including the T that base-paired with the deaminated A) is nicked, facilitating removal of the T that base-paired with the deaminated A and resulting in a A-T base pair being mutated to a G-C base pair. Nicking the non-edited strand, having the T, facilitates removal of the T via mismatch repair mechanisms.

In other embodiments, dCas9 variants having mutations other than D10A and H840A are provided, which, e.g., result in nuclease inactivated Cas9 (dCas9). Such mutations, by way of example, include other amino acid substitutions at D10 and H820, or other substitutions within the nuclease domains of Cas9 (e.g., substitutions in the HNH nuclease subdomain and/or the RuvCl subdomain) with reference to a wild type sequence such as Cas9 from Streptococcus pyogenes (NCBI Reference Sequence: NC_0I7053.1). In some embodiments, variants or homologues of dCas9 (e.g., variants of Cas9 from Streptococcus pyogenes (NCBI Reference Sequence: NC_0I7053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_0I7053. I. In some embodiments, variants of dCas9 (e.g., variants of NCBI Reference Sequence: NC_0I7053. I) are provided having amino acid sequences which are shorter, or longer than NC_0I7053. I by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, the base editors as provided herein comprise the full-length amino acid sequence of a Cas9 protein, e.g., one of the Cas9 sequences provided herein. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of skill in the art.

It should be appreciated that additional Cas9 proteins including variants and homologs thereof, are within the scope of this disclosure. PCT Application Publication WO2020051360A1, which is incorporated herein by reference in its entirety, discloses some suitable Cas9 variants, nickases and deactivated Cas9 proteins. Exemplary Cas9 proteins include, without limitation, those provided below. Illustrative amino acid sequences and encoding nucleic acid sequences of these exemplary nickases or deactivated Cas9 proteins are provided in Tables 3 and 4 below.

In various aspects, the Cas9 nickase or deactivated Cas9 endonuclease is selected from SpRY, SpG, SpCas9-NG, SpCas9-VRQR or a variant thereof. In various aspects, the Cas9 nickase or deactivated Cas9 endonuclease comprises an amino acid sequence having at least 85%, at least 90%, at least 95%, at least 96%, at least 97%, at least 98%, at least 99% sequence homology with any one of SEQ ID NOs: 15, 17, 19, and 21. For example, in some aspects, the Cas9 nickase or deactivated Cas9 endonuclease comprises an amino acid sequence comprising any one of SEQ ID NOs: 15, 17, 19, and 21. In some aspects, the Cas9 nickase or deactivated Cas9 endonuclease comprises an amino acid sequence comprising SEQ ID NO: 15. In some aspects, the Cas9 nickase or deactivated Cas9 endonuclease comprises an amino acid sequence comprising SEQ ID NO: 17. In some aspects, the Cas9 nickase or deactivated Cas9 endonuclease comprises an amino acid sequence comprising SEQ ID NO: 19. In some aspects, the Cas9 nickase or deactivated Cas9 endonuclease comprises an amino acid sequence comprising SEQ ID NO: 21.

In various aspects, the Cas9 nickase or deactivated Cas9 endonuclease may further comprise a nuclear localization signal. In some aspects, the nuclear localization signal comprises KRTADGSEFEPKKKRKV (SEQ ID NO: 32). In some aspects, the nuclear localization signal is connected to the Cas9 nickase or deactivated Cas9 endonuclease via a short peptide linker. Accordingly, in some aspects, the Cas9 nickase or deactivated Cas9 endonuclease comprising an NLS via a linker may comprise an amino acid sequence having at least 85%, at least 90%, at least 95%, at least 96%, at least 97%, at least 98%, or at least 99% sequence homology with any one of SEQ ID NOs: 16, 18, 20 and 22. In some aspects, the Cas9 nickase or deactivated Cas9 endonuclease comprising an NLS via a inker may comprise an amino acid sequence comprising any one of SEQ ID NOs: 16, 18, 20 and 22. In various aspects, the Cas9 nickase or deactivated Cas9 endonuclease comprising an NLS via a inker may comprise an amino acid sequence of SEQ ID NO: 16. In various aspects, the Cas9 nickase or deactivated Cas9 endonuclease comprising an NLS via a inker may comprise an amino acid sequence of SEQ ID NOs: 18. In various aspects, the Cas9 nickase or deactivated Cas9 endonuclease comprising an NLS via a inker may comprise an amino acid sequence of SEQ ID NO: 20. In various aspects, the Cas9 nickase or deactivated Cas9 endonuclease comprising an NLS via a inker may comprise an amino acid sequence of SEQ ID NO: 22.

TABLE 3
Exemplary SpCas9 nickases or deactivated
Cas9 endonucleases

| | | SEQ |
|---|---|---|
| SpCas9 | | ID |
| nickase | Amino Acid Sequence | NO: |
| SpCas9- | MDKKYSIGLAIGTNSVGWAVITDEYKVPSK | 15 |
| VRQR | KFKVLGNTDRHSIKKNLIGALLFDSGETAE | |
| Variant | ATRLKRTARRRYTRRKNRICYLQEIFSNEM | |
| | AKVDDSFFHRLEESFLVEEDKKHERHPIFG | |
| | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | LPKRNSDKLIARKKDWDPKKYGGFVSPTVA | |
| | YSVLVVAKVEKGKSKKLKSVKELLGITIME | |
| | RSSFEKNPIDFLEAKGYKEVKKDLIIKLPK | |
| | YSLFELENGRKRMLASARELQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTNLGA | |
| | PAAFKYFDTTIDRKQYRSTKEVLDATLIHQ | |
| | SITGLYETRIDLSQLGGD | |
| SpCas9- | MDKKYSIGLAIGTNSVGWAVITDEYKVPSK | 16 |
| VRQR | KFKVLGNTDRHSIKKNLIGALLFDSGETAE | |
| Variant with | ATRLKRTARRRYTRRKNRICYLQEIFSNEM | |
| linker and | AKVDDSFFHRLEESFLVEEDKKHERHPIFG | |
| NLS | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | LPKRNSDKLIARKKDWDPKKYGGFVSPTVA | |
| | YSVLWVAKVEKGKSKKLKSVKELLGITIME | |
| | RSSFEKNPIDFLEAKGYKEVKKDLIIKLPK | |
| | YSLFELENGRKRMLASARELQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTNLGA | |
| | PAAFKYFDTTIDRKQYRSTKEVLDATLIHQ | |
| | SITGLYETRIDLSQLGGDSGGSKRTADGSE | |
| | FEPKKKRKV | |
| SpRY Cas9 | MDKKYSIGLAIGTNSVGWAVITDEYKVPSK | 17 |
| | KFKVLGNTDRHSIKKNLIGALLFDSGETAE | |
| | RTRLKRTARRRYTRRKNRICYLQEIFSNEM | |
| | AKVDDSFFHRLEESFLVEEDKKHERHPIFG | |
| | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | RPKRNSDKLIARKKDWDPKKYGGFLWPTVA | |
| | YSVLVVAKVEKGKSKKLKSVKELLGITIME | |
| | RSSFEKNPIDFLEAKGYKEVKKDLIIKLPK | |
| | YSLFELENGRKRMLASAKQLQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTRLGA | |
| | PRAFKYFDTTIDPKQYRSTKEVLDATLIHQ | |
| | SITGLYETRIDLSQLGGD | |
| SpRY Cas9 | MDKKYSIGLAIGTNSVGWAVITDEYKVPSK | 18 |
| with linker | KFKVLGNTDRHSIKKNLIGALLFDSGETAE | |
| and NLS | RTRLKRTARRRYTRRKNRICYLQEIFSNEM | |
| (Protein) | AKVDDSFFHRLEESFLVEEDKKHERHPIFG | |
| | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | RPKRNSDKLIARKKDWDPKKYGGFLWPTVA | |
| | YSVLWVAKVEKGKSKKLKSVKELLGITIME | |
| | RSSFEKNPIDFLEAKGYKEVKKDLIIKLPK | |
| | YSLFELENGRKRMLASAKQLQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTRLGA | |
| | PRAFKYFDTTIDPKQYRSTKEVLDATLIHQ | |
| | SITGLYETRIDLSQLGGDSGGSKRTADGSE | |
| | FEPKKKRKV | |
| SpG Variant | MDKKYSIGLAIGTNSVGWAVITDEYKVPSK | 19 |
| | KFKVLGNTDRHSIKKNLIGALLFDSGETAE | |
| | ATRLKRTARRRYTRRKNRICYLQEIFSNEM | |
| | AKVDDSFFHRLEESFLVEEDKKHERHPIFG | |
| | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | LPKRNSDKLIARKKDWDPKKYGGFLWPTVA | |
| | YSVLWVAKVEKGKSKKLKSVKELLGITIME | |
| | RSSFEKNPIDFLEAKGYKEVKKDLIIKLPK | |
| | YSLFELENGRKRMLASAKQLQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTNLGA | |
| | PAAFKYFDTTIDRKQYRSTKEVLDATLIHQ | |
| | SITGLYETRIDLSQLGGD | |
| SpG Variant | MDKKYSIGLAIGTNSVGWAVITDEYKVPSK | 20 |
| with linker | KFKVLGNTDRHSIKKNLIGALLFDSGETAE | |
| and NLS | ATRLKRTARRRYTRRKNRICYLQEIFSNEM | |
| | AKVDDSFFHRLEESFLVEEDKKHERHPIFG | |
| | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | LPKRNSDKLIARKKDWDPKKYGGFLWPTVA | |
| | YSVLWVAKVEKGKSKKLKSVKELLGITIME | |
| | RSSFEKNPIDFLEAKGYKEVKKDLIIKLPK | |
| | YSLFELENGRKRMLASAKQLQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTNLGA | |
| | PAAFKYFDTTIDRKQYRSTKEVLDATLIHQ | |
| | SITGLYETRIDLSQLGGDSGGSKRTADGSE | |
| | FEPKKKRKV | |
| SpCas9-NG | MDKKYSIGLAIGTNSVGWAVITDEYKVPSK | 21 |
| Variant | KFKVLGNTDRHSIKKNLIGALLFDSGETAE | |
| | ATRLKRTARRRYTRRKNRICYLQEIFSNEM | |
| | AKVDDSFFHRLEESFLVEEDKKHERHPIFG | |
| | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | RPKRNSDKLIARKKDWDPKKYGGFVSPTVA | |
| | YSVLVVAKVEKGKSKKLKSVKELLGITIME | |
| | RSSFEKNPIDFLEAKGYKEVKKDLIIKLPK | |
| | YSLFELENGRKRMLASARFLQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTNLGA | |
| | PRAFKYFDTTIDRKVYRSTKEVLDATLIHQ | |
| | SITGLYETRIDLSQLGGD | |
| SpCas9- | MDKKYSIGLAIGTNSVGWAVITDEYKVPSK | 22 |
| NG Variant | KFKVLGNTDRHSIKKNLIGALLFDSGETAE | |
| with linker | ATRLKRTARRRYTRRKNRICYLQEIFSNEM | |
| and NLS | AKVDDSFFHRLEESFLVEEDKKHERHPIFG | |
| | NIVDEVAYHEKYPTIYHLRKKLVDSTDKAD | |
| | LRLIYLALAHMIKFRGHFLIEGDLNPDNSD | |
| | VDKLFIQLVQTYNQLFEENPINASGVDAKA | |
| | ILSARLSKSRRLENLIAQLPGEKKNGLFGN | |
| | LIALSLGLTPNFKSNFDLAEDAKLQLSKDT | |
| | YDDDLDNLLAQIGDQYADLFLAAKNLSDAI | |
| | LLSDILRVNTEITKAPLSASMIKRYDEHHQ | |
| | DLTLLKALVRQQLPEKYKEIFFDQSKNGYA | |
| | GYIDGGASQEEFYKFIKPILEKMDGTEELL | |
| | VKLNREDLLRKQRTFDNGSIPHQIHLGELH | |
| | AILRRQEDFYPFLKDNREKIEKILTFRIPY | |
| | YVGPLARGNSRFAWMTRKSEETITPWNFEE | |
| | VVDKGASAQSFIERMTNFDKNLPNEKVLPK | |
| | HSLLYEYFTVYNELTKVKYVTEGMRKPAFL | |
| | SGEQKKAIVDLLFKTNRKVTVKQLKEDYFK | |
| | KIECFDSVEISGVEDRFNASLGTYHDLLKI | |
| | IKDKDFLDNEENEDILEDIVLTLTLFEDRE | |
| | MIEERLKTYAHLFDDKVMKQLKRRRYTGWG | |
| | RLSRKLINGIRDKQSGKTILDFLKSDGFAN | |
| | RNFMQLIHDDSLTFKEDIQKAQVSGQGDSL | |
| | HEHIANLAGSPAIKKGILQTVKVVDELVKV | |
| | MGRHKPENIVIEMARENQTTQKGQKNSRER | |
| | MKRIEEGIKELGSQILKEHPVENTQLQNEK | |
| | LYLYYLQNGRDMYVDQELDINRLSDYDVDH | |
| | IVPQSFLKDDSIDNKVLTRSDKNRGKSDNV | |
| | PSEEVVKKMKNYWRQLLNAKLITQRKFDNL | |
| | TKAERGGLSELDKAGFIKRQLVETRQITKH | |
| | VAQILDSRMNTKYDENDKLIREVKVITLKS | |
| | KLVSDFRKDFQFYKVREINNYHHAHDAYLN | |
| | AVVGTALIKKYPKLESEFVYGDYKVYDVRK | |
| | MIAKSEQEIGKATAKYFFYSNIMNFFKTEI | |
| | TLANGEIRKRPLIETNGETGEIVWDKGRDF | |
| | ATVRKVLSMPQVNIVKKTEVQTGGFSKESI | |
| | RPKRNSDKLIARKKDWDPKKYGGFVSPTVA | |
| | YSVLWVAKVEKGKSKKLKSVKELLGITIME | |
| | RSSFEKNPIDFLEAKGYKEVKKDLIIKLPK | |
| | YSLFELENGRKRMLASARFLQKGNELALPS | |
| | KYVNFLYLASHYEKLKGSPEDNEQKQLFVE | |
| | QHKHYLDEIIEQISEFSKRVILADANLDKV | |
| | LSAYNKHRDKPIREQAENIIHLFTLTNLGA | |
| | PRAFKYFDTTIDRKVYRSTKEVLDATLIHQ | |
| | SITGLYETRIDLSQLGGDSGGSKRTADGSE | |
| | FEPKKKRKV | |

In various aspects, the SpCas9 nickase or deactivated Cas9 endonuclease is encoded by a nucleic acid comprising any one of SEQ ID NOs: 23-26, 83 and 100-102. As shown in Table 4, below, SEQ ID NOs: 23-26 correspond to SpCas9-VRQR, SpRY, SpG, and SpCas9—NG each further comprising a nuclear localization signal (NLS) attached to the 3′ end of each nucleic acid via a nucleic acid encoding a linker. In each of these sequences, the nucleic acid encoding the linker is underlined and the nucleic acid encoding the NLS is bolded. SEQ ID NOs: 83 and 100-102 encode the same proteins (SpCas9-VRQR, SpRY, SpG, and SpCas9—NG) without the linker or NLS.

In some aspects, the SpCas9 nickase or deactivated Cas9 endonuclease in the fusion protein provided herein is encoded by a nucleic acid comprising SEQ ID NO: 83. In some aspects, the SpCas9 nickase or deactivated Cas9 endonuclease in the fusion protein provided herein is encoded by a nucleic acid comprising SEQ ID NO: 100. In some aspects, the SpCas9 nickase or deactivated Cas9 endonuclease in the fusion protein provided herein is encoded by a nucleic acid comprising SEQ ID NO: 101. In some aspects, the SpCas9 nickase or deactivated Cas9 endonuclease in the fusion protein provided herein is encoded by a nucleic acid comprising SEQ ID NO: 102.

In some aspects, the SpCas9 nickase or deactivated Cas9 endonuclease in the fusion protein provided herein further comprises a nuclear localization signal (NLS) and is encoded by a nucleic acid comprising SEQ ID NO: 23. In some aspects, the SpCas9 nickase or deactivated Cas9 endonuclease in the fusion protein provided herein further comprises a nuclear localization signal (NLS) and is encoded by a nucleic acid comprising SEQ ID NO: 24. In some aspects, the SpCas9 nickase or deactivated Cas9 endonuclease in the fusion protein provided herein further comprises a nuclear localization signal (NLS) and is encoded by a nucleic acid comprising SEQ ID NO: 25. In some aspects, the SpCas9 nickase or deactivated Cas9 endonuclease in the fusion protein provided herein further comprises a nuclear localization signal (NLS) and is encoded by a nucleic acid comprising SEQ ID NO: 26.

TABLE 4
Exemplary Nucleic Acids Encoding SpCas9 Nickases or Deactivated SpCas9

| SpCas9 | | SEQ ID |
|---|---|---|
| nickase | Nucleic Acid Sequence | NO: |
| SpCas9- | atggacaagaagtacagcatcggcctggccatcggcaccaactctgtggg | 23 |
| VRQR | ctgggccgtgatcaccgacgagtacaaggtgcccagcaagaaattcaagg | |
| Variant | tgctgggcaacaccgaccggcacagcatcaagaagaacctgatcggagcc | |
| Encoding | ctgctgttcgacagcggcgaaacagccgaggccacccggctgaagagaac | |
| sequence | cgccagaagaagatacaccagacggaagaaccggatctgctatctgcaag | |
| for linker | agatcttcagcaacgagatggccaaggtggacgacagcttcttccacaga | |
| and NLS | ctggaagagtccttcctggtggaagaggataagaagcacgagcggcaccc | |
| are | catcttcggcaacatcgtggacgaggtggcctaccacgagaagtacccca | |
| underlined | ccatctaccacctgagaaagaaactggtggacagcaccgacaaggccgac | |
| and bolded, | ctgcggctgatctatctggccctggcccacatgatcaagttccggggcca | |
| respectfully. | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctggaaaagatgg | |
| | acggcaccgaggaactgctcgtgaagctgaacagagaggacctgctgcgg | |
| | aagcagcggaccttcgacaacggcagcatcccccaccagatccacctggg | |
| | agagctgcacgccattctgcggcggcaggaagatttttacccattcctga | |
| | aggacaaccgggaaaagatcgagaagatcctgaccttccgcatcccctac | |
| | tacgtgggccctctggccaggggaaacagcagattcgcctggatgaccag | |
| | aaagagcgaggaaaccatcaccccctggaacttcgaggaagtggtggaca | |
| | agggcgcttccgcccagagcttcatcgagcggatgaccaacttcgataag | |
| | aacctgcccaacgagaaggtgctgcccaagcacagcctgctgtacgagta | |
| | cttcaccgtgtataacgagctgaccaaagtgaaatacgtgaccgagggaa | |
| | tgagaaagcccgccttcctgagcggcgagcagaaaaaggccatcgtggac | |
| | ctgctgttcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatgggcgggatatgtacgtggaccaggaactggacatcaaccggctgt | |
| | ccgactacgatgtggaccatatcgtgcctcagagctttctgaaggacgac | |
| | tccatcgacaacaaggtgctgaccagaagcgacaagaaccggggcaagag | |
| | cgacaacgtgccctccgaagaggtcgtgaagaagatgaagaactactggc | |
| | ggcagctgctgaacgccaagctgattacccagagaaagttcgacaatctg | |
| | accaaggccgagagaggcggcctgagcgaactggataaggccggcttcat | |
| | caagagacagctggtggaaacccggcagatcacaaagcacgtggcacaga | |
| | tcctggactcccggatgaacactaagtacgacgagaatgacaagctgatc | |
| | cgggaagtgaaagtgatcaccctgaagtccaagctggtgtccgatttccg | |
| | gaaggatttccagttttacaaagtgcgcgagatcaacaactaccaccacg | |
| | cccacgacgcctacctgaacgccgtcgtgggaaccgccctgatcaaaaag | |
| | taccctaagctggaaagcgagttcgtgtacggcgactacaaggtgtacga | |
| | cgtgcggaagatgatcgccaagagcgagcaggaaatcggcaaggctaccg | |
| | ccaagtacttcttctacagcaacatcatgaactttttcaagaccgagatt | |
| | accctggccaacggcgagatccggaagcggcctctgatcgagacaaacgg | |
| | cgaaaccggggagatcgtgtgggataagggccgggattttgccaccgtgc | |
| | ggaaagtgctgagcatgccccaagtgaatatcgtgaaaaagaccgaggtg | |
| | cagacaggcggcttcagcaaagagtctatcctgcccaagaggaacagcga | |
| | taagctgatcgccagaaagaaggactgggaccctaagaagtacggcggct | |
| | tcgtgagccccaccgtggcctattctgtgctggtggtggccaaagtggaa | |
| | aagggcaagtccaagaaactgaagagtgtgaaagagctgctggggatcac | |
| | catcatggaaagaagcagcttcgagaagaatcccatcgactttctggaag | |
| | ccaagggctacaaagaagtgaaaaaggacctgatcatcaagctgcctaag | |
| | tactccctgttcgagctggaaaacggccggaagagaatgctggcctcagc | |
| | cagagaactgcagaagggaaacgaactggccctgccctccaaatatgtga | |
| | acttcctgtacctggccagccactatgagaagctgaagggctcccccgag | |
| | gataatgagcagaaacagctgtttgtggaacagcacaagcactacctgga | |
| | cgagatcatcgagcagatcagcgagttctccaagagagtgatcctggccg | |
| | acgctaatctggacaaagtgctgtccgcctacaacaagcaccgggataag | |
| | cccatcagagagcaggccgagaatatcatccacctgtttaccctgaccaa | |
| | tctgggagcccctgccgccttcaagtactttgacaccaccatcgaccgga | |
| | agcagtacagaagcaccaaagaggtgctggacgccaccctgatccaccag | |
| | agcatcaccggcctgtacgagacacggatcgacctgtctcagctgggagg | |
| | tgactctggcggctcaaaaagaaccgccgacggcagcgaattcgagccca | |
| | agaagaagaggaaagtc | |
| SpRY Cas9 | atggacaagaagtacagcatcggcctggccatcggcaccaactctgtggg | 24 |
| Encoding | ctgggccgtgatcaccgacgagtacaaggtgcccagcaagaaattcaagg | |
| sequence | tgctgggcaacaccgaccggcacagcatcaagaagaacctgatcggagcc | |
| for linker | ctgctgttcgacagcggcgaaacagccgagagaacccggctgaagagaac | |
| and NLS | cgccagaagaagatacaccagacggaagaaccggatctgctatctgcaag | |
| are | agatcttcagcaacgagatggccaaggtggacgacagcttcttccacaga | |
| underlined | ctggaagagtccttcctggtggaagaggataagaagcacgagcggcaccc | |
| and bolded, | catcttcggcaacatcgtggacgaggtggcctaccacgagaagtacccca | |
| respectfully. | ccatctaccacctgagaaagaaactggtggacagcaccgacaaggccgac | |
| | ctgcggctgatctatctggccctggcccacatgatcaagttccggggcca | |
| | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctggaaaagatgg | |
| | acggcaccgaggaactgctcgtgaagctgaacagagaggacctgctgcgg | |
| | aagcagcggaccttcgacaacggcagcatcccccaccagatccacctggg | |
| | agagctgcacgccattctgcggcggcaggaagatttttacccattcctga | |
| | aggacaaccgggaaaagatcgagaagatcctgaccttccgcatcccctac | |
| | tacgtgggccctctggccaggggaaacagcagattcgcctggatgaccag | |
| | aaagagcgaggaaaccatcaccccctggaacttcgaggaagtggtggaca | |
| | agggcgcttccgcccagagcttcatcgagcggatgaccaacttcgataag | |
| | aacctgcccaacgagaaggtgctgcccaagcacagcctgctgtacgagta | |
| | cttcaccgtgtataacgagctgaccaaagtgaaatacgtgaccgagggaa | |
| | tgagaaagcccgccttcctgagcggcgagcagaaaaaggccatcgtggac | |
| | ctgctgttcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatgggcgggatatgtacgtggaccaggaactggacatcaaccggctgt | |
| | ccgactacgatgtggaccatatcgtgcctcagagctttctgaaggacgac | |
| | tccatcgacaacaaggtgctgaccagaagcgacaagaaccggggcaagag | |
| | cgacaacgtgccctccgaagaggtcgtgaagaagatgaagaactactggc | |
| | ggcagctgctgaacgccaagctgattacccagagaaagttcgacaatctg | |
| | accaaggccgagagaggcggcctgagcgaactggataaggccggcttcat | |
| | caagagacagctggtggaaacccggcagatcacaaagcacgtggcacaga | |
| | tcctggactcccggatgaacactaagtacgacgagaatgacaagctgatc | |
| | cgggaagtgaaagtgatcaccctgaagtccaagctggtgtccgatttccg | |
| | gaaggatttccagttttacaaagtgcgcgagatcaacaactaccaccacg | |
| | cccacgacgcctacctgaacgccgtcgtgggaaccgccctgatcaaaaag | |
| | taccctaagctggaaagcgagttcgtgtacggcgactacaaggtgtacga | |
| | cgtgcggaagatgatcgccaagagcgagcaggaaatcggcaaggctaccg | |
| | ccaagtacttcttctacagcaacatcatgaactttttcaagaccgagatt | |
| | accctggccaacggcgagatccggaagcggcctctgatcgagacaaacgg | |
| | cgaaaccggggagatcgtgtgggataagggccgggattttgccaccgtgc | |
| | ggaaagtgctgagcatgccccaagtgaatatcgtgaaaaagaccgaggtg | |
| | cagacaggcggcttcagcaaagagtctatcagacccaagaggaacagcga | |
| | taagctgatcgccagaaagaaggactgggaccctaagaagtacggcggct | |
| | tcctgtggcccaccgtggcctattctgtgctggtggtggccaaagtggaa | |
| | aagggcaagtccaagaaactgaagagtgtgaaagagctgctggggatcac | |
| | catcatggaaagaagcagcttcgagaagaatcccatcgactttctggaag | |
| | ccaagggctacaaagaagtgaaaaaggacctgatcatcaagctgcctaag | |
| | tactccctgttcgagctggaaaacggccggaagagaatgctggcctctgc | |
| | caagcagctgcagaagggaaacgaactggccctgccctccaaatatgtga | |
| | acttcctgtacctggccagccactatgagaagctgaagggctcccccgag | |
| | gataatgagcagaaacagctgtttgtggaacagcacaagcactacctgga | |
| | cgagatcatcgagcagatcagcgagttctccaagagagtgatcctggccg | |
| | acgctaatctggacaaagtgctgtccgcctacaacaagcaccgggataag | |
| | cccatcagagagcaggccgagaatatcatccacctgtttaccctgaccag | |
| | actgggagcccctagagccttcaagtactttgacaccaccatcgacccca | |
| | agcagtacagaagcaccaaagaggtgctggacgccaccctgatccaccag | |
| | agcatcaccggcctgtacgagacacggatcgacctgtctcagctgggagg | |
| | tgactctggcggctcaaaaagaaccgccgacggcagcgaattcgagccca | |
| | agaagaagaggaaagtc | |
| SpG | atggacaagaagtacagcatcggcctggccatcggcaccaactctgtggg | 25 |
| Variant) | ctgggccgtgatcaccgacgagtacaaggtgcccagcaagaaattcaagg | |
| Encoding | tgctgggcaacaccgaccggcacagcatcaagaagaacctgatcggagcc | |
| sequence | ctgctgttcgacagcggcgaaacagccgaggccacccggctgaagagaac | |
| for linker | cgccagaagaagatacaccagacggaagaaccggatctgctatctgcaag | |
| and NLS | agatcttcagcaacgagatggccaaggtggacgacagcttcttccacaga | |
| are | ctggaagagtccttcctggtggaagaggataagaagcacgagcggcaccc | |
| underlined | catcttcggcaacatcgtggacgaggtggcctaccacgagaagtacccca | |
| and bolded, | ccatctaccacctgagaaagaaactggtggacagcaccgacaaggccgac | |
| respectfully. | ctgcggctgatctatctggccctggcccacatgatcaagttccggggcca | |
| | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctggaaaagatgg | |
| | acggcaccgaggaactgctcgtgaagctgaacagagaggacctgctgcgg | |
| | aagcagcggaccttcgacaacggcagcatcccccaccagatccacctggg | |
| | agagctgcacgccattctgcggcggcaggaagatttttacccattcctga | |
| | aggacaaccgggaaaagatcgagaagatcctgaccttccgcatcccctac | |
| | tacgtgggccctctggccaggggaaacagcagattcgcctggatgaccag | |
| | aaagagcgaggaaaccatcaccccctggaacttcgaggaagtggtggaca | |
| | agggcgcttccgcccagagcttcatcgagcggatgaccaacttcgataag | |
| | aacctgcccaacgagaaggtgctgcccaagcacagcctgctgtacgagta | |
| | cttcaccgtgtataacgagctgaccaaagtgaaatacgtgaccgagggaa | |
| | tgagaaagcccgccttcctgagcggcgagcagaaaaaggccatcgtggac | |
| | ctgctgttcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatgggcgggatatgtacgtggaccaggaactggacatcaaccggctgt | |
| | ccgactacgatgtggaccatatcgtgcctcagagctttctgaaggacgac | |
| | tccatcgacaacaaggtgctgaccagaagcgacaagaaccggggcaagag | |
| | cgacaacgtgccctccgaagaggtcgtgaagaagatgaagaactactggc | |
| | ggcagctgctgaacgccaagctgattacccagagaaagttcgacaatctg | |
| | accaaggccgagagaggcggcctgagcgaactggataaggccggcttcat | |
| | caagagacagctggtggaaacccggcagatcacaaagcacgtggcacaga | |
| | tcctggactcccggatgaacactaagtacgacgagaatgacaagctgatc | |
| | cgggaagtgaaagtgatcaccctgaagtccaagctggtgtccgatttccg | |
| | gaaggatttccagttttacaaagtgcgcgagatcaacaactaccaccacg | |
| | cccacgacgcctacctgaacgccgtcgtgggaaccgccctgatcaaaaag | |
| | taccctaagctggaaagcgagttcgtgtacggcgactacaaggtgtacga | |
| | cgtgcggaagatgatcgccaagagcgagcaggaaatcggcaaggctaccg | |
| | ccaagtacttcttctacagcaacatcatgaactttttcaagaccgagatt | |
| | accctggccaacggcgagatccggaagcggcctctgatcgagacaaacgg | |
| | cgaaaccggggagatcgtgtgggataagggccgggattttgccaccgtgc | |
| | ggaaagtgctgagcatgccccaagtgaatatcgtgaaaaagaccgaggtg | |
| | cagacaggcggcttcagcaaagagtctatcctgcccaagaggaacagcga | |
| | taagctgatcgccagaaagaaggactgggaccctaagaagtacggcggct | |
| | tcctgtggcccaccgtggcctattctgtgctggtggtggccaaagtggaa | |
| | aagggcaagtccaagaaactgaagagtgtgaaagagctgctggggatcac | |
| | catcatggaaagaagcagcttcgagaagaatcccatcgactttctggaag | |
| | ccaagggctacaaagaagtgaaaaaggacctgatcatcaagctgcctaag | |
| | tactccctgttcgagctggaaaacggccggaagagaatgctggcctctgc | |
| | caagcagctgcagaagggaaacgaactggccctgccctccaaatatgtga | |
| | acttcctgtacctggccagccactatgagaagctgaagggctcccccgag | |
| | gataatgagcagaaacagctgtttgtggaacagcacaagcactacctgga | |
| | cgagatcatcgagcagatcagcgagttctccaagagagtgatcctggccg | |
| | acgctaatctggacaaagtgctgtccgcctacaacaagcaccgggataag | |
| | cccatcagagagcaggccgagaatatcatccacctgtttaccctgaccaa | |
| | tctgggagcccctgccgccttcaagtactttgacaccaccatcgaccgga | |
| | agcagtacagaagcaccaaagaggtgctggacgccaccctgatccaccag | |
| | agcatcaccggcctgtacgagacacggatcgacctgtctcagctgggagg | |
| | tgactctggcggctcaaaaagaaccgccgacggcagcgaattcgagccca | |
| | agaagaagaggaaagtc | |
| SpCas9- | atggacaagaagtacagcatcggcctggccatcggcaccaactctgtggg | 26 |
| NG. | ctgggccgtgatcaccgacgagtacaaggtgcccagcaagaaattcaagg | |
| Encoding | tgctgggcaacaccgaccggcacagcatcaagaagaacctgatcggagcc | |
| sequence | ctgctgttcgacagcggcgaaacagccgaggccacccggctgaagagaac | |
| for linker | cgccagaagaagatacaccagacggaagaaccggatctgctatctgcaag | |
| and NLS | agatcttcagcaacgagatggccaaggtggacgacagcttcttccacaga | |
| are | ctggaagagtccttcctggtggaagaggataagaagcacgagcggcaccc | |
| underlined | catcttcggcaacatcgtggacgaggtggcctaccacgagaagtacccca | |
| and bolded, | ccatctaccacctgagaaagaaactggtggacagcaccgacaaggccgac | |
| respectfully. | ctgcggctgatctatctggccctggcccacatgatcaagttccggggcca | |
| | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctgacctgcccaa | |
| | cgagaaggtgctgcccaagcacagcctgctgtacgagtacttcaccgtgt | |
| | ataacgagctgaccaaagtgaaatacgtgaccgagggaatgagagaaaag | |
| | atggacggcaccgaggaactgctcgtgaagctgaacagagaggacctgct | |
| | gcggaagcagcggaccttcgacaacggcagcatcccccaccagatccacc | |
| | tgggagagctgcacgccattctgcggcggcaggaagatttttacccattt | |
| | actacgtgggccctctggccaggggaaacagcagattcgcctggatgacc | |
| | aaagggcgcttccgcccagagcttcatcgagcggatgaccaacttcgata | |
| | agaaagcccgccttcctgagcggcgagcagaaaaaggccatcgtggacct | |
| | gctgtcctgaaggacaaccgggaaaagatcgagaagatcctgaccttccg | |
| | catccccgaaagagcgaggaaaccatcaccccctggaacttcgaggaagt | |
| | ggtggactcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatgggcgggatatgtacgtggaccaggaactggacatcaaccggctgt | |
| | ccgactacgatgtggaccatatcgtgcctcagagctttctgaaggacgac | |
| | tccatcgacaacaaggtgctgaccagaagcgacaagaaccggggcaagag | |
| | cgacaacgtgccctccgaagaggtcgtgaagaagatgaagaactactggc | |
| | ggcagctgctgaacgccaagctgattacccagagaaagttcgacaatctg | |
| | accaaggccgagagaggcggcctgagcgaactggataaggccggcttcat | |
| | caagagacagctggtggaaacccggcagatcacaaagcacgtggcacaga | |
| | tcctggactcccggatgaacactaagtacgacgagaatgacaagctgatc | |
| | cgggaagtgaaagtgatcaccctgaagtccaagctggtgtccgatttccg | |
| | gaaggatttccagttttacaaagtgcgcgagatcaacaactaccaccacg | |
| | cccacgacgcctacctgaacgccgtcgtgggaaccgccctgatcaaaaag | |
| | taccctaagctggaaagcgagttcgtgtacggcgactacaaggtgtacga | |
| | cgtgcggaagatgatcgccaagagcgagcaggaaatcggcaaggctaccg | |
| | ccaagtacttcttctacagcaacatcatgaactttttcaagaccgagatt | |
| | accctggccaacggcgagatccggaagcggcctctgatcgagacaaacgg | |
| | cgaaaccggggagatcgtgtgggataagggccgggattttgccaccgtgc | |
| | ggaaagtgctgagcatgccccaagtgaatatcgtgaaaaagaccgaggtg | |
| | cagacaggcggcttcagcaaagagtctatcaggcccaagaggaacagcga | |
| | taagctgatcgccagaaagaaggactgggaccctaagaagtacggcggct | |
| | tcgtcagccccaccgtggcctattctgtgctggtggtggccaaagtggaa | |
| | aagggcaagtccaagaaactgaagagtgtgaaagagctgctggggatcac | |
| | catcatggaaagaagcagcttcgagaagaatcccatcgactttctggaag | |
| | ccaagggctacaaagaagtgaaaaaggacctgatcatcaagctgcctaag | |
| | tactccctgttcgagctggaaaacggccggaagagaatgctggcctctgc | |
| | cagattcctgcagaagggaaacgaactggccctgccctccaaatatgtga | |
| | acttcctgtacctggccagccactatgagaagctgaagggctcccccgag | |
| | gataatgagcagaaacagctgtttgtggaacagcacaagcactacctgga | |
| | cgagatcatcgagcagatcagcgagttctccaagagagtgatcctggccg | |
| | acgctaatctggacaaagtgctgtccgcctacaacaagcaccgggataag | |
| | cccatcagagagcaggccgagaatatcatccacctgtttaccctgaccaa | |
| | tctgggagcccctagggccttcaagtactttgacaccaccatcgaccgga | |
| | aggtgtacaggagcaccaaagaggtgctggacgccaccctgatccaccag | |
| | agcatcaccggcctgtacgagacacggatcgacctgtctcagctgggagg | |
| | tgactctggcggctcaaaaagaaccgccgacggcagcgaattcgagccca | |
| | agaagaagaggaaagtc | |
| SpCas9- | atggacaagaagtacagcatcggcctggccatcggcaccaactctgtggg | 83 |
| VRQR | ctgggccgtgatcaccgacgagtacaaggtgcccagcaagaaattcaagg | |
| Variant | tgctgggcaacaccgaccggcacagcatcaagaagaacctgatcggagcc | |
| coding | ctgctgttcgacagcggcgaaacagccgaggccacccggctgaagagaac | |
| sequence | cgccagaagaagatacaccagacggaagaaccggatctgctatctgcaag | |
| alone | agatcttcagcaacgagatggccaaggtggacgacagcttcttccacaga | |
| | ctggaagagtccttcctggtggaagaggataagaagcacgagcggcaccc | |
| | catcttcggcaacatcgtggacgaggtggcctaccacgagaagtacccca | |
| | ccatctaccacctgagaaagaaactggtggacagcaccgacaaggccgac | |
| | ctgcggctgatctatctggccctggcccacatgatcaagttccggggcca | |
| | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctggaaaagatgg | |
| | acggcaccgaggaactgctcgtgaagctgaacagagaggacctgctgcgg | |
| | aagcagcggaccttcgacaacggcagcatcccccaccagatccacctggg | |
| | agagctgcacgccattctgcggcggcaggaagatttttacccattcctga | |
| | aggacaaccgggaaaagatcgagaagatcctgaccttccgcatcccctac | |
| | tacgtgggccctctggccaggggaaacagcagattcgcctggatgaccag | |
| | aaagagcgaggaaaccatcaccccctggaacttcgaggaagtggtggaca | |
| | agggcgcttccgcccagagcttcatcgagcggatgaccaacttcgataag | |
| | aacctgcccaacgagaaggtgctgcccaagcacagcctgctgtacgagta | |
| | cttcaccgtgtataacgagctgaccaaagtgaaatacgtgaccgagggaa | |
| | tgagaaagcccgccttcctgagcggcgagcagaaaaaggccatcgtggac | |
| | ctgctgttcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatggggggatatgtacgtggaccaggaactggacatcaaccggctgtc | |
| | cgactacgatgtggaccatatcgtgcctcagagctttctgaaggacgact | |
| | ccatcgacaacaaggtgctgaccagaagcgacaagaaccggggcaagagc | |
| | gacaacgtgccctccgaagaggtcgtgaagaagatgaagaactactggcg | |
| | gcagctgctgaacgccaagctgattacccagagaaagttcgacaatctga | |
| | ccaaggccgagagaggcggcctgagcgaactggataaggccggcttcatc | |
| | aagagacagctggtggaaacccggcagatcacaaagcacgtggcacagat | |
| | cctggactcccggatgaacactaagtacgacgagaatgacaagctgatcc | |
| | gggaagtgaaagtgatcaccctgaagtccaagctggtgtccgatttccgg | |
| | aaggatttccagttttacaaagtgcgcgagatcaacaactaccaccacgc | |
| | ccacgacgcctacctgaacgccgtcgtgggaaccgccctgatcaaaaagt | |
| | accctaagctggaaagcgagttcgtgtacggcgactacaaggtgtacgac | |
| | gtgcggaagatgatcgccaagagcgagcaggaaatcggcaaggctaccgc | |
| | caagtacttcttctacagcaacatcatgaactttttcaagaccgagatta | |
| | ccctggccaacggcgagatccggaagcggcctctgatcgagacaaacggc | |
| | gaaaccggggagatcgtgtgggataagggccgggattttgccaccgtgcg | |
| | gaaagtgctgagcatgccccaagtgaatatcgtgaaaaagaccgaggtgc | |
| | agacaggcggcttcagcaaagagtctatcctgcccaagaggaacagcgat | |
| | aagctgatcgccagaaagaaggactgggaccctaagaagtacggcggctt | |
| | cgtgagccccaccgtggcctattctgtgctggtggtggccaaagtggaaa | |
| | agggcaagtccaagaaactgaagagtgtgaaagagctgctggggatcacc | |
| | atcatggaaagaagcagcttcgagaagaatcccatcgactttctggaagc | |
| | caagggctacaaagaagtgaaaaaggacctgatcatcaagctgcctaagt | |
| | actccctgttcgagctggaaaacggccggaagagaatgctggcctcagcc | |
| | agagaactgcagaagggaaacgaactggccctgccctccaaatatgtgaa | |
| | cttcctgtacctggccagccactatgagaagctgaagggctcccccgagg | |
| | ataatgagcagaaacagctgtttgtggaacagcacaagcactacctggac | |
| | gagatcatcgagcagatcagcgagttctccaagagagtgatcctggccga | |
| | cgctaatctggacaaagtgctgtccgcctacaacaagcaccgggataagc | |
| | ccatcagagagcaggccgagaatatcatccacctgtttaccctgaccaat | |
| | ctgggagcccctgccgccttcaagtactttgacaccaccatcgaccggaa | |
| | gcagtacagaagcaccaaagaggtgctggacgccaccctgatccaccaga | |
| | gcatcaccggcctgtacgagacacggatcgacctgtctcagctgggaggt | |
| | gac | |
| SpRY Cas9 | atggacaagaagtacagcatcggcctggccatcggcaccaactctgtggg | 100 |
| sequence | ctgggccgtgatcaccgacgagtacaaggtgcccagcaagaaattcaagg | |
| coding | tgctgggcaacaccgaccggcacagcatcaagaagaacctgatcggagcc | |
| alone | ctgctgttcgacagcggcgaaacagccgagagaacccggctgaagagaac | |
| | cgccagaagaagatacaccagacggaagaaccggatctgctatctgcaag | |
| | agatcttcagcaacgagatggccaaggtggacgacagcttcttccacaga | |
| | ctggaagagtccttcctggtggaagaggataagaagcacgagcggcaccc | |
| | catcttcggcaacatcgtggacgaggtggcctaccacgagaagtacccca | |
| | ccatctaccacctgagaaagaaactggtggacagcaccgacaaggccgac | |
| | ctgcggctgatctatctggccctggcccacatgatcaagttccggggcca | |
| | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctggaaaagatgg | |
| | acggcaccgaggaactgctcgtgaagctgaacagagaggacctgctgcgg | |
| | aagcagcggaccttcgacaacggcagcatcccccaccagatccacctggg | |
| | agagctgcacgccattctgcggcggcaggaagatttttacccattcctga | |
| | aggacaaccgggaaaagatcgagaagatcctgaccttccgcatcccctac | |
| | tacgtgggccctctggccaggggaaacagcagattcgcctggatgaccag | |
| | aaagagcgaggaaaccatcaccccctggaacttcgaggaagtggtggaca | |
| | agggcgcttccgcccagagcttcatcgagcggatgaccaacttcgataag | |
| | aacctgcccaacgagaaggtgctgcccaagcacagcctgctgtacgagta | |
| | cttcaccgtgtataacgagctgaccaaagtgaaatacgtgaccgagggaa | |
| | tgagaaagcccgccttcctgagcggcgagcagaaaaaggccatcgtggac | |
| | ctgctgttcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatgggcgggatatgtacgtggaccaggaactggacatcaaccggctgt | |
| | ccgactacgatgtggaccatatcgtgcctcagagctttctgaaggacgac | |
| | tccatcgacaacaaggtgctgaccagaagcgacaagaaccggggcaagag | |
| | cgacaacgtgccctccgaagaggtcgtgaagaagatgaagaactactggc | |
| | ggcagctgctgaacgccaagctgattacccagagaaagttcgacaatctg | |
| | accaaggccgagagaggcggcctgagcgaactggataaggccggcttcat | |
| | caagagacagctggtggaaacccggcagatcacaaagcacgtggcacaga | |
| | tcctggactcccggatgaacactaagtacgacgagaatgacaagctgatc | |
| | cgggaagtgaaagtgatcaccctgaagtccaagctggtgtccgatttccg | |
| | gaaggatttccagttttacaaagtgcgcgagatcaacaactaccaccacg | |
| | cccacgacgcctacctgaacgccgtcgtgggaaccgccctgatcaaaaag | |
| | taccctaagctggaaagcgagttcgtgtacggcgactacaaggtgtacga | |
| | cgtgcggaagatgatcgccaagagcgagcaggaaatcggcaaggctaccg | |
| | ccaagtacttcttctacagcaacatcatgaactttttcaagaccgagatt | |
| | accctggccaacggcgagatccggaagcggcctctgatcgagacaaacgg | |
| | cgaaaccggggagatcgtgtgggataagggccgggattttgccaccgtgc | |
| | ggaaagtgctgagcatgccccaagtgaatatcgtgaaaaagaccgaggtg | |
| | cagacaggcggcttcagcaaagagtctatcagacccaagaggaacagcga | |
| | taagctgatcgccagaaagaaggactgggaccctaagaagtacggcggct | |
| | tcctgtggcccaccgtggcctattctgtgctggtggtggccaaagtggaa | |
| | aagggcaagtccaagaaactgaagagtgtgaaagagctgctggggatcac | |
| | catcatggaaagaagcagcttcgagaagaatcccatcgactttctggaag | |
| | ccaagggctacaaagaagtgaaaaaggacctgatcatcaagctgcctaag | |
| | tactccctgttcgagctggaaaacggccggaagagaatgctggcctctgc | |
| | caagcagctgcagaagggaaacgaactggccctgccctccaaatatgtga | |
| | acttcctgtacctggccagccactatgagaagctgaagggctcccccgag | |
| | gataatgagcagaaacagctgtttgtggaacagcacaagcactacctgga | |
| | cgagatcatcgagcagatcagcgagttctccaagagagtgatcctggccg | |
| | acgctaatctggacaaagtgctgtccgcctacaacaagcaccgggataag | |
| | cccatcagagagcaggccgagaatatcatccacctgtttaccctgaccag | |
| | actgggagcccctagagccttcaagtactttgacaccaccatcgacccca | |
| | agcagtacagaagcaccaaagaggtgctggacgccaccctgatccaccag | |
| | agcatcaccggcctgtacgagacacggatcgacctgtctcagctgggagg | |
| | tgac | |
| SpG | atggacaagaagtacagcatcggcctggccatcggcaccaactctgtggg | 101 |
| Variant) | ctgggccgtgatcaccgacgagtacaaggtgcccagcaagaaattcaagg | |
| coding | tgctgggcaacaccgaccggcacagcatcaagaagaacctgatcggagcc | |
| sequence | ctgctgttcgacagcggcgaaacagccgaggccacccggctgaagagaac | |
| alone | cgccagaagaagatacaccagacggaagaaccggatctgctatctgcaag | |
| | agatcttcagcaacgagatggccaaggtggacgacagcttcttccacaga | |
| | ctggaagagtccttcctggtggaagaggataagaagcacgagcggcaccc | |
| | catcttcggcaacatcgtggacgaggtggcctaccacgagaagtacccca | |
| | ccatctaccacctgagaaagaaactggtggacagcaccgacaaggccgac | |
| | ctgcggctgatctatctggccctggcccacatgatcaagttccggggcca | |
| | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctggaaaagatgg | |
| | acggcaccgaggaactgctcgtgaagctgaacagagaggacctgctgcgg | |
| | aagcagcggaccttcgacaacggcagcatcccccaccagatccacctggg | |
| | agagctgcacgccattctgcggcggcaggaagatttttacccattcctga | |
| | aggacaaccgggaaaagatcgagaagatcctgaccttccgcatcccctac | |
| | tacgtgggccctctggccaggggaaacagcagattcgcctggatgaccag | |
| | aaagagcgaggaaaccatcaccccctggaacttcgaggaagtggtggaca | |
| | agggcgcttccgcccagagcttcatcgagcggatgaccaacttcgataag | |
| | aacctgcccaacgagaaggtgctgcccaagcacagcctgctgtacgagta | |
| | cttcaccgtgtataacgagctgaccaaagtgaaatacgtgaccgagggaa | |
| | tgagaaagcccgccttcctgagcggcgagcagaaaaaggccatcgtggac | |
| | ctgctgttcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatgggcgggatatgtacgtggaccaggaactggacatcaaccggctgt | |
| | ccgactacgatgtggaccatatcgtgcctcagagctttctgaaggacgac | |
| | tccatcgacaacaaggtgctgaccagaagcgacaagaaccggggcaagag | |
| | cgacaacgtgccctccgaagaggtcgtgaagaagatgaagaactactggc | |
| | ggcagctgctgaacgccaagctgattacccagagaaagttcgacaatctg | |
| | accaaggccgagagaggcggcctgagcgaactggataaggccggcttcat | |
| | caagagacagctggtggaaacccggcagatcacaaagcacgtggcacaga | |
| | tcctggactcccggatgaacactaagtacgacgagaatgacaagctgatc | |
| | cgggaagtgaaagtgatcaccctgaagtccaagctggtgtccgatttccg | |
| | gaaggatttccagttttacaaagtgcgcgagatcaacaactaccaccacg | |
| | cccacgacgcctacctgaacgccgtcgtgggaaccgccctgatcaaaaag | |
| | taccctaagctggaaagcgagttcgtgtacggcgactacaaggtgtacga | |
| | cgtgcggaagatgatcgccaagagcgagcaggaaatcggcaaggctaccg | |
| | ccaagtacttcttctacagcaacatcatgaactttttcaagaccgagatt | |
| | accctggccaacggcgagatccggaagcggcctctgatcgagacaaacgg | |
| | cgaaaccggggagatcgtgtgggataagggccgggattttgccaccgtgc | |
| | ggaaagtgctgagcatgccccaagtgaatatcgtgaaaaagaccgaggtg | |
| | cagacaggcggcttcagcaaagagtctatcctgcccaagaggaacagcga | |
| | taagctgatcgccagaaagaaggactgggaccctaagaagtacggcggct | |
| | tcctgtggcccaccgtggcctattctgtgctggtggtggccaaagtggaa | |
| | aagggcaagtccaagaaactgaagagtgtgaaagagctgctggggatcac | |
| | catcatggaaagaagcagcttcgagaagaatcccatcgactttctggaag | |
| | ccaagggctacaaagaagtgaaaaaggacctgatcatcaagctgcctaag | |
| | tactccctgttcgagctggaaaacggccggaagagaatgctggcctctgc | |
| | caagcagctgcagaagggaaacgaactggccctgccctccaaatatgtga | |
| | acttcctgtacctggccagccactatgagaagctgaagggctcccccgag | |
| | gataatgagcagaaacagctgtttgtggaacagcacaagcactacctgga | |
| | cgagatcatcgagcagatcagcgagttctccaagagagtgatcctggccg | |
| | acgctaatctggacaaagtgctgtccgcctacaacaagcaccgggataag | |
| | cccatcagagagcaggccgagaatatcatccacctgtttaccctgaccaa | |
| | tctgggagcccctgccgccttcaagtactttgacaccaccatcgaccgga | |
| | agcagtacagaagcaccaaagaggtgctggacgccaccctgatccaccag | |
| | agcatcaccggcctgtacgagacacggatcgacctgtctcagctgggagg | |
| | tgac | |
| SpCas9- | atggacaagaagtacagcatcggcctggccatcggcaccaactctgtggg | 102 |
| NG. | ctgggccgtgatcaccgacgagtacaaggtgcccagcaagaaattcaagg | |
| coding | tgctgggcaacaccgaccggcacagcatcaagaagaacctgatcggagcc | |
| sequence | ctgctgttcgacagcggcgaaacagccgaggccacccggctgaagagaac | |
| alone | cgccagaagaagatacaccagacggaagaaccggatctgctatctgcaag | |
| | agatcttcagcaacgagatggccaaggtggacgacagcttcttccacaga | |
| | ctggaagagtccttcctggtggaagaggataagaagcacgagcggcaccc | |
| | catcttcggcaacatcgtggacgaggtggcctaccacgagaagtacccca | |
| | ccatctaccacctgagaaagaaactggtggacagcaccgacaaggccgac | |
| | ctgcggctgatctatctggccctggcccacatgatcaagttccggggcca | |
| | cttcctgatcgagggcgacctgaaccccgacaacagcgacgtggacaagc | |
| | tgttcatccagctggtgcagacctacaaccagctgttcgaggaaaacccc | |
| | atcaacgccagcggcgtggacgccaaggccatcctgtctgccagactgag | |
| | caagagcagacggctggaaaatctgatcgcccagctgcccggcgagaaga | |
| | agaatggcctgttcggaaacctgattgccctgagcctgggcctgaccccc | |
| | aacttcaagagcaacttcgacctggccgaggatgccaaactgcagctgag | |
| | caaggacacctacgacgacgacctggacaacctgctggcccagatcggcg | |
| | accagtacgccgacctgtttctggccgccaagaacctgtccgacgccatc | |
| | ctgctgagcgacatcctgagagtgaacaccgagatcaccaaggcccccct | |
| | gagcgcctctatgatcaagagatacgacgagcaccaccaggacctgaccc | |
| | tgctgaaagctctcgtgcggcagcagctgcctgagaagtacaaagagatt | |
| | ttcttcgaccagagcaagaacggctacgccggctacattgacggcggagc | |
| | cagccaggaagagttctacaagttcatcaagcccatcctggaaaagatgg | |
| | acggcaccgaggaactgctcgtgaagctgaacagagaggacctgctgcgg | |
| | aagcagcggaccttcgacaacggcagcatcccccaccagatccacctggg | |
| | agagctgcacgccattctgcggcggcaggaagatttttacccattcctga | |
| | aggacaaccgggaaaagatcgagaagatcctgaccttccgcatcccctac | |
| | tacgtgggccctctggccaggggaaacagcagattcgcctggatgaccag | |
| | aaagagcgaggaaaccatcaccccctggaacttcgaggaagtggtggaca | |
| | agggcgcttccgcccagagcttcatcgagcggatgaccaacttcgataag | |
| | aacctgcccaacgagaaggtgctgcccaagcacagcctgctgtacgagta | |
| | cttcaccgtgtataacgagctgaccaaagtgaaatacgtgaccgagggaa | |
| | tgagaaagcccgccttcctgagcggcgagcagaaaaaggccatcgtggac | |
| | ctgctgttcaagaccaaccggaaagtgaccgtgaagcagctgaaagagga | |
| | ctacttcaagaaaatcgagtgcttcgactccgtggaaatctccggcgtgg | |
| | aagatcggttcaacgcctccctgggcacataccacgatctgctgaaaatt | |
| | atcaaggacaaggacttcctggacaatgaggaaaacgaggacattctgga | |
| | agatatcgtgctgaccctgacactgtttgaggacagagagatgatcgagg | |
| | aacggctgaaaacctatgcccacctgttcgacgacaaagtgatgaagcag | |
| | ctgaagcggcggagatacaccggctggggcaggctgagccggaagctgat | |
| | caacggcatccgggacaagcagtccggcaagacaatcctggatttcctga | |
| | agtccgacggcttcgccaacagaaacttcatgcagctgatccacgacgac | |
| | agcctgacctttaaagaggacatccagaaagcccaggtgtccggccaggg | |
| | cgatagcctgcacgagcacattgccaatctggccggcagccccgccatta | |
| | agaagggcatcctgcagacagtgaaggtggtggacgagctcgtgaaagtg | |
| | atgggccggcacaagcccgagaacatcgtgatcgaaatggccagagagaa | |
| | ccagaccacccagaagggacagaagaacagccgcgagagaatgaagcgga | |
| | tcgaagagggcatcaaagagctgggcagccagatcctgaaagaacacccc | |
| | gtggaaaacacccagctgcagaacgagaagctgtacctgtactacctgca | |
| | gaatgggcgggatatgtacgtggaccaggaactggacatcaaccggctgt | |
| | ccgactacgatgtggaccatatcgtgcctcagagctttctgaaggacgac | |
| | tccatcgacaacaaggtgctgaccagaagcgacaagaaccggggcaagag | |
| | cgacaacgtgccctccgaagaggtcgtgaagaagatgaagaactactggc | |
| | ggcagctgctgaacgccaagctgattacccagagaaagttcgacaatctg | |
| | accaaggccgagagaggcggcctgagcgaactggataaggccggcttcat | |
| | caagagacagctggtggaaacccggcagatcacaaagcacgtggcacaga | |
| | tcctggactcccggatgaacactaagtacgacgagaatgacaagctgatc | |
| | cgggaagtgaaagtgatcaccctgaagtccaagctggtgtccgatttccg | |
| | gaaggatttccagttttacaaagtgcgcgagatcaacaactaccaccacg | |
| | cccacgacgcctacctgaacgccgtcgtgggaaccgccctgatcaaaaag | |
| | taccctaagctggaaagcgagttcgtgtacggcgactacaaggtgtacga | |
| | cgtgcggaagatgatcgccaagagcgagcaggaaatcggcaaggctaccg | |
| | ccaagtacttcttctacagcaacatcatgaactttttcaagaccgagatt | |
| | accctggccaacggcgagatccggaagcggcctctgatcgagacaaacgg | |
| | cgaaaccggggagatcgtgtgggataagggccgggattttgccaccgtgc | |
| | ggaaagtgctgagcatgccccaagtgaatatcgtgaaaaagaccgaggtg | |
| | cagacaggcggcttcagcaaagagtctatcaggcccaagaggaacagcga | |
| | taagctgatcgccagaaagaaggactgggaccctaagaagtacggcggct | |
| | tcgtcagccccaccgtggcctattctgtgctggtggtggccaaagtggaa | |
| | aagggcaagtccaagaaactgaagagtgtgaaagagctgctggggatcac | |
| | catcatggaaagaagcagcttcgagaagaatcccatcgactttctggaag | |
| | ccaagggctacaaagaagtgaaaaaggacctgatcatcaagctgcctaag | |
| | tactccctgttcgagctggaaaacggccggaagagaatgctggcctctgc | |
| | cagattcctgcagaagggaaacgaactggccctgccctccaaatatgtga | |
| | acttcctgtacctggccagccactatgagaagctgaagggctcccccgag | |
| | gataatgagcagaaacagctgtttgtggaacagcacaagcactacctgga | |
| | cgagatcatcgagcagatcagcgagttctccaagagagtgatcctggccg | |
| | acgctaatctggacaaagtgctgtccgcctacaacaagcaccgggataag | |
| | cccatcagagagcaggccgagaatatcatccacctgtttaccctgaccaa | |
| | tctgggagcccctagggccttcaagtactttgacaccaccatcgaccgga | |
| | aggtgtacaggagcaccaaagaggtgctggacgccaccctgatccaccag | |
| | agcatcaccggcctgtacgagacacggatcgacctgtctcagctgggagg | |
| | tgact | |

In some embodiments, a Cas9 enzyme herein may be from Streptococcus, Staphylococcus, or variants thereof. It should be understood, that wild-type Cas9 may be used or modified versions of Cas9 may be used (e.g., evolved versions of Cas9, or Cas9 orthologues or variants), as provided herein. In some aspects, a Cas9 enzyme herein may be a Streptococcus pyogenes Cas9 (SpCas9) variant. In some aspects, a Cas9 enzyme herein may be a Streptococcus pyogenes Cas9 (SpCas9) variant compatible with NGG PAMs. The canonical PAM is the sequence 5′-NGG-3′, where “N” is any nucleobase followed by two guanine (“G”) nucleobases. In some aspects, a Cas9 enzyme herein may be a Streptococcus pyogenes Cas9 (SpCas9) variant compatible with non-NGG PAMs. In some aspects, a Cas9 enzyme herein may be a Streptococcus pyogenes Cas9 (SpCas9) variant compatible with non-NGG PAMs selected from TGAG and/or CGAG. In some aspects, a Cas9 enzyme herein may be a variant of the adenine base editor (ABE) ABEmax, which uses Streptococcus pyogenes Cas9 (SpCas9) variants compatible with non-NGG PAMs. In some examples, a Cas9 enzyme herein may be ABEmax-SpCas9-NG.

In some embodiments, the ability of an active Cas9 molecule to interact with and cleave a target nucleic acid is PAM sequence dependent. A PAM sequence is a sequence in the target nucleic acid. In some embodiments, a PAM herein may have a polynucleotide sequence having at least 85% (e.g., about 85%, 90%, 95%, 99%, 100%) sequence identity with the nucleotide sequence of TGAG or CGAG. In some embodiments, a PAM herein may have the nucleotide sequence of TGAG or CGAG. In some embodiments, cleavage of the target nucleic acid occurs upstream from the PAM sequence. Active Cas9 molecules from different bacterial species can recognize different sequence motifs (e.g., PAM sequences). In some embodiments, an active Cas9 molecule of S. pyogenes can recognize the sequence motif “NGG” and directs cleavage of a target nucleic acid sequence 1 to 10, e.g., 3 to 5, base pairs upstream from that sequence. In some embodiments, an active Cas9 molecule of S. pyogenes can recognize a non-NGG sequence motif and directs cleavage of a target nucleic acid sequence 1 to 10, e.g., 3 to 5, base pairs upstream from that sequence.

(iii) Additional Elements in the Fusion Proteins

In various aspects, the fusion proteins may contain one or more additional elements. In various examples, the fusion protein may further comprise a peptide linker to, for example, covalently link the deaminase and the SpCas9 nickase or deactivated Cas9 endonuclease or link each protein to one or more nuclear localization signals. Likewise, nuclear localization signals are additional elements that may be included in the fusion protein as part of either the deaminase and/or the SpCas9 nickase or deactivated Cas9 endonuclease.

Accordingly, in various aspects, the fusion protein further comprises a flexible peptide linker. Suitable linkers are provided in Table 5 below. In some aspects, the flexible linker may covalently link the deaminase and the SpCas9 nickase or deactivated Cas9 endonuclease. For example, in some aspects, the linker may comprise SEQ ID NO: 27. In various aspects, the flexible linker may connect a nuclear localization signal to an N or C terminus of either the deaminase or SpCas9 nickase or deactivated Cas9 endonuclease. For example, the linker may comprise SGGS (SEQ ID NO: 103). The flexible peptide linker may be encoded by a nucleic acid. Suitable nucleic acids that can encode the linkers are provided in Table 6 below. In some aspects, the linker may be encoded by a nucleic acid comprising SEQ ID NO: 29 or 30. In some aspects, the linker may be encoded by a nucleic acid comprising SEQ ID NO: 78.

TABLE 5
Exemplary Linkers (Amino Acid Sequences)

| Flexible Linkers | Amino Acid Sequence | SEQ ID NO: |
|---|---|---|
| Linker 1 | SGGSSGGSSGSETPGTSESATPESSGGSSGGS | 27 |
| Linker 2 | SGGS | 103 |

TABLE 6
Exemplary Linkers (Nucleic Acid Sequences)

| Flexible | | SEQ ID |
|---|---|---|
| Linkers | Nucleic Acid Sequence | NO: |
| Linker 1 | tccggaggatctagcggaggctcctctggctc | 29 |
| | tgagacacctggcacaagcgagagcgcaacac | |
| | ctgaaagcagcgggggcagcagcggggggtca | |
| Linker 1 | tctggtggttcttctggtggttctagcggcag | 30 |
| | cgagactcccgggacctcagagtccgccacac | |
| | ccgaaagttctggtggttcttctggtggttct | |
| Linker 2 | gagattttcgagcgggagctggacctgatgag | 78 |
| | agtggataacctgcctaatagcggaggcagta | |

In further aspects, the fusion protein may further comprise one or more nuclear localization signals (NLS). One or more NLS may be covalently attached or linked to either or both of the deaminase and/or Cas9 nickase or deactivated Cas9 endonuclease. For example, in some aspects, an NLS may be linked to the N- or C-terminus of the deaminase. In other aspects, an NLS may be linked to the N- or C-terminus of the Cas9 nickase or deactivated Cas9 endonuclease. For example in some aspects, an NLS may be linked to the N-terminus of the deaminase and another NLS may be linked to the C-terminus of the Cas9 nickase or deactivated Cas9 endonuclease.

Exemplary NLS include the c-myc NLS, the SV40 NLS, the hnRNPAI M9 NLS, the nucleoplasmin NLS, the sequence RMRKFKNKGKDTAELRRRRVEVSVELRKAKKDEQILKRRNV (SEQ ID NO: 33) of the IBB domain from importin-alpha, the sequences VSRKRPRP (SEQ ID NO: 34) and PPKKARED (SEQ ID NO: 35) of the myoma T protein, the sequence PQPKKKP (SEQ ID NO: 104) of human p53, the sequence SALIKKKKKMAP (SEQ ID NO: 36) of mouse c-abl IV, the sequences DRLRR (SEQ ID NO: 37) and PKQKKRK (SEQ ID NO: 38) of the influenza virus NS1, the sequence RKLKKKIKK (SEQ ID NO: 39) of the Hepatitis virus delta antigen and the sequence REKKKFLKRR (SEQ ID NO: 40) of the mouse Mx1 protein. Further acceptable nuclear localization signals include bipartite nuclear localization sequences such as the sequence KRKGDEVDGVDEVAKKKSKK (SEQ ID NO: 41) of the human poly(ADP-ribose) polymerase or the sequence RKCLQAGMNLEARKTKK (SEQ ID NO: 42) of the steroid hormone receptors (human) glucocorticoid. Additional exemplary NLS include MKRTADGSEFESPKKKRKV (SEQ ID NO: 31) and KRTADGSEFEPKKKRKV (SEQ ID NO: 32). Other suitable nuclear localization signals (NLSs) are known by those of skill in the art.

(iii) Exemplary Fusion Proteins

In accordance with the previous disclosure, exemplary fusion proteins may be provided by combining at least one deaminase and at least one Cas9 nickase or deactivated Cas9 endonuclease provided above. Non-limiting combinations that may be envisioned include: ABEmax-VRQR, ABEmax-SpCas9-NG, ABEmax-SpRY, ABEmax-SpG, ABE8e-VRQR, ABE8e-SpCas9-NG, ABE8e-SpRY, and ABE8e-SpG. Each of these fusion proteins may further comprise a linker (e.g., SEQ ID NO: 27 or 28) connecting the deaminase and the Cas9 protein. Further, each of these fusion proteins may further comprise one or more nuclear localization signals (NLS). Exemplary amino acid sequences for these fusion proteins, with and without nuclear localization signals, are provided in Table 7, below.

In various aspects, the fusion protein comprises an amino acid sequence having at least 85%, at least 90%, at least 95%, at least 96%, at least 97%, at least 98%, or at least 99% sequence homology to any one of SEQ ID NOs: 45-60. In some aspects, the fusion protein comprises an amino acid sequence having at least 85%, at least 90%, at least 95%, at least 96%, at least 97%, at least 98%, or at least 99% sequence homology to any one of SEQ ID NOs: 45, 47, 49, 51, 53, 55, 57, and 59. In some aspects, the fusion protein comprises an amino acid sequence comprising any one of SEQ ID NOs: 45, 47, 49, 51, 53, 55, 57, and 59. In some aspects, the fusion protein does further comprise one or more nuclear localization sequences (NLSs). In various instances, therefore, the fusion protein may comprise an amino acid sequence having at least 85%, at least 90%, at least 95%, at least 96%, at least 97%, at least 98%, or at least 99% sequence homology to any one of SEQ ID NOs: 46, 48, 50, 52, 54, 56, 58, and 60. In various aspects, the fusion protein may comprise an amino acid sequence comprising any one of SEQ ID NOs: 46, 48, 50, 52, 54, 56, 58 and 60. In some aspects, the fusion protein may comprise an amino acid sequence consisting of any one of SEQ ID NOs: 46, 48, 50, 52, 54, 56, 58 and 60.

TABLE 7
Exemplary Fusion Proteins (Amino Acid Sequences)

| Fusion Protein | Amino Acid Sequence | SEQ ID NO: |
|---|---|---|
| ABEmax-VRQR | SEVEFSHEYWMRHALTLAKRAWDEREVPVGAVLVHNN | 45 |
| Linker connecting | RVIGEGWNRPIGRHDPTAHAEIMALRQGGLVMQNYRLI | |
| ABEmax and | DATLYVTLEPCVMCAGAMIHSRIGRVVFGARDAKTGAA | |
| SpCas9 - VRQR | GSLMDVLHHPGMNHRVEITEGILADECAALLSDFFRMR | |
| underlined | RQEIKAQKKAQSSTDSGGSSGGSSGSETPGTSESATP | |
| | ESSGGSSGGSSEVEFSHEYWMRHALTLAKRARDERE | |
| | VPVGAVLVLNNRVIGEGWNRAIGLHDPTAHAEIMALRQ | |
| | GGLVMQNYRLIDATLYVTFEPCVMCAGAMIHSRIGRVV | |
| | FGVRNAKTGAAGSLMDVLHYPGMNHRVEITEGILADEC | |
| | AALLCYFFRMPRQVFNAQKKAQSSTDSGGSSGGSSGS | |
| | ETPGTSESATPESSGGSSGGSDKKYSIGLAIGTNSVGW | |
| | AVITDEYKVPSKKFKVLGNTDRHSIKKNLIGALLFDSGET | |
| | AEATRLKRTARRRYTRRKNRICYLQEIFSNEMAKVDDS | |
| | FFHRLEESFLVEEDKKHERHPIFGNIVDEVAYHEKYPTI | |
| | YHLRKKLVDSTDKADLRLIYLALAHMIKFRGHFLIEGDLN | |
| | PDNSDVDKLFIQLVQTYNQLFEENPINASGVDAKAILSA | |
| | RLSKSRRLENLIAQLPGEKKNGLFGNLIALSLGLTPNFK | |
| | SNFDLAEDAKLQLSKDTYDDDLDNLLAQIGDQYADLFL | |
| | AAKNLSDAILLSDILRVNTEITKAPLSASMIKRYDEHHQD | |
| | LTLLKALVRQQLPEKYKEIFFDQSKNGYAGYIDGGASQ | |
| | EEFYKFIKPILEKMDGTEELLVKLNREDLLRKQRTFDNG | |
| | SIPHQIHLGELHAILRRQEDFYPFLKDNREKIEKILTFRIP | |
| | YYVGPLARGNSRFAWMTRKSEETITPWNFEEVVDKGA | |
| | SAQSFIERMTNFDKNLPNEKVLPKHSLLYEYFTVYNELT | |
| | KVKYVTEGMRKPAFLSGEQKKAIVDLLFKTNRKVTVKQ | |
| | LKEDYFKKIECFDSVEISGVEDRFNASLGTYHDLLKIIKD | |
| | KDFLDNEENEDILEDIVLTLTLFEDREMIEERLKTYAHLF | |
| | DDKVMKQLKRRRYTGWGRLSRKLINGIRDKQSGKTILD | |
| | FLKSDGFANRNFMQLIHDDSLTFKEDIQKAQVSGQGDS | |
| | LHEHIANLAGSPAIKKGILQTVKVVDELVKVMGRHKPEN | |
| | IVIEMARENQTTQKGQKNSRERMKRIEEGIKELGSQILK | |
| | EHPVENTQLQNEKLYLYYLQNGRDMYVDQELDINRLSD | |
| | YDVDHIVPQSFLKDDSIDNKVLTRSDKNRGKSDNVPSE | |
| | EVVKKMKNYWRQLLNAKLITQRKFDNLTKAERGGLSEL | |
| | DKAGFIKRQLVETRQITKHVAQILDSRMNTKYDENDKLI | |
| | REVKVITLKSKLVSDFRKDFQFYKVREINNYHHAHDAYL | |
| | NAVVGTALIKKYPKLESEFVYGDYKVYDVRKMIAKSEQ | |
| | EIGKATAKYFFYSNIMNFFKTEITLANGEIRKRPLIETNGE | |
| | TGEIVWDKGRDFATVRKVLSMPQVNIVKKTEVQTGGFS | |
| | KESILPKRNSDKLIARKKDWDPKKYGGFVSPTVAYSVL | |
| | VVAKVEKGKSKKLKSVKELLGITIMERSSFEKNPIDFLEA | |
| | KGYKEVKKDLIIKLPKYSLFELENGRKRMLASARELQKG | |
| | NELALPSKYVNFLYLASHYEKLKGSPEDNEQKQLFVEQ | |
| | HKHYLDEIIEQISEFSKRVILADANLDKVLSAYNKHRDKP | |
| | IREQAENIIHLFTLTNLGAPAAFKYFDTTIDRKQYRSTKE | |
| | VLDATLIHQSITGLYETRIDLSQLGGD | |
| ABEmax-VRQR | MKRTADGSEFESPKKKRKVSEVEFSHEYWMRHALTL | 46 |
| with NLSs | AKRAWDEREVPVGAVLVHNNRVIGEGWNRPIGRHDPT | |
| NLS bolded. | AHAEIMALRQGGLVMQNYRLIDATLYVTLEPCVMCAGA | |
| Linkers | MIHSRIGRVVFGARDAKTGAAGSLMDVLHHPGMNHRV | |
| connecting | EITEGILADECAALLSDFFRMRRQEIKAQKKAQSSTDSG | |
| ABEmax to | GSSGGSSGSETPGTSESATPESSGGSSGGSSEVEFSH | |
| VRQR and | EYWMRHALTLAKRARDEREVPVGAVLVLNNRVIGEGW | |
| VRQR to NLS | NRAIGLHDPTAHAEIMALRQGGLVMQNYRLIDATLYVTF | |
| underlined | EPCVMCAGAMIHSRIGRVVFGVRNAKTGAAGSLMDVL | |
| | HYPGMNHRVEITEGILADECAALLCYFFRMPRQVFNAQ | |
| | KKAQSSTDSGGSSGGSSGSETPGTSESATPESSGGSS | |
| | GGSDKKYSIGLAIGTNSVGWAVITDEYKVPSKKFKVLG | |
| | NTDRHSIKKNLIGALLFDSGETAEATRLKRTARRRYTRR | |
| | KNRICYLQEIFSNEMAKVDDSFFHRLEESFLVEEDKKHE | |
| | RHPIFGNIVDEVAYHEKYPTIYHLRKKLVDSTDKADLRLI | |
| | YLALAHMIKFRGHFLIEGDLNPDNSDVDKLFIQLVQTYN | |
| | QLFEENPINASGVDAKAILSARLSKSRRLENLIAQLPGE | |
| | KKNGLFGNLIALSLGLTPNFKSNFDLAEDAKLQLSKDTY | |
| | DDDLDNLLAQIGDQYADLFLAAKNLSDAILLSDILRVNTE | |
| | ITKAPLSASMIKRYDEHHQDLTLLKALVRQQLPEKYKEIF | |
| | FDQSKNGYAGYIDGGASQEEFYKFIKPILEKMDGTEELL | |
| | VKLNREDLLRKQRTFDNGSIPHQIHLGELHAILRRQEDF | |
| | YPFLKDNREKIEKILTFRIPYYVGPLARGNSRFAWMTRK | |
| | SEETITPWNFEEVVDKGASAQSFIERMTNFDKNLPNEK | |
| | VLPKHSLLYEYFTVYNELTKVKYVTEGMRKPAFLSGEQ | |
| | KKAIVDLLFKTNRKVTVKQLKEDYFKKIECFDSVEISGVE | |
| | DRFNASLGTYHDLLKIIKDKDFLDNEENEDILEDIVLTLTL | |
| | FEDREMIEERLKTYAHLFDDKVMKQLKRRRYTGWGRL | |
| | SRKLINGIRDKQSGKTILDFLKSDGFANRNFMQLIHDDS | |
| | LTFKEDIQKAQVSGQGDSLHEHIANLAGSPAIKKGILQT | |
| | VKVVDELVKVMGRHKPENIVIEMARENQTTQKGQKNS | |
| | RERMKRIEEGIKELGSQILKEHPVENTQLQNEKLYLYYL | |
| | QNGRDMYVDQELDINRLSDYDVDHIVPQSFLKDDSIDN | |
| | KVLTRSDKNRGKSDNVPSEEVVKKMKNYWRQLLNAKL | |
| | ITQRKFDNLTKAERGGLSELDKAGFIKRQLVETRQITKH | |
| | VAQILDSRMNTKYDENDKLIREVKVITLKSKLVSDFRKD | |
| | FQFYKVREINNYHHAHDAYLNAVVGTALIKKYPKLESEF | |
| | VYGDYKVYDVRKMIAKSEQEIGKATAKYFFYSNIMNFFK | |
| | TEITLANGEIRKRPLIETNGETGEIVWDKGRDFATVRKV | |
| | LSMPQVNIVKKTEVQTGGFSKESILPKRNSDKLIARKKD | |
| | WDPKKYGGFVSPTVAYSVLVVAKVEKGKSKKLKSVKEL | |
| | LGITIMERSSFEKNPIDFLEAKGYKEVKKDLIIKLPKYSLF | |
| | ELENGRKRMLASARELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAP | |
| | AAFKYFDTTIDRKQYRSTKEVLDATLIHQSITGLYETRID | |
| | LSQLGGDSGGSKRTADGSEFEPKKKRKV | |
| ABEmax- | SEVEFSHEYWMRHALTLAKRAWDEREVPVGAVLVHNN | 47 |
| SpCas9-NG | RVIGEGWNRPIGRHDPTAHAEIMALRQGGLVMQNYRLI | |
| Linker connecting | DATLYVTLEPCVMCAGAMIHSRIGRVVFGARDAKTGAA | |
| ABEmax and | GSLMDVLHHPGMNHRVEITEGILADECAALLSDFFRMR | |
| SpCas9 - NG | RQEIKAQKKAQSSTDSGGSSGGSSGSETPGTSESATP | |
| underlined | ESSGGSSGGSSEVEFSHEYWMRHALTLAKRARDERE | |
| | VPVGAVLVLNNRVIGEGWNRAIGLHDPTAHAEIMALRQ | |
| | GGLVMQNYRLIDATLYVTFEPCVMCAGAMIHSRIGRVV | |
| | FGVRNAKTGAAGSLMDVLHYPGMNHRVEITEGILADEC | |
| | AALLCYFFRMPRQVFNAQKKAQSSTDSGGSSGGSSGS | |
| | ETPGTSESATPESSGGSSGGSDKKYSIGLAIGTNSVGW | |
| | AVITDEYKVPSKKFKVLGNTDRHSIKKNLIGALLFDSGET | |
| | AEATRLKRTARRRYTRRKNRICYLQEIFSNEMAKVDDS | |
| | FFHRLEESFLVEEDKKHERHPIFGNIVDEVAYHEKYPTI | |
| | YHLRKKLVDSTDKADLRLIYLALAHMIKFRGHFLIEGDLN | |
| | PDNSDVDKLFIQLVQTYNQLFEENPINASGVDAKAILSA | |
| | RLSKSRRLENLIAQLPGEKKNGLFGNLIALSLGLTPNFK | |
| | SNFDLAEDAKLQLSKDTYDDDLDNLLAQIGDQYADLFL | |
| | AAKNLSDAILLSDILRVNTEITKAPLSASMIKRYDEHHQD | |
| | LTLLKALVRQQLPEKYKEIFFDQSKNGYAGYIDGGASQ | |
| | EEFYKFIKPILEKMDGTEELLVKLNREDLLRKQRTFDNG | |
| | SIPHQIHLGELHAILRRQEDFYPFLKDNREKIEKILTFRIP | |
| | YYVGPLARGNSRFAWMTRKSEETITPWNFEEVVDKGA | |
| | SAQSFIERMTNFDKNLPNEKVLPKHSLLYEYFTVYNELT | |
| | KVKYVTEGMRKPAFLSGEQKKAIVDLLFKTNRKVTVKQ | |
| | LKEDYFKKIECFDSVEISGVEDRFNASLGTYHDLLKIIKD | |
| | KDFLDNEENEDILEDIVLTLTLFEDREMIEERLKTYAHLF | |
| | DDKVMKQLKRRRYTGWGRLSRKLINGIRDKQSGKTILD | |
| | FLKSDGFANRNFMQLIHDDSLTFKEDIQKAQVSGQGDS | |
| | LHEHIANLAGSPAIKKGILQTVKVVDELVKVMGRHKPEN | |
| | IVIEMARENQTTQKGQKNSRERMKRIEEGIKELGSQILK | |
| | EHPVENTQLQNEKLYLYYLQNGRDMYVDQELDINRLSD | |
| | YDVDHIVPQSFLKDDSIDNKVLTRSDKNRGKSDNVPSE | |
| | EVVKKMKNYWRQLLNAKLITQRKFDNLTKAERGGLSEL | |
| | DKAGFIKRQLVETRQITKHVAQILDSRMNTKYDENDKLI | |
| | REVKVITLKSKLVSDFRKDFQFYKVREINNYHHAHDAYL | |
| | NAVVGTALIKKYPKLESEFVYGDYKVYDVRKMIAKSEQ | |
| | EIGKATAKYFFYSNIMNFFKTEITLANGEIRKRPLIETNGE | |
| | TGEIVWDKGRDFATVRKVLSMPQVNIVKKTEVQTGGFS | |
| | KESIRPKRNSDKLIARKKDWDPKKYGGFVSPTVAYSVL | |
| | VVAKVEKGKSKKLKSVKELLGITIMERSSFEKNPIDFLEA | |
| | KGYKEVKKDLIIKLPKYSLFELENGRKRMLASARFLQKG | |
| | NELALPSKYVNFLYLASHYEKLKGSPEDNEQKQLFVEQ | |
| | HKHYLDEIIEQISEFSKRVILADANLDKVLSAYNKHRDKP | |
| | IREQAENIIHLFTLTNLGAPRAFKYFDTTIDRKVYRSTKE | |
| | VLDATLIHQSITGLYETRIDLSQLGGD | |
| ABEmax- | MKRTADGSEFESPKKKRKVSEVEFSHEYWMRHALTL | 48 |
| SpCas9-NG | AKRAWDEREVPVGAVLVHNNRVIGEGWNRPIGRHDPT | |
| NLS bolded. | AHAEIMALRQGGLVMQNYRLIDATLYVTLEPCVMCAGA | |
| Linker connecting | MIHSRIGRVVFGARDAKTGAAGSLMDVLHHPGMNHRV | |
| ABEmax to | EITEGILADECAALLSDFFRMRRQEIKAQKKAQSSTDSG | |
| VRQR and | GSSGGSSGSETPGTSESATPESSGGSSGGSSEVEFSH | |
| VRQR to NLS | EYWMRHALTLAKRARDEREVPVGAVLVLNNRVIGEGW | |
| underlined | NRAIGLHDPTAHAEIMALRQGGLVMQNYRLIDATLYVTF | |
| | EPCVMCAGAMIHSRIGRVVFGVRNAKTGAAGSLMDVL | |
| | HYPGMNHRVEITEGILADECAALLCYFFRMPRQVFNAQ | |
| | KKAQSSTDSGGSSGGSSGSETPGTSESATPESSGGSS | |
| | GGSDKKYSIGLAIGTNSVGWAVITDEYKVPSKKFKVLG | |
| | NTDRHSIKKNLIGALLFDSGETAEATRLKRTARRRYTRR | |
| | KNRICYLQEIFSNEMAKVDDSFFHRLEESFLVEEDKKHE | |
| | RHPIFGNIVDEVAYHEKYPTIYHLRKKLVDSTDKADLRLI | |
| | YLALAHMIKFRGHFLIEGDLNPDNSDVDKLFIQLVQTYN | |
| | QLFEENPINASGVDAKAILSARLSKSRRLENLIAQLPGE | |
| | KKNGLFGNLIALSLGLTPNFKSNFDLAEDAKLQLSKDTY | |
| | DDDLDNLLAQIGDQYADLFLAAKNLSDAILLSDILRVNTE | |
| | ITKAPLSASMIKRYDEHHQDLTLLKALVRQQLPEKYKEIF | |
| | FDQSKNGYAGYIDGGASQEEFYKFIKPILEKMDGTEELL | |
| | VKLNREDLLRKQRTFDNGSIPHQIHLGELHAILRRQEDF | |
| | YPFLKDNREKIEKILTFRIPYYVGPLARGNSRFAWMTRK | |
| | SEETITPWNFEEVVDKGASAQSFIERMTNFDKNLPNEK | |
| | VLPKHSLLYEYFTVYNELTKVKYVTEGMRKPAFLSGEQ | |
| | KKAIVDLLFKTNRKVTVKQLKEDYFKKIECFDSVEISGVE | |
| | DRFNASLGTYHDLLKIIKDKDFLDNEENEDILEDIVLTLTL | |
| | FEDREMIEERLKTYAHLFDDKVMKQLKRRRYTGWGRL | |
| | SRKLINGIRDKQSGKTILDFLKSDGFANRNFMQLIHDDS | |
| | LTFKEDIQKAQVSGQGDSLHEHIANLAGSPAIKKGILQT | |
| | VKVVDELVKVMGRHKPENIVIEMARENQTTQKGQKNS | |
| | RERMKRIEEGIKELGSQILKEHPVENTQLQNEKLYLYYL | |
| | QNGRDMYVDQELDINRLSDYDVDHIVPQSFLKDDSIDN | |
| | KVLTRSDKNRGKSDNVPSEEVVKKMKNYWRQLLNAKL | |
| | ITQRKFDNLTKAERGGLSELDKAGFIKRQLVETRQITKH | |
| | VAQILDSRMNTKYDENDKLIREVKVITLKSKLVSDFRKD | |
| | FQFYKVREINNYHHAHDAYLNAVVGTALIKKYPKLESEF | |
| | VYGDYKVYDVRKMIAKSEQEIGKATAKYFFYSNIMNFFK | |
| | TEITLANGEIRKRPLIETNGETGEIVWDKGRDFATVRKV | |
| | LSMPQVNIVKKTEVQTGGFSKESIRPKRNSDKLIARKKD | |
| | WDPKKYGGFVSPTVAYSVLVVAKVEKGKSKKLKSVKEL | |
| | LGITIMERSSFEKNPIDFLEAKGYKEVKKDLIIKLPKYSLF | |
| | ELENGRKRMLASARFLQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAP | |
| | RAFKYFDTTIDRKVYRSTKEVLDATLIHQSITGLYETRID | |
| | LSQLGGDSGGSKRTADGSEFEPKKKRKV | |
| ABEmax-SpRY | SEVEFSHEYWMRHALTLAKRAWDEREVPVGAVLVHNN | 49 |
| Linker connecting | RVIGEGWNRPIGRHDPTAHAEIMALRQGGLVMQNYRLI | |
| ABEmax and | DATLYVTLEPCVMCAGAMIHSRIGRVVFGARDAKTGAA | |
| SpRY underlined | GSLMDVLHHPGMNHRVEITEGILADECAALLSDFFRMR | |
| | RQEIKAQKKAQSSTDSGGSSGGSSGSETPGTSESATP | |
| | ESSGGSSGGSSEVEFSHEYWMRHALTLAKRARDERE | |
| | VPVGAVLVLNNRVIGEGWNRAIGLHDPTAHAEIMALRQ | |
| | GGLVMQNYRLIDATLYVTFEPCVMCAGAMIHSRIGRVV | |
| | FGVRNAKTGAAGSLMDVLHYPGMNHRVEITEGILADEC | |
| | AALLCYFFRMPRQVFNAQKKAQSSTDSGGSSGGSSGS | |
| | ETPGTSESATPESSGGSSGGSDKKYSIGLAIGTNSVGW | |
| | AVITDEYKVPSKKFKVLGNTDRHSIKKNLIGALLFDSGET | |
| | AERTRLKRTARRRYTRRKNRICYLQEIFSNEMAKVDDS | |
| | FFHRLEESFLVEEDKKHERHPIFGNIVDEVAYHEKYPTI | |
| | YHLRKKLVDSTDKADLRLIYLALAHMIKFRGHFLIEGDLN | |
| | PDNSDVDKLFIQLVQTYNQLFEENPINASGVDAKAILSA | |
| | RLSKSRRLENLIAQLPGEKKNGLFGNLIALSLGLTPNFK | |
| | SNFDLAEDAKLQLSKDTYDDDLDNLLAQIGDQYADLFL | |
| | AAKNLSDAILLSDILRVNTEITKAPLSASMIKRYDEHHQD | |
| | LTLLKALVRQQLPEKYKEIFFDQSKNGYAGYIDGGASQ | |
| | EEFYKFIKPILEKMDGTEELLVKLNREDLLRKQRTFDNG | |
| | SIPHQIHLGELHAILRRQEDFYPFLKDNREKIEKILTFRIP | |
| | YYVGPLARGNSRFAWMTRKSEETITPWNFEEVVDKGA | |
| | SAQSFIERMTNFDKNLPNEKVLPKHSLLYEYFTVYNELT | |
| | KVKYVTEGMRKPAFLSGEQKKAIVDLLFKTNRKVTVKQ | |
| | LKEDYFKKIECFDSVEISGVEDRFNASLGTYHDLLKIIKD | |
| | KDFLDNEENEDILEDIVLTLTLFEDREMIEERLKTYAHLF | |
| | DDKVMKQLKRRRYTGWGRLSRKLINGIRDKQSGKTILD | |
| | FLKSDGFANRNFMQLIHDDSLTFKEDIQKAQVSGQGDS | |
| | LHEHIANLAGSPAIKKGILQTVKVVDELVKVMGRHKPEN | |
| | IVIEMARENQTTQKGQKNSRERMKRIEEGIKELGSQILK | |
| | EHPVENTQLQNEKLYLYYLQNGRDMYVDQELDINRLSD | |
| | YDVDHIVPQSFLKDDSIDNKVLTRSDKNRGKSDNVPSE | |
| | EVVKKMKNYWRQLLNAKLITQRKFDNLTKAERGGLSEL | |
| | DKAGFIKRQLVETRQITKHVAQILDSRMNTKYDENDKLI | |
| | REVKVITLKSKLVSDFRKDFQFYKVREINNYHHAHDAYL | |
| | NAVVGTALIKKYPKLESEFVYGDYKVYDVRKMIAKSEQ | |
| | EIGKATAKYFFYSNIMNFFKTEITLANGEIRKRPLIETNGE | |
| | TGEIVWDKGRDFATVRKVLSMPQVNIVKKTEVQTGGFS | |
| | KESIRPKRNSDKLIARKKDWDPKKYGGFLWPTVAYSVL | |
| | VVAKVEKGKSKKLKSVKELLGITIMERSSFEKNPIDFLEA | |
| | KGYKEVKKDLIIKLPKYSLFELENGRKRMLASAKQLQKG | |
| | NELALPSKYVNFLYLASHYEKLKGSPEDNEQKQLFVEQ | |
| | HKHYLDEIIEQISEFSKRVILADANLDKVLSAYNKHRDKP | |
| | IREQAENIIHLFTLTRLGAPRAFKYFDTTIDPKQYRSTKE | |
| | VLDATLIHQSITGLYETRIDLSQLGGD | |
| ABEmax-SpRY | MKRTADGSEFESPKKKRKVSEVEFSHEYWMRHALTL | 50 |
| with NLSs | AKRAWDEREVPVGAVLVHNNRVIGEGWNRPIGRHDPT | |
| (protein) | AHAEIMALRQGGLVMQNYRLIDATLYVTLEPCVMCAGA | |
| NLS bolded. | MIHSRIGRVVFGARDAKTGAAGSLMDVLHHPGMNHRV | |
| Linker connecting | EITEGILADECAALLSDFFRMRRQEIKAQKKAQSSTDSG | |
| ABEmax to SpRY | GSSGGSSGSETPGTSESATPESSGGSSGGSSEVEFSH | |
| and SpRY to NLS | EYWMRHALTLAKRARDEREVPVGAVLVLNNRVIGEGW | |
| underlined | NRAIGLHDPTAHAEIMALRQGGLVMQNYRLIDATLYVTF | |
| | EPCVMCAGAMIHSRIGRVVFGVRNAKTGAAGSLMDVL | |
| | HYPGMNHRVEITEGILADECAALLCYFFRMPRQVFNAQ | |
| | KKAQSSTDSGGSSGGSSGSETPGTSESATPESSGGSS | |
| | GGSDKKYSIGLAIGTNSVGWAVITDEYKVPSKKFKVLG | |
| | NTDRHSIKKNLIGALLFDSGETAERTRLKRTARRRYTRR | |
| | KNRICYLQEIFSNEMAKVDDSFFHRLEESFLVEEDKKHE | |
| | RHPIFGNIVDEVAYHEKYPTIYHLRKKLVDSTDKADLRLI | |
| | YLALAHMIKFRGHFLIEGDLNPDNSDVDKLFIQLVQTYN | |
| | QLFEENPINASGVDAKAILSARLSKSRRLENLIAQLPGE | |
| | KKNGLFGNLIALSLGLTPNFKSNFDLAEDAKLQLSKDTY | |
| | DDDLDNLLAQIGDQYADLFLAAKNLSDAILLSDILRVNTE | |
| | ITKAPLSASMIKRYDEHHQDLTLLKALVRQQLPEKYKEIF | |
| | FDQSKNGYAGYIDGGASQEEFYKFIKPILEKMDGTEELL | |
| | VKLNREDLLRKQRTFDNGSIPHQIHLGELHAILRRQEDF | |
| | YPFLKDNREKIEKILTFRIPYYVGPLARGNSRFAWMTRK | |
| | SEETITPWNFEEVVDKGASAQSFIERMTNFDKNLPNEK | |
| | VLPKHSLLYEYFTVYNELTKVKYVTEGMRKPAFLSGEQ | |
| | KKAIVDLLFKTNRKVTVKQLKEDYFKKIECFDSVEISGVE | |
| | DRFNASLGTYHDLLKIIKDKDFLDNEENEDILEDIVLTLTL | |
| | FEDREMIEERLKTYAHLFDDKVMKQLKRRRYTGWGRL | |
| | SRKLINGIRDKQSGKTILDFLKSDGFANRNFMQLIHDDS | |
| | LTFKEDIQKAQVSGQGDSLHEHIANLAGSPAIKKGILQT | |
| | VKVVDELVKVMGRHKPENIVIEMARENQTTQKGQKNS | |
| | RERMKRIEEGIKELGSQILKEHPVENTQLQNEKLYLYYL | |
| | QNGRDMYVDQELDINRLSDYDVDHIVPQSFLKDDSIDN | |
| | KVLTRSDKNRGKSDNVPSEEVVKKMKNYWRQLLNAKL | |
| | ITQRKFDNLTKAERGGLSELDKAGFIKRQLVETRQITKH | |
| | VAQILDSRMNTKYDENDKLIREVKVITLKSKLVSDFRKD | |
| | FQFYKVREINNYHHAHDAYLNAVVGTALIKKYPKLESEF | |
| | VYGDYKVYDVRKMIAKSEQEIGKATAKYFFYSNIMNFFK | |
| | TEITLANGEIRKRPLIETNGETGEIVWDKGRDFATVRKV | |
| | LSMPQVNIVKKTEVQTGGFSKESIRPKRNSDKLIARKKD | |
| | WDPKKYGGFLWPTVAYSVLVVAKVEKGKSKKLKSVKE | |
| | LLGITIMERSSFEKNPIDFLEAKGYKEVKKDLIIKLPKYSL | |
| | FELENGRKRMLASAKQLQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTRLGAP | |
| | RAFKYFDTTIDPKQYRSTKEVLDATLIHQSITGLYETRID | |
| | LSQLGGDSGGSKRTADGSEFEPKKKRKV | |
| ABEmax-SpG | SEVEFSHEYWMRHALTLAKRAWDEREVPVGAVLVHNN | 51 |
| Linker connecting | RVIGEGWNRPIGRHDPTAHAEIMALRQGGLVMQNYRLI | |
| ABEmax and | DATLYVTLEPCVMCAGAMIHSRIGRVVFGARDAKTGAA | |
| SpG underlined | GSLMDVLHHPGMNHRVEITEGILADECAALLSDFFRMR | |
| | RQEIKAQKKAQSSTDSGGSSGGSSGSETPGTSESATP | |
| | ESSGGSSGGSSEVEFSHEYWMRHALTLAKRARDERE | |
| | VPVGAVLVLNNRVIGEGWNRAIGLHDPTAHAEIMALRQ | |
| | GGLVMQNYRLIDATLYVTFEPCVMCAGAMIHSRIGRVV | |
| | FGVRNAKTGAAGSLMDVLHYPGMNHRVEITEGILADEC | |
| | AALLCYFFRMPRQVFNAQKKAQSSTDSGGSSGGSSGS | |
| | ETPGTSESATPESSGGSSGGSDKKYSIGLAIGTNSVGW | |
| | AVITDEYKVPSKKFKVLGNTDRHSIKKNLIGALLFDSGET | |
| | AEATRLKRTARRRYTRRKNRICYLQEIFSNEMAKVDDS | |
| | FFHRLEESFLVEEDKKHERHPIFGNIVDEVAYHEKYPTI | |
| | YHLRKKLVDSTDKADLRLIYLALAHMIKFRGHFLIEGDLN | |
| | PDNSDVDKLFIQLVQTYNQLFEENPINASGVDAKAILSA | |
| | RLSKSRRLENLIAQLPGEKKNGLFGNLIALSLGLTPNFK | |
| | SNFDLAEDAKLQLSKDTYDDDLDNLLAQIGDQYADLFL | |
| | AAKNLSDAILLSDILRVNTEITKAPLSASMIKRYDEHHQD | |
| | LTLLKALVRQQLPEKYKEIFFDQSKNGYAGYIDGGASQ | |
| | EEFYKFIKPILEKMDGTEELLVKLNREDLLRKQRTFDNG | |
| | SIPHQIHLGELHAILRRQEDFYPFLKDNREKIEKILTFRIP | |
| | YYVGPLARGNSRFAWMTRKSEETITPWNFEEVVDKGA | |
| | SAQSFIERMTNFDKNLPNEKVLPKHSLLYEYFTVYNELT | |
| | KVKYVTEGMRKPAFLSGEQKKAIVDLLFKTNRKVTVKQ | |
| | LKEDYFKKIECFDSVEISGVEDRFNASLGTYHDLLKIIKD | |
| | KDFLDNEENEDILEDIVLTLTLFEDREMIEERLKTYAHLF | |
| | DDKVMKQLKRRRYTGWGRLSRKLINGIRDKQSGKTILD | |
| | FLKSDGFANRNFMQLIHDDSLTFKEDIQKAQVSGQGDS | |
| | LHEHIANLAGSPAIKKGILQTVKVVDELVKVMGRHKPEN | |
| | IVIEMARENQTTQKGQKNSRERMKRIEEGIKELGSQILK | |
| | EHPVENTQLQNEKLYLYYLQNGRDMYVDQELDINRLSD | |
| | YDVDHIVPQSFLKDDSIDNKVLTRSDKNRGKSDNVPSE | |
| | EVVKKMKNYWRQLLNAKLITQRKFDNLTKAERGGLSEL | |
| | DKAGFIKRQLVETRQITKHVAQILDSRMNTKYDENDKLI | |
| | REVKVITLKSKLVSDFRKDFQFYKVREINNYHHAHDAYL | |
| | NAVVGTALIKKYPKLESEFVYGDYKVYDVRKMIAKSEQ | |
| | EIGKATAKYFFYSNIMNFFKTEITLANGEIRKRPLIETNGE | |
| | TGEIVWDKGRDFATVRKVLSMPQVNIVKKTEVQTGGFS | |
| | KESILPKRNSDKLIARKKDWDPKKYGGFLWPTVAYSVL | |
| | VVAKVEKGKSKKLKSVKELLGITIMERSSFEKNPIDFLEA | |
| | KGYKEVKKDLIIKLPKYSLFELENGRKRMLASAKQLQKG | |
| | NELALPSKYVNFLYLASHYEKLKGSPEDNEQKQLFVEQ | |
| | HKHYLDEIIEQISEFSKRVILADANLDKVLSAYNKHRDKP | |
| | IREQAENIIHLFTLTNLGAPAAFKYFDTTIDRKQYRSTKE | |
| | VLDATLIHQSITGLYETRIDLSQLGGD | |
| ABEmax-SpG | MKRTADGSEFESPKKKRKVSEVEFSHEYWMRHALTL | 52 |
| NLS bolded. | AKRAWDEREVPVGAVLVHNNRVIGEGWNRPIGRHDPT | |
| Linker connecting | AHAEIMALRQGGLVMQNYRLIDATLYVTLEPCVMCAGA | |
| ABEmax to SpG | MIHSRIGRVVFGARDAKTGAAGSLMDVLHHPGMNHRV | |
| and SpG to NLS | EITEGILADECAALLSDFFRMRRQEIKAQKKAQSSTDSG | |
| underlined | GSSGGSSGSETPGTSESATPESSGGSSGGSSEVEFSH | |
| | EYWMRHALTLAKRARDEREVPVGAVLVLNNRVIGEGW | |
| | NRAIGLHDPTAHAEIMALRQGGLVMQNYRLIDATLYVTF | |
| | EPCVMCAGAMIHSRIGRVVFGVRNAKTGAAGSLMDVL | |
| | HYPGMNHRVEITEGILADECAALLCYFFRMPRQVFNAQ | |
| | KKAQSSTDSGGSSGGSSGSETPGTSESATPESSGGSS | |
| | GGSDKKYSIGLAIGTNSVGWAVITDEYKVPSKKFKVLG | |
| | NTDRHSIKKNLIGALLFDSGETAEATRLKRTARRRYTRR | |
| | KNRICYLQEIFSNEMAKVDDSFFHRLEESFLVEEDKKHE | |
| | RHPIFGNIVDEVAYHEKYPTIYHLRKKLVDSTDKADLRLI | |
| | YLALAHMIKFRGHFLIEGDLNPDNSDVDKLFIQLVQTYN | |
| | QLFEENPINASGVDAKAILSARLSKSRRLENLIAQLPGE | |
| | KKNGLFGNLIALSLGLTPNFKSNFDLAEDAKLQLSKDTY | |
| | DDDLDNLLAQIGDQYADLFLAAKNLSDAILLSDILRVNTE | |
| | ITKAPLSASMIKRYDEHHQDLTLLKALVRQQLPEKYKEIF | |
| | FDQSKNGYAGYIDGGASQEEFYKFIKPILEKMDGTEELL | |
| | VKLNREDLLRKQRTFDNGSIPHQIHLGELHAILRRQEDF | |
| | YPFLKDNREKIEKILTFRIPYYVGPLARGNSRFAWMTRK | |
| | SEETITPWNFEEVVDKGASAQSFIERMTNFDKNLPNEK | |
| | VLPKHSLLYEYFTVYNELTKVKYVTEGMRKPAFLSGEQ | |
| | KKAIVDLLFKTNRKVTVKQLKEDYFKKIECFDSVEISGVE | |
| | DRFNASLGTYHDLLKIIKDKDFLDNEENEDILEDIVLTLTL | |
| | FEDREMIEERLKTYAHLFDDKVMKQLKRRRYTGWGRL | |
| | SRKLINGIRDKQSGKTILDFLKSDGFANRNFMQLIHDDS | |
| | LTFKEDIQKAQVSGQGDSLHEHIANLAGSPAIKKGILQT | |
| | VKVVDELVKVMGRHKPENIVIEMARENQTTQKGQKNS | |
| | RERMKRIEEGIKELGSQILKEHPVENTQLQNEKLYLYYL | |
| | QNGRDMYVDQELDINRLSDYDVDHIVPQSFLKDDSIDN | |
| | KVLTRSDKNRGKSDNVPSEEVVKKMKNYWRQLLNAKL | |
| | ITQRKFDNLTKAERGGLSELDKAGFIKRQLVETRQITKH | |
| | VAQILDSRMNTKYDENDKLIREVKVITLKSKLVSDFRKD | |
| | FQFYKVREINNYHHAHDAYLNAVVGTALIKKYPKLESEF | |
| | VYGDYKVYDVRKMIAKSEQEIGKATAKYFFYSNIMNFFK | |
| | TEITLANGEIRKRPLIETNGETGEIVWDKGRDFATVRKV | |
| | LSMPQVNIVKKTEVQTGGFSKESILPKRNSDKLIARKKD | |
| | WDPKKYGGFLWPTVAYSVLVVAKVEKGKSKKLKSVKE | |
| | LLGITIMERSSFEKNPIDFLEAKGYKEVKKDLIIKLPKYSL | |
| | FELENGRKRMLASAKQLQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTNLGAP | |
| | AAFKYFDTTIDRKQYRSTKEVLDATLIHQSITGLYETRID | |
| | LSQLGGDSGGSKRTADGSEFEPKKKRKV | |
| ABE8e-VRQR | SEVEFSHEYWMRHALTLAKRARDEREVPVGAVLVLNN | 53 |
| Linker connecting | RVIGEGWNRAIGLHDPTAHAEIMALRQGGLVMQNYRLI | |
| ABE8e and | DATLYVTFEPCVMCAGAMIHSRIGRVVFGVRNSKRGAA | |
| VRQR underlined | GSLMNVLNYPGMNHRVEITEGILADECAALLCDFYRMP | |
| | RQVFNAQKKAQSSINSGGSSGGSSGSETPGTSESATP | |
| | ESSGGSSGGSDKKYSIGLAIGTNSVGWAVITDEYKVPS | |
| | KKFKVLGNTDRHSIKKNLIGALLFDSGETAEATRLKRTA | |
| | RRRYTRRKNRICYLQEIFSNEMAKVDDSFFHRLEESFL | |
| | VEEDKKHERHPIFGNIVDEVAYHEKYPTIYHLRKKLVDS | |
| | TDKADLRLIYLALAHMIKFRGHFLIEGDLNPDNSDVDKLF | |
| | IQLVQTYNQLFEENPINASGVDAKAILSARLSKSRRLEN | |
| | LIAQLPGEKKNGLFGNLIALSLGLTPNFKSNFDLAEDAKL | |
| | QLSKDTYDDDLDNLLAQIGDQYADLFLAAKNLSDAILLS | |
| | DILRVNTEITKAPLSASMIKRYDEHHQDLTLLKALVRQQL | |
| | PEKYKEIFFDQSKNGYAGYIDGGASQEEFYKFIKPILEK | |
| | MDGTEELLVKLNREDLLRKQRTFDNGSIPHQIHLGELH | |
| | AILRRQEDFYPFLKDNREKIEKILTFRIPYYVGPLARGNS | |
| | RFAWMTRKSEETITPWNFEEVVDKGASAQSFIERMTNF | |
| | DKNLPNEKVLPKHSLLYEYFTVYNELTKVKYVTEGMRK | |
| | PAFLSGEQKKAIVDLLFKTNRKVTVKQLKEDYFKKIECF | |
| | DSVEISGVEDRFNASLGTYHDLLKIIKDKDFLDNEENEDI | |
| | LEDIVLTLTLFEDREMIEERLKTYAHLFDDKVMKQLKRR | |
| | RYTGWGRLSRKLINGIRDKQSGKTILDFLKSDGFANRN | |
| | FMQLIHDDSLTFKEDIQKAQVSGQGDSLHEHIANLAGS | |
| | PAIKKGILQTVKVVDELVKVMGRHKPENIVIEMARENQT | |
| | TQKGQKNSRERMKRIEEGIKELGSQILKEHPVENTQLQ | |
| | NEKLYLYYLQNGRDMYVDQELDINRLSDYDVDHIVPQS | |
| | FLKDDSIDNKVLTRSDKNRGKSDNVPSEEVVKKMKNY | |
| | WRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIK | |
| | KYPKLESEFVYGDYKVYDVRKMIAKSEQEIGKATAKYFF | |
| | YSNIMNFFKTEITLANGEIRKRPLIETNGETGEIVWDKGR | |
| | DFATVRKVLSMPQVNIVKKTEVQTGGFSKESILPKRNS | |
| | DKLIARKKDWDPKKYGGFVSPTVAYSVLVVAKVEKGKS | |
| | KKLKSVKELLGITIMERSSFEKNPIDFLEAKGYKEVKKDL | |
| | IIKLPKYSLFELENGRKRMLASARELQKGNELALPSKYV | |
| | NFLYLASHYEKLKGSPEDNEQKQLFVEQHKHYLDEIIEQ | |
| | ISEFSKRVILADANLDKVLSAYNKHRDKPIREQAENIIHLF | |
| | TLTNLGAPAAFKYFDTTIDRKQYRSTKEVLDATLIHQSIT | |
| | GLYETRIDLSQLGGD | |
| ABE8e-VRQR | MKRTADGSEFESPKKKRKVSEVEFSHEYWMRHALTL | 54 |
| NLS bolded. | AKRARDEREVPVGAVLVLNNRVIGEGWNRAIGLHDPTA | |
| Linker connecting | HAEIMALRQGGLVMQNYRLIDATLYVTFEPCVMCAGAM | |
| ABE8e to VRQR | IHSRIGRVVFGVRNSKRGAAGSLMNVLNYPGMNHRVEI | |
| and VRQR to | TEGILADECAALLCDFYRMPRQVFNAQKKAQSSINSGG | |
| NLS underlined | SSGGSSGSETPGTSESATPESSGGSSGGSDKKYSIGL | |
| | AIGTNSVGWAVITDEYKVPSKKFKVLGNTDRHSIKKNLI | |
| | GALLFDSGETAEATRLKRTARRRYTRRKNRICYLQEIFS | |
| | NEMAKVDDSFFHRLEESFLVEEDKKHERHPIFGNIVDE | |
| | VAYHEKYPTIYHLRKKLVDSTDKADLRLIYLALAHMIKFR | |
| | GHFLIEGDLNPDNSDVDKLFIQLVQTYNQLFEENPINAS | |
| | GVDAKAILSARLSKSRRLENLIAQLPGEKKNGLFGNLIAL | |
| | SLGLTPNFKSNFDLAEDAKLQLSKDTYDDDLDNLLAQIG | |
| | DQYADLFLAAKNLSDAILLSDILRVNTEITKAPLSASMIKR | |
| | YDEHHQDLTLLKALVRQQLPEKYKEIFFDQSKNGYAGYI | |
| | DGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEV | |
| | VDKGASAQSFIERMTNFDKNLPNEKVLPKHSLLYEYFT | |
| | VYNELTKVKYVTEGMRKPAFLSGEQKKAIVDLLFKTNR | |
| | KVTVKQLKEDYFKKIECFDSVEISGVEDRFNASLGTYHD | |
| | LLKIIKDKDFLDNEENEDILEDIVLTLTLFEDREMIEERLK | |
| | TYAHLFDDKVMKQLKRRRYTGWGRLSRKLINGIRDKQS | |
| | GKTILDFLKSDGFANRNFMQLIHDDSLTFKEDIQKAQVS | |
| | GQGDSLHEHIANLAGSPAIKKGILQTVKVVDELVKVMG | |
| | RHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRKMI | |
| | AKSEQEIGKATAKYFFYSNIMNFFKTEITLANGEIRKRPLI | |
| | ETNGETGEIVWDKGRDFATVRKVLSMPQVNIVKKTEVQ | |
| | TGGFSKESILPKRNSDKLIARKKDWDPKKYGGFVSPTV | |
| | AYSVLVVAKVEKGKSKKLKSVKELLGITIMERSSFEKNPI | |
| | DFLEAKGYKEVKKDLIIKLPKYSLFELENGRKRMLASAR | |
| | ELQKGNELALPSKYVNFLYLASHYEKLKGSPEDNEQKQ | |
| | LFVEQHKHYLDEIIEQISEFSKRVILADANLDKVLSAYNK | |
| | HRDKPIREQAENIIHLFTLTNLGAPAAFKYFDTTIDRKQY | |
| | RSTKEVLDATLIHQSITGLYETRIDLSQLGGDSGGSKRT | |
| | ADGSEFEPKKKRKV | |
| ABE8e-SpCas9- | SEVEFSHEYWMRHALTLAKRARDEREVPVGAVLVLNN | 55 |
| NG | RVIGEGWNRAIGLHDPTAHAEIMALRQGGLVMQNYRLI | |
| Linker connecting | DATLYVTFEPCVMCAGAMIHSRIGRVVFGVRNSKRGAA | |
| ABE8e and | GSLMNVLNYPGMNHRVEITEGILADECAALLCDFYRMP | |
| SpCas9 -NG | RQVFNAQKKAQSSINSGGSSGGSSGSETPGTSESATP | |
| underlined | ESSGGSSGGSDKKYSIGLAIGTNSVGWAVITDEYKVPS | |
| | KKFKVLGNTDRHSIKKNLIGALLFDSGETAEATRLKRTA | |
| | RRRYTRRKNRICYLQEIFSNEMAKVDDSFFHRLEESFL | |
| | VEEDKKHERHPIFGNIVDEVAYHEKYPTIYHLRKKLVDS | |
| | TDKADLRLIYLALAHMIKFRGHFLIEGDLNPDNSDVDKLF | |
| | IQLVQTYNQLFEENPINASGVDAKAILSARLSKSRRLEN | |
| | LIAQLPGEKKNGLFGNLIALSLGLTPNFKSNFDLAEDAKL | |
| | QLSKDTYDDDLDNLLAQIGDQYADLFLAAKNLSDAILLS | |
| | DILRVNTEITKAPLSASMIKRYDEHHQDLTLLKALVRQQL | |
| | PEKYKEIFFDQSKNGYAGYIDGGASQEEFYKFIKPILEK | |
| | MDGTEELLVKLNREDLLRKQRTFDNGSIPHQIHLGELH | |
| | AILRRQEDFYPFLKDNREKIEKILTFRIPYYVGPLARGNS | |
| | RFAWMTRKSEETITPWNFEEVVDKGASAQSFIERMTNF | |
| | DKNLPNEKVLPKHSLLYEYFTVYNELTKVKYVTEGMRK | |
| | PAFLSGEQKKAIVDLLFKTNRKVTVKQLKEDYFKKIECF | |
| | DSVEISGVEDRFNASLGTYHDLLKIIKDKDFLDNEENEDI | |
| | LEDIVLTLTLFEDREMIEERLKTYAHLFDDKVMKQLKRR | |
| | RYTGWGRLSRKLINGIRDKQSGKTILDFLKSDGFANRN | |
| | FMQLIHDDSLTFKEDIQKAQVSGQGDSLHEHIANLAGS | |
| | PAIKKGILQTVKVVDELVKVMGRHKPENIVIEMARENQT | |
| | TQKGQKNSRERMKRIEEGIKELGSQILKEHPVENTQLQ | |
| | NEKLYLYYLQNGRDMYVDQELDINRLSDYDVDHIVPQS | |
| | FLKDDSIDNKVLTRSDKNRGKSDNVPSEEVVKKMKNY | |
| | WRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIK | |
| | KYPKLESEFVYGDYKVYDVRKMIAKSEQEIGKATAKYFF | |
| | YSNIMNFFKTEITLANGEIRKRPLIETNGETGEIVWDKGR | |
| | DFATVRKVLSMPQVNIVKKTEVQTGGFSKESIRPKRNS | |
| | DKLIARKKDWDPKKYGGFVSPTVAYSVLVVAKVEKGKS | |
| | KKLKSVKELLGITIMERSSFEKNPIDFLEAKGYKEVKKDL | |
| | IIKLPKYSLFELENGRKRMLASARFLQKGNELALPSKYV | |
| | NFLYLASHYEKLKGSPEDNEQKQLFVEQHKHYLDEIIEQ | |
| | ISEFSKRVILADANLDKVLSAYNKHRDKPIREQAENIIHLF | |
| | TLTNLGAPRAFKYFDTTIDRKVYRSTKEVLDATLIHQSIT | |
| | GLYETRIDLSQLGGD | |
| ABE8e-SpCas9- | MKRTADGSEFESPKKKRKVSEVEFSHEYWMRHALTL | 56 |
| NG | AKRARDEREVPVGAVLVLNNRVIGEGWNRAIGLHDPTA | |
| NLS bolded. | HAEIMALRQGGLVMQNYRLIDATLYVTFEPCVMCAGAM | |
| Linker connecting | IHSRIGRVVFGVRNSKRGAAGSLMNVLNYPGMNHRVEI | |
| ABE8e to | TEGILADECAALLCDFYRMPRQVFNAQKKAQSSINSGG | |
| SpCas9-NG and | SSGGSSGSETPGTSESATPESSGGSSGGSDKKYSIGL | |
| SpCas9-NG to | AIGTNSVGWAVITDEYKVPSKKFKVLGNTDRHSIKKNLI | |
| NLS underlined | GALLFDSGETAEATRLKRTARRRYTRRKNRICYLQEIFS | |
| | NEMAKVDDSFFHRLEESFLVEEDKKHERHPIFGNIVDE | |
| | VAYHEKYPTIYHLRKKLVDSTDKADLRLIYLALAHMIKFR | |
| | GHFLIEGDLNPDNSDVDKLFIQLVQTYNQLFEENPINAS | |
| | GVDAKAILSARLSKSRRLENLIAQLPGEKKNGLFGNLIAL | |
| | SLGLTPNFKSNFDLAEDAKLQLSKDTYDDDLDNLLAQIG | |
| | DQYADLFLAAKNLSDAILLSDILRVNTEITKAPLSASMIKR | |
| | YDEHHQDLTLLKALVRQQLPEKYKEIFFDQSKNGYAGYI | |
| | DGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEV | |
| | VDKGASAQSFIERMTNFDKNLPNEKVLPKHSLLYEYFT | |
| | VYNELTKVKYVTEGMRKPAFLSGEQKKAIVDLLFKTNR | |
| | KVTVKQLKEDYFKKIECFDSVEISGVEDRFNASLGTYHD | |
| | LLKIIKDKDFLDNEENEDILEDIVLTLTLFEDREMIEERLK | |
| | TYAHLFDDKVMKQLKRRRYTGWGRLSRKLINGIRDKQS | |
| | GKTILDFLKSDGFANRNFMQLIHDDSLTFKEDIQKAQVS | |
| | GQGDSLHEHIANLAGSPAIKKGILQTVKVVDELVKVMG | |
| | RHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRKMI | |
| | AKSEQEIGKATAKYFFYSNIMNFFKTEITLANGEIRKRPLI | |
| | ETNGETGEIVWDKGRDFATVRKVLSMPQVNIVKKTEVQ | |
| | TGGFSKESIRPKRNSDKLIARKKDWDPKKYGGFVSPTV | |
| | AYSVLVVAKVEKGKSKKLKSVKELLGITIMERSSFEKNPI | |
| | DFLEAKGYKEVKKDLIIKLPKYSLFELENGRKRMLASAR | |
| | FLQKGNELALPSKYVNFLYLASHYEKLKGSPEDNEQKQ | |
| | LFVEQHKHYLDEIIEQISEFSKRVILADANLDKVLSAYNK | |
| | HRDKPIREQAENIIHLFTLTNLGAPRAFKYFDTTIDRKVY | |
| | RSTKEVLDATLIHQSITGLYETRIDLSQLGGDSGGSKRT | |
| | ADGSEFEPKKKRKV | |
| ABE8e-SpRY | SEVEFSHEYWMRHALTLAKRARDEREVPVGAVLVLNN | 57 |
| Linker connecting | RVIGEGWNRAIGLHDPTAHAEIMALRQGGLVMQNYRLI | |
| ABE8e and SpRY | DATLYVTFEPCVMCAGAMIHSRIGRVVFGVRNSKRGAA | |
| underlined | GSLMNVLNYPGMNHRVEITEGILADECAALLCDFYRMP | |
| | RQVFNAQKKAQSSINSGGSSGGSSGSETPGTSESATP | |
| | ESSGGSSGGSDKKYSIGLAIGTNSVGWAVITDEYKVPS | |
| | KKFKVLGNTDRHSIKKNLIGALLFDSGETAERTRLKRTA | |
| | RRRYTRRKNRICYLQEIFSNEMAKVDDSFFHRLEESFL | |
| | VEEDKKHERHPIFGNIVDEVAYHEKYPTIYHLRKKLVDS | |
| | TDKADLRLIYLALAHMIKFRGHFLIEGDLNPDNSDVDKLF | |
| | IQLVQTYNQLFEENPINASGVDAKAILSARLSKSRRLEN | |
| | LIAQLPGEKKNGLFGNLIALSLGLTPNFKSNFDLAEDAKL | |
| | QLSKDTYDDDLDNLLAQIGDQYADLFLAAKNLSDAILLS | |
| | DILRVNTEITKAPLSASMIKRYDEHHQDLTLLKALVRQQL | |
| | PEKYKEIFFDQSKNGYAGYIDGGASQEEFYKFIKPILEK | |
| | MDGTEELLVKLNREDLLRKQRTFDNGSIPHQIHLGELH | |
| | AILRRQEDFYPFLKDNREKIEKILTFRIPYYVGPLARGNS | |
| | RFAWMTRKSEETITPWNFEEVVDKGASAQSFIERMTNF | |
| | DKNLPNEKVLPKHSLLYEYFTVYNELTKVKYVTEGMRK | |
| | PAFLSGEQKKAIVDLLFKTNRKVTVKQLKEDYFKKIECF | |
| | DSVEISGVEDRFNASLGTYHDLLKIIKDKDFLDNEENEDI | |
| | LEDIVLTLTLFEDREMIEERLKTYAHLFDDKVMKQLKRR | |
| | RYTGWGRLSRKLINGIRDKQSGKTILDFLKSDGFANRN | |
| | FMQLIHDDSLTFKEDIQKAQVSGQGDSLHEHIANLAGS | |
| | PAIKKGILQTVKVVDELVKVMGRHKPENIVIEMARENQT | |
| | TQKGQKNSRERMKRIEEGIKELGSQILKEHPVENTQLQ | |
| | NEKLYLYYLQNGRDMYVDQELDINRLSDYDVDHIVPQS | |
| | FLKDDSIDNKVLTRSDKNRGKSDNVPSEEVVKKMKNY | |
| | WRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIK | |
| | KYPKLESEFVYGDYKVYDVRKMIAKSEQEIGKATAKYFF | |
| | YSNIMNFFKTEITLANGEIRKRPLIETNGETGEIVWDKGR | |
| | DFATVRKVLSMPQVNIVKKTEVQTGGFSKESIRPKRNS | |
| | DKLIARKKDWDPKKYGGFLWPTVAYSVLVVAKVEKGK | |
| | SKKLKSVKELLGITIMERSSFEKNPIDFLEAKGYKEVKKD | |
| | LIIKLPKYSLFELENGRKRMLASAKQLQKGNELALPSKY | |
| | VNFLYLASHYEKLKGSPEDNEQKQLFVEQHKHYLDEIIE | |
| | QISEFSKRVILADANLDKVLSAYNKHRDKPIREQAENIIH | |
| | LFTLTRLGAPRAFKYFDTTIDPKQYRSTKEVLDATLIHQS | |
| | ITGLYETRIDLSQLGGD | |
| ABE8e-SpRY | MKRTADGSEFESPKKKRKVSEVEFSHEYWMRHALTL | 58 |
| NLS bolded. | AKRARDEREVPVGAVLVLNNRVIGEGWNRAIGLHDPTA | |
| Linker connecting | HAEIMALRQGGLVMQNYRLIDATLYVTFEPCVMCAGAM | |
| ABE8e to SpRY | IHSRIGRVVFGVRNSKRGAAGSLMNVLNYPGMNHRVEI | |
| and SpRY to NLS | TEGILADECAALLCDFYRMPRQVFNAQKKAQSSINSGG | |
| underlined | SSGGSSGSETPGTSESATPESSGGSSGGSDKKYSIGL | |
| | AIGTNSVGWAVITDEYKVPSKKFKVLGNTDRHSIKKNLI | |
| | GALLFDSGETAERTRLKRTARRRYTRRKNRICYLQEIFS | |
| | NEMAKVDDSFFHRLEESFLVEEDKKHERHPIFGNIVDE | |
| | VAYHEKYPTIYHLRKKLVDSTDKADLRLIYLALAHMIKFR | |
| | GHFLIEGDLNPDNSDVDKLFIQLVQTYNQLFEENPINAS | |
| | GVDAKAILSARLSKSRRLENLIAQLPGEKKNGLFGNLIAL | |
| | SLGLTPNFKSNFDLAEDAKLQLSKDTYDDDLDNLLAQIG | |
| | DQYADLFLAAKNLSDAILLSDILRVNTEITKAPLSASMIKR | |
| | YDEHHQDLTLLKALVRQQLPEKYKEIFFDQSKNGYAGYI | |
| | DGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEV | |
| | VDKGASAQSFIERMTNFDKNLPNEKVLPKHSLLYEYFT | |
| | VYNELTKVKYVTEGMRKPAFLSGEQKKAIVDLLFKTNR | |
| | KVTVKQLKEDYFKKIECFDSVEISGVEDRFNASLGTYHD | |
| | LLKIIKDKDFLDNEENEDILEDIVLTLTLFEDREMIEERLK | |
| | TYAHLFDDKVMKQLKRRRYTGWGRLSRKLINGIRDKQS | |
| | GKTILDFLKSDGFANRNFMQLIHDDSLTFKEDIQKAQVS | |
| | GQGDSLHEHIANLAGSPAIKKGILQTVKVVDELVKVMG | |
| | RHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRKMI | |
| | AKSEQEIGKATAKYFFYSNIMNFFKTEITLANGEIRKRPLI | |
| | ETNGETGEIVWDKGRDFATVRKVLSMPQVNIVKKTEVQ | |
| | TGGFSKESIRPKRNSDKLIARKKDWDPKKYGGFLWPTV | |
| | AYSVLVVAKVEKGKSKKLKSVKELLGITIMERSSFEKNPI | |
| | DFLEAKGYKEVKKDLIIKLPKYSLFELENGRKRMLASAK | |
| | QLQKGNELALPSKYVNFLYLASHYEKLKGSPEDNEQKQ | |
| | LFVEQHKHYLDEIIEQISEFSKRVILADANLDKVLSAYNK | |
| | HRDKPIREQAENIIHLFTLTRLGAPRAFKYFDTTIDPKQY | |
| | RSTKEVLDATLIHQSITGLYETRIDLSQLGGDSGGSKRT | |
| | ADGSEFEPKKKRKV | |
| ABE8e-SpG | SEVEFSHEYWMRHALTLAKRARDEREVPVGAVLVLNN | 59 |
| Linker connecting | RVIGEGWNRAIGLHDPTAHAEIMALRQGGLVMQNYRLI | |
| ABE8e and SpG | DATLYVTFEPCVMCAGAMIHSRIGRVVFGVRNSKRGAA | |
| underlined | GSLMNVLNYPGMNHRVEITEGILADECAALLCDFYRMP | |
| | RQVFNAQKKAQSSINSGGSSGGSSGSETPGTSESATP | |
| | ESSGGSSGGSDKKYSIGLAIGTNSVGWAVITDEYKVPS | |
| | KKFKVLGNTDRHSIKKNLIGALLFDSGETAEATRLKRTA | |
| | RRRYTRRKNRICYLQEIFSNEMAKVDDSFFHRLEESFL | |
| | VEEDKKHERHPIFGNIVDEVAYHEKYPTIYHLRKKLVDS | |
| | TDKADLRLIYLALAHMIKFRGHFLIEGDLNPDNSDVDKLF | |
| | IQLVQTYNQLFEENPINASGVDAKAILSARLSKSRRLEN | |
| | LIAQLPGEKKNGLFGNLIALSLGLTPNFKSNFDLAEDAKL | |
| | QLSKDTYDDDLDNLLAQIGDQYADLFLAAKNLSDAILLS | |
| | DILRVNTEITKAPLSASMIKRYDEHHQDLTLLKALVRQQL | |
| | PEKYKEIFFDQSKNGYAGYIDGGASQEEFYKFIKPILEK | |
| | MDGTEELLVKLNREDLLRKQRTFDNGSIPHQIHLGELH | |
| | AILRRQEDFYPFLKDNREKIEKILTFRIPYYVGPLARGNS | |
| | RFAWMTRKSEETITPWNFEEVVDKGASAQSFIERMTNF | |
| | DKNLPNEKVLPKHSLLYEYFTVYNELTKVKYVTEGMRK | |
| | PAFLSGEQKKAIVDLLFKTNRKVTVKQLKEDYFKKIECF | |
| | DSVEISGVEDRFNASLGTYHDLLKIIKDKDFLDNEENEDI | |
| | LEDIVLTLTLFEDREMIEERLKTYAHLFDDKVMKQLKRR | |
| | RYTGWGRLSRKLINGIRDKQSGKTILDFLKSDGFANRN | |
| | FMQLIHDDSLTFKEDIQKAQVSGQGDSLHEHIANLAGS | |
| | PAIKKGILQTVKVVDELVKVMGRHKPENIVIEMARENQT | |
| | TQKGQKNSRERMKRIEEGIKELGSQILKEHPVENTQLQ | |
| | NEKLYLYYLQNGRDMYVDQELDINRLSDYDVDHIVPQS | |
| | FLKDDSIDNKVLTRSDKNRGKSDNVPSEEVVKKMKNY | |
| | WRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIK | |
| | KYPKLESEFVYGDYKVYDVRKMIAKSEQEIGKATAKYFF | |
| | YSNIMNFFKTEITLANGEIRKRPLIETNGETGEIVWDKGR | |
| | DFATVRKVLSMPQVNIVKKTEVQTGGFSKESILPKRNS | |
| | DKLIARKKDWDPKKYGGFLWPTVAYSVLVVAKVEKGK | |
| | SKKLKSVKELLGITIMERSSFEKNPIDFLEAKGYKEVKKD | |
| | LIIKLPKYSLFELENGRKRMLASAKQLQKGNELALPSKY | |
| | VNFLYLASHYEKLKGSPEDNEQKQLFVEQHKHYLDEIIE | |
| | QISEFSKRVILADANLDKVLSAYNKHRDKPIREQAENIIH | |
| | LFTLTNLGAPAAFKYFDTTIDRKQYRSTKEVLDATLIHQS | |
| | ITGLYETRIDLSQLGGD | |
| ABE8e-SpG | MKRTADGSEFESPKKKRKVSEVEFSHEYWMRHALTL | 60 |
| NLS bolded. | AKRARDEREVPVGAVLVLNNRVIGEGWNRAIGLHDPTA | |
| Linker connecting | HAEIMALRQGGLVMQNYRLIDATLYVTFEPCVMCAGAM | |
| ABE8eto SpG | IHSRIGRVVFGVRNSKRGAAGSLMNVLNYPGMNHRVEI | |
| and SpG to NLS | TEGILADECAALLCDFYRMPRQVFNAQKKAQSSINSGG | |
| underlined | SSGGSSGSETPGTSESATPESSGGSSGGSDKKYSIGL | |
| | AIGTNSVGWAVITDEYKVPSKKFKVLGNTDRHSIKKNLI | |
| | GALLFDSGETAEATRLKRTARRRYTRRKNRICYLQEIFS | |
| | NEMAKVDDSFFHRLEESFLVEEDKKHERHPIFGNIVDE | |
| | VAYHEKYPTIYHLRKKLVDSTDKADLRLIYLALAHMIKFR | |
| | GHFLIEGDLNPDNSDVDKLFIQLVQTYNQLFEENPINAS | |
| | GVDAKAILSARLSKSRRLENLIAQLPGEKKNGLFGNLIAL | |
| | SLGLTPNFKSNFDLAEDAKLQLSKDTYDDDLDNLLAQIG | |
| | DQYADLFLAAKNLSDAILLSDILRVNTEITKAPLSASMIKR | |
| | YDEHHQDLTLLKALVRQQLPEKYKEIFFDQSKNGYAGYI | |
| | DGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEV | |
| | VDKGASAQSFIERMTNFDKNLPNEKVLPKHSLLYEYFT | |
| | VYNELTKVKYVTEGMRKPAFLSGEQKKAIVDLLFKTNR | |
| | KVTVKQLKEDYFKKIECFDSVEISGVEDRFNASLGTYHD | |
| | LLKIIKDKDFLDNEENEDILEDIVLTLTLFEDREMIEERLK | |
| | TYAHLFDDKVMKQLKRRRYTGWGRLSRKLINGIRDKQS | |
| | GKTILDFLKSDGFANRNFMQLIHDDSLTFKEDIQKAQVS | |
| | GQGDSLHEHIANLAGSPAIKKGILQTVKVVDELVKVMG | |
| | RHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRKMI | |
| | AKSEQEIGKATAKYFFYSNIMNFFKTEITLANGEIRKRPLI | |
| | ETNGETGEIVWDKGRDFATVRKVLSMPQVNIVKKTEVQ | |
| | TGGFSKESILPKRNSDKLIARKKDWDPKKYGGFLWPTV | |
| | AYSVLVVAKVEKGKSKKLKSVKELLGITIMERSSFEKNPI | |
| | DFLEAKGYKEVKKDLIIKLPKYSLFELENGRKRMLASAK | |
| | QLQKGNELALPSKYVNFLYLASHYEKLKGSPEDNEQKQ | |
| | LFVEQHKHYLDEIIEQISEFSKRVILADANLDKVLSAYNK | |
| | HRDKPIREQAENIIHLFTLTNLGAPAAFKYFDTTIDRKQY | |
| | RSTKEVLDATLIHQSITGLYETRIDLSQLGGDSGGSKRT | |
| | ADGSEFEPKKKRKV | |

In various aspects, the fusion proteins provided herein may be encoded by one or more nucleic acids. In some aspects, the fusion proteins may be encoded by a single nucleic acid. Suitable nucleic acids that encode the full fusion proteins described above (including the linkers and NL-Ss) are provided in Table 8 herein. In some aspects, the fusion protein may be encoded by a nucleic acid comprising any one of SEQ ID NOs: 61 to 68. In some aspects, the fusion protein may be encoded by a nucleic acid comprising any one of SEQ ID NOs: 73, 79 and 147-152.

TABLE 8
Exemplary Fusion Proteins (Nucleic Acid Sequences)

| Fusion Protein | Nucleic Acid Sequence | SEQ ID NO: |
|---|---|---|
| ABEmax-VRQR | atgaaacggacagccgacggaagcgagttcgagtcaccaaagaagaagcg | 61 |
| Encoding | gaaagtctctgaagtcgagtttagccacgagtattggatgaggcacgcac | |
| sequences for | tgaccctggcaaagcgagcatgggatgaaagagaagtccccgtgggcgcc | |
| NLS are bolded | gtgctggtgcacaacaatagagtgatcggagagggatggaacaggccaat | |
| and linkers are | cggccgccacgaccctaccgcacacgcagagatcatggcactgaggcagg | |
| underlined | gaggcctggtcatgcagaattaccgcctgatcgatgccaccctgtatgtg | |
| | acactggagccatgcgtgatgtgcgcaggagcaatgatccacagcaggat | |
| | cggaagagtggtgttcggagcacgggacgccaagaccggcgcagcaggct | |
| | ccctgatggatgtgctgcaccaccccggcatgaaccaccgggtggagatc | |
| | acagagggaatcctggcagacgagtgcgccgccctgctgagcgatttctt | |
| | tagaatgcggagacaggagatcaaggcccagaagaaggcacagagctcca | |
| | ccgactctggaggatctagcggaggatcctctggaagcgagacaccaggc | |
| | acaagcgagtccgccacaccagagagctccggcggctcctccggaggatc | |
| | ctctgaggtggagttttcccacgagtactggatgagacatgccctgaccc | |
| | tggccaagagggcacgcgatgagagggaggtgcctgtgggagccgtgctg | |
| | gtgctgaacaatagagtgatcggcgagggctggaacagagccatcggcct | |
| | gcacgacccaacagcccatgccgaaattatggccctgagacagggcggcc | |
| | tggtcatgcagaactacagactgattgacgccaccctgtacgtgacattc | |
| | gagccttgcgtgatgtgcgccggcgccatgatccactctaggatcggccg | |
| | cgtggtgtttggcgtgaggaacgcaaaaaccggcgccgcaggctccctga | |
| | tggacgtgctgcactaccccggcatgaatcaccgcgtcgaaattaccgag | |
| | ggaatcctggcagatgaatgtgccgccctgctgtgctatttctttcggat | |
| | gcctagacaggtgttcaatgctcagaagaaggcccagagctccaccgact | |
| | ccggaggatctagcggaggctcctctggctctgagacacctggcacaagc | |
| | gagagcgcaacacctgaaagcagcgggggcagcagcggggggtcagacaa | |
| | gaagtacagcatcggcctggccatcggcaccaactctgtgggctgggccg | |
| | tgatcaccgacgagtacaaggtgcccagcaagaaattcaaggtgctgggc | |
| | aacaccgaccggcacagcatcaagaagaacctgatcggagccctgctgtt | |
| | cgacagcggcgaaacagccgaggccacccggctgaagagaaccgccagaa | |
| | gaagatacaccagacggaagaaccggatctgctatctgcaagagatcttc | |
| | agcaacgagatggccaaggtggacgacagcttcttccacagactggaaga | |
| | gtccttcctggtggaagaggataagaagcacgagcggcaccccatcttcg | |
| | gcaacatcgtggacgaggtggcctaccacgagaagtaccccaccatctac | |
| | cacctgagaaagaaactggtggacagcaccgacaaggccgacctgcggct | |
| | gatctatctggccctggcccacatgatcaagttccggggccacttcctga | |
| | tcgagggcgacctgaaccccgacaacagcgacgtggacaagctgttcatc | |
| | cagctggtgcagacctacaaccagctgttcgaggaaaaccccatcaacgc | |
| | cagcggcgtggacgccaaggccatcctgtctgccagactgagcaagagca | |
| | gacggctggaaaatctgatcgcccagctgcccggcgagaagaagaatggc | |
| | ctgttcggaaacctgattgccctgagcctgggcctgacccccaacttcaa | |
| | gagcaacttcgacctggccgaggatgccaaactgcagctgagcaaggaca | |
| | cctacgacgacgacctggacaacctgctggcccagatcggcgaccagtac | |
| | gccgacctgtttctggccgccaagaacctgtccgacgccatcctgctgag | |
| | cgacatcctgagagtgaacaccgagatcaccaaggcccccctgagcgcct | |
| | ctatgatcaagagatacgacgagcaccaccaggacctgaccctgctgaaa | |
| | gctctcgtgcggcagcagctgcctgagaagtacaaagagattttcttcga | |
| | ccagagcaagaacggctacgccggctacattgacggcggagccagccagg | |
| | aagagttctacaagttcatcaagcccatcctggaaaagatggacggcacc | |
| | gaggaactgctcgtgaagctgaacagagaggacctgctgcggaagcagcg | |
| | gaccttcgacaacggcagcatcccccaccagatccacctgggagagctgc | |
| | acgccattctgcggcggcaggaagatttttacccattcctgaaggacaac | |
| | cgggaaaagatcgagaagatcctgaccttccgcatcccctactacgtggg | |
| | ccctctggccaggggaaacagcagattcgcctggatgaccagaaagagcg | |
| | aggaaaccatcaccccctggaacttcgaggaagtggtggacaagggcgct | |
| | tccgcccagagcttcatcgagcggatgaccaacttcgataagaacctgcc | |
| | caacgagaaggtgctgcccaagcacagcctgctgtacgagtacttcaccg | |
| | tgtataacgagctgaccaaagtgaaatacgtgaccgagggaatgagaaag | |
| | cccgccttcctgagcggcgagcagaaaaaggccatcgtggacctgctgtt | |
| | caagaccaaccggaaagtgaccgtgaagcagctgaaagaggactacttca | |
| | agaaaatcgagtgcttcgactccgtggaaatctccggcgtggaagatcgg | |
| | ttcaacgcctccctgggcacataccacgatctgctgaaaattatcaagga | |
| | caaggacttcctggacaatgaggaaaacgaggacattctggaagatatcg | |
| | tgctgaccctgacactgtttgaggacagagagatgatcgaggaacggctg | |
| | aaaacctatgcccacctgttcgacgacaaagtgatgaagcagctgaagcg | |
| | gcggagatacaccggctggggcaggctgagccggaagctgatcaacggca | |
| | tccgggacaagcagtccggcaagacaatcctggatttcctgaagtccgac | |
| | ggcttcgccaacagaaacttcatgcagctgatccacgacgacagcctgac | |
| | ctttaaagaggacatccagaaagcccaggtgtccggccagggcgatagcc | |
| | tgcacgagcacattgccaatctggccggcagccccgccattaagaagggc | |
| | atcctgcagacagtgaaggtggtggacgagctcgtgaaagtgatgggccg | |
| | gcacaagcccgagaacatcgtgatcgaaatggccagagagaaccagacca | |
| | cccagaagggacagaagaacagccgcgagagaatgaagcggatcgaagag | |
| | ggcatcaaagagctgggcagccagatcctgaaagaacaccccgtggaaaa | |
| | cacccagctgcagaacgagaagctgtacctgtactacctgcagaatgggg | |
| | ggatatgtacgtggaccaggaactggacatcaaccggctgtccgactacg | |
| | atgtggaccatatcgtgcctcagagctttctgaaggacgactccatcgac | |
| | aacaaggtgctgaccagaagcgacaagaaccggggcaagagcgacaacgt | |
| | gccctccgaagaggtcgtgaagaagatgaagaactactggcggcagctgc | |
| | tgaacgccaagctgattacccagagaaagttcgacaatctgaccaaggcc | |
| | gagagaggcggcctgagcgaactggataaggccggcttcatcaagagaca | |
| | gctggtggaaacccggcagatcacaaagcacgtggcacagatcctggact | |
| | cccggatgaacactaagtacgacgagaatgacaagctgatccgggaagtg | |
| | aaagtgatcaccctgaagtccaagctggtgtccgatttccggaaggattt | |
| | ccagttttacaaagtgcgcgagatcaacaactaccaccacgcccacgacg | |
| | cctacctgaacgccgtcgtgggaaccgccctgatcaaaaagtaccctaag | |
| | ctggaaagcgagttcgtgtacggcgactacaaggtgtacgacgtgcggaa | |
| | gatgatcgccaagagcgagcaggaaatcggcaaggctaccgccaagtact | |
| | tcttctacagcaacatcatgaactttttcaagaccgagattaccctggcc | |
| | aacggcgagatccggaagcggcctctgatcgagacaaacggcgaaaccgg | |
| | ggagatcgtgtgggataagggccgggattttgccaccgtgcggaaagtgc | |
| | tgagcatgccccaagtgaatatcgtgaaaaagaccgaggtgcagacaggc | |
| | ggcttcagcaaagagtctatcctgcccaagaggaacagcgataagctgat | |
| | cgccagaaagaaggactgggaccctaagaagtacggcggcttcgtgagcc | |
| | ccaccgtggcctattctgtgctggtggtggccaaagtggaaaagggcaag | |
| | tccaagaaactgaagagtgtgaaagagctgctggggatcaccatcatgga | |
| | aagaagcagcttcgagaagaatcccatcgactttctggaagccaagggct | |
| | acaaagaagtgaaaaaggacctgatcatcaagctgcctaagtactccctg | |
| | ttcgagctggaaaacggccggaagagaatgctggcctcagccagagaact | |
| | gcagaagggaaacgaactggccctgccctccaaatatgtgaacttcctgt | |
| | acctggccagccactatgagaagctgaagggctcccccgaggataatgag | |
| | cagaaacagctgtttgtggaacagcacaagcactacctggacgagatcat | |
| | cgagcagatcagcgagttctccaagagagtgatcctggccgacgctaatc | |
| | tggacaaagtgctgtccgcctacaacaagcaccgggataagcccatcaga | |
| | gagcaggccgagaatatcatccacctgtttaccctgaccaatctgggagc | |
| | ccctgccgccttcaagtactttgacaccaccatcgaccggaagcagtaca | |
| | gaagcaccaaagaggtgctggacgccaccctgatccaccagagcatcacc | |
| | ggcctgtacgagacacggatcgacctgtctcagctgggaggtgactctgg | |
| | cggctcaaaaagaaccgccgacggcagcgaattcgagcccaagaagaaga | |
| | ggaaagtc | |
| ABEmax-SpCas9- | atgaaacggacagccgacggaagcgagttcgagtcaccaaagaagaagcg | 62 |
| NG (DNA) | gaaagtctctgaagtcgagtttagccacgagtattggatgaggcacgcac | |
| Encoding | tgaccctggcaaagcgagcatgggatgaaagagaagtccccgtgggcgcc | |
| sequences for | gtgctggtgcacaacaatagagtgatcggagagggatggaacaggccaat | |
| NLS are bolded | cggccgccacgaccctaccgcacacgcagagatcatggcactgaggcagg | |
| and linkers are | gaggcctggtcatgcagaattaccgcctgatcgatgccaccctgtatgtg | |
| underlined | acactggagccatgcgtgatgtgcgcaggagcaatgatccacagcaggat | |
| | cggaagagtggtgttcggagcacgggacgccaagaccggcgcagcaggct | |
| | ccctgatggatgtgctgcaccaccccggcatgaaccaccgggtggagatc | |
| | acagagggaatcctggcagacgagtgcgccgccctgctgagcgatttctt | |
| | tagaatgcggagacaggagatcaaggcccagaagaaggcacagagctcca | |
| | ccgactctggaggatctagcggaggatcctctggaagcgagacaccaggc | |
| | acaagcgagtccgccacaccagagagctccggcggctcctccggaggatc | |
| | ctctgaggtggagttttcccacgagtactggatgagacatgccctgaccc | |
| | tggccaagagggcacgcgatgagagggaggtgcctgtgggagccgtgctg | |
| | gtgctgaacaatagagtgatcggcgagggctggaacagagccatcggcct | |
| | gcacgacccaacagcccatgccgaaattatggccctgagacagggcggcc | |
| | tggtcatgcagaactacagactgattgacgccaccctgtacgtgacattc | |
| | gagccttgcgtgatgtgcgccggcgccatgatccactctaggatcggccg | |
| | cgtggtgtttggcgtgaggaacgcaaaaaccggcgccgcaggctccctga | |
| | tggacgtgctgcactaccccggcatgaatcaccgcgtcgaaattaccgag | |
| | ggaatcctggcagatgaatgtgccgccctgctgtgctatttctttcggat | |
| | gcctagacaggtgttcaatgctcagaagaaggcccagagctccaccgact | |
| | ccggaggatctagcggaggctcctctggctctgagacacctggcacaagc | |
| | gagagcgcaacacctgaaagcagcgggggcagcagcggggggtcagacaa | |
| | gaagtacagcatcggcctggccatcggcaccaactctgtgggctgggccg | |
| | tgatcaccgacgagtacaaggtgcccagcaagaaattcaaggtgctgggc | |
| | aacaccgaccggcacagcatcaagaagaacctgatcggagccctgctgtt | |
| | cgacagcggcgaaacagccgaggccacccggctgaagagaaccgccagaa | |
| | gaagatacaccagacggaagaaccggatctgctatctgcaagagatcttc | |
| | agcaacgagatggccaaggtggacgacagcttcttccacagactggaaga | |
| | gtccttcctggtggaagaggataagaagcacgagcggcaccccatcttcg | |
| | gcaacatcgtggacgaggtggcctaccacgagaagtaccccaccatctac | |
| | cacctgagaaagaaactggtggacagcaccgacaaggccgacctgcggct | |
| | gatctatctggccctggcccacatgatcaagttccggggccacttcctga | |
| | tcgagggcgacctgaaccccgacaacagcgacgtggacaagctgttcatc | |
| | cagctggtgcagacctacaaccagctgttcgaggaaaaccccatcaacgc | |
| | cagcggcgtggacgccaaggccatcctgtctgccagactgagcaagagca | |
| | gacggctggaaaatctgatcgcccagctgcccggcgagaagaagaatggc | |
| | ctgttcggaaacctgattgccctgagcctgggcctgacccccaacttcaa | |
| | gagcaacttcgacctggccgaggatgccaaactgcagctgagcaaggaca | |
| | cctacgacgacgacctggacaacctgctggcccagatcggcgaccagtac | |
| | gccgacctgtttctggccgccaagaacctgtccgacgccatcctgctgag | |
| | cgacatcctgagagtgaacaccgagatcaccaaggcccccctgagcgcct | |
| | ctatgatcaagagatacgacgagcaccaccaggacctgaccctgctgaaa | |
| | gctctcgtgcggcagcagctgcctgagaagtacaaagagattttcttcga | |
| | ccagagcaagaacggctacgccggctacattgacggcggagccagccagg | |
| | aagagttctacaagttcatcaagcccatcctggaaaagatggacggcacc | |
| | gaggaactgctcgtgaagctgaacagagaggacctgctgcggaagcagcg | |
| | gaccttcgacaacggcagcatcccccaccagatccacctgggagagctgc | |
| | acgccattctgcggcggcaggaagatttttacccattcctgaaggacaac | |
| | cgggaaaagatcgagaagatcctgaccttccgcatcccctactacgtggg | |
| | ccctctggccaggggaaacagcagattcgcctggatgaccagaaagagcg | |
| | aggaaaccatcaccccctggaacttcgaggaagtggtggacaagggcgct | |
| | tccgcccagagcttcatcgagcggatgaccaacttcgataagaacctgcc | |
| | caacgagaaggtgctgcccaagcacagcctgctgtacgagtacttcaccg | |
| | tgtataacgagctgaccaaagtgaaatacgtgaccgagggaatgagaaag | |
| | cccgccttcctgagcggcgagcagaaaaaggccatcgtggacctgctgtt | |
| | caagaccaaccggaaagtgaccgtgaagcagctgaaagaggactacttca | |
| | agaaaatcgagtgcttcgactccgtggaaatctccggcgtggaagatcgg | |
| | ttcaacgcctccctgggcacataccacgatctgctgaaaattatcaagga | |
| | caaggacttcctggacaatgaggaaaacgaggacattctggaagatatcg | |
| | tgctgaccctgacactgtttgaggacagagagatgatcgaggaacggctg | |
| | aaaacctatgcccacctgttcgacgacaaagtgatgaagcagctgaagcg | |
| | gcggagatacaccggctggggcaggctgagccggaagctgatcaacggca | |
| | tccgggacaagcagtccggcaagacaatcctggatttcctgaagtccgac | |
| | ggcttcgccaacagaaacttcatgcagctgatccacgacgacagcctgac | |
| | ctttaaagaggacatccagaaagcccaggtgtccggccagggcgatagcc | |
| | tgcacgagcacattgccaatctggccggcagccccgccattaagaagggc | |
| | atcctgcagacagtgaaggtggtggacgagctcgtgaaagtgatgggccg | |
| | gcacaagcccgagaacatcgtgatcgaaatggccagagagaaccagacca | |
| | cccagaagggacagaagaacagccgcgagagaatgaagcggatcgaagag | |
| | ggcatcaaagagctgggcagccagatcctgaaagaacaccccgtggaaaa | |
| | cacccagctgcagaacgagaagctgtacctgtactacctgcagaatgggc | |
| | gggatatgtacgtggaccaggaactggacatcaaccggctgtccgactac | |
| | gatgtggaccatatcgtgcctcagagctttctgaaggacgactccatcga | |
| | caacaaggtgctgaccagaagcgacaagaaccggggcaagagcgacaacg | |
| | tgccctccgaagaggtcgtgaagaagatgaagaactactggcggcagctg | |
| | ctgaacgccaagctgattacccagagaaagttcgacaatctgaccaaggc | |
| | cgagagaggcggcctgagcgaactggataaggccggcttcatcaagagac | |
| | agctggtggaaacccggcagatcacaaagcacgtggcacagatcctggac | |
| | tcccggatgaacactaagtacgacgagaatgacaagctgatccgggaagt | |
| | gaaagtgatcaccctgaagtccaagctggtgtccgatttccggaaggatt | |
| | tccagttttacaaagtgcgcgagatcaacaactaccaccacgcccacgac | |
| | gcctacctgaacgccgtcgtgggaaccgccctgatcaaaaagtaccctaa | |
| | gctggaaagcgagttcgtgtacggcgactacaaggtgtacgacgtgcgga | |
| | agatgatcgccaagagcgagcaggaaatcggcaaggctaccgccaagtac | |
| | ttcttctacagcaacatcatgaactttttcaagaccgagattaccctggc | |
| | caacggcgagatccggaagcggcctctgatcgagacaaacggcgaaaccg | |
| | gggagatcgtgtgggataagggccgggattttgccaccgtgcggaaagtg | |
| | ctgagcatgccccaagtgaatatcgtgaaaaagaccgaggtgcagacagg | |
| | cggcttcagcaaagagtctatcaggcccaagaggaacagcgataagctga | |
| | tcgccagaaagaaggactgggaccctaagaagtacggcggcttcgtcagc | |
| | cccaccgtggcctattctgtgctggtggtggccaaagtggaaaagggcaa | |
| | gtccaagaaactgaagagtgtgaaagagctgctggggatcaccatcatgg | |
| | aaagaagcagcttcgagaagaatcccatcgactttctggaagccaagggc | |
| | tacaaagaagtgaaaaaggacctgatcatcaagctgcctaagtactccct | |
| | gttcgagctggaaaacggccggaagagaatgctggcctctgccagattcc | |
| | tgcagaagggaaacgaactggccctgccctccaaatatgtgaacttcctg | |
| | tacctggccagccactatgagaagctgaagggctcccccgaggataatga | |
| | gcagaaacagctgtttgtggaacagcacaagcactacctggacgagatca | |
| | tcgagcagatcagcgagttctccaagagagtgatcctggccgacgctaat | |
| | ctggacaaagtgctgtccgcctacaacaagcaccgggataagcccatcag | |
| | agagcaggccgagaatatcatccacctgtttaccctgaccaatctgggag | |
| | cccctagggccttcaagtactttgacaccaccatcgaccggaaggtgtac | |
| | aggagcaccaaagaggtgctggacgccaccctgatccaccagagcatcac | |
| | cggcctgtacgagacacggatcgacctgtctcagctgggaggtgactctg | |
| | gcggctcaaaaagaaccgccgacggcagcgaattcgagcccaagaagaag | |
| | aggaaagtc | |
| ABEmax-SpRY | atgaaacggacagccgacggaagcgagttcgagtcaccaaagaagaagcg | 63 |
| Encoding | gaaagtctctgaagtcgagtttagccacgagtattggatgaggcacgcac | |
| sequences for | tgaccctggcaaagcgagcatgggatgaaagagaagtccccgtgggcgcc | |
| NLS are bolded | gtgctggtgcacaacaatagagtgatcggagagggatggaacaggccaat | |
| and linkers are | cggccgccacgaccctaccgcacacgcagagatcatggcactgaggcagg | |
| underlined | gaggcctggtcatgcagaattaccgcctgatcgatgccaccctgtatgtg | |
| | acactggagccatgcgtgatgtgcgcaggagcaatgatccacagcaggat | |
| | cggaagagtggtgttcggagcacgggacgccaagaccggcgcagcaggct | |
| | ccctgatggatgtgctgcaccaccccggcatgaaccaccgggtggagatc | |
| | acagagggaatcctggcagacgagtgcgccgccctgctgagcgatttctt | |
| | tagaatgcggagacaggagatcaaggcccagaagaaggcacagagctcca | |
| | ccgactctggaggatctagcggaggatcctctggaagcgagacaccaggc | |
| | acaagcgagtccgccacaccagagagctccggcggctcctccggaggatc | |
| | ctctgaggtggagttttcccacgagtactggatgagacatgccctgaccc | |
| | tggccaagagggcacgcgatgagagggaggtgcctgtgggagccgtgctg | |
| | gtgctgaacaatagagtgatcggcgagggctggaacagagccatcggcct | |
| | gcacgacccaacagcccatgccgaaattatggccctgagacagggcggcc | |
| | tggtcatgcagaactacagactgattgacgccaccctgtacgtgacattc | |
| | gagccttgcgtgatgtgcgccggcgccatgatccactctaggatcggccg | |
| | cgtggtgtttggcgtgaggaacgcaaaaaccggcgccgcaggctccctga | |
| | tggacgtgctgcactaccccggcatgaatcaccgcgtcgaaattaccgag | |
| | ggaatcctggcagatgaatgtgccgccctgctgtgctatttctttcggat | |
| | gcctagacaggtgttcaatgctcagaagaaggcccagagctccaccgact | |
| | ccggaggatctagcggaggctcctctggctctgagacacctggcacaagc | |
| | gagagcgcaacacctgaaagcagcgggggcagcagcggggggtcaatgga | |
| | caagaagtacagcatcggcctggccatcggcaccaactctgtgggctggg | |
| | ccgtgatcaccgacgagtacaaggtgcccagcaagaaattcaaggtgctg | |
| | ggcaacaccgaccggcacagcatcaagaagaacctgatcggagccctgct | |
| | gttcgacagcggcgaaacagccgagagaacccggctgaagagaaccgcca | |
| | gaagaagatacaccagacggaagaaccggatctgctatctgcaagagatc | |
| | ttcagcaacgagatggccaaggtggacgacagcttcttccacagactgga | |
| | agagtccttcctggtggaagaggataagaagcacgagcggcaccccatct | |
| | tcggcaacatcgtggacgaggtggcctaccacgagaagtaccccaccatc | |
| | taccacctgagaaagaaactggtggacagcaccgacaaggccgacctgcg | |
| | gctgatctatctggccctggcccacatgatcaagttccggggccacttcc | |
| | tgatcgagggcgacctgaaccccgacaacagcgacgtggacaagctgttc | |
| | atccagctggtgcagacctacaaccagctgttcgaggaaaaccccatcaa | |
| | cgccagcggcgtggacgccaaggccatcctgtctgccagactgagcaaga | |
| | gcagacggctggaaaatctgatcgcccagctgcccggcgagaagaagaat | |
| | ggcctgttcggaaacctgattgccctgagcctgggcctgacccccaactt | |
| | caagagcaacttcgacctggccgaggatgccaaactgcagctgagcaagg | |
| | acacctacgacgacgacctggacaacctgctggcccagatcggcgaccag | |
| | tacgccgacctgtttctggccgccaagaacctgtccgacgccatcctgct | |
| | gagcgacatcctgagagtgaacaccgagatcaccaaggcccccctgagcg | |
| | cctctatgatcaagagatacgacgagcaccaccaggacctgaccctgctg | |
| | aaagctctcgtgcggcagcagctgcctgagaagtacaaagagattttctt | |
| | cgaccagagcaagaacggctacgccggctacattgacggcggagccagcc | |
| | aggaagagttctacaagttcatcaagcccatcctggaaaagatggacggc | |
| | accgaggaactgctcgtgaagctgaacagagaggacctgctgcggaagca | |
| | gcggaccttcgacaacggcagcatcccccaccagatccacctgggagagc | |
| | tgcacgccattctgcggcggcaggaagatttttacccattcctgaaggac | |
| | aaccgggaaaagatcgagaagatcctgaccttccgcatcccctactacgt | |
| | gggccctctggccaggggaaacagcagattcgcctggatgaccagaaaga | |
| | gcgaggaaaccatcaccccctggaacttcgaggaagtggtggacaagggc | |
| | gcttccgcccagagcttcatcgagcggatgaccaacttcgataagaacct | |
| | gcccaacgagaaggtgctgcccaagcacagcctgctgtacgagtacttca | |
| | ccgtgtataacgagctgaccaaagtgaaatacgtgaccgagggaatgaga | |
| | aagcccgccttcctgagcggcgagcagaaaaaggccatcgtggacctgct | |
| | gttcaagaccaaccggaaagtgaccgtgaagcagctgaaagaggactact | |
| | tcaagaaaatcgagtgcttcgactccgtggaaatctccggcgtggaagat | |
| | cggttcaacgcctccctgggcacataccacgatctgctgaaaattatcaa | |
| | ggacaaggacttcctggacaatgaggaaaacgaggacattctggaagata | |
| | tcgtgctgaccctgacactgtttgaggacagagagatgatcgaggaacgg | |
| | ctgaaaacctatgcccacctgttcgacgacaaagtgatgaagcagctgaa | |
| | gcggcggagatacaccggctggggcaggctgagccggaagctgatcaacg | |
| | gcatccgggacaagcagtccggcaagacaatcctggatttcctgaagtcc | |
| | gacggcttcgccaacagaaacttcatgcagctgatccacgacgacagcct | |
| | gacctttaaagaggacatccagaaagcccaggtgtccggccagggcgata | |
| | gcctgcacgagcacattgccaatctggccggcagccccgccattaagaag | |
| | ggcatcctgcagacagtgaaggtggtggacgagctcgtgaaagtgatggg | |
| | ccggcacaagcccgagaacatcgtgatcgaaatggccagagagaaccaga | |
| | ccacccagaagggacagaagaacagccgcgagagaatgaagcggatcgaa | |
| | gagggcatcaaagagctgggcagccagatcctgaaagaacaccccgtgga | |
| | aaacacccagctgcagaacgagaagctgtacctgtactacctgcagaatg | |
| | ggcgggatatgtacgtggaccaggaactggacatcaaccggctgtccgac | |
| | tacgatgtggaccatatcgtgcctcagagctttctgaaggacgactccat | |
| | cgacaacaaggtgctgaccagaagcgacaagaaccggggcaagagcgaca | |
| | acgtgccctccgaagaggtcgtgaagaagatgaagaactactggcggcag | |
| | ctgctgaacgccaagctgattacccagagaaagttcgacaatctgaccaa | |
| | ggccgagagaggcggcctgagcgaactggataaggccggcttcatcaaga | |
| | gacagctggtggaaacccggcagatcacaaagcacgtggcacagatcctg | |
| | gactcccggatgaacactaagtacgacgagaatgacaagctgatccggga | |
| | agtgaaagtgatcaccctgaagtccaagctggtgtccgatttccggaagg | |
| | atttccagttttacaaagtgcgcgagatcaacaactaccaccacgcccac | |
| | gacgcctacctgaacgccgtcgtgggaaccgccctgatcaaaaagtaccc | |
| | taagctggaaagcgagttcgtgtacggcgactacaaggtgtacgacgtgc | |
| | ggaagatgatcgccaagagcgagcaggaaatcggcaaggctaccgccaag | |
| | tacttcttctacagcaacatcatgaactttttcaagaccgagattaccct | |
| | ggccaacggcgagatccggaagcggcctctgatcgagacaaacggcgaaa | |
| | ccggggagatcgtgtgggataagggccgggattttgccaccgtgcggaaa | |
| | gtgctgagcatgccccaagtgaatatcgtgaaaaagaccgaggtgcagac | |
| | aggcggcttcagcaaagagtctatcagacccaagaggaacagcgataagc | |
| | tgatcgccagaaagaaggactgggaccctaagaagtacggcggcttcctg | |
| | tggcccaccgtggcctattctgtgctggtggtggccaaagtggaaaaggg | |
| | caagtccaagaaactgaagagtgtgaaagagctgctggggatcaccatca | |
| | tggaaagaagcagcttcgagaagaatcccatcgactttctggaagccaag | |
| | ggctacaaagaagtgaaaaaggacctgatcatcaagctgcctaagtactc | |
| | cctgttcgagctggaaaacggccggaagagaatgctggcctctgccaagc | |
| | agctgcagaagggaaacgaactggccctgccctccaaatatgtgaacttc | |
| | ctgtacctggccagccactatgagaagctgaagggctcccccgaggataa | |
| | tgagcagaaacagctgtttgtggaacagcacaagcactacctggacgaga | |
| | tcatcgagcagatcagcgagttctccaagagagtgatcctggccgacgct | |
| | aatctggacaaagtgctgtccgcctacaacaagcaccgggataagcccat | |
| | cagagagcaggccgagaatatcatccacctgtttaccctgaccagactgg | |
| | gagcccctagagccttcaagtactttgacaccaccatcgaccccaagcag | |
| | tacagaagcaccaaagaggtgctggacgccaccctgatccaccagagcat | |
| | caccggcctgtacgagacacggatcgacctgtctcagctgggaggtgact | |
| | ctggcggctcaaaaagaaccgccgacggcagcgaattcgagcccaagaag | |
| | aagaggaaagtc | |
| ABEmax-SpG | atgaaacggacagccgacggaagcgagttcgagtcaccaaagaagaagcg | 64 |
| Encoding | gaaagtctctgaagtcgagtttagccacgagtattggatgaggcacgcac | |
| sequences for | tgaccctggcaaagcgagcatgggatgaaagagaagtccccgtgggcgcc | |
| NLS are bolded | gtgctggtgcacaacaatagagtgatcggagagggatggaacaggccaat | |
| and linkers are | cggccgccacgaccctaccgcacacgcagagatcatggcactgaggcagg | |
| underlined | gaggcctggtcatgcagaattaccgcctgatcgatgccaccctgtatgtg | |
| | acactggagccatgcgtgatgtgcgcaggagcaatgatccacagcaggat | |
| | cggaagagtggtgttcggagcacgggacgccaagaccggcgcagcaggct | |
| | ccctgatggatgtgctgcaccaccccggcatgaaccaccgggtggagatc | |
| | acagagggaatcctggcagacgagtgcgccgccctgctgagcgatttctt | |
| | tagaatgcggagacaggagatcaaggcccagaagaaggcacagagctcca | |
| | ccgactctggaggatctagcggaggatcctctggaagcgagacaccaggc | |
| | acaagcgagtccgccacaccagagagctccggcggctcctccggaggatc | |
| | ctctgaggtggagttttcccacgagtactggatgagacatgccctgaccc | |
| | tggccaagagggcacgcgatgagagggaggtgcctgtgggagccgtgctg | |
| | gtgctgaacaatagagtgatcggcgagggctggaacagagccatcggcct | |
| | gcacgacccaacagcccatgccgaaattatggccctgagacagggcggcc | |
| | tggtcatgcagaactacagactgattgacgccaccctgtacgtgacattc | |
| | gagccttgcgtgatgtgcgccggcgccatgatccactctaggatcggccg | |
| | cgtggtgtttggcgtgaggaacgcaaaaaccggcgccgcaggctccctga | |
| | tggacgtgctgcactaccccggcatgaatcaccgcgtcgaaattaccgag | |
| | ggaatcctggcagatgaatgtgccgccctgctgtgctatttctttcggat | |
| | gcctagacaggtgttcaatgctcagaagaaggcccagagctccaccgact | |
| | ccggaggatctagcggaggctcctctggctctgagacacctggcacaagc | |
| | gagagcgcaacacctgaaagcagcgggggcagcagcggggggtcagacaa | |
| | gaagtacagcatcggcctggccatcggcaccaactctgtgggctgggccg | |
| | tgatcaccgacgagtacaaggtgcccagcaagaaattcaaggtgctgggc | |
| | aacaccgaccggcacagcatcaagaagaacctgatcggagccctgctgtt | |
| | cgacagcggcgaaacagccgaggccacccggctgaagagaaccgccagaa | |
| | gaagatacaccagacggaagaaccggatctgctatctgcaagagatcttc | |
| | agcaacgagatggccaaggtggacgacagcttcttccacagactggaaga | |
| | gtccttcctggtggaagaggataagaagcacgagcggcaccccatcttcg | |
| | gcaacatcgtggacgaggtggcctaccacgagaagtaccccaccatctac | |
| | cacctgagaaagaaactggtggacagcaccgacaaggccgacctgcggct | |
| | gatctatctggccctggcccacatgatcaagttccggggccacttcctga | |
| | tcgagggcgacctgaaccccgacaacagcgacgtggacaagctgttcatc | |
| | cagctggtgcagacctacaaccagctgttcgaggaaaaccccatcaacgc | |
| | cagcggcgtggacgccaaggccatcctgtctgccagactgagcaagagca | |
| | gacggctggaaaatctgatcgcccagctgcccggcgagaagaagaatggc | |
| | ctgttcggaaacctgattgccctgagcctgggcctgacccccaacttcaa | |
| | gagcaacttcgacctggccgaggatgccaaactgcagctgagcaaggaca | |
| | cctacgacgacgacctggacaacctgctggcccagatcggcgaccagtac | |
| | gccgacctgtttctggccgccaagaacctgtccgacgccatcctgctgag | |
| | cgacatcctgagagtgaacaccgagatcaccaaggcccccctgagcgcct | |
| | ctatgatcaagagatacgacgagcaccaccaggacctgaccctgctgaaa | |
| | gctctcgtgcggcagcagctgcctgagaagtacaaagagattttcttcga | |
| | ccagagcaagaacggctacgccggctacattgacggcggagccagccagg | |
| | aagagttctacaagttcatcaagcccatcctggaaaagatggacggcacc | |
| | gaggaactgctcgtgaagctgaacagagaggacctgctgcggaagcagcg | |
| | gaccttcgacaacggcagcatcccccaccagatccacctgggagagctgc | |
| | acgccattctgcggcggcaggaagatttttacccattcctgaaggacaac | |
| | cgggaaaagatcgagaagatcctgaccttccgcatcccctactacgtggg | |
| | ccctctggccaggggaaacagcagattcgcctggatgaccagaaagagcg | |
| | aggaaaccatcaccccctggaacttcgaggaagtggtggacaagggcgct | |
| | tccgcccagagcttcatcgagcggatgaccaacttcgataagaacctgcc | |
| | caacgagaaggtgctgcccaagcacagcctgctgtacgagtacttcaccg | |
| | tgtataacgagctgaccaaagtgaaatacgtgaccgagggaatgagaaag | |
| | cccgccttcctgagcggcgagcagaaaaaggccatcgtggacctgctgtt | |
| | caagaccaaccggaaagtgaccgtgaagcagctgaaagaggactacttca | |
| | agaaaatcgagtgcttcgactccgtggaaatctccggcgtggaagatcgg | |
| | ttcaacgcctccctgggcacataccacgatctgctgaaaattatcaagga | |
| | caaggacttcctggacaatgaggaaaacgaggacattctggaagatatcg | |
| | tgctgaccctgacactgtttgaggacagagagatgatcgaggaacggctg | |
| | aaaacctatgcccacctgttcgacgacaaagtgatgaagcagctgaagcg | |
| | gcggagatacaccggctggggcaggctgagccggaagctgatcaacggca | |
| | tccgggacaagcagtccggcaagacaatcctggatttcctgaagtccgac | |
| | ggcttcgccaacagaaacttcatgcagctgatccacgacgacagcctgac | |
| | ctttaaagaggacatccagaaagcccaggtgtccggccagggcgatagcc | |
| | tgcacgagcacattgccaatctggccggcagccccgccattaagaagggc | |
| | atcctgcagacagtgaaggtggtggacgagctcgtgaaagtgatgggccg | |
| | gcacaagcccgagaacatcgtgatcgaaatggccagagagaaccagacca | |
| | cccagaagggacagaagaacagccgcgagagaatgaagcggatcgaagag | |
| | ggcatcaaagagctgggcagccagatcctgaaagaacaccccgtggaaaa | |
| | cacccagctgcagaacgagaagctgtacctgtactacctgcagaatgggc | |
| | gggatatgtacgtggaccaggaactggacatcaaccggctgtccgactac | |
| | gatgtggaccatatcgtgcctcagagctttctgaaggacgactccatcga | |
| | caacaaggtgctgaccagaagcgacaagaaccggggcaagagcgacaacg | |
| | tgccctccgaagaggtcgtgaagaagatgaagaactactggcggcagctg | |
| | ctgaacgccaagctgattacccagagaaagttcgacaatctgaccaaggc | |
| | cgagagaggcggcctgagcgaactggataaggccggcttcatcaagagac | |
| | agctggtggaaacccggcagatcacaaagcacgtggcacagatcctggac | |
| | tcccggatgaacactaagtacgacgagaatgacaagctgatccgggaagt | |
| | gaaagtgatcaccctgaagtccaagctggtgtccgatttccggaaggatt | |
| | tccagttttacaaagtgcgcgagatcaacaactaccaccacgcccacgac | |
| | gcctacctgaacgccgtcgtgggaaccgccctgatcaaaaagtaccctaa | |
| | gctggaaagcgagttcgtgtacggcgactacaaggtgtacgacgtgcgga | |
| | agatgatcgccaagagcgagcaggaaatcggcaaggctaccgccaagtac | |
| | ttcttctacagcaacatcatgaactttttcaagaccgagattaccctggc | |
| | caacggcgagatccggaagcggcctctgatcgagacaaacggcgaaaccg | |
| | gggagatcgtgtgggataagggccgggattttgccaccgtgcggaaagtg | |
| | ctgagcatgccccaagtgaatatcgtgaaaaagaccgaggtgcagacagg | |
| | cggcttcagcaaagagtctatcctgcccaagaggaacagcgataagctga | |
| | tcgccagaaagaaggactgggaccctaagaagtacggcggcttcctgtgg | |
| | cccaccgtggcctattctgtgctggtggtggccaaagtggaaaagggcaa | |
| | gtccaagaaactgaagagtgtgaaagagctgctggggatcaccatcatgg | |
| | aaagaagcagcttcgagaagaatcccatcgactttctggaagccaagggc | |
| | tacaaagaagtgaaaaaggacctgatcatcaagctgcctaagtactccct | |
| | gttcgagctggaaaacggccggaagagaatgctggcctctgccaagcagc | |
| | tgcagaagggaaacgaactggccctgccctccaaatatgtgaacttcctg | |
| | tacctggccagccactatgagaagctgaagggctcccccgaggataatga | |
| | gcagaaacagctgtttgtggaacagcacaagcactacctggacgagatca | |
| | tcgagcagatcagcgagttctccaagagagtgatcctggccgacgctaat | |
| | ctggacaaagtgctgtccgcctacaacaagcaccgggataagcccatcag | |
| | agagcaggccgagaatatcatccacctgtttaccctgaccaatctgggag | |
| | cccctgccgccttcaagtactttgacaccaccatcgaccggaagcagtac | |
| | agaagcaccaaagaggtgctggacgccaccctgatccaccagagcatcac | |
| | cggcctgtacgagacacggatcgacctgtctcagctgggaggtgactctg | |
| | gcggctcaaaaagaaccgccgacggcagcgaattcgagcccaagaagaag | |
| | aggaaagtc | |
| ABE8e-VRQR | Atgaaacggacagccgacggaagcgagttcgagtcaccaaagaagaagcg | 65 |
| Encoding | gaaagtctctgaggtggagttttcccacgagtactggatgagacatgccc | |
| sequences for | tgaccctggccaagagggcacgggatgagagggaggtgcctgtgggagcc | |
| NLS are bolded | gtgctggtgctgaacaatagagtgatcggcgagggctggaacagagccat | |
| and linkers are | cggcctgcacgacccaacagcccatgccgaaattatggccctgagacagg | |
| underlined | gcggcctggtcatgcagaactacagactgattgacgccaccctgtacgtg | |
| | acattcgagccttgcgtgatgtgcgccggcgccatgatccactctaggat | |
| | cggccgcgtggtgtttggcgtgaggaactcaaaaagaggcgccgcaggct | |
| | ccctgatgaacgtgctgaactaccccggcatgaatcaccgcgtcgaaatt | |
| | accgagggaatcctggcagatgaatgtgccgccctgctgtgcgatttcta | |
| | tcggatgcctagacaggtgttcaatgctcagaagaaggcccagagctcca | |
| | tcaactccggaggatctagcggaggctcctctggctctgagacacctggc | |
| | acaagcgagagcgcaacacctgaaagcagcgggggcagcagcggggggtc | |
| | agacaagaagtacagcatcggcctggccatcggcaccaactctgtgggct | |
| | gggccgtgatcaccgacgagtacaaggtgcccagcaagaaattcaaggtg | |
| | ctgggcaacaccgaccggcacagcatcaagaagaacctgatcggagccct | |
| | gctgttcgacagcggcgaaacagccgaggccacccggctgaagagaaccg | |
| | ccagaagaagatacaccagacggaagaaccggatctgctatctgcaagag | |
| | atcttcagcaacgagatggccaaggtggacgacagcttcttccacagact | |
| | ggaagagtccttcctggtggaagaggataagaagcacgagcggcacccca | |
| | tcttcggcaacatcgtggacgaggtggcctaccacgagaagtaccccacc | |
| | atctaccacctgagaaagaaactggtggacagcaccgacaaggccgacct | |
| | gcggctgatctatctggccctggcccacatgatcaagttccggggccact | |
| | tcctgatcgagggcgacctgaaccccgacaacagcgacgtggacaagctg | |
| | ttcatccagctggtgcagacctacaaccagctgttcgaggaaaaccccat | |
| | caacgccagcggcgtggacgccaaggccatcctgtctgccagactgagca | |
| | agagcagacggctggaaaatctgatcgcccagctgcccggcgagaagaag | |
| | aatggcctgttcggaaacctgattgccctgagcctgggcctgacccccaa | |
| | cttcaagagcaacttcgacctggccgaggatgccaaactgcagctgagca | |
| | aggacacctacgacgacgacctggacaacctgctggcccagatcggcgac | |
| | cagtacgccgacctgtttctggccgccaagaacctgtccgacgccatcct | |
| | gctgagcgacatcctgagagtgaacaccgagatcaccaaggcccccctga | |
| | gcgcctctatgatcaagagatacgacgagcaccaccaggacctgaccctg | |
| | ctgaaagctctcgtgcggcagcagctgcctgagaagtacaaagagatttt | |
| | cttcgaccagagcaagaacggctacgccggctacattgacggcggagcca | |
| | gccaggaagagttctacaagttcatcaagcccatcctggaaaagatggac | |
| | ggcaccgaggaactgctcgtgaagctgaacagagaggacctgctgcggaa | |
| | gcagcggaccttcgacaacggcagcatcccccaccagatccacctgggag | |
| | agctgcacgccattctgcggcggcaggaagatttttacccattcctgaag | |
| | gacaaccgggaaaagatcgagaagatcctgaccttccgcatcccctacta | |
| | cgtgggccctctggccaggggaaacagcagattcgcctggatgaccagaa | |
| | agagcgaggaaaccatcaccccctggaacttcgaggaagtggtggacaag | |
| | ggcgcttccgcccagagcttcatcgagcggatgaccaacttcgataagaa | |
| | cctgcccaacgagaaggtgctgcccaagcacagcctgctgtacgagtact | |
| | tcaccgtgtataacgagctgaccaaagtgaaatacgtgaccgagggaatg | |
| | agaaagcccgccttcctgagcggcgagcagaaaaaggccatcgtggacct | |
| | gctgttcaagaccaaccggaaagtgaccgtgaagcagctgaaagaggact | |
| | acttcaagaaaatcgagtgcttcgactccgtggaaatctccggcgtggaa | |
| | gatcggttcaacgcctccctgggcacataccacgatctgctgaaaattat | |
| | caaggacaaggacttcctggacaatgaggaaaacgaggacattctggaag | |
| | atatcgtgctgaccctgacactgtttgaggacagagagatgatcgaggaa | |
| | cggctgaaaacctatgcccacctgttcgacgacaaagtgatgaagcagct | |
| | gaagcggcggagatacaccggctggggcaggctgagccggaagctgatca | |
| | acggcatccgggacaagcagtccggcaagacaatcctggatttcctgaag | |
| | tccgacggcttcgccaacagaaacttcatgcagctgatccacgacgacag | |
| | cctgacctttaaagaggacatccagaaagcccaggtgtccggccagggcg | |
| | atagcctgcacgagcacattgccaatctggccggcagccccgccattaag | |
| | aagggcatcctgcagacagtgaaggtggtggacgagctcgtgaaagtgat | |
| | gggccggcacaagcccgagaacatcgtgatcgaaatggccagagagaacc | |
| | agaccacccagaagggacagaagaacagccgcgagagaatgaagcggatc | |
| | gaagagggcatcaaagagctgggcagccagatcctgaaagaacaccccgt | |
| | ggaaaacacccagctgcagaacgagaagctgtacctgtactacctgcaga | |
| | atgggcgggatatgtacgtggaccaggaactggacatcaaccggctgtcc | |
| | gactacgatgtggaccatatcgtgcctcagagctttctgaaggacgactc | |
| | catcgacaacaaggtgctgaccagaagcgacaagaaccggggcaagagcg | |
| | acaacgtgccctccgaagaggtcgtgaagaagatgaagaactactggcgg | |
| | cagctgctgaacgccaagctgattacccagagaaagttcgacaatctgac | |
| | caaggccgagagaggcggcctgagcgaactggataaggccggcttcatca | |
| | agagacagctggtggaaacccggcagatcacaaagcacgtggcacagatc | |
| | ctggactcccggatgaacactaagtacgacgagaatgacaagctgatccg | |
| | ggaagtgaaagtgatcaccctgaagtccaagctggtgtccgatttccgga | |
| | aggatttccagttttacaaagtgcgcgagatcaacaactaccaccacgcc | |
| | cacgacgcctacctgaacgccgtcgtgggaaccgccctgatcaaaaagta | |
| | ccctaagctggaaagcgagttcgtgtacggcgactacaaggtgtacgacg | |
| | tgcggaagatgatcgccaagagcgagcaggaaatcggcaaggctaccgcc | |
| | aagtacttcttctacagcaacatcatgaactttttcaagaccgagattac | |
| | cctggccaacggcgagatccggaagcggcctctgatcgagacaaacggcg | |
| | aaaccggggagatcgtgtgggataagggccgggattttgccaccgtgcgg | |
| | aaagtgctgagcatgccccaagtgaatatcgtgaaaaagaccgaggtgca | |
| | gacaggcggcttcagcaaagagtctatcctgcccaagaggaacagcgata | |
| | agctgatcgccagaaagaaggactgggaccctaagaagtacggcggcttc | |
| | gtgagccccaccgtggcctattctgtgctggtggtggccaaagtggaaaa | |
| | gggcaagtccaagaaactgaagagtgtgaaagagctgctggggatcacca | |
| | tcatggaaagaagcagcttcgagaagaatcccatcgactttctggaagcc | |
| | aagggctacaaagaagtgaaaaaggacctgatcatcaagctgcctaagta | |
| | ctccctgttcgagctggaaaacggccggaagagaatgctggcctcagcca | |
| | gagaactgcagaagggaaacgaactggccctgccctccaaatatgtgaac | |
| | ttcctgtacctggccagccactatgagaagctgaagggctcccccgagga | |
| | taatgagcagaaacagctgtttgtggaacagcacaagcactacctggacg | |
| | agatcatcgagcagatcagcgagttctccaagagagtgatcctggccgac | |
| | gctaatctggacaaagtgctgtccgcctacaacaagcaccgggataagcc | |
| | catcagagagcaggccgagaatatcatccacctgtttaccctgaccaatc | |
| | tgggagcccctgccgccttcaagtactttgacaccaccatcgaccggaag | |
| | cagtacagaagcaccaaagaggtgctggacgccaccctgatccaccagag | |
| | catcaccggcctgtacgagacacggatcgacctgtctcagctgggaggtg | |
| | actctggcggctcaaaaagaaccgccgacggcagcgaattcgagcccaag | |
| | aagaagaggaaagtc | |
| ABE8e-SpCas9 | Atgaaacggacagccgacggaagcgagttcgagtcaccaaagaagaagcg | 66 |
| Encoding | gaaagtctctgaggtggagttttcccacgagtactggatgagacatgccc | |
| sequences for | tgaccctggccaagagggcacgggatgagagggaggtgcctgtgggagcc | |
| NLS are bolded | gtgctggtgctgaacaatagagtgatcggcgagggctggaacagagccat | |
| and linkers are | cggcctgcacgacccaacagcccatgccgaaattatggccctgagacagg | |
| underlined | gcggcctggtcatgcagaactacagactgattgacgccaccctgtacgtg | |
| | acattcgagccttgcgtgatgtgcgccggcgccatgatccactctaggat | |
| | cggccgcgtggtgtttggcgtgaggaactcaaaaagaggcgccgcaggct | |
| | ccctgatgaacgtgctgaactaccccggcatgaatcaccgcgtcgaaatt | |
| | accgagggaatcctggcagatgaatgtgccgccctgctgtgcgatttcta | |
| | tcggatgcctagacaggtgttcaatgctcagaagaaggcccagagctcca | |
| | tcaactccggaggatctagcggaggctcctctggctctgagacacctggc | |
| | acaagcgagagcgcaacacctgaaagcagcgggggcagcagcggggggtc | |
| | agacaagaagtacagcatcggcctggccatcggcaccaactctgtgggct | |
| | gggccgtgatcaccgacgagtacaaggtgcccagcaagaaattcaaggtg | |
| | ctgggcaacaccgaccggcacagcatcaagaagaacctgatcggagccct | |
| | gctgttcgacagcggcgaaacagccgaggccacccggctgaagagaaccg | |
| | ccagaagaagatacaccagacggaagaaccggatctgctatctgcaagag | |
| | atcttcagcaacgagatggccaaggtggacgacagcttcttccacagact | |
| | ggaagagtccttcctggtggaagaggataagaagcacgagcggcacccca | |
| | tcttcggcaacatcgtggacgaggtggcctaccacgagaagtaccccacc | |
| | atctaccacctgagaaagaaactggtggacagcaccgacaaggccgacct | |
| | gcggctgatctatctggccctggcccacatgatcaagttccggggccact | |
| | tcctgatcgagggcgacctgaaccccgacaacagcgacgtggacaagctg | |
| | ttcatccagctggtgcagacctacaaccagctgttcgaggaaaaccccat | |
| | caacgccagcggcgtggacgccaaggccatcctgtctgccagactgagca | |
| | agagcagacggctggaaaatctgatcgcccagctgcccggcgagaagaag | |
| | aatggcctgttcggaaacctgattgccctgagcctgggcctgacccccaa | |
| | cttcaagagcaacttcgacctggccgaggatgccaaactgcagctgagca | |
| | aggacacctacgacgacgacctggacaacctgctggcccagatcggcgac | |
| | cagtacgccgacctgtttctggccgccaagaacctgtccgacgccatcct | |
| | gctgagcgacatcctgagagtgaacaccgagatcaccaaggcccccctga | |
| | gcgcctctatgatcaagagatacgacgagcaccaccaggacctgaccctg | |
| | ctgaaagctctcgtgcggcagcagctgcctgagaagtacaaagagatttt | |
| | cttcgaccagagcaagaacggctacgccggctacattgacggcggagcca | |
| | gccaggaagagttctacaagttcatcaagcccatcctggaaaagatggac | |
| | ggcaccgaggaactgctcgtgaagctgaacagagaggacctgctgcggaa | |
| | gcagcggaccttcgacaacggcagcatcccccaccagatccacctgggag | |
| | agctgcacgccattctgcggcggcaggaagatttttacccattcctgaag | |
| | gacaaccgggaaaagatcgagaagatcctgaccttccgcatcccctacta | |
| | cgtgggccctctggccaggggaaacagcagattcgcctggatgaccagaa | |
| | agagcgaggaaaccatcaccccctggaacttcgaggaagtggtggacaag | |
| | ggcgcttccgcccagagcttcatcgagcggatgaccaacttcgataagaa | |
| | cctgcccaacgagaaggtgctgcccaagcacagcctgctgtacgagtact | |
| | tcaccgtgtataacgagctgaccaaagtgaaatacgtgaccgagggaatg | |
| | agaaagcccgccttcctgagcggcgagcagaaaaaggccatcgtggacct | |
| | gctgttcaagaccaaccggaaagtgaccgtgaagcagctgaaagaggact | |
| | acttcaagaaaatcgagtgcttcgactccgtggaaatctccggcgtggaa | |
| | gatcggttcaacgcctccctgggcacataccacgatctgctgaaaattat | |
| | caaggacaaggacttcctggacaatgaggaaaacgaggacattctggaag | |
| | atatcgtgctgaccctgacactgtttgaggacagagagatgatcgaggaa | |
| | cggctgaaaacctatgcccacctgttcgacgacaaagtgatgaagcagct | |
| | gaagcggcggagatacaccggctggggcaggctgagccggaagctgatca | |
| | acggcatccgggacaagcagtccggcaagacaatcctggatttcctgaag | |
| | tccgacggcttcgccaacagaaacttcatgcagctgatccacgacgacag | |
| | cctgacctttaaagaggacatccagaaagcccaggtgtccggccagggcg | |
| | atagcctgcacgagcacattgccaatctggccggcagccccgccattaag | |
| | aagggcatcctgcagacagtgaaggtggtggacgagctcgtgaaagtgat | |
| | gggccggcacaagcccgagaacatcgtgatcgaaatggccagagagaacc | |
| | agaccacccagaagggacagaagaacagccgcgagagaatgaagcggatc | |
| | gaagagggcatcaaagagctgggcagccagatcctgaaagaacaccccgt | |
| | ggaaaacacccagctgcagaacgagaagctgtacctgtactacctgcaga | |
| | atggggggatatgtacgtggaccaggaactggacatcaaccggctgtccg | |
| | actacgatgtggaccatatcgtgcctcagagctttctgaaggacgactcc | |
| | atcgacaacaaggtgctgaccagaagcgacaagaaccggggcaagagcga | |
| | caacgtgccctccgaagaggtcgtgaagaagatgaagaactactggcggc | |
| | agctgctgaacgccaagctgattacccagagaaagttcgacaatctgacc | |
| | aaggccgagagaggcggcctgagcgaactggataaggccggcttcatcaa | |
| | gagacagctggtggaaacccggcagatcacaaagcacgtggcacagatcc | |
| | tggactcccggatgaacactaagtacgacgagaatgacaagctgatccgg | |
| | gaagtgaaagtgatcaccctgaagtccaagctggtgtccgatttccggaa | |
| | ggatttccagttttacaaagtgcgcgagatcaacaactaccaccacgccc | |
| | acgacgcctacctgaacgccgtcgtgggaaccgccctgatcaaaaagtac | |
| | cctaagctggaaagcgagttcgtgtacggcgactacaaggtgtacgacgt | |
| | gcggaagatgatcgccaagagcgagcaggaaatcggcaaggctaccgcca | |
| | agtacttcttctacagcaacatcatgaactttttcaagaccgagattacc | |
| | ctggccaacggcgagatccggaagcggcctctgatcgagacaaacggcga | |
| | aaccggggagatcgtgtgggataagggccgggattttgccaccgtgcgga | |
| | aagtgctgagcatgccccaagtgaatatcgtgaaaaagaccgaggtgcag | |
| | acaggcggcttcagcaaagagtctatcaggcccaagaggaacagcgataa | |
| | gctgatcgccagaaagaaggactgggaccctaagaagtacggcggcttcg | |
| | tcagccccaccgtggcctattctgtgctggtggtggccaaagtggaaaag | |
| | ggcaagtccaagaaactgaagagtgtgaaagagctgctggggatcaccat | |
| | catggaaagaagcagcttcgagaagaatcccatcgactttctggaagcca | |
| | agggctacaaagaagtgaaaaaggacctgatcatcaagctgcctaagtac | |
| | tccctgttcgagctggaaaacggccggaagagaatgctggcctctgccag | |
| | attcctgcagaagggaaacgaactggccctgccctccaaatatgtgaact | |
| | tcctgtacctggccagccactatgagaagctgaagggctcccccgaggat | |
| | aatgagcagaaacagctgtttgtggaacagcacaagcactacctggacga | |
| | gatcatcgagcagatcagcgagttctccaagagagtgatcctggccgacg | |
| | ctaatctggacaaagtgctgtccgcctacaacaagcaccgggataagccc | |
| | atcagagagcaggccgagaatatcatccacctgtttaccctgaccaatct | |
| | gggagcccctagggccttcaagtactttgacaccaccatcgaccggaagg | |
| | tgtacaggagcaccaaagaggtgctggacgccaccctgatccaccagagc | |
| | atcaccggcctgtacgagacacggatcgacctgtctcagctgggaggtga | |
| | ctctggcggctcaaaaagaaccgccgacggcagcgaattcgagcccaaga | |
| | agaagaggaaagtc | |
| ABE8e-SpRY | Atgaaacggacagccgacggaagcgagttcgagtcaccaaagaagaagcg | 67 |
| Encoding | gaaagtctctgaggtggagttttcccacgagtactggatgagacatgccc | |
| sequences for | tgaccctggccaagagggcacgggatgagagggaggtgcctgtgggagcc | |
| NLS are bolded | gtgctggtgctgaacaatagagtgatcggcgagggctggaacagagccat | |
| and linkers are | cggcctgcacgacccaacagcccatgccgaaattatggccctgagacagg | |
| underlined | gcggcctggtcatgcagaactacagactgattgacgccaccctgtacgtg | |
| | acattcgagccttgcgtgatgtgcgccggcgccatgatccactctaggat | |
| | cggccgcgtggtgtttggcgtgaggaactcaaaaagaggcgccgcaggct | |
| | ccctgatgaacgtgctgaactaccccggcatgaatcaccgcgtcgaaatt | |
| | accgagggaatcctggcagatgaatgtgccgccctgctgtgcgatttcta | |
| | tcggatgcctagacaggtgttcaatgctcagaagaaggcccagagctcca | |
| | tcaactccggaggatctagcggaggctcctctggctctgagacacctggc | |
| | acaagcgagagcgcaacacctgaaagcagcgggggcagcagcggggggtc | |
| | agacaagaagtacagcatcggcctggccatcggcaccaactctgtgggct | |
| | gggccgtgatcaccgacgagtacaaggtgcccagcaagaaattcaaggtg | |
| | ctgggcaacaccgaccggcacagcatcaagaagaacctgatcggagccct | |
| | gctgttcgacagcggcgaaacagccgagagaacccggctgaagagaaccg | |
| | ccagaagaagatacaccagacggaagaaccggatctgctatctgcaagag | |
| | atcttcagcaacgagatggccaaggtggacgacagcttcttccacagact | |
| | ggaagagtccttcctggtggaagaggataagaagcacgagcggcacccca | |
| | tcttcggcaacatcgtggacgaggtggcctaccacgagaagtaccccacc | |
| | atctaccacctgagaaagaaactggtggacagcaccgacaaggccgacct | |
| | gcggctgatctatctggccctggcccacatgatcaagttccggggccact | |
| | tcctgatcgagggcgacctgaaccccgacaacagcgacgtggacaagctg | |
| | ttcatccagctggtgcagacctacaaccagctgttcgaggaaaaccccat | |
| | caacgccagcggcgtggacgccaaggccatcctgtctgccagactgagca | |
| | agagcagacggctggaaaatctgatcgcccagctgcccggcgagaagaag | |
| | aatggcctgttcggaaacctgattgccctgagcctgggcctgacccccaa | |
| | cttcaagagcaacttcgacctggccgaggatgccaaactgcagctgagca | |
| | aggacacctacgacgacgacctggacaacctgctggcccagatcggcgac | |
| | cagtacgccgacctgtttctggccgccaagaacctgtccgacgccatcct | |
| | gctgagcgacatcctgagagtgaacaccgagatcaccaaggcccccctga | |
| | gcgcctctatgatcaagagatacgacgagcaccaccaggacctgaccctg | |
| | ctgaaagctctcgtgcggcagcagctgcctgagaagtacaaagagatttt | |
| | cttcgaccagagcaagaacggctacgccggctacattgacggcggagcca | |
| | gccaggaagagttctacaagttcatcaagcccatcctggaaaagatggac | |
| | ggcaccgaggaactgctcgtgaagctgaacagagaggacctgctgcggaa | |
| | gcagcggaccttcgacaacggcagcatcccccaccagatccacctgggag | |
| | agctgcacgccattctgcggcggcaggaagatttttacccattcctgaag | |
| | gacaaccgggaaaagatcgagaagatcctgaccttccgcatcccctacta | |
| | cgtgggccctctggccaggggaaacagcagattcgcctggatgaccagaa | |
| | agagcgaggaaaccatcaccccctggaacttcgaggaagtggtggacaag | |
| | ggcgcttccgcccagagcttcatcgagcggatgaccaacttcgataagaa | |
| | cctgcccaacgagaaggtgctgcccaagcacagcctgctgtacgagtact | |
| | tcaccgtgtataacgagctgaccaaagtgaaatacgtgaccgagggaatg | |
| | agaaagcccgccttcctgagcggcgagcagaaaaaggccatcgtggacct | |
| | gctgttcaagaccaaccggaaagtgaccgtgaagcagctgaaagaggact | |
| | acttcaagaaaatcgagtgcttcgactccgtggaaatctccggcgtggaa | |
| | gatcggttcaacgcctccctgggcacataccacgatctgctgaaaattat | |
| | caaggacaaggacttcctggacaatgaggaaaacgaggacattctggaag | |
| | atatcgtgctgaccctgacactgtttgaggacagagagatgatcgaggaa | |
| | cggctgaaaacctatgcccacctgttcgacgacaaagtgatgaagcagct | |
| | gaagcggcggagatacaccggctggggcaggctgagccggaagctgatca | |
| | acggcatccgggacaagcagtccggcaagacaatcctggatttcctgaag | |
| | tccgacggcttcgccaacagaaacttcatgcagctgatccacgacgacag | |
| | cctgacctttaaagaggacatccagaaagcccaggtgtccggccagggcg | |
| | atagcctgcacgagcacattgccaatctggccggcagccccgccattaag | |
| | aagggcatcctgcagacagtgaaggtggtggacgagctcgtgaaagtgat | |
| | gggccggcacaagcccgagaacatcgtgatcgaaatggccagagagaacc | |
| | agaccacccagaagggacagaagaacagccgcgagagaatgaagcggatc | |
| | gaagagggcatcaaagagctgggcagccagatcctgaaagaacaccccgt | |
| | ggaaaacacccagctgcagaacgagaagctgtacctgtactacctgcaga | |
| | atggggggatatgtacgtggaccaggaactggacatcaaccggctgtccg | |
| | actacgatgtggaccatatcgtgcctcagagctttctgaaggacgactcc | |
| | atcgacaacaaggtgctgaccagaagcgacaagaaccggggcaagagcga | |
| | caacgtgccctccgaagaggtcgtgaagaagatgaagaactactggcggc | |
| | agctgctgaacgccaagctgattacccagagaaagttcgacaatctgacc | |
| | aaggccgagagaggcggcctgagcgaactggataaggccggcttcatcaa | |
| | gagacagctggtggaaacccggcagatcacaaagcacgtggcacagatcc | |
| | tggactcccggatgaacactaagtacgacgagaatgacaagctgatccgg | |
| | gaagtgaaagtgatcaccctgaagtccaagctggtgtccgatttccggaa | |
| | ggatttccagttttacaaagtgcgcgagatcaacaactaccaccacgccc | |
| | acgacgcctacctgaacgccgtcgtgggaaccgccctgatcaaaaagtac | |
| | cctaagctggaaagcgagttcgtgtacggcgactacaaggtgtacgacgt | |
| | gcggaagatgatcgccaagagcgagcaggaaatcggcaaggctaccgcca | |
| | agtacttcttctacagcaacatcatgaactttttcaagaccgagattacc | |
| | ctggccaacggcgagatccggaagcggcctctgatcgagacaaacggcga | |
| | aaccggggagatcgtgtgggataagggccgggattttgccaccgtgcgga | |
| | aagtgctgagcatgccccaagtgaatatcgtgaaaaagaccgaggtgcag | |
| | acaggcggcttcagcaaagagtctatcagacccaagaggaacagcgataa | |
| | gctgatcgccagaaagaaggactgggaccctaagaagtacggcggcttcc | |
| | tgtggcccaccgtggcctattctgtgctggtggtggccaaagtggaaaag | |
| | ggcaagtccaagaaactgaagagtgtgaaagagctgctggggatcaccat | |
| | catggaaagaagcagcttcgagaagaatcccatcgactttctggaagcca | |
| | agggctacaaagaagtgaaaaaggacctgatcatcaagctgcctaagtac | |
| | tccctgttcgagctggaaaacggccggaagagaatgctggcctctgccaa | |
| | gcagctgcagaagggaaacgaactggccctgccctccaaatatgtgaact | |
| | tcctgtacctggccagccactatgagaagctgaagggctcccccgaggat | |
| | aatgagcagaaacagctgtttgtggaacagcacaagcactacctggacga | |
| | gatcatcgagcagatcagcgagttctccaagagagtgatcctggccgacg | |
| | ctaatctggacaaagtgctgtccgcctacaacaagcaccgggataagccc | |
| | atcagagagcaggccgagaatatcatccacctgtttaccctgaccagact | |
| | gggagcccctagagccttcaagtactttgacaccaccatcgaccccaagc | |
| | agtacagaagcaccaaagaggtgctggacgccaccctgatccaccagagc | |
| | atcaccggcctgtacgagacacggatcgacctgtctcagctgggaggtga | |
| | ctctggcggctcaaaaagaaccgccgacggcagcgaattcgagcccaaga | |
| | agaagaggaaagtc | |
| ABE8e-SpG | Atgaaacggacagccgacggaagcgagttcgagtcaccaaagaagaagcg | 68 |
| Encoding | gaaagtctctgaggtggagttttcccacgagtactggatgagacatgccc | |
| sequences for | tgaccctggccaagagggcacgggatgagagggaggtgcctgtgggagcc | |
| NLS are bolded | gtgctggtgctgaacaatagagtgatcggcgagggctggaacagagccat | |
| and linkers are | cggcctgcacgacccaacagcccatgccgaaattatggccctgagacagg | |
| underlined | gggcctggtcatgcagaactacagactgattgacgccaccctgtacgtga | |
| | cattcgagccttgcgtgatgtgcgccggcgccatgatccactctaggatc | |
| | ggccgcgtggtgtttggcgtgaggaactcaaaaagaggcgccgcaggctc | |
| | cctgatgaacgtgctgaactaccccggcatgaatcaccgcgtcgaaatta | |
| | ccgagggaatcctggcagatgaatgtgccgccctgctgtgcgatttctat | |
| | cggatgcctagacaggtgttcaatgctcagaagaaggcccagagctccat | |
| | caactccggaggatctagcggaggctcctctggctctgagacacctggca | |
| | caagcgagagcgcaacacctgaaagcagcgggggcagcagcggggggtca | |
| | gacaagaagtacagcatcggcctggccatcggcaccaactctgtgggctg | |
| | ggccgtgatcaccgacgagtacaaggtgcccagcaagaaattcaaggtgc | |
| | tgggcaacaccgaccggcacagcatcaagaagaacctgatcggagccctg | |
| | ctgttcgacagcggcgaaacagccgaggccacccggctgaagagaaccgc | |
| | cagaagaagatacaccagacggaagaaccggatctgctatctgcaagaga | |
| | tcttcagcaacgagatggccaaggtggacgacagcttcttccacagactg | |
| | gaagagtccttcctggtggaagaggataagaagcacgagcggcaccccat | |
| | cttcggcaacatcgtggacgaggtggcctaccacgagaagtaccccacca | |
| | tctaccacctgagaaagaaactggtggacagcaccgacaaggccgacctg | |
| | cggctgatctatctggccctggcccacatgatcaagttccggggccactt | |
| | cctgatcgagggcgacctgaaccccgacaacagcgacgtggacaagctgt | |
| | tcatccagctggtgcagacctacaaccagctgttcgaggaaaaccccatc | |
| | aacgccagcggcgtggacgccaaggccatcctgtctgccagactgagcaa | |
| | gagcagacggctggaaaatctgatcgcccagctgcccggcgagaagaaga | |
| | atggcctgttcggaaacctgattgccctgagcctgggcctgacccccaac | |
| | ttcaagagcaacttcgacctggccgaggatgccaaactgcagctgagcaa | |
| | ggacacctacgacgacgacctggacaacctgctggcccagatcggcgacc | |
| | agtacgccgacctgtttctggccgccaagaacctgtccgacgccatcctg | |
| | ctgagcgacatcctgagagtgaacaccgagatcaccaaggcccccctgag | |
| | cgcctctatgatcaagagatacgacgagcaccaccaggacctgaccctgc | |
| | tgaaagctctcgtgcggcagcagctgcctgagaagtacaaagagattttc | |
| | ttcgaccagagcaagaacggctacgccggctacattgacggcggagccag | |
| | ccaggaagagttctacaagttcatcaagcccatcctggaaaagatggacg | |
| | gcaccgaggaactgctcgtgaagctgaacagagaggacctgctgcggaag | |
| | cagcggaccttcgacaacggcagcatcccccaccagatccacctgggaga | |
| | gctgcacgccattctgcggcggcaggaagatttttacccattcctgaagg | |
| | acaaccgggaaaagatcgagaagatcctgaccttccgcatcccctactac | |
| | gtgggccctctggccaggggaaacagcagattcgcctggatgaccagaaa | |
| | gagcgaggaaaccatcaccccctggaacttcgaggaagtggtggacaagg | |
| | gcgcttccgcccagagcttcatcgagcggatgaccaacttcgataagaac | |
| | ctgcccaacgagaaggtgctgcccaagcacagcctgctgtacgagtactt | |
| | caccgtgtataacgagctgaccaaagtgaaatacgtgaccgagggaatga | |
| | gaaagcccgccttcctgagcggcgagcagaaaaaggccatcgtggacctg | |
| | ctgttcaagaccaaccggaaagtgaccgtgaagcagctgaaagaggacta | |
| | cttcaagaaaatcgagtgcttcgactccgtggaaatctccggcgtggaag | |
| | atcggttcaacgcctccctgggcacataccacgatctgctgaaaattatc | |
| | aaggacaaggacttcctggacaatgaggaaaacgaggacattctggaaga | |
| | tatcgtgctgaccctgacactgtttgaggacagagagatgatcgaggaac | |
| | ggctgaaaacctatgcccacctgttcgacgacaaagtgatgaagcagctg | |
| | aagcggcggagatacaccggctggggcaggctgagccggaagctgatcaa | |
| | cggcatccgggacaagcagtccggcaagacaatcctggatttcctgaagt | |
| | ccgacggcttcgccaacagaaacttcatgcagctgatccacgacgacagc | |
| | ctgacctttaaagaggacatccagaaagcccaggtgtccggccagggcga | |
| | tagcctgcacgagcacattgccaatctggccggcagccccgccattaaga | |
| | agggcatcctgcagacagtgaaggtggtggacgagctcgtgaaagtgatg | |
| | ggccggcacaagcccgagaacatcgtgatcgaaatggccagagagaacca | |
| | gaccacccagaagggacagaagaacagccgcgagagaatgaagcggatcg | |
| | aagagggcatcaaagagctgggcagccagatcctgaaagaacaccccgtg | |
| | gaaaacacccagctgcagaacgagaagctgtacctgtactacctgcagaa | |
| | tgggcgggatatgtacgtggaccaggaactggacatcaaccggctgtccg | |
| | actacgatgtggaccatatcgtgcctcagagctttctgaaggacgactcc | |
| | atcgacaacaaggtgctgaccagaagcgacaagaaccggggcaagagcga | |
| | caacgtgccctccgaagaggtcgtgaagaagatgaagaactactggcggc | |
| | agctgctgaacgccaagctgattacccagagaaagttcgacaatctgacc | |
| | aaggccgagagaggcggcctgagcgaactggataaggccggcttcatcaa | |
| | gagacagctggtggaaacccggcagatcacaaagcacgtggcacagatcc | |
| | tggactcccggatgaacactaagtacgacgagaatgacaagctgatccgg | |
| | gaagtgaaagtgatcaccctgaagtccaagctggtgtccgatttccggaa | |
| | ggatttccagttttacaaagtgcgcgagatcaacaactaccaccacgccc | |
| | acgacgcctacctgaacgccgtcgtgggaaccgccctgatcaaaaagtac | |
| | cctaagctggaaagcgagttcgtgtacggcgactacaaggtgtacgacgt | |
| | gcggaagatgatcgccaagagcgagcaggaaatcggcaaggctaccgcca | |
| | agtacttcttctacagcaacatcatgaactttttcaagaccgagattacc | |
| | ctggccaacggcgagatccggaagcggcctctgatcgagacaaacggcga | |
| | aaccggggagatcgtgtgggataagggccgggattttgccaccgtgcgga | |
| | aagtgctgagcatgccccaagtgaatatcgtgaaaaagaccgaggtgcag | |
| | acaggcggcttcagcaaagagtctatcctgcccaagaggaacagcgataa | |
| | gctgatcgccagaaagaaggactgggaccctaagaagtacggcggcttcc | |
| | tgtggcccaccgtggcctattctgtgctggtggtggccaaagtggaaaag | |
| | ggcaagtccaagaaactgaagagtgtgaaagagctgctggggatcaccat | |
| | catggaaagaagcagcttcgagaagaatcccatcgactttctggaagcca | |
| | agggctacaaagaagtgaaaaaggacctgatcatcaagctgcctaagtac | |
| | tccctgttcgagctggaaaacggccggaagagaatgctggcctctgccaa | |
| | gcagctgcagaagggaaacgaactggccctgccctccaaatatgtgaact | |
| | tcctgtacctggccagccactatgagaagctgaagggctcccccgaggat | |
| | aatgagcagaaacagctgtttgtggaacagcacaagcactacctggacga | |
| | gatcatcgagcagatcagcgagttctccaagagagtgatcctggccgacg | |
| | ctaatctggacaaagtgctgtccgcctacaacaagcaccgggataagccc | |
| | atcagagagcaggccgagaatatcatccacctgtttaccctgaccaatct | |
| | gggagcccctgccgccttcaagtactttgacaccaccatcgaccggaagc | |
| | agtacagaagcaccaaagaggtgctggacgccaccctgatccaccagagc | |
| | atcaccggcctgtacgagacacggatcgacctgtctcagctgggaggtga | |
| | ctctggcggctcaaaaagaaccgccgacggcagcgaattcgagcccaaga | |
| | agaagaggaaagtc | |
| ABEmax-VRQR | tctgaagtcgagtttagccacgagtattggatgaggcacgcactgaccct | 73 |
| Encoding | ggcaaagcgagcatgggatgaaagagaagtccccgtgggcgccgtgctgg | |
| sequences for | tgcacaacaatagagtgatcggagagggatggaacaggccaatcggccgc | |
| linkers are | cacgaccctaccgcacacgcagagatcatggcactgaggcagggaggcct | |
| underlined | ggtcatgcagaattaccgcctgatcgatgccaccctgtatgtgacactgg | |
| | agccatgcgtgatgtgcgcaggagcaatgatccacagcaggatcggaaga | |
| | gtggtgttcggagcacgggacgccaagaccggcgcagcaggctccctgat | |
| | ggatgtgctgcaccaccccggcatgaaccaccgggtggagatcacagagg | |
| | gaatcctggcagacgagtgcgccgccctgctgagcgatttctttagaatg | |
| | cggagacaggagatcaaggcccagaagaaggcacagagctccaccgactc | |
| | tggaggatctagcggaggatcctctggaagcgagacaccaggcacaagcg | |
| | agtccgccacaccagagagctccggcggctcctccggaggatcctctgag | |
| | gtggagttttcccacgagtactggatgagacatgccctgaccctggccaa | |
| | gagggcacgcgatgagagggaggtgcctgtgggagccgtgctggtgctga | |
| | acaatagagtgatcggcgagggctggaacagagccatcggcctgcacgac | |
| | ccaacagcccatgccgaaattatggccctgagacagggcggcctggtcat | |
| | gcagaactacagactgattgacgccaccctgtacgtgacattcgagcctt | |
| | gcgtgatgtgcgccggcgccatgatccactctaggatcggccgcgtggtg | |
| | tttggcgtgaggaacgcaaaaaccggcgccgcaggctccctgatggacgt | |
| | gctgcactaccccggcatgaatcaccgcgtcgaaattaccgagggaatcc | |
| | tggcagatgaatgtgccgccctgctgtgctatttctttcggatgcctaga | |
| | caggtgttcaatgctcagaagaaggcccagagctccaccgactccggagg | |
| | atctagcggaggctcctctggctctgagacacctggcacaagcgagagcg | |
| | caacacctgaaagcagcgggggcagcagcggggggtcagacaagaagtac | |
| | agcatcggcctggccatcggcaccaactctgtgggctgggccgtgatcac | |
| | cgacgagtacaaggtgcccagcaagaaattcaaggtgctgggcaacaccg | |
| | accggcacagcatcaagaagaacctgatcggagccctgctgttcgacagc | |
| | ggcgaaacagccgaggccacccggctgaagagaaccgccagaagaagata | |
| | caccagacggaagaaccggatctgctatctgcaagagatcttcagcaacg | |
| | agatggccaaggtggacgacagcttcttccacagactggaagagtccttc | |
| | ctggtggaagaggataagaagcacgagcggcaccccatcttcggcaacat | |
| | cgtggacgaggtggcctaccacgagaagtaccccaccatctaccacctga | |
| | gaaagaaactggtggacagcaccgacaaggccgacctgcggctgatctat | |
| | ctggccctggcccacatgatcaagttccggggccacttcctgatcgaggg | |
| | cgacctgaaccccgacaacagcgacgtggacaagctgttcatccagctgg | |
| | tgcagacctacaaccagctgttcgaggaaaaccccatcaacgccagcggc | |
| | gtggacgccaaggccatcctgtctgccagactgagcaagagcagacggct | |
| | ggaaaatctgatcgcccagctgcccggcgagaagaagaatggcctgttcg | |
| | gaaacctgattgccctgagcctgggcctgacccccaacttcaagagcaac | |
| | ttcgacctggccgaggatgccaaactgcagctgagcaaggacacctacga | |
| | cgacgacctggacaacctgctggcccagatcggcgaccagtacgccgacc | |
| | tgtttctggccgccaagaacctgtccgacgccatcctgctgagcgacatc | |
| | ctgagagtgaacaccgagatcaccaaggcccccctgagcgcctctatgat | |
| | caagagatacgacgagcaccaccaggacctgaccctgctgaaagctctcg | |
| | tgcggcagcagctgcctgagaagtacaaagagattttcttcgaccagagc | |
| | aagaacggctacgccggctacattgacggcggagccagccaggaagagtt | |
| | ctacaagttcatcaagcccatcctggaaaagatggacggcaccgaggaac | |
| | tgctcgtgaagctgaacagagaggacctgctgcggaagcagcggaccttc | |
| | gacaacggcagcatcccccaccagatccacctgggagagctgcacgccat | |
| | tctgcggcggcaggaagatttttacccattcctgaaggacaaccgggaaa | |
| | agatcgagaagatcctgaccttccgcatcccctactacgtgggccctctg | |
| | gccaggggaaacagcagattcgcctggatgaccagaaagagcgaggaaac | |
| | catcaccccctggaacttcgaggaagtggtggacaagggcgcttccgccc | |
| | agagcttcatcgagcggatgaccaacttcgataagaacctgcccaacgag | |
| | aaggtgctgcccaagcacagcctgctgtacgagtacttcaccgtgtataa | |
| | cgagctgaccaaagtgaaatacgtgaccgagggaatgagaaagcccgcct | |
| | tcctgagcggcgagcagaaaaaggccatcgtggacctgctgttcaagacc | |
| | aaccggaaagtgaccgtgaagcagctgaaagaggactacttcaagaaaat | |
| | cgagtgcttcgactccgtggaaatctccggcgtggaagatcggttcaacg | |
| | cctccctgggcacataccacgatctgctgaaaattatcaaggacaaggac | |
| | ttcctggacaatgaggaaaacgaggacattctggaagatatcgtgctgac | |
| | cctgacactgtttgaggacagagagatgatcgaggaacggctgaaaacct | |
| | atgcccacctgttcgacgacaaagtgatgaagcagctgaagcggcggaga | |
| | tacaccggctggggcaggctgagccggaagctgatcaacggcatccggga | |
| | caagcagtccggcaagacaatcctggatttcctgaagtccgacggcttcg | |
| | ccaacagaaacttcatgcagctgatccacgacgacagcctgacctttaaa | |
| | gaggacatccagaaagcccaggtgtccggccagggcgatagcctgcacga | |
| | gcacattgccaatctggccggcagccccgccattaagaagggcatcctgc | |
| | agacagtgaaggtggtggacgagctcgtgaaagtgatgggccggcacaag | |
| | cccgagaacatcgtgatcgaaatggccagagagaaccagaccacccagaa | |
| | gggacagaagaacagccgcgagagaatgaagcggatcgaagagggcatca | |
| | aagagctgggcagccagatcctgaaagaacaccccgtggaaaacacccag | |
| | ctgcagaacgagaagctgtacctgtactacctgcagaatgggcgggatat | |
| | gtacgtggaccaggaactggacatcaaccggctgtccgactacgatgtgg | |
| | accatatcgtgcctcagagctttctgaaggacgactccatcgacaacaag | |
| | gtgctgaccagaagcgacaagaaccggggcaagagcgacaacgtgccctc | |
| | cgaagaggtcgtgaagaagatgaagaactactggcggcagctgctgaacg | |
| | ccaagctgattacccagagaaagttcgacaatctgaccaaggccgagaga | |
| | ggcggcctgagcgaactggataaggccggcttcatcaagagacagctggt | |
| | ggaaacccggcagatcacaaagcacgtggcacagatcctggactcccgga | |
| | tgaacactaagtacgacgagaatgacaagctgatccgggaagtgaaagtg | |
| | atcaccctgaagtccaagctggtgtccgatttccggaaggatttccagtt | |
| | ttacaaagtgcgcgagatcaacaactaccaccacgcccacgacgcctacc | |
| | tgaacgccgtcgtgggaaccgccctgatcaaaaagtaccctaagctggaa | |
| | agcgagttcgtgtacggcgactacaaggtgtacgacgtgcggaagatgat | |
| | cgccaagagcgagcaggaaatcggcaaggctaccgccaagtacttcttct | |
| | acagcaacatcatgaactttttcaagaccgagattaccctggccaacggc | |
| | gagatccggaagcggcctctgatcgagacaaacggcgaaaccggggagat | |
| | cgtgtgggataagggccgggattttgccaccgtgcggaaagtgctgagca | |
| | tgccccaagtgaatatcgtgaaaaagaccgaggtgcagacaggcggcttc | |
| | agcaaagagtctatcctgcccaagaggaacagcgataagctgatcgccag | |
| | aaagaaggactgggaccctaagaagtacggcggcttcgtgagccccaccg | |
| | tggcctattctgtgctggtggtggccaaagtggaaaagggcaagtccaag | |
| | aaactgaagagtgtgaaagagctgctggggatcaccatcatggaaagaag | |
| | cagcttcgagaagaatcccatcgactttctggaagccaagggctacaaag | |
| | aagtgaaaaaggacctgatcatcaagctgcctaagtactccctgttcgag | |
| | ctggaaaacggccggaagagaatgctggcctcagccagagaactgcagaa | |
| | gggaaacgaactggccctgccctccaaatatgtgaacttcctgtacctgg | |
| | ccagccactatgagaagctgaagggctcccccgaggataatgagcagaaa | |
| | cagctgtttgtggaacagcacaagcactacctggacgagatcatcgagca | |
| | gatcagcgagttctccaagagagtgatcctggccgacgctaatctggaca | |
| | aagtgctgtccgcctacaacaagcaccgggataagcccatcagagagcag | |
| | gccgagaatatcatccacctgtttaccctgaccaatctgggagcccctgc | |
| | cgccttcaagtactttgacaccaccatcgaccggaagcagtacagaagca | |
| | ccaaagaggtgctggacgccaccctgatccaccagagcatcaccggcctg | |
| | tacgagacacggatcgacctgtctcagctgggaggtgac | |
| ABEmax-SpCas9- | tctgaagtcgagtttagccacgagtattggatgaggcacgcactgaccct | 79 |
| NG (DNA) | ggcaaagcgagcatgggatgaaagagaagtccccgtgggcgccgtgctgg | |
| Encoding | tgcacaacaatagagtgatcggagagggatggaacaggccaatcggccgc | |
| sequences for | cacgaccctaccgcacacgcagagatcatggcactgaggcagggaggcct | |
| linkers are | ggtcatgcagaattaccgcctgatcgatgccaccctgtatgtgacactgg | |
| underlined | agccatgcgtgatgtgcgcaggagcaatgatccacagcaggatcggaaga | |
| | gtggtgttcggagcacgggacgccaagaccggcgcagcaggctccctgat | |
| | ggatgtgctgcaccaccccggcatgaaccaccgggtggagatcacagagg | |
| | gaatcctggcagacgagtgcgccgccctgctgagcgatttctttagaatg | |
| | cggagacaggagatcaaggcccagaagaaggcacagagctccaccgactc | |
| | tggaggatctagcggaggatcctctggaagcgagacaccaggcacaagcg | |
| | agtccgccacaccagagagctccggcggctcctccggaggatcctctgag | |
| | gtggagttttcccacgagtactggatgagacatgccctgaccctggccaa | |
| | gagggcacgcgatgagagggaggtgcctgtgggagccgtgctggtgctga | |
| | acaatagagtgatcggcgagggctggaacagagccatcggcctgcacgac | |
| | ccaacagcccatgccgaaattatggccctgagacagggcggcctggtcat | |
| | gcagaactacagactgattgacgccaccctgtacgtgacattcgagcctt | |
| | gcgtgatgtgcgccggcgccatgatccactctaggatcggccgcgtggtg | |
| | tttggcgtgaggaacgcaaaaaccggcgccgcaggctccctgatggacgt | |
| | gctgcactaccccggcatgaatcaccgcgtcgaaattaccgagggaatcc | |
| | tggcagatgaatgtgccgccctgctgtgctatttctttcggatgcctaga | |
| | caggtgttcaatgctcagaagaaggcccagagctccaccgactccggagg | |
| | atctagcggaggctcctctggctctgagacacctggcacaagcgagagcg | |
| | caacacctgaaagcagcgggggcagcagcggggggtcagacaagaagtac | |
| | agcatcggcctggccatcggcaccaactctgtgggctgggccgtgatcac | |
| | cgacgagtacaaggtgcccagcaagaaattcaaggtgctgggcaacaccg | |
| | accggcacagcatcaagaagaacctgatcggagccctgctgttcgacagc | |
| | ggcgaaacagccgaggccacccggctgaagagaaccgccagaagaagata | |
| | caccagacggaagaaccggatctgctatctgcaagagatcttcagcaacg | |
| | agatggccaaggtggacgacagcttcttccacagactggaagagtccttc | |
| | ctggtggaagaggataagaagcacgagcggcaccccatcttcggcaacat | |
| | cgtggacgaggtggcctaccacgagaagtaccccaccatctaccacctga | |
| | gaaagaaactggtggacagcaccgacaaggccgacctgcggctgatctat | |
| | ctggccctggcccacatgatcaagttccggggccacttcctgatcgaggg | |
| | cgacctgaaccccgacaacagcgacgtggacaagctgttcatccagctgg | |
| | tgcagacctacaaccagctgttcgaggaaaaccccatcaacgccagcggc | |
| | gtggacgccaaggccatcctgtctgccagactgagcaagagcagacggct | |
| | ggaaaatctgatcgcccagctgcccggcgagaagaagaatggcctgttcg | |
| | gaaacctgattgccctgagcctgggcctgacccccaacttcaagagcaac | |
| | ttcgacctggccgaggatgccaaactgcagctgagcaaggacacctacga | |
| | cgacgacctggacaacctgctggcccagatcggcgaccagtacgccgacc | |
| | tgtttctggccgccaagaacctgtccgacgccatcctgctgagcgacatc | |
| | ctgagagtgaacaccgagatcaccaaggcccccctgagcgcctctatgat | |
| | caagagatacgacgagcaccaccaggacctgaccctgctgaaagctctcg | |
| | tgcggcagcagctgcctgagaagtacaaagagattttcttcgaccagagc | |
| | aagaacggctacgccggctacattgacggcggagccagccaggaagagtt | |
| | ctacaagttcatcaagcccatcctggaaaagatggacggcaccgaggaac | |
| | tgctcgtgaagctgaacagagaggacctgctgcggaagcagcggaccttc | |
| | gacaacggcagcatcccccaccagatccacctgggagagctgcacgccat | |
| | tctgcggcggcaggaagatttttacccattcctgaaggacaaccgggaaa | |
| | agatcgagaagatcctgaccttccgcatcccctactacgtgggccctctg | |
| | gccaggggaaacagcagattcgcctggatgaccagaaagagcgaggaaac | |
| | catcaccccctggaacttcgaggaagtggtggacaagggcgcttccgccc | |
| | agagcttcatcgagcggatgaccaacttcgataagaacctgcccaacgag | |
| | aaggtgctgcccaagcacagcctgctgtacgagtacttcaccgtgtataa | |
| | cgagctgaccaaagtgaaatacgtgaccgagggaatgagaaagcccgcct | |
| | tcctgagcggcgagcagaaaaaggccatcgtggacctgctgttcaagacc | |
| | aaccggaaagtgaccgtgaagcagctgaaagaggactacttcaagaaaat | |
| | cgagtgcttcgactccgtggaaatctccggcgtggaagatcggttcaacg | |
| | cctccctgggcacataccacgatctgctgaaaattatcaaggacaaggac | |
| | ttcctggacaatgaggaaaacgaggacattctggaagatatcgtgctgac | |
| | cctgacactgtttgaggacagagagatgatcgaggaacggctgaaaacct | |
| | atgcccacctgttcgacgacaaagtgatgaagcagctgaagcggcggaga | |
| | tacaccggctggggcaggctgagccggaagctgatcaacggcatccggga | |
| | caagcagtccggcaagacaatcctggatttcctgaagtccgacggcttcg | |
| | ccaacagaaacttcatgcagctgatccacgacgacagcctgacctttaaa | |
| | gaggacatccagaaagcccaggtgtccggccagggcgatagcctgcacga | |
| | gcacattgccaatctggccggcagccccgccattaagaagggcatcctgc | |
| | agacagtgaaggtggtggacgagctcgtgaaagtgatgggccggcacaag | |
| | cccgagaacatcgtgatcgaaatggccagagagaaccagaccacccagaa | |
| | gggacagaagaacagccgcgagagaatgaagcggatcgaagagggcatca | |
| | aagagctgggcagccagatcctgaaagaacaccccgtggaaaacacccag | |
| | ctgcagaacgagaagctgtacctgtactacctgcagaatgggcgggatat | |
| | gtacgtggaccaggaactggacatcaaccggctgtccgactacgatgtgg | |
| | accatatcgtgcctcagagctttctgaaggacgactccatcgacaacaag | |
| | gtgctgaccagaagcgacaagaaccggggcaagagcgacaacgtgccctc | |
| | cgaagaggtcgtgaagaagatgaagaactactggcggcagctgctgaacg | |
| | ccaagctgattacccagagaaagttcgacaatctgaccaaggccgagaga | |
| | ggcggcctgagcgaactggataaggccggcttcatcaagagacagctggt | |
| | ggaaacccggcagatcacaaagcacgtggcacagatcctggactcccgga | |
| | tgaacactaagtacgacgagaatgacaagctgatccgggaagtgaaagtg | |
| | atcaccctgaagtccaagctggtgtccgatttccggaaggatttccagtt | |
| | ttacaaagtgcgcgagatcaacaactaccaccacgcccacgacgcctacc | |
| | tgaacgccgtcgtgggaaccgccctgatcaaaaagtaccctaagctggaa | |
| | agcgagttcgtgtacggcgactacaaggtgtacgacgtgcggaagatgat | |
| | cgccaagagcgagcaggaaatcggcaaggctaccgccaagtacttcttct | |
| | acagcaacatcatgaactttttcaagaccgagattaccctggccaacggc | |
| | gagatccggaagcggcctctgatcgagacaaacggcgaaaccggggagat | |
| | cgtgtgggataagggccgggattttgccaccgtgcggaaagtgctgagca | |
| | tgccccaagtgaatatcgtgaaaaagaccgaggtgcagacaggcggcttc | |
| | agcaaagagtctatcaggcccaagaggaacagcgataagctgatcgccag | |
| | aaagaaggactgggaccctaagaagtacggcggcttcgtcagccccaccg | |
| | tggcctattctgtgctggtggtggccaaagtggaaaagggcaagtccaag | |
| | aaactgaagagtgtgaaagagctgctggggatcaccatcatggaaagaag | |
| | cagcttcgagaagaatcccatcgactttctggaagccaagggctacaaag | |
| | aagtgaaaaaggacctgatcatcaagctgcctaagtactccctgttcgag | |
| | ctggaaaacggccggaagagaatgctggcctctgccagattcctgcagaa | |
| | gggaaacgaactggccctgccctccaaatatgtgaacttcctgtacctgg | |
| | ccagccactatgagaagctgaagggctcccccgaggataatgagcagaaa | |
| | cagctgtttgtggaacagcacaagcactacctggacgagatcatcgagca | |
| | gatcagcgagttctccaagagagtgatcctggccgacgctaatctggaca | |
| | aagtgctgtccgcctacaacaagcaccgggataagcccatcagagagcag | |
| | gccgagaatatcatccacctgtttaccctgaccaatctgggagcccctag | |
| | ggccttcaagtactttgacaccaccatcgaccggaaggtgtacaggagca | |
| | ccaaagaggtgctggacgccaccctgatccaccagagcatcaccggcctg | |
| | tacgagacacggatcgacctgtctcagctgggaggtgac | |
| ABEmax-SpRY | tctgaagtcgagtttagccacgagtattggatgaggcacgcactgaccct | 147 |
| Encoding | ggcaaagcgagcatgggatgaaagagaagtccccgtgggcgccgtgctgg | |
| sequences for | tgcacaacaatagagtgatcggagagggatggaacaggccaatcggccgc | |
| linkers are | cacgaccctaccgcacacgcagagatcatggcactgaggcagggaggcct | |
| underlined | ggtcatgcagaattaccgcctgatcgatgccaccctgtatgtgacactgg | |
| | agccatgcgtgatgtgcgcaggagcaatgatccacagcaggatcggaaga | |
| | gtggtgttcggagcacgggacgccaagaccggcgcagcaggctccctgat | |
| | ggatgtgctgcaccaccccggcatgaaccaccgggtggagatcacagagg | |
| | gaatcctggcagacgagtgcgccgccctgctgagcgatttctttagaatg | |
| | cggagacaggagatcaaggcccagaagaaggcacagagctccaccgactc | |
| | tggaggatctagcggaggatcctctggaagcgagacaccaggcacaagcg | |
| | agtccgccacaccagagagctccggcggctcctccggaggatcctctgag | |
| | gtggagttttcccacgagtactggatgagacatgccctgaccctggccaa | |
| | gagggcacgcgatgagagggaggtgcctgtgggagccgtgctggtgctga | |
| | acaatagagtgatcggcgagggctggaacagagccatcggcctgcacgac | |
| | ccaacagcccatgccgaaattatggccctgagacagggggcctggtcatg | |
| | cagaactacagactgattgacgccaccctgtacgtgacattcgagccttg | |
| | cgtgatgtgcgccggcgccatgatccactctaggatcggccgcgtggtgt | |
| | ttggcgtgaggaacgcaaaaaccggcgccgcaggctccctgatggacgtg | |
| | ctgcactaccccggcatgaatcaccgcgtcgaaattaccgagggaatcct | |
| | ggcagatgaatgtgccgccctgctgtgctatttctttcggatgcctagac | |
| | aggtgttcaatgctcagaagaaggcccagagctccaccgactccggagga | |
| | tctagcggaggctcctctggctctgagacacctggcacaagcgagagcgc | |
| | aacacctgaaagcagcgggggcagcagcggggggtcaatggacaagaagt | |
| | acagcatcggcctggccatcggcaccaactctgtgggctgggccgtgatc | |
| | accgacgagtacaaggtgcccagcaagaaattcaaggtgctgggcaacac | |
| | cgaccggcacagcatcaagaagaacctgatcggagccctgctgttcgaca | |
| | gcggcgaaacagccgagagaacccggctgaagagaaccgccagaagaaga | |
| | tacaccagacggaagaaccggatctgctatctgcaagagatcttcagcaa | |
| | cgagatggccaaggtggacgacagcttcttccacagactggaagagtcct | |
| | tcctggtggaagaggataagaagcacgagcggcaccccatcttcggcaac | |
| | atcgtggacgaggtggcctaccacgagaagtaccccaccatctaccacct | |
| | gagaaagaaactggtggacagcaccgacaaggccgacctgcggctgatct | |
| | atctggccctggcccacatgatcaagttccggggccacttcctgatcgag | |
| | ggcgacctgaaccccgacaacagcgacgtggacaagctgttcatccagct | |
| | ggtgcagacctacaaccagctgttcgaggaaaaccccatcaacgccagcg | |
| | gcgtggacgccaaggccatcctgtctgccagactgagcaagagcagacgg | |
| | ctggaaaatctgatcgcccagctgcccggcgagaagaagaatggcctgtt | |
| | cggaaacctgattgccctgagcctgggcctgacccccaacttcaagagca | |
| | acttcgacctggccgaggatgccaaactgcagctgagcaaggacacctac | |
| | gacgacgacctggacaacctgctggcccagatcggcgaccagtacgccga | |
| | cctgtttctggccgccaagaacctgtccgacgccatcctgctgagcgaca | |
| | tcctgagagtgaacaccgagatcaccaaggcccccctgagcgcctctatg | |
| | atcaagagatacgacgagcaccaccaggacctgaccctgctgaaagctct | |
| | cgtgcggcagcagctgcctgagaagtacaaagagattttcttcgaccaga | |
| | gcaagaacggctacgccggctacattgacggcggagccagccaggaagag | |
| | ttctacaagttcatcaagcccatcctggaaaagatggacggcaccgagga | |
| | actgctcgtgaagctgaacagagaggacctgctgcggaagcagcggacct | |
| | tcgacaacggcagcatcccccaccagatccacctgggagagctgcacgcc | |
| | attctgcggcggcaggaagatttttacccattcctgaaggacaaccggga | |
| | aaagatcgagaagatcctgaccttccgcatcccctactacgtgggccctc | |
| | tggccaggggaaacagcagattcgcctggatgaccagaaagagcgaggaa | |
| | accatcaccccctggaacttcgaggaagtggtggacaagggcgcttccgc | |
| | ccagagcttcatcgagcggatgaccaacttcgataagaacctgcccaacg | |
| | agaaggtgctgcccaagcacagcctgctgtacgagtacttcaccgtgtat | |
| | aacgagctgaccaaagtgaaatacgtgaccgagggaatgagaaagcccgc | |
| | cttcctgagcggcgagcagaaaaaggccatcgtggacctgctgttcaaga | |
| | ccaaccggaaagtgaccgtgaagcagctgaaagaggactacttcaagaaa | |
| | atcgagtgcttcgactccgtggaaatctccggcgtggaagatcggttcaa | |
| | cgcctccctgggcacataccacgatctgctgaaaattatcaaggacaagg | |
| | acttcctggacaatgaggaaaacgaggacattctggaagatatcgtgctg | |
| | accctgacactgtttgaggacagagagatgatcgaggaacggctgaaaac | |
| | ctatgcccacctgttcgacgacaaagtgatgaagcagctgaagcggcgga | |
| | gatacaccggctggggcaggctgagccggaagctgatcaacggcatccgg | |
| | gacaagcagtccggcaagacaatcctggatttcctgaagtccgacggctt | |
| | cgccaacagaaacttcatgcagctgatccacgacgacagcctgaccttta | |
| | aagaggacatccagaaagcccaggtgtccggccagggcgatagcctgcac | |
| | gagcacattgccaatctggccggcagccccgccattaagaagggcatcct | |
| | gcagacagtgaaggtggtggacgagctcgtgaaagtgatgggccggcaca | |
| | agcccgagaacatcgtgatcgaaatggccagagagaaccagaccacccag | |
| | aagggacagaagaacagccgcgagagaatgaagcggatcgaagagggcat | |
| | caaagagctgggcagccagatcctgaaagaacaccccgtggaaaacaccc | |
| | agctgcagaacgagaagctgtacctgtactacctgcagaatgggcgggat | |
| | atgtacgtggaccaggaactggacatcaaccggctgtccgactacgatgt | |
| | ggaccatatcgtgcctcagagctttctgaaggacgactccatcgacaaca | |
| | aggtgctgaccagaagcgacaagaaccggggcaagagcgacaacgtgccc | |
| | tccgaagaggtcgtgaagaagatgaagaactactggcggcagctgctgaa | |
| | cgccaagctgattacccagagaaagttcgacaatctgaccaaggccgaga | |
| | gaggcggcctgagcgaactggataaggccggcttcatcaagagacagctg | |
| | gtggaaacccggcagatcacaaagcacgtggcacagatcctggactcccg | |
| | gatgaacactaagtacgacgagaatgacaagctgatccgggaagtgaaag | |
| | tgatcaccctgaagtccaagctggtgtccgatttccggaaggatttccag | |
| | ttttacaaagtgcgcgagatcaacaactaccaccacgcccacgacgccta | |
| | cctgaacgccgtcgtgggaaccgccctgatcaaaaagtaccctaagctgg | |
| | aaagcgagttcgtgtacggcgactacaaggtgtacgacgtgcggaagatg | |
| | atcgccaagagcgagcaggaaatcggcaaggctaccgccaagtacttctt | |
| | ctacagcaacatcatgaactttttcaagaccgagattaccctggccaacg | |
| | gcgagatccggaagcggcctctgatcgagacaaacggcgaaaccggggag | |
| | atcgtgtgggataagggccgggattttgccaccgtgcggaaagtgctgag | |
| | catgccccaagtgaatatcgtgaaaaagaccgaggtgcagacaggcggct | |
| | tcagcaaagagtctatcagacccaagaggaacagcgataagctgatcgcc | |
| | agaaagaaggactgggaccctaagaagtacggcggcttcctgtggcccac | |
| | cgtggcctattctgtgctggtggtggccaaagtggaaaagggcaagtcca | |
| | agaaactgaagagtgtgaaagagctgctggggatcaccatcatggaaaga | |
| | agcagcttcgagaagaatcccatcgactttctggaagccaagggctacaa | |
| | agaagtgaaaaaggacctgatcatcaagctgcctaagtactccctgttcg | |
| | agctggaaaacggccggaagagaatgctggcctctgccaagcagctgcag | |
| | aagggaaacgaactggccctgccctccaaatatgtgaacttcctgtacct | |
| | ggccagccactatgagaagctgaagggctcccccgaggataatgagcaga | |
| | aacagctgtttgtggaacagcacaagcactacctggacgagatcatcgag | |
| | cagatcagcgagttctccaagagagtgatcctggccgacgctaatctgga | |
| | caaagtgctgtccgcctacaacaagcaccgggataagcccatcagagagc | |
| | aggccgagaatatcatccacctgtttaccctgaccagactgggagcccct | |
| | agagccttcaagtactttgacaccaccatcgaccccaagcagtacagaag | |
| | caccaaagaggtgctggacgccaccctgatccaccagagcatcaccggcc | |
| | tgtacgagacacggatcgacctgtctcagctgggaggtgac | |
| ABEmax-SpG | tctgaagtcgagtttagccacgagtattggatgaggcacgcactgaccct | 148 |
| Encoding | ggcaaagcgagcatgggatgaaagagaagtccccgtgggcgccgtgctgg | |
| sequences for | tgcacaacaatagagtgatcggagagggatggaacaggccaatcggccgc | |
| linkers are | cacgaccctaccgcacacgcagagatcatggcactgaggcagggaggcct | |
| underlined | ggtcatgcagaattaccgcctgatcgatgccaccctgtatgtgacactgg | |
| | agccatgcgtgatgtgcgcaggagcaatgatccacagcaggatcggaaga | |
| | gtggtgttcggagcacgggacgccaagaccggcgcagcaggctccctgat | |
| | ggatgtgctgcaccaccccggcatgaaccaccgggtggagatcacagagg | |
| | gaatcctggcagacgagtgcgccgccctgctgagcgatttctttagaatg | |
| | cggagacaggagatcaaggcccagaagaaggcacagagctccaccgactc | |
| | tggaggatctagcggaggatcctctggaagcgagacaccaggcacaagcg | |
| | agtccgccacaccagagagctccggcggctcctccggaggatcctctgag | |
| | gtggagttttcccacgagtactggatgagacatgccctgaccctggccaa | |
| | gagggcacgcgatgagagggaggtgcctgtgggagccgtgctggtgctga | |
| | acaatagagtgatcggcgagggctggaacagagccatcggcctgcacgac | |
| | ccaacagcccatgccgaaattatggccctgagacagggcggcctggtcat | |
| | gcagaactacagactgattgacgccaccctgtacgtgacattcgagcctt | |
| | gcgtgatgtgcgccggcgccatgatccactctaggatcggccgcgtggtg | |
| | tttggcgtgaggaacgcaaaaaccggcgccgcaggctccctgatggacgt | |
| | gctgcactaccccggcatgaatcaccgcgtcgaaattaccgagggaatcc | |
| | tggcagatgaatgtgccgccctgctgtgctatttctttcggatgcctaga | |
| | caggtgttcaatgctcagaagaaggcccagagctccaccgactccggagg | |
| | atctagcggaggctcctctggctctgagacacctggcacaagcgagagcg | |
| | caacacctgaaagcagcgggggcagcagcggggggtcagacaagaagtac | |
| | agcatcggcctggccatcggcaccaactctgtgggctgggccgtgatcac | |
| | cgacgagtacaaggtgcccagcaagaaattcaaggtgctgggcaacaccg | |
| | accggcacagcatcaagaagaacctgatcggagccctgctgttcgacagc | |
| | ggcgaaacagccgaggccacccggctgaagagaaccgccagaagaagata | |
| | caccagacggaagaaccggatctgctatctgcaagagatcttcagcaacg | |
| | agatggccaaggtggacgacagcttcttccacagactggaagagtccttc | |
| | ctggtggaagaggataagaagcacgagcggcaccccatcttcggcaacat | |
| | cgtggacgaggtggcctaccacgagaagtaccccaccatctaccacctga | |
| | gaaagaaactggtggacagcaccgacaaggccgacctgcggctgatctat | |
| | ctggccctggcccacatgatcaagttccggggccacttcctgatcgaggg | |
| | cgacctgaaccccgacaacagcgacgtggacaagctgttcatccagctgg | |
| | tgcagacctacaaccagctgttcgaggaaaaccccatcaacgccagcggc | |
| | gtggacgccaaggccatcctgtctgccagactgagcaagagcagacggct | |
| | ggaaaatctgatcgcccagctgcccggcgagaagaagaatggcctgttcg | |
| | gaaacctgattgccctgagcctgggcctgacccccaacttcaagagcaac | |
| | ttcgacctggccgaggatgccaaactgcagctgagcaaggacacctacga | |
| | cgacgacctggacaacctgctggcccagatcggcgaccagtacgccgacc | |
| | tgtttctggccgccaagaacctgtccgacgccatcctgctgagcgacatc | |
| | ctgagagtgaacaccgagatcaccaaggcccccctgagcgcctctatgat | |
| | caagagatacgacgagcaccaccaggacctgaccctgctgaaagctctcg | |
| | tgcggcagcagctgcctgagaagtacaaagagattttcttcgaccagagc | |
| | aagaacggctacgccggctacattgacggcggagccagccaggaagagtt | |
| | ctacaagttcatcaagcccatcctggaaaagatggacggcaccgaggaac | |
| | tgctcgtgaagctgaacagagaggacctgctgcggaagcagcggaccttc | |
| | gacaacggcagcatcccccaccagatccacctgggagagctgcacgccat | |
| | tctgcggcggcaggaagatttttacccattcctgaaggacaaccgggaaa | |
| | agatcgagaagatcctgaccttccgcatcccctactacgtgggccctctg | |
| | gccaggggaaacagcagattcgcctggatgaccagaaagagcgaggaaac | |
| | catcaccccctggaacttcgaggaagtggtggacaagggcgcttccgccc | |
| | agagcttcatcgagcggatgaccaacttcgataagaacctgcccaacgag | |
| | aaggtgctgcccaagcacagcctgctgtacgagtacttcaccgtgtataa | |
| | cgagctgaccaaagtgaaatacgtgaccgagggaatgagaaagcccgcct | |
| | tcctgagcggcgagcagaaaaaggccatcgtggacctgctgttcaagacc | |
| | aaccggaaagtgaccgtgaagcagctgaaagaggactacttcaagaaaat | |
| | cgagtgcttcgactccgtggaaatctccggcgtggaagatcggttcaacg | |
| | cctccctgggcacataccacgatctgctgaaaattatcaaggacaaggac | |
| | ttcctggacaatgaggaaaacgaggacattctggaagatatcgtgctgac | |
| | cctgacactgtttgaggacagagagatgatcgaggaacggctgaaaacct | |
| | atgcccacctgttcgacgacaaagtgatgaagcagctgaagcggcggaga | |
| | tacaccggctggggcaggctgagccggaagctgatcaacggcatccggga | |
| | caagcagtccggcaagacaatcctggatttcctgaagtccgacggcttcg | |
| | ccaacagaaacttcatgcagctgatccacgacgacagcctgacctttaaa | |
| | gaggacatccagaaagcccaggtgtccggccagggcgatagcctgcacga | |
| | gcacattgccaatctggccggcagccccgccattaagaagggcatcctgc | |
| | agacagtgaaggtggtggacgagctcgtgaaagtgatgggccggcacaag | |
| | cccgagaacatcgtgatcgaaatggccagagagaaccagaccacccagaa | |
| | gggacagaagaacagccgcgagagaatgaagcggatcgaagagggcatca | |
| | aagagctgggcagccagatcctgaaagaacaccccgtggaaaacacccag | |
| | ctgcagaacgagaagctgtacctgtactacctgcagaatgggcgggatat | |
| | gtacgtggaccaggaactggacatcaaccggctgtccgactacgatgtgg | |
| | accatatcgtgcctcagagctttctgaaggacgactccatcgacaacaag | |
| | gtgctgaccagaagcgacaagaaccggggcaagagcgacaacgtgccctc | |
| | cgaagaggtcgtgaagaagatgaagaactactggcggcagctgctgaacg | |
| | ccaagctgattacccagagaaagttcgacaatctgaccaaggccgagaga | |
| | ggcggcctgagcgaactggataaggccggcttcatcaagagacagctggt | |
| | ggaaacccggcagatcacaaagcacgtggcacagatcctggactcccgga | |
| | tgaacactaagtacgacgagaatgacaagctgatccgggaagtgaaagtg | |
| | atcaccctgaagtccaagctggtgtccgatttccggaaggatttccagtt | |
| | ttacaaagtgcgcgagatcaacaactaccaccacgcccacgacgcctacc | |
| | tgaacgccgtcgtgggaaccgccctgatcaaaaagtaccctaagctggaa | |
| | agcgagttcgtgtacggcgactacaaggtgtacgacgtgcggaagatgat | |
| | cgccaagagcgagcaggaaatcggcaaggctaccgccaagtacttcttct | |
| | acagcaacatcatgaactttttcaagaccgagattaccctggccaacggc | |
| | gagatccggaagcggcctctgatcgagacaaacggcgaaaccggggagat | |
| | cgtgtgggataagggccgggattttgccaccgtgcggaaagtgctgagca | |
| | tgccccaagtgaatatcgtgaaaaagaccgaggtgcagacaggcggcttc | |
| | agcaaagagtctatcctgcccaagaggaacagcgataagctgatcgccag | |
| | aaagaaggactgggaccctaagaagtacggcggcttcctgtggcccaccg | |
| | tggcctattctgtgctggtggtggccaaagtggaaaagggcaagtccaag | |
| | aaactgaagagtgtgaaagagctgctggggatcaccatcatggaaagaag | |
| | cagcttcgagaagaatcccatcgactttctggaagccaagggctacaaag | |
| | aagtgaaaaaggacctgatcatcaagctgcctaagtactccctgttcgag | |
| | ctggaaaacggccggaagagaatgctggcctctgccaagcagctgcagaa | |
| | gggaaacgaactggccctgccctccaaatatgtgaacttcctgtacctgg | |
| | ccagccactatgagaagctgaagggctcccccgaggataatgagcagaaa | |
| | cagctgtttgtggaacagcacaagcactacctggacgagatcatcgagca | |
| | gatcagcgagttctccaagagagtgatcctggccgacgctaatctggaca | |
| | aagtgctgtccgcctacaacaagcaccgggataagcccatcagagagcag | |
| | gccgagaatatcatccacctgtttaccctgaccaatctgggagcccctgc | |
| | cgccttcaagtactttgacaccaccatcgaccggaagcagtacagaagca | |
| | ccaaagaggtgctggacgccaccctgatccaccagagcatcaccggcctg | |
| | tacgagacacggatcgacctgtctcagctgggaggtgac | |
| ABE8e-VRQR | tctgaggtggagttttcccacgagtactggatgagacatgccctgaccct | 149 |
| Encoding | ggccaagagggcacgggatgagagggaggtgcctgtgggagccgtgctgg | |
| sequences for | tgctgaacaatagagtgatcggcgagggctggaacagagccatcggcctg | |
| linkers are | cacgacccaacagcccatgccgaaattatggccctgagacagggcggcct | |
| underlined | ggtcatgcagaactacagactgattgacgccaccctgtacgtgacattcg | |
| | agccttgcgtgatgtgcgccggcgccatgatccactctaggatcggccgc | |
| | gtggtgtttggcgtgaggaactcaaaaagaggcgccgcaggctccctgat | |
| | gaacgtgctgaactaccccggcatgaatcaccgcgtcgaaattaccgagg | |
| | gaatcctggcagatgaatgtgccgccctgctgtgcgatttctatcggatg | |
| | cctagacaggtgttcaatgctcagaagaaggcccagagctccatcaactc | |
| | cggaggatctagcggaggctcctctggctctgagacacctggcacaagcg | |
| | agagcgcaacacctgaaagcagcgggggcagcagcggggggtcagacaag | |
| | aagtacagcatcggcctggccatcggcaccaactctgtgggctgggccgt | |
| | gatcaccgacgagtacaaggtgcccagcaagaaattcaaggtgctgggca | |
| | acaccgaccggcacagcatcaagaagaacctgatcggagccctgctgttc | |
| | gacagcggcgaaacagccgaggccacccggctgaagagaaccgccagaag | |
| | aagatacaccagacggaagaaccggatctgctatctgcaagagatcttca | |
| | gcaacgagatggccaaggtggacgacagcttcttccacagactggaagag | |
| | tccttcctggtggaagaggataagaagcacgagcggcaccccatcttcgg | |
| | caacatcgtggacgaggtggcctaccacgagaagtaccccaccatctacc | |
| | acctgagaaagaaactggtggacagcaccgacaaggccgacctgcggctg | |
| | atctatctggccctggcccacatgatcaagttccggggccacttcctgat | |
| | cgagggcgacctgaaccccgacaacagcgacgtggacaagctgttcatcc | |
| | agctggtgcagacctacaaccagctgttcgaggaaaaccccatcaacgcc | |
| | agcggcgtggacgccaaggccatcctgtctgccagactgagcaagagcag | |
| | acggctggaaaatctgatcgcccagctgcccggcgagaagaagaatggcc | |
| | tgttcggaaacctgattgccctgagcctgggcctgacccccaacttcaag | |
| | agcaacttcgacctggccgaggatgccaaactgcagctgagcaaggacac | |
| | ctacgacgacgacctggacaacctgctggcccagatcggcgaccagtacg | |
| | ccgacctgtttctggccgccaagaacctgtccgacgccatcctgctgagc | |
| | gacatcctgagagtgaacaccgagatcaccaaggcccccctgagcgcctc | |
| | tatgatcaagagatacgacgagcaccaccaggacctgaccctgctgaaag | |
| | ctctcgtgcggcagcagctgcctgagaagtacaaagagattttcttcgac | |
| | cagagcaagaacggctacgccggctacattgacggcggagccagccagga | |
| | agagttctacaagttcatcaagcccatcctggaaaagatggacggcaccg | |
| | aggaactgctcgtgaagctgaacagagaggacctgctgcggaagcagcgg | |
| | accttcgacaacggcagcatcccccaccagatccacctgggagagctgca | |
| | cgccattctgcggcggcaggaagatttttacccattcctgaaggacaacc | |
| | gggaaaagatcgagaagatcctgaccttccgcatcccctactacgtgggc | |
| | cctctggccaggggaaacagcagattcgcctggatgaccagaaagagcga | |
| | ggaaaccatcaccccctggaacttcgaggaagtggtggacaagggcgctt | |
| | ccgcccagagcttcatcgagcggatgaccaacttcgataagaacctgccc | |
| | aacgagaaggtgctgcccaagcacagcctgctgtacgagtacttcaccgt | |
| | gtataacgagctgaccaaagtgaaatacgtgaccgagggaatgagaaagc | |
| | ccgccttcctgagcggcgagcagaaaaaggccatcgtggacctgctgttc | |
| | aagaccaaccggaaagtgaccgtgaagcagctgaaagaggactacttcaa | |
| | gaaaatcgagtgcttcgactccgtggaaatctccggcgtggaagatcggt | |
| | tcaacgcctccctgggcacataccacgatctgctgaaaattatcaaggac | |
| | aaggacttcctggacaatgaggaaaacgaggacattctggaagatatcgt | |
| | gctgaccctgacactgtttgaggacagagagatgatcgaggaacggctga | |
| | aaacctatgcccacctgttcgacgacaaagtgatgaagcagctgaagcgg | |
| | cggagatacaccggctggggcaggctgagccggaagctgatcaacggcat | |
| | ccgggacaagcagtccggcaagacaatcctggatttcctgaagtccgacg | |
| | gcttcgccaacagaaacttcatgcagctgatccacgacgacagcctgacc | |
| | tttaaagaggacatccagaaagcccaggtgtccggccagggcgatagcct | |
| | gcacgagcacattgccaatctggccggcagccccgccattaagaagggca | |
| | tcctgcagacagtgaaggtggtggacgagctcgtgaaagtgatgggccgg | |
| | cacaagcccgagaacatcgtgatcgaaatggccagagagaaccagaccac | |
| | ccagaagggacagaagaacagccgcgagagaatgaagcggatcgaagagg | |
| | gcatcaaagagctgggcagccagatcctgaaagaacaccccgtggaaaac | |
| | acccagctgcagaacgagaagctgtacctgtactacctgcagaatggggg | |
| | gatatgtacgtggaccaggaactggacatcaaccggctgtccgactacga | |
| | tgtggaccatatcgtgcctcagagctttctgaaggacgactccatcgaca | |
| | acaaggtgctgaccagaagcgacaagaaccggggcaagagcgacaacgtg | |
| | ccctccgaagaggtcgtgaagaagatgaagaactactggcggcagctgct | |
| | gaacgccaagctgattacccagagaaagttcgacaatctgaccaaggccg | |
| | agagaggcggcctgagcgaactggataaggccggcttcatcaagagacag | |
| | ctggtggaaacccggcagatcacaaagcacgtggcacagatcctggactc | |
| | ccggatgaacactaagtacgacgagaatgacaagctgatccgggaagtga | |
| | aagtgatcaccctgaagtccaagctggtgtccgatttccggaaggatttc | |
| | cagttttacaaagtgcgcgagatcaacaactaccaccacgcccacgacgc | |
| | ctacctgaacgccgtcgtgggaaccgccctgatcaaaaagtaccctaagc | |
| | tggaaagcgagttcgtgtacggcgactacaaggtgtacgacgtgcggaag | |
| | atgatcgccaagagcgagcaggaaatcggcaaggctaccgccaagtactt | |
| | cttctacagcaacatcatgaactttttcaagaccgagattaccctggcca | |
| | acggcgagatccggaagcggcctctgatcgagacaaacggcgaaaccggg | |
| | gagatcgtgtgggataagggccgggattttgccaccgtgcggaaagtgct | |
| | gagcatgccccaagtgaatatcgtgaaaaagaccgaggtgcagacaggcg | |
| | gcttcagcaaagagtctatcctgcccaagaggaacagcgataagctgatc | |
| | gccagaaagaaggactgggaccctaagaagtacggcggcttcgtgagccc | |
| | caccgtggcctattctgtgctggtggtggccaaagtggaaaagggcaagt | |
| | ccaagaaactgaagagtgtgaaagagctgctggggatcaccatcatggaa | |
| | agaagcagcttcgagaagaatcccatcgactttctggaagccaagggcta | |
| | caaagaagtgaaaaaggacctgatcatcaagctgcctaagtactccctgt | |
| | tcgagctggaaaacggccggaagagaatgctggcctcagccagagaactg | |
| | cagaagggaaacgaactggccctgccctccaaatatgtgaacttcctgta | |
| | cctggccagccactatgagaagctgaagggctcccccgaggataatgagc | |
| | agaaacagctgtttgtggaacagcacaagcactacctggacgagatcatc | |
| | gagcagatcagcgagttctccaagagagtgatcctggccgacgctaatct | |
| | ggacaaagtgctgtccgcctacaacaagcaccgggataagcccatcagag | |
| | agcaggccgagaatatcatccacctgtttaccctgaccaatctgggagcc | |
| | cctgccgccttcaagtactttgacaccaccatcgaccggaagcagtacag | |
| | aagcaccaaagaggtgctggacgccaccctgatccaccagagcatcaccg | |
| | gcctgtacgagacacggatcgacctgtctcagctgggaggtgac | |
| ABE8e-SpCas9 | tctgaggtggagttttcccacgagtactggatgagacatgccctgaccct | 150 |
| Encoding | ggccaagagggcacgggatgagagggaggtgcctgtgggagccgtgctgg | |
| sequences for | tgctgaacaatagagtgatcggcgagggctggaacagagccatcggcctg | |
| linkers are | cacgacccaacagcccatgccgaaattatggccctgagacagggcggcct | |
| underlined | ggtcatgcagaactacagactgattgacgccaccctgtacgtgacattcg | |
| | agccttgcgtgatgtgcgccggcgccatgatccactctaggatcggccgc | |
| | gtggtgtttggcgtgaggaactcaaaaagaggcgccgcaggctccctgat | |
| | gaacgtgctgaactaccccggcatgaatcaccgcgtcgaaattaccgagg | |
| | gaatcctggcagatgaatgtgccgccctgctgtgcgatttctatcggatg | |
| | cctagacaggtgttcaatgctcagaagaaggcccagagctccatcaactc | |
| | cggaggatctagcggaggctcctctggctctgagacacctggcacaagcg | |
| | agagcgcaacacctgaaagcagcgggggcagcagcggggggtcagacaag | |
| | aagtacagcatcggcctggccatcggcaccaactctgtgggctgggccgt | |
| | gatcaccgacgagtacaaggtgcccagcaagaaattcaaggtgctgggca | |
| | acaccgaccggcacagcatcaagaagaacctgatcggagccctgctgttc | |
| | gacagcggcgaaacagccgaggccacccggctgaagagaaccgccagaag | |
| | aagatacaccagacggaagaaccggatctgctatctgcaagagatcttca | |
| | gcaacgagatggccaaggtggacgacagcttcttccacagactggaagag | |
| | tccttcctggtggaagaggataagaagcacgagcggcaccccatcttcgg | |
| | caacatcgtggacgaggtggcctaccacgagaagtaccccaccatctacc | |
| | acctgagaaagaaactggtggacagcaccgacaaggccgacctgcggctg | |
| | atctatctggccctggcccacatgatcaagttccggggccacttcctgat | |
| | cgagggcgacctgaaccccgacaacagcgacgtggacaagctgttcatcc | |
| | agctggtgcagacctacaaccagctgttcgaggaaaaccccatcaacgcc | |
| | agcggcgtggacgccaaggccatcctgtctgccagactgagcaagagcag | |
| | acggctggaaaatctgatcgcccagctgcccggcgagaagaagaatggcc | |
| | tgttcggaaacctgattgccctgagcctgggcctgacccccaacttcaag | |
| | agcaacttcgacctggccgaggatgccaaactgcagctgagcaaggacac | |
| | ctacgacgacgacctggacaacctgctggcccagatcggcgaccagtacg | |
| | ccgacctgtttctggccgccaagaacctgtccgacgccatcctgctgagc | |
| | gacatcctgagagtgaacaccgagatcaccaaggcccccctgagcgcctc | |
| | tatgatcaagagatacgacgagcaccaccaggacctgaccctgctgaaag | |
| | ctctcgtgcggcagcagctgcctgagaagtacaaagagattttcttcgac | |
| | cagagcaagaacggctacgccggctacattgacggcggagccagccagga | |
| | agagttctacaagttcatcaagcccatcctggaaaagatggacggcaccg | |
| | aggaactgctcgtgaagctgaacagagaggacctgctgcggaagcagcgg | |
| | accttcgacaacggcagcatcccccaccagatccacctgggagagctgca | |
| | cgccattctgcggcggcaggaagatttttacccattcctgaaggacaacc | |
| | gggaaaagatcgagaagatcctgaccttccgcatcccctactacgtgggc | |
| | cctctggccaggggaaacagcagattcgcctggatgaccagaaagagcga | |
| | ggaaaccatcaccccctggaacttcgaggaagtggtggacaagggcgctt | |
| | ccgcccagagcttcatcgagcggatgaccaacttcgataagaacctgccc | |
| | aacgagaaggtgctgcccaagcacagcctgctgtacgagtacttcaccgt | |
| | gtataacgagctgaccaaagtgaaatacgtgaccgagggaatgagaaagc | |
| | ccgccttcctgagcggcgagcagaaaaaggccatcgtggacctgctgttc | |
| | aagaccaaccggaaagtgaccgtgaagcagctgaaagaggactacttcaa | |
| | gaaaatcgagtgcttcgactccgtggaaatctccggcgtggaagatcggt | |
| | tcaacgcctccctgggcacataccacgatctgctgaaaattatcaaggac | |
| | aaggacttcctggacaatgaggaaaacgaggacattctggaagatatcgt | |
| | gctgaccctgacactgtttgaggacagagagatgatcgaggaacggctga | |
| | aaacctatgcccacctgttcgacgacaaagtgatgaagcagctgaagcgg | |
| | cggagatacaccggctggggcaggctgagccggaagctgatcaacggcat | |
| | ccgggacaagcagtccggcaagacaatcctggatttcctgaagtccgacg | |
| | gcttcgccaacagaaacttcatgcagctgatccacgacgacagcctgacc | |
| | tttaaagaggacatccagaaagcccaggtgtccggccagggcgatagcct | |
| | gcacgagcacattgccaatctggccggcagccccgccattaagaagggca | |
| | tcctgcagacagtgaaggtggtggacgagctcgtgaaagtgatgggccgg | |
| | cacaagcccgagaacatcgtgatcgaaatggccagagagaaccagaccac | |
| | ccagaagggacagaagaacagccgcgagagaatgaagcggatcgaagagg | |
| | gcatcaaagagctgggcagccagatcctgaaagaacaccccgtggaaaac | |
| | acccagctgcagaacgagaagctgtacctgtactacctgcagaatggggg | |
| | gatatgtacgtggaccaggaactggacatcaaccggctgtccgactacga | |
| | tgtggaccatatcgtgcctcagagctttctgaaggacgactccatcgaca | |
| | acaaggtgctgaccagaagcgacaagaaccggggcaagagcgacaacgtg | |
| | ccctccgaagaggtcgtgaagaagatgaagaactactggcggcagctgct | |
| | gaacgccaagctgattacccagagaaagttcgacaatctgaccaaggccg | |
| | agagaggcggcctgagcgaactggataaggccggcttcatcaagagacag | |
| | ctggtggaaacccggcagatcacaaagcacgtggcacagatcctggactc | |
| | ccggatgaacactaagtacgacgagaatgacaagctgatccgggaagtga | |
| | aagtgatcaccctgaagtccaagctggtgtccgatttccggaaggatttc | |
| | cagttttacaaagtgcgcgagatcaacaactaccaccacgcccacgacgc | |
| | ctacctgaacgccgtcgtgggaaccgccctgatcaaaaagtaccctaagc | |
| | tggaaagcgagttcgtgtacggcgactacaaggtgtacgacgtgcggaag | |
| | atgatcgccaagagcgagcaggaaatcggcaaggctaccgccaagtactt | |
| | cttctacagcaacatcatgaactttttcaagaccgagattaccctggcca | |
| | acggcgagatccggaagcggcctctgatcgagacaaacggcgaaaccggg | |
| | gagatcgtgtgggataagggccgggattttgccaccgtgcggaaagtgct | |
| | gagcatgccccaagtgaatatcgtgaaaaagaccgaggtgcagacaggcg | |
| | gcttcagcaaagagtctatcaggcccaagaggaacagcgataagctgatc | |
| | gccagaaagaaggactgggaccctaagaagtacggcggcttcgtcagccc | |
| | caccgtggcctattctgtgctggtggtggccaaagtggaaaagggcaagt | |
| | ccaagaaactgaagagtgtgaaagagctgctggggatcaccatcatggaa | |
| | agaagcagcttcgagaagaatcccatcgactttctggaagccaagggcta | |
| | caaagaagtgaaaaaggacctgatcatcaagctgcctaagtactccctgt | |
| | tcgagctggaaaacggccggaagagaatgctggcctctgccagattcctg | |
| | cagaagggaaacgaactggccctgccctccaaatatgtgaacttcctgta | |
| | cctggccagccactatgagaagctgaagggctcccccgaggataatgagc | |
| | agaaacagctgtttgtggaacagcacaagcactacctggacgagatcatc | |
| | gagcagatcagcgagttctccaagagagtgatcctggccgacgctaatct | |
| | ggacaaagtgctgtccgcctacaacaagcaccgggataagcccatcagag | |
| | agcaggccgagaatatcatccacctgtttaccctgaccaatctgggagcc | |
| | cctagggccttcaagtactttgacaccaccatcgaccggaaggtgtacag | |
| | gagcaccaaagaggtgctggacgccaccctgatccaccagagcatcaccg | |
| | gcctgtacgagacacggatcgacctgtctcagctgggaggtgac | |
| ABE8e-SpRY | tctgaggtggagttttcccacgagtactggatgagacatgccctgaccct | 151 |
| Encoding | ggccaagagggcacgggatgagagggaggtgcctgtgggagccgtgctgg | |
| sequences for | tgctgaacaatagagtgatcggcgagggctggaacagagccatcggcctg | |
| linkers are | cacgacccaacagcccatgccgaaattatggccctgagacagggcggcct | |
| underlined | ggtcatgcagaactacagactgattgacgccaccctgtacgtgacattcg | |
| | agccttgcgtgatgtgcgccggcgccatgatccactctaggatcggccgc | |
| | gtggtgtttggcgtgaggaactcaaaaagaggcgccgcaggctccctgat | |
| | gaacgtgctgaactaccccggcatgaatcaccgcgtcgaaattaccgagg | |
| | gaatcctggcagatgaatgtgccgccctgctgtgcgatttctatcggatg | |
| | cctagacaggtgttcaatgctcagaagaaggcccagagctccatcaactc | |
| | cggaggatctagcggaggctcctctggctctgagacacctggcacaagcg | |
| | agagcgcaacacctgaaagcagcgggggcagcagcggggggtcagacaag | |
| | aagtacagcatcggcctggccatcggcaccaactctgtgggctgggccgt | |
| | gatcaccgacgagtacaaggtgcccagcaagaaattcaaggtgctgggca | |
| | acaccgaccggcacagcatcaagaagaacctgatcggagccctgctgttc | |
| | gacagcggcgaaacagccgagagaacccggctgaagagaaccgccagaag | |
| | aagatacaccagacggaagaaccggatctgctatctgcaagagatcttca | |
| | gcaacgagatggccaaggtggacgacagcttcttccacagactggaagag | |
| | tccttcctggtggaagaggataagaagcacgagcggcaccccatcttcgg | |
| | caacatcgtggacgaggtggcctaccacgagaagtaccccaccatctacc | |
| | acctgagaaagaaactggtggacagcaccgacaaggccgacctgcggctg | |
| | atctatctggccctggcccacatgatcaagttccggggccacttcctgat | |
| | cgagggcgacctgaaccccgacaacagcgacgtggacaagctgttcatcc | |
| | agctggtgcagacctacaaccagctgttcgaggaaaaccccatcaacgcc | |
| | agcggcgtggacgccaaggccatcctgtctgccagactgagcaagagcag | |
| | acggctggaaaatctgatcgcccagctgcccggcgagaagaagaatggcc | |
| | tgttcggaaacctgattgccctgagcctgggcctgacccccaacttcaag | |
| | agcaacttcgacctggccgaggatgccaaactgcagctgagcaaggacac | |
| | ctacgacgacgacctggacaacctgctggcccagatcggcgaccagtacg | |
| | ccgacctgtttctggccgccaagaacctgtccgacgccatcctgctgagc | |
| | gacatcctgagagtgaacaccgagatcaccaaggcccccctgagcgcctc | |
| | tatgatcaagagatacgacgagcaccaccaggacctgaccctgctgaaag | |
| | ctctcgtgcggcagcagctgcctgagaagtacaaagagattttcttcgac | |
| | cagagcaagaacggctacgccggctacattgacggcggagccagccagga | |
| | agagttctacaagttcatcaagcccatcctggaaaagatggacggcaccg | |
| | aggaactgctcgtgaagctgaacagagaggacctgctgcggaagcagcgg | |
| | accttcgacaacggcagcatcccccaccagatccacctgggagagctgca | |
| | cgccattctgcggcggcaggaagatttttacccattcctgaaggacaacc | |
| | gggaaaagatcgagaagatcctgaccttccgcatcccctactacgtgggc | |
| | cctctggccaggggaaacagcagattcgcctggatgaccagaaagagcga | |
| | ggaaaccatcaccccctggaacttcgaggaagtggtggacaagggcgctt | |
| | ccgcccagagcttcatcgagcggatgaccaacttcgataagaacctgccc | |
| | aacgagaaggtgctgcccaagcacagcctgctgtacgagtacttcaccgt | |
| | gtataacgagctgaccaaagtgaaatacgtgaccgagggaatgagaaagc | |
| | ccgccttcctgagcggcgagcagaaaaaggccatcgtggacctgctgttc | |
| | aagaccaaccggaaagtgaccgtgaagcagctgaaagaggactacttcaa | |
| | gaaaatcgagtgcttcgactccgtggaaatctccggcgtggaagatcggt | |
| | tcaacgcctccctgggcacataccacgatctgctgaaaattatcaaggac | |
| | aaggacttcctggacaatgaggaaaacgaggacattctggaagatatcgt | |
| | gctgaccctgacactgtttgaggacagagagatgatcgaggaacggctga | |
| | aaacctatgcccacctgttcgacgacaaagtgatgaagcagctgaagcgg | |
| | cggagatacaccggctggggcaggctgagccggaagctgatcaacggcat | |
| | ccgggacaagcagtccggcaagacaatcctggatttcctgaagtccgacg | |
| | gcttcgccaacagaaacttcatgcagctgatccacgacgacagcctgacc | |
| | tttaaagaggacatccagaaagcccaggtgtccggccagggcgatagcct | |
| | gcacgagcacattgccaatctggccggcagccccgccattaagaagggca | |
| | tcctgcagacagtgaaggtggtggacgagctcgtgaaagtgatgggccgg | |
| | cacaagcccgagaacatcgtgatcgaaatggccagagagaaccagaccac | |
| | ccagaagggacagaagaacagccgcgagagaatgaagcggatcgaagagg | |
| | gcatcaaagagctgggcagccagatcctgaaagaacaccccgtggaaaac | |
| | acccagctgcagaacgagaagctgtacctgtactacctgcagaatggggg | |
| | gatatgtacgtggaccaggaactggacatcaaccggctgtccgactacga | |
| | tgtggaccatatcgtgcctcagagctttctgaaggacgactccatcgaca | |
| | acaaggtgctgaccagaagcgacaagaaccggggcaagagcgacaacgtg | |
| | ccctccgaagaggtcgtgaagaagatgaagaactactggcggcagctgct | |
| | gaacgccaagctgattacccagagaaagttcgacaatctgaccaaggccg | |
| | agagaggcggcctgagcgaactggataaggccggcttcatcaagagacag | |
| | ctggtggaaacccggcagatcacaaagcacgtggcacagatcctggactc | |
| | ccggatgaacactaagtacgacgagaatgacaagctgatccgggaagtga | |
| | aagtgatcaccctgaagtccaagctggtgtccgatttccggaaggatttc | |
| | cagttttacaaagtgcgcgagatcaacaactaccaccacgcccacgacgc | |
| | ctacctgaacgccgtcgtgggaaccgccctgatcaaaaagtaccctaagc | |
| | tggaaagcgagttcgtgtacggcgactacaaggtgtacgacgtgcggaag | |
| | atgatcgccaagagcgagcaggaaatcggcaaggctaccgccaagtactt | |
| | cttctacagcaacatcatgaactttttcaagaccgagattaccctggcca | |
| | acggcgagatccggaagcggcctctgatcgagacaaacggcgaaaccggg | |
| | gagatcgtgtgggataagggccgggattttgccaccgtgcggaaagtgct | |
| | gagcatgccccaagtgaatatcgtgaaaaagaccgaggtgcagacaggcg | |
| | gcttcagcaaagagtctatcagacccaagaggaacagcgataagctgatc | |
| | gccagaaagaaggactgggaccctaagaagtacggcggcttcctgtggcc | |
| | caccgtggcctattctgtgctggtggtggccaaagtggaaaagggcaagt | |
| | ccaagaaactgaagagtgtgaaagagctgctggggatcaccatcatggaa | |
| | agaagcagcttcgagaagaatcccatcgactttctggaagccaagggcta | |
| | caaagaagtgaaaaaggacctgatcatcaagctgcctaagtactccctgt | |
| | tcgagctggaaaacggccggaagagaatgctggcctctgccaagcagctg | |
| | cagaagggaaacgaactggccctgccctccaaatatgtgaacttcctgta | |
| | cctggccagccactatgagaagctgaagggctcccccgaggataatgagc | |
| | agaaacagctgtttgtggaacagcacaagcactacctggacgagatcatc | |
| | gagcagatcagcgagttctccaagagagtgatcctggccgacgctaatct | |
| | ggacaaagtgctgtccgcctacaacaagcaccgggataagcccatcagag | |
| | agcaggccgagaatatcatccacctgtttaccctgaccagactgggagcc | |
| | cctagagccttcaagtactttgacaccaccatcgaccccaagcagtacag | |
| | aagcaccaaagaggtgctggacgccaccctgatccaccagagcatcaccg | |
| | gcctgtacgagacacggatcgacctgtctcagctgggaggtgac | |
| ABE8e-SpG | tctgaggtggagttttcccacgagtactggatgagacatgccctgaccct | 152 |
| Encoding | ggccaagagggcacgggatgagagggaggtgcctgtgggagccgtgctgg | |
| sequences for | tgctgaacaatagagtgatcggcgagggctggaacagagccatcggcctg | |
| linkers are | cacgacccaacagcccatgccgaaattatggccctgagacagggcggcct | |
| underlined | ggtcatgcagaactacagactgattgacgccaccctgtacgtgacattcg | |
| | agccttgcgtgatgtgcgccggcgccatgatccactctaggatcggccgc | |
| | gtggtgtttggcgtgaggaactcaaaaagaggcgccgcaggctccctgat | |
| | gaacgtgctgaactaccccggcatgaatcaccgcgtcgaaattaccgagg | |
| | gaatcctggcagatgaatgtgccgccctgctgtgcgatttctatcggatg | |
| | cctagacaggtgttcaatgctcagaagaaggcccagagctccatcaactc | |
| | cggaggatctagcggaggctcctctggctctgagacacctggcacaagcg | |
| | agagcgcaacacctgaaagcagcgggggcagcagcggggggtcagacaag | |
| | aagtacagcatcggcctggccatcggcaccaactctgtgggctgggccgt | |
| | gatcaccgacgagtacaaggtgcccagcaagaaattcaaggtgctgggca | |
| | acaccgaccggcacagcatcaagaagaacctgatcggagccctgctgttc | |
| | gacagcggcgaaacagccgaggccacccggctgaagagaaccgccagaag | |
| | aagatacaccagacggaagaaccggatctgctatctgcaagagatcttca | |
| | gcaacgagatggccaaggtggacgacagcttcttccacagactggaagag | |
| | tccttcctggtggaagaggataagaagcacgagcggcaccccatcttcgg | |
| | caacatcgtggacgaggtggcctaccacgagaagtaccccaccatctacc | |
| | acctgagaaagaaactggtggacagcaccgacaaggccgacctgcggctg | |
| | atctatctggccctggcccacatgatcaagttccggggccacttcctgat | |
| | cgagggcgacctgaaccccgacaacagcgacgtggacaagctgttcatcc | |
| | agctggtgcagacctacaaccagctgttcgaggaaaaccccatcaacgcc | |
| | agcggcgtggacgccaaggccatcctgtctgccagactgagcaagagcag | |
| | acggctggaaaatctgatcgcccagctgcccggcgagaagaagaatggcc | |
| | tgttcggaaacctgattgccctgagcctgggcctgacccccaacttcaag | |
| | agcaacttcgacctggccgaggatgccaaactgcagctgagcaaggacac | |
| | ctacgacgacgacctggacaacctgctggcccagatcggcgaccagtacg | |
| | ccgacctgtttctggccgccaagaacctgtccgacgccatcctgctgagc | |
| | gacatcctgagagtgaacaccgagatcaccaaggcccccctgagcgcctc | |
| | tatgatcaagagatacgacgagcaccaccaggacctgaccctgctgaaag | |
| | ctctcgtgcggcagcagctgcctgagaagtacaaagagattttcttcgac | |
| | cagagcaagaacggctacgccggctacattgacggcggagccagccagga | |
| | agagttctacaagttcatcaagcccatcctggaaaagatggacggcaccg | |
| | aggaactgctcgtgaagctgaacagagaggacctgctgcggaagcagcgg | |
| | accttcgacaacggcagcatcccccaccagatccacctgggagagctgca | |
| | cgccattctgcggcggcaggaagatttttacccattcctgaaggacaacc | |
| | gggaaaagatcgagaagatcctgaccttccgcatcccctactacgtgggc | |
| | cctctggccaggggaaacagcagattcgcctggatgaccagaaagagcga | |
| | ggaaaccatcaccccctggaacttcgaggaagtggtggacaagggcgctt | |
| | ccgcccagagcttcatcgagcggatgaccaacttcgataagaacctgccc | |
| | aacgagaaggtgctgcccaagcacagcctgctgtacgagtacttcaccgt | |
| | gtataacgagctgaccaaagtgaaatacgtgaccgagggaatgagaaagc | |
| | ccgccttcctgagcggcgagcagaaaaaggccatcgtggacctgctgttc | |
| | aagaccaaccggaaagtgaccgtgaagcagctgaaagaggactacttcaa | |
| | gaaaatcgagtgcttcgactccgtggaaatctccggcgtggaagatcggt | |
| | tcaacgcctccctgggcacataccacgatctgctgaaaattatcaaggac | |
| | aaggacttcctggacaatgaggaaaacgaggacattctggaagatatcgt | |
| | gctgaccctgacactgtttgaggacagagagatgatcgaggaacggctga | |
| | aaacctatgcccacctgttcgacgacaaagtgatgaagcagctgaagcgg | |
| | cggagatacaccggctggggcaggctgagccggaagctgatcaacggcat | |
| | ccgggacaagcagtccggcaagacaatcctggatttcctgaagtccgacg | |
| | gcttcgccaacagaaacttcatgcagctgatccacgacgacagcctgacc | |
| | tttaaagaggacatccagaaagcccaggtgtccggccagggcgatagcct | |
| | gcacgagcacattgccaatctggccggcagccccgccattaagaagggca | |
| | tcctgcagacagtgaaggtggtggacgagctcgtgaaagtgatgggccgg | |
| | cacaagcccgagaacatcgtgatcgaaatggccagagagaaccagaccac | |
| | ccagaagggacagaagaacagccgcgagagaatgaagcggatcgaagagg | |
| | gcatcaaagagctgggcagccagatcctgaaagaacaccccgtggaaaac | |
| | acccagctgcagaacgagaagctgtacctgtactacctgcagaatgggcg | |
| | ggatatgtacgtggaccaggaactggacatcaaccggctgtccgactacg | |
| | atgtggaccatatcgtgcctcagagctttctgaaggacgactccatcgac | |
| | aacaaggtgctgaccagaagcgacaagaaccggggcaagagcgacaacgt | |
| | gccctccgaagaggtcgtgaagaagatgaagaactactggcggcagctgc | |
| | tgaacgccaagctgattacccagagaaagttcgacaatctgaccaaggcc | |
| | gagagaggcggcctgagcgaactggataaggccggcttcatcaagagaca | |
| | gctggtggaaacccggcagatcacaaagcacgtggcacagatcctggact | |
| | cccggatgaacactaagtacgacgagaatgacaagctgatccgggaagtg | |
| | aaagtgatcaccctgaagtccaagctggtgtccgatttccggaaggattt | |
| | ccagttttacaaagtgcgcgagatcaacaactaccaccacgcccacgacg | |
| | cctacctgaacgccgtcgtgggaaccgccctgatcaaaaagtaccctaag | |
| | ctggaaagcgagttcgtgtacggcgactacaaggtgtacgacgtgcggaa | |
| | gatgatcgccaagagcgagcaggaaatcggcaaggctaccgccaagtact | |
| | tcttctacagcaacatcatgaactttttcaagaccgagattaccctggcc | |
| | aacggcgagatccggaagcggcctctgatcgagacaaacggcgaaaccgg | |
| | ggagatcgtgtgggataagggccgggattttgccaccgtgcggaaagtgc | |
| | tgagcatgccccaagtgaatatcgtgaaaaagaccgaggtgcagacaggc | |
| | ggcttcagcaaagagtctatcctgcccaagaggaacagcgataagctgat | |
| | cgccagaaagaaggactgggaccctaagaagtacggcggcttcctgtggc | |
| | ccaccgtggcctattctgtgctggtggtggccaaagtggaaaagggcaag | |
| | tccaagaaactgaagagtgtgaaagagctgctggggatcaccatcatgga | |
| | aagaagcagcttcgagaagaatcccatcgactttctggaagccaagggct | |
| | acaaagaagtgaaaaaggacctgatcatcaagctgcctaagtactccctg | |
| | ttcgagctggaaaacggccggaagagaatgctggcctctgccaagcagct | |
| | gcagaagggaaacgaactggccctgccctccaaatatgtgaacttcctgt | |
| | acctggccagccactatgagaagctgaagggctcccccgaggataatgag | |
| | cagaaacagctgtttgtggaacagcacaagcactacctggacgagatcat | |
| | cgagcagatcagcgagttctccaagagagtgatcctggccgacgctaatc | |
| | tggacaaagtgctgtccgcctacaacaagcaccgggataagcccatcaga | |
| | gagcaggccgagaatatcatccacctgtttaccctgaccaatctgggagc | |
| | ccctgccgccttcaagtactttgacaccaccatcgaccggaagcagtaca | |
| | gaagcaccaaagaggtgctggacgccaccctgatccaccagagcatcacc | |
| | ggcctgtacgagacacggatcgacctgtctcagctgggaggtgac | |

### (c) CRISPR Gene Editing Systems

In some embodiments, engineered CRISPR gene editing systems herein (e.g., for gene editing in mammalian cells) can include (1) a guide RNA molecule (gRNA) as disclosed herein comprising a targeting domain (which is capable of hybridizing to the genomic DNA target sequence), and sequence which is capable of binding to a Cas, e.g., Cas9 enzyme, and (2) a base editor (e.g., a fusion protein of a deaminase and a Cas9 nickase or deactivated Cas9 endonuclease). In some aspects, the engineered CRISPR gene editing system comprises a gRNA targeting a sequence of SEQ ID NO: 1 or 2 and a fusion protein comprising any one of SEQ ID NOs: 45 to 60. In some aspects, the engineered CRISPR gene editing system comprises a gRNA targeting a sequence of SEQ ID NO: 1 (i.e., comprising a spacer sequence of SEQ ID NO: 5) and a fusion protein comprising SEQ ID NO: 45 or 46. In some aspects, the engineered CRISPR gene editing system comprises a gRNA targeting a sequence of SEQ ID NO: 2 (i.e., comprising a spacer sequence of SEQ ID NO: 6) and a fusion protein comprising SEQ ID NO: 45 or 46.

### (i) Further Elements of CRISPR Systems

The gRNA may comprise a domain referred to as a tracr domain. The targeting domain and the sequence which is capable of binding to a Cas, e.g., Cas9 enzyme, may be disposed on the same (sometimes referred to as a single gRNA, chimeric gRNA or sgRNA) or different molecules (sometimes referred to as a dual gRNA or dgRNA). If disposed on different molecules, each includes a hybridization domain which allows the molecules to associate, e.g., through hybridization.

In certain embodiments, to generate a double stranded break in the target sequence, CRISPR-Cas9 systems herein can bind to a target sequence as determined by the guide nucleic acid (gRNA), and the nuclease recognizes a protospacer adjacent motif (PAM) sequence adjacent to the target sequence in order to cut the target sequence. In some embodiments, CRISPR-Cas9 systems herein can include a scaffold sequence compatible with the nucleic acid-guided nuclease. In other embodiments, the guide sequence can be engineered to be complementary to any desired target sequence for efficient editing of the target sequence. In other embodiments, the guide sequence can be engineered to hybridize to any desired target sequence. In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length.

In some embodiments, a target sequence of CRISPR-Cas9 systems herein can be any polynucleotide endogenous or exogenous to a prokaryotic or eukaryotic cell, or in an in vitro system for verification or otherwise. In other embodiments, a target sequence can be a polynucleotide residing in the nucleus of the eukaryotic cell. A target sequence can be a sequence coding a gene product (e.g., a protein) or a non-coding sequence (e.g., a regulatory polynucleotide or a junk DNA). It is contemplated herein that the target sequence should be associated with a PAM; that is, a short sequence recognized by CRISPR-Cas9 systems herein. In some embodiments, sequence and length requirements for a PAM differ depending on the nucleic acid-guided nuclease selected. In certain embodiments, PAM sequences can be about 2-5 base pair sequences adjacent the target sequence or longer, depending on the PAM desired. Examples of PAM sequences are given in the Examples section below, and the skilled person will be able to identify further PAM sequences for use with a given nucleic acid-guided nuclease as these are not intended to limit this aspect of the present inventive concept. Further, engineering of a PAM Interacting (PI) domain can allow programming of PAM specificity, improve target site recognition fidelity, and increase the versatility of a nucleic acid-guided nuclease genome engineering platform.

### (d) Isolated Nucleic Acids and Vectors

In various aspects, one or more components of the CRISPR gene editing system provided herein (e.g., the gRNA and/or the fusion protein (base editor) may be encoded by a nucleic acid (e.g., those described above). Accordingly, provided herein are isolated nucleic acids encoding one or more gRNAs described above. Also provided are isolated nucleic acids encoding a fusion protein comprising a deaminase and a Cas9 nickase or Cas9 endonuclease. Exemplary nucleic acids that may be provided as isolated nucleic acids according to the present disclosure are described in the tables above.

Polynucleotide sequences encoding a component of CRISPR-Cas9 systems herein can include one or more vectors. The term “vector” as used herein can refer to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Other vectors (e.g., non-episomal mammalian vectors) can be integrated into the genome of a host cell upon introduction into the host cell. Recombinant expression vectors can include a nucleic acid of the present inventive concept in a form suitable for expression of the nucleic acid in a host cell, can mean that the recombinant expression vectors include one or more regulatory elements, which can be selected on the basis of the host cells to be used for expression, that is operatively-linked to the nucleic acid sequence to be expressed.

In some embodiments, a regulatory element can be operably linked to one or more elements of a targetable CRISPR-Cas9 system herein so as to drive expression of the one or more components of the targetable CRISPR-Cas9 system.

In some embodiments, a vector can include a regulatory element operably linked to a polynucleotide sequence encoding a Cas9 nuclease herein. The polynucleotide sequence encoding the Cas9 nuclease herein can be codon optimized for expression in particular cells, such as prokaryotic or eukaryotic cells. Eukaryotic cells can be yeast, fungi, algae, plant, animal, or human cells. Eukaryotic cells can be those derived from a particular organism, such as a mammal, including but not limited to human, mouse, rat, rabbit, dog, or non-human mammal including non-human primate. Plant cells can include, without limitation, cells from seeds, suspension cultures, embryos, meristematic regions, callus tissue, leaves, roots, shoots, gametophytes, sporophytes, pollen and microspores.

As used herein, ‘codon optimization’ can refer to a process of modifying a nucleic acid sequence for enhanced expression in the host cells of interest by replacing at least one codon or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.”

In some embodiments, a Cas9 nuclease herein and one or more guide nucleic acids (e.g., gRNA) can be delivered either as DNA or RNA. Delivery of a Cas9 nuclease herein and guide nucleic acid both as RNA (unmodified or containing base or backbone modifications) molecules can be used to reduce the amount of time that the nucleic acid-guided nuclease persist in the cell (e.g. reduced half-life). This can reduce the level of off-target cleavage activity in the target cell. Since delivery of a Cas9 nuclease as mRNA takes time to be translated into protein, an aspect herein can include delivering a guide nucleic acid several hours following the delivery of the Cas9 mRNA, to maximize the level of guide nucleic acid available for interaction with the nucleic acid-guided nuclease protein. In other cases, the Cas9 mRNA and guide nucleic acid can be delivered concomitantly. In other examples, the guide nucleic acid can be delivered sequentially, such as 0.5, 1, 2, 3, 4, or more hours after the Cas9 mRNA.

In some embodiments, guide nucleic acid (e.g., gRNA) in the form of RNA or encoded on a DNA expression cassette can be introduced into a host cell that includes a nucleic acid-guided nuclease encoded on a vector or chromosome. The guide nucleic acid can be provided in the cassette having one or more polynucleotides, which can be contiguous or non-contiguous in the cassette. In some embodiments, the guide nucleic acid can be provided in the cassette as a single contiguous polynucleotide. In other embodiments, a tracking agent can be added to the guide nucleic acid in order to track distribution and activity.

In other embodiments, a variety of delivery systems can be used to introduce a gRNA and/or Cas9 nuclease into a host cell. In accordance with these embodiments, systems of use for embodiments disclosed herein can include, but are not limited to, yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, and/or exosomes.

In some embodiments, methods are provided for delivering one or more polynucleotides, such as or one or more vectors or linear polynucleotides as described herein, one or more transcripts thereof, and/or one or proteins transcribed therefrom, to a host cell. In some aspects, the present inventive concept further provides cells produced by such methods, and organisms can include or produced from such cells. In some embodiments, an engineered nuclease in combination with (and optionally complexed with) a guide nucleic acid is delivered to a cell.

In certain embodiments, conventional viral and non-viral based gene transfer methods can be used to introduce nucleic acids in cells, such as prokaryotic cells, eukaryotic cells, plant cells, mammalian cells, or target tissues. Such methods can be used to administer nucleic acids encoding components of an CRISPR-Cas9 system herein to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Any gene therapy method known in the art is contemplated of use herein. Methods of non-viral delivery of nucleic acids include are contemplated herein. Adeno-associated virus (“AAV”) vectors can also be used to transduce cells with target nucleic acids, e.g., in the in vitro production of nucleic acids and peptides, and for in vivo and ex vivo gene therapy procedures.

In some embodiments, a nucleic acid encoding any of the constructs herein (e.g., gRNA, fusion proteins comprising the deaminase and Cas9 nickase or deactivated Cas9 protein) can be delivered to a cell using an adeno-associated virus (AAV). AAVs are small viruses which integrate site-specifically into the host genome and can therefore deliver a transgene. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, any mammalian AAV serotypes can be used herein for delivering the encoding nucleic acids described herein. Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

In some embodiments, polynucleotides disclosed herein (e.g., gRNA, Cas9) can be delivered to a cell using at least one AAV vector. An AAV vector typically comprises a protein-based capsid, and a nucleic acid encapsidated by the capsid. The nucleic acid may be, for example, a vector genome comprising a transgene flanked by inverted terminal repeats. The AAV “capsid” is a near-spherical protein shell that comprises individual “capsid proteins” or “subunits.” AAV capsids typically comprise about 60 capsid protein subunits, associated and arranged with T=1 icosahedral symmetry. When an AAV vector is described herein as comprising an AAV capsid protein, it will be understood that the AAV vector comprises a capsid, wherein the capsid comprises one or more AAV capsid proteins (i.e., subunits). Also described herein are “viral-like particles” or “virus-like particles,” which refers to a capsid that does not comprise any vector genome or nucleic acid comprising a transgene. The virus vectors of the present disclosure can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619. The virus vectors of the present disclosure can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the present inventive concept. Further, the viral capsid or genomic elements can contain other modifications, including insertions, deletions and/or substitutions.

In some embodiments, the isolated nucleic acids encoding a gRNA and/or the fusion proteins herein may be packaged into an AAV vector (e.g., a AAV-Cas9 vector). In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11 or any combination thereof.

Exemplary AAV-Cas9 vectors contain two ITR (inverted terminal repeat) sequences which flank a central sequence region comprising the Cas9 sequence. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-Cas9 vector may contain one or more nuclear localization signals (NLS). In some embodiments, the AAV-Cas9 vector contains 1, 2, 3, 4, or 5 nuclear localization signals. Exemplary NLS include SEQ ID NOs: 31 and 32. Other exemplary NLS include the c-myc NLS, the SV40 NLS, the hnRNPAI M9 NLS, the nucleoplasmin NLS, the sequence RMRKFKNKGKDTAELRRRRVEVSVELRKAKKDEQILKRRNV (SEQ ID NO: 33) of the IBB domain from importin-alpha, the sequences VSRKRPRP (SEQ ID NO: 34) and PPKKARED (SEQ ID NO: 35) of the myoma T protein, the sequence PQPKKKPL (SEQ ID NO: 104) of human p53, the sequence SALIKKKKKMAP (SEQ ID NO: 36) of mouse c-abl IV, the sequences DRLRR (SEQ ID NO: 37) and PKQKKRK (SEQ ID NO:38) of the influenza virus NS1, the sequence RKLKKKIKKL (SEQ ID NO: 39) of the Hepatitis virus delta antigen and the sequence REKKKFLKRR (SEQ ID NO: 40) of the mouse Mx1 protein. Further acceptable nuclear localization signals include bipartite nuclear localization sequences such as the sequence KRKGDEVDGVDEVAKKKSKK (SEQ ID NO: 41) of the human poly(ADP-ribose) polymerase or the sequence RKCLQAGMNLEARKTKK (SEQ ID NO: 42) of the steroid hormone receptors (human) glucocorticoid.

In some embodiments, the AAV-Cas9 vector may comprise additional elements to facilitate packaging of the vector and expression of the fusion protein and/or gRNA. In some embodiments, the AAV-Cas9 vector may comprise a polyA sequence. In some embodiments, the polyA sequence may be a bgHi-polyA sequence. In some embodiments, the AAV-Cas9 vector may comprise a regulator element. In some embodiments, the regulator element is an activator or a repressor. In some embodiments, a regulator element is a posttranscriptional regulatory element (e.g., WPRE-3-Woodchuck Hepatitis Virus Posttranscriptional Regulatory Element-3)

In some embodiments, the AAV-Cas9 may contain one or more promoters. In some embodiments, the one or more promoters drive expression of the Cas9. In some embodiments, the one or more promoters are muscle-specific promoters. Exemplary muscle-specific promoters include myosin light chain-2 promoter, the α-actin promoter, the troponin 1 promoter, the Na+/Ca2+ exchanger promoter, the dystrophin promoter, the α7 integrin promoter, the brain natriuretic peptide promoter, the αB-crystallin/small heat shock protein promoter, α-myosin heavy chain promoter, the ANF promoter, the CK8 promoter and the CK8e promoter. In some embodiments, the one or more promoters are cardiac-specific promoters. Exemplary cardiac-specific promoters include cardiac troponin T and the α-myosin heavy chain promoter.

In some embodiments, the AAV-Cas9 vector may be optimized for production in yeast, bacteria, insect cells, or mammalian cells. In some embodiments, the AAV-Cas9 vector may be optimized for expression in human cells. In some embodiments, the AAV-Cas9 vector may be optimized for expression in a baculovirus expression system.

In some embodiments of the gene editing constructs of the disclosure, the construct comprises or consists of a promoter and a nucleic acid encoding the fusion protein described herein. In some embodiments, the construct comprises or consists of a cardiac troponin T promoter and a nucleic acid encoding a fusion protein comprising a deaminase and Cas9 nuclease. In some embodiments, the construct comprises or consists of a cardiac troponin T promoter and a nucleic acid encoding a fusion protein comprising a deaminase and Cas9 nickase isolated or derived from Staphylococcus pyogenes (“SpCas9”). An exemplary promoter that may be used in the AAV vectors herein can comprise SEQ ID NO: 72.

In some embodiments, the construct comprising a promoter and a nuclease further comprises at least two inverted terminal repeat (ITR) sequences. In some embodiments, the construct comprising a promoter and a nuclease further comprises at least two ITR sequences from isolated or derived from an AAV of serotype 2 (AAV2). In some embodiments, the construct comprising a promoter and a nuclease further comprises at least two ITR sequences each comprising or consisting of a nucleotide sequence of SEQ ID NO: 71 or 85. In some embodiments, the construct comprising a promoter and a nuclease further comprises at least two ITR sequences, wherein the first ITR sequence comprises or consists of a nucleotide sequence of SEQ ID NO: 71 and the second ITR sequence comprises or consist of a nucleotide sequence 85. In some embodiments, the construct comprises or consists of, from 5′ to 3′ a first ITR, a sequence encoding a promoter (e.g., a Cardiac Troponin T promoter), a sequence encoding a nuclear localization signal, a sequence encoding a deaminase, a sequence encoding a flexible peptide linker, a sequence encoding a fragment of a SpCas9 nickase (e.g., an N-terminal half), a sequence encoding a gRNA, and a second ITR. In some embodiments, the construct comprises or consists of, from 5′ to 3′ a first ITR, a sequence encoding a promoter (e.g., a Cardiac Troponin T promoter), a sequence encoding a nuclear localization signal, a sequence encoding a second fragment of a SpCas9 nickase (e.g., a C-terminal half), a sequence encoding a gRNA and a second ITR.

(e) AAV Delivery of Base Editors and gRNAs

Some aspects of the present disclosure relate to the delivery of base editors (and their associated gRNAs) using a split-base editor dual AAV strategy. One impediment to the delivery of base editors in animals has been an inability to package base editors in adeno-associated virus (AAV), an efficient and widely used delivery agent that remains the only FDA-approved in vivo gene therapy vector. The large size of the DNA encoding base editors (5.2 kb for base editors containing S. pyogenes Cas9, not including any guide RNA or regulatory sequences) can preclude packaging in AAV, which has a genome packaging size limit of <5 kb 12.

To bypass this packaging size limit and deliver base editors using AAVs, a split-base editor dual AAV strategy was devised, in which the adenine base editor (ABE) is divided into an N-terminal and C-terminal half. This strategy is described in PCT Patent Application Publication WO2020236982A1; the entire contents of which are hereby incorporated by reference. Each base editor half is fused to half of a fast-splicing split-intein. Following co-infection by AAV particles expressing each base editor-split intein half, protein splicing in trans reconstitutes full-length base editor. Unlike other approaches utilizing small molecules or sgRNA to bridge split Cas9, intein splicing removes all exogenous sequences and regenerates a native peptide bond at the split site, resulting in a single reconstituted protein identical in sequence to the unmodified base editor.

Described in PCT Patent Application Publication WO2020236982A1 further provides nucleic acid molecules, compositions, recombinant AAV (rAAV) particles, kits, and methods for delivering a Cas9 protein or a nucleobase editor to cells, e.g., via rAAV vectors. Typically, a Cas9 protein or a nucleobase editor is“split” into an N-terminal portion and a C-terminal portion. The N-terminal portion or C-terminal portion of a Cas9 protein or a nucleobase editor may be fused to one member of the intein system, respectively. The resulting fusion proteins, when delivered on separate vectors (e.g., separate rAAV vectors) into one cell and co-expressed, may be joined to form a complete and functional Cas9 protein or nucleobase editor (e.g., via intein-mediated protein splicing). Further provided herein are empirical testing of regulatory elements in the delivery vectors for high expression levels of the split Cas9 protein or the nucleobase editor.

In some embodiments, the adenine base editor (ABE) is split within the Cas9 domain of the ABE. In some embodiments, the ABE is split between the Glu 573 and the Cys 574 residue of a Cas9 (e.g., Cas9-VRQR) having the sequence:

(SEQ ID NO: 15)
MDKKYSIGLAIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFVSPTVAYSVLVVAKVEKGKSKKLKSVKELLGITIME
RSSFEKNPIDFLEAKGYKEVKKDLIIKLPKYSLFELENGRKRMLA
SARELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKQYRSTKEVLDATLIHQ
SITGLYETRIDLSQLGGD.

For the purpose of clarity, residues E573 and C574 are indicated in bold and underlined in the above sequence of SEQ ID NO: 15. It should be appreciated that ABEs having different Cas9 sequences (e.g., SEQ ID NOs 16-22 listed above) could be split at the same or a different residue (e.g., a residue that is at least 1, 2, 3, 4, 5, 6, 7, 8, 9, 10, 15, 20, 25, 30, 35, 40, 45, 50, 60, 70, 80, 90, or 100 residues from the 573 or 574 residue of SEQ ID NO: 15, as exemplified herein) as compared to the Cas9 of SEQ ID NO: 15. It is also understood that SEQ ID NO: 15 contains a methionine as an initial amino acid residue as a start codon. When this amino acid is omitted, such as when the Cas9 protein is expressed with a nuclear localization sequence at the N terminus, the corresponding residues that are split are E572 and C573. It can also be understood that full fusion proteins comprising a deaminase covalently linked to the Cas9 protein (as described herein) may also be split at an equivalent location in the Cas9 protein. For example, a fusion protein comprising SEQ ID NO: 46 may be split at E987 and C988 according to SEQ ID NO: 46. Tools (e.g., BLAST) useful for identifying corresponding residues in other Cas9 sequences and in the fusion proteins (e.g., base editors) described herein are known in the art and a skilled artisan would understand how to determine such corresponding residues. In some embodiments, the intein used to split the base editor is an Npu intein. In some embodiments, the intein comprises the amino acid sequence of SEQ ID NO: 153 or 154, wherein SEQ ID NO: 153 is an Npu DnaE N-terminal protein and wherein SEQ ID NO: 154 is an Npu DnaE C-terminal protein.

### Npu DnaE N-Terminal Protein:

(SEQ ID NO: 153)
CLSYETEILTVEYGLLPIGKIVEKRIECTVYSVDNNGNIYTQPVA
QWHDRGEQEVFEYCLEDGSLIRATKDHKFMTVDGQMLPID

### Npu DnaE C-Terminal Protein:

(SEQ ID NO: 154)
IKIATRKYLGKQNVYDIGVERDHNFALKNGFIASN.

In some embodiments, the construct comprising or consisting of, from 5′ to 3′ a first ITR, a sequence encoding a promoter, a sequence encoding a gRNA and/or Cas9 nickase or fragment thereof and a second ITR, further comprises a poly A sequence. In some embodiments, the polyA sequence comprises or consists of a bGH sequence. Exemplary bGH sequences of the disclosure comprise or consist of a nucleotide sequence of SEQ ID NO: 81 (ctgtgccttctagttgccagccatctgttgtttgcccctcccccgtgccttccttgaccctggaaggtgccactcccactgtcctttccta ataaaatgaggaaattgcatcgcattgtctgagtaggtgtcattctattctggggggtggggtggggcaggacagcaaggggga ggattgggaagacaatagcaggcatgctggggatgcggtgggctctatgg). In some embodiments, the construct comprises or consists of, from 5′ to 3′ a first ITR, a sequence encoding a promoter, a sequence encoding a fusion protein (hereinafter—“base editor”) or fragment thereof, a poly A sequence, a sequence encoding a gRNA, and a second ITR. In some embodiments, the construct comprises or consists of, from 5′ to 3′ a first ITR, a sequence encoding a promoter, a sequence encoding a fusion protein (hereinafter—“base editor”) or fragment thereof, a bgH polyA sequence, a sequence encoding a gRNA, and a second ITR. In some embodiments, the construct comprises or consists of, from 5′ to 3′ a first AAV2 ITR, a sequence encoding an cardiac troponin T promoter, a sequence encoding a fusion protein (hereinafter—“base editor”) or fragment thereof, a bgH polyA sequence, a sequence encoding a gRNA, and a second AAV2 ITR. In some embodiments, the construct comprising, from 5′ to 3′ a first ITR, a sequence encoding a promoter, a sequence encoding a fusion protein (hereinafter—“base editor”) or fragment thereof, a poly A sequence, a sequence encoding a gRNA, and a second ITR, further comprises at least one nuclear localization signal. In some embodiments, the construct comprising, from 5′ to 3′ a first ITR, a sequence encoding a promoter, a sequence encoding a fusion protein (hereinafter—“base editor”) or fragment thereof, a poly A sequence, a sequence encoding a gRNA, and a second ITR, further comprises at least two nuclear localization signals. Exemplary sequences encoding nuclear localization signals of the disclosure comprise or consist of any of SEQ ID NO: 43, 44 and 90. In some embodiments, the construct comprises or consists of, from 5′ to 3′ a first ITR, a sequence encoding a promoter, a sequence encoding a first nuclear localization signal, a sequence encoding a fusion protein (hereinafter—“base editor”) or fragment thereof, a poly A sequence, a sequence encoding a gRNA, and a second ITR. In some embodiments, the construct comprises or consists of, from 5′ to 3′ a first ITR, a sequence encoding a promoter, a sequence encoding a first nuclear localization signal, a sequence encoding a fusion protein (hereinafter—“base editor”) or fragment thereof, a sequence encoding a second nuclear localization signal, a sequence encoding a poly A sequence, a sequence encoding a gRNA, and a second ITR. In some embodiments, the construct comprising, from 5′ to 3′ a first ITR, a sequence encoding a promoter, a sequence encoding a first nuclear localization signal, a sequence encoding a fusion protein (hereinafter—“base editor”) or fragment thereof, a sequence encoding a second nuclear localization signal, a poly A sequence, a sequence encoding a gRNA and a second ITR, further comprises a stop codon. The stop codon may have a sequence of TAG, TAA, or 5 TGA. In some embodiments, the construct comprises or consists of, from 5′ to 3′ a first ITR, a sequence encoding a promoter, a sequence encoding a first nuclear localization signal, a sequence encoding a fusion protein (hereinafter—“base editor”) or fragment thereof, a sequence encoding a second nuclear localization signal, a stop codon, a poly A sequence, a sequence encoding a gRNA, and a second ITR. In some embodiments, the construct comprising or consisting of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR, further comprises a regulatory sequence. The regulatory sequence may encode a posttranslational regulatory element. For example, an exemplary regulatory sequences of the disclosure comprise or consist of a nucleotide sequence of SEQ ID NO: 80 (which encodes for WPRE-3 (Woodchuck Hepatitis Virus Posttranscriptional Regulatory Element-3)). In some embodiments, the construct comprises or consists of, from 5′ to 3′ a first ITR, a sequence encoding a promoter, a sequence encoding a first nuclear localization signal, a sequence encoding a fusion protein (hereinafter “base editor”) or fragment thereof, a sequence encoding a second nuclear localization signal, a stop codon, a sequence encoding a regulatory element (e.g., SEQ ID NO: 80), a poly A sequence, a sequence encoding a gRNA, and a second ITR. In some embodiments, the construct comprising or consisting of, from 5′ to 3′ a first ITR, a sequence encoding a promoter, a sequence encoding a first nuclear localization signal, a sequence encoding a fusion protein (hereinafter “base editor”) or fragment thereof, a sequence encoding a second nuclear localization signal, a stop codon, a regulatory sequence, a poly A sequence, a sequence encoding a gRNA, and a second ITR, further comprises one or more gRNA scaffold sequences. Suitable gRNA scaffold sequences may include any of SEQ ID NOs: 82, 84, 165 and/or 166.

SEQ ID NO: 82:
GAGGGCCTATTTCCCATGATTCCTTCATATTTGCATATACGATACA
AGGCTGTTAGAGAGATAATTAGAATTAATTTGACTGTAAACACAA
AGATATTAGTACAAAATACGTGACGTAGAAAGTAATAATTTCTTG
GGTAGTTTGCAGTTTTAAAATTATGTTTTAAAATGGACTATCATA
TGCTTACCGTAACTTGAAAGTATTTCGATTTCTTGGCTTTATATA
TCTTGTGGAAAGGACGAAACACCG
SEQ ID NO: 84:
GCTTAAGAGCTATGCTGGAAACAGCATAGCAAGTTTAAGTAAGGC
TAGTCCGTTATCAACTTGAAAAAGTGGCACCGAGTCGGTGC
SEQ ID NO: 165:
GTTTTAGAGCTAGAAATAGCAAGTTAAAATAAGGCTAGTCCGTTA
TCAACTTGAAAAAGTGGCACCGAGTCGGTGC
SEQ ID NO: 166:
GTTTAAGAGCTATGCTGGAAACAGCATAGCAAGTTTAAATAAGGC
TAGTCCGTTATCAACTTGAAAAAGTGGCACCGAGTCGGTGCTTTT

Accordingly, in some embodiments, the construct may comprise or consist of, from 5′ to 3′, first ITR, a sequence encoding a promoter, a sequence encoding a first nuclear localization signal, a sequence encoding a fusion protein (hereinafter “base editor”) or fragment thereof, a sequence encoding a second nuclear localization signal, a stop codon, a regulatory sequence, a poly A sequence, a sequence encoding a first gRNA scaffold sequence, a sequence encoding a gRNA, a sequence encoding a second gRNA scaffold sequence and a second ITR.

In some embodiments, the construct may further comprise one or more spacer sequences. Exemplary spacer sequences of the disclosure have length from 1-1500 nucleotides, inclusive of all ranges therebetween. In some embodiments, the spacer sequences may be located either 5′ to or 3′ to an ITR, a promoter, a nuclear localization sequence, a sequence encoding a fusion protein (hereinafter “base editor”), a stop codon, a polyA sequence, a gRNA scaffold, a nucleic acid encoding a gRNA, and/or a regulator element.

In accord with the disclosure herein, exemplary viral vectors comprising one or more of the nucleic acids encoding the gRNA and/or fusion protein (base editors), or fragment thereof are provided. Also provided are a pair of viral vectors, comprising a first viral vector encoding for a first fragment of the fusion protein described herein and a second viral vector encoding a second fragment of the fusion protein, wherein the first and second fragment may recombine in a cell via post-translational splicing to form a functional fusion protein (as described above). Two exemplary vectors are described in Tables 9 and 10 below, along with key components.

TABLE 9
Exemplary Vector Encoding N- Terminus
of ABEmax-VRQR Fusion Protein

| | | |
|---|---|---|
| Vector Element | Location (bp) | SEQ ID NO: |

| | | | |
|---|---|---|---|
| AAV ITR | 1-130 | bp | 71 |
| Cardiac Troponin T promoter | 198-610 | bp | 72 |

| | | |
|---|---|---|
| Nuclear Localization Signals | 623-679 | 43 |

(Bipartite NLS)

| | | |
|---|---|---|
| ABEmax | 680-1,771 | 74 |
| Linker | 1,772-1,867 | 29 |
| SpCas9-VRQR N-terminal | 1,868-3,583 | 76 |

half

| | | |
|---|---|---|
| Npu N-terminal fragment | 3,584-3,838 | 77 |
| linker | 3,839-3,902 | 78 |
| Nuclear Localization Signal | 3,903-3,955 | 44 |
| WPRE-3 (Woodchuck Hepatitis | 3,961-4,209 | 80 |

Virus Posttranscriptional
Regulatory Element-3)

| | | |
|---|---|---|
| bGH poly(A) signal (bovine | 4,213-4,437 | 81 |

growth hormone
polyadenylation signal)

| | | |
|---|---|---|
| hU6 promoter-sgRNA | 4,444-4,693 | 82 |

scaffold - 1

| | | |
|---|---|---|
| h403_sgRNA | 4,694-4,713 | 1 |
| hU6 promoter-sgRNA | 4,714-4,799 | 84 |

scaffold - 2

| | | |
|---|---|---|
| AAV ITR | 4,868-4,997 | 85 |

| | | | |
|---|---|---|---|
| Full Vector | 4,997 | bp | 86 |

TABLE 10
Exemplary Vector Encoding C- Terminus
of ABEmax-VRQR Fusion Protein

| | | |
|---|---|---|
| Vector Element | Location (bp) | SEQ ID NO: |

| | | | |
|---|---|---|---|
| AAV ITR | 1-130 | bp | 71 |
| Cardiac Troponin T promoter | 198-610 | bp | 72 |

| | | |
|---|---|---|
| Nuclear Localization Signals | 623-679 | 43 |

(Bipartite NLS)

| | | |
|---|---|---|
| Npu C-terminal fragment | 680-784 | 87 |
| SpCas9-VRQR C-terminal | 785-3,169 | 88 |

half

| | | |
|---|---|---|
| Linker | 3,170-3,181 | 89 |
| Nuclear Localization Signal | 3,182-3,232 | 90 |
| WPRE-3 (Woodchuck Hepatitis | 3,241-3,489 | 80 |

Virus Posttranscriptional
Regulatory Element-3)

| | | |
|---|---|---|
| bGH poly(A) signal (bovine | 3,493-3,717 | 81 |

growth hormone
polyadenylation signal)

| | | |
|---|---|---|
| hU6 promoter-sgRNA | 3,723-3,972 | 82 |

scaffold - 1

| | | |
|---|---|---|
| h403_sgRNA | 3,973-3,992 | 1 |
| hU6 promoter-sgRNA | 3,993-4,078 | 84 |

scaffold - 2

| | | |
|---|---|---|
| AAV ITR | 4,147-4,276 | 85 |

| | | | |
|---|---|---|---|
| Full Vector | 4,276 | bp | 91 |

In some aspects, each AAV vector provided in the tables above expresses either an N-terminal half (SEQ ID NO: 69) or C-terminal half (SEQ ID NO: 70) of ABEmax-VRQR. When the two protein halves come in contact, they undergo protein trans-splicing to form the complete protein. SEQ ID NO: 69 and 70 are provided in table 12 below. Each sequence has an “NPU intein fragment” underlined (SEQ ID NOs: 153 and 154). This fragment is removed from the final protein construct to form the complete fusion protein.

TABLE 12
Fusion Protein Fragments Expressed by
AAV Vectors

| | | SEQ |
|---|---|---|
| | | ID |
| Fusion Protein | SEQUENCE | NO: |
| Fragment | MKRTADGSEFESPKKKRKVSEVEFS | 69 |
| N-Terminus half | HEYWMRHALTLAKRAWDEREVPVGA | |
| NPU Fragment | VLVHNNRVIGEGWNRPIGRHDPTAH | |
| spliced out upon | AEIMALRQGGLVMQNYRLIDATLYV | |
| recombination is | TLEPCVMCAGAMIHSRIGRVVFGAR | |
| underlined and | DAKTGAAGSLMDVLHHPGMNHRVEI | |
| bolded | TEGILADECAALLSDFFRMRRQEIK | |
| | AQKKAQSSTDSGGSSGGSSGSETPG | |
| | TSESATPESSGGSSGGSSEVEFSHE | |
| | YWMRHALTLAKRARDEREVPVGAVL | |
| | VLNNRVIGEGWNRAIGLHDPTAHAE | |
| | IMALRQGGLVMQNYRLIDATLYVTF | |
| | EPCVMCAGAMIHSRIGRVVFGVRNA | |
| | KTGAAGSLMDVLHYPGMNHRVEITE | |
| | GILADECAALLCYFFRMPRQVFNAQ | |
| | KKAQSSTDSGGSSGGSSGSETPGTS | |
| | ESATPESSGGSSGGSDKKYSIGLAI | |
| | GTNSVGWAVITDEYKVPSKKFKVLG | |
| | NTDRHSIKKNLIGALLFDSGETAEA | |
| | TRLKRTARRRYTRRKNRICYLQEIF | |
| | SNEMAKVDDSFFHRLEESFLVEEDK | |
| | KHERHPIFGNIVDEVAYHEKYPTIY | |
| | HLRKKLVDSTDKADLRLIYLALAHM | |
| | IKFRGHFLIEGDLNPDNSDVDKLFI | |
| | QLVQTYNQLFEENPINASGVDAKAI | |
| | LSARLSKSRRLENLIAQLPGEKKNG | |
| | LFGNLIALSLGLTPNFKSNFDLAED | |
| | AKLQLSKDTYDDDLDNLLAQIGDQY | |
| | ADLFLAAKNLSDAILLSDILRVNTE | |
| | ITKAPLSASMIKRYDEHHQDLTLLK | |
| | ALVRQQLPEKYKEIFFDQSKNGYAG | |
| | YIDGGASQEEFYKFIKPILEKMDGT | |
| | EELLVKLNREDLLRKQRTFDNGSIP | |
| | HQIHLGELHAILRRQEDFYPFLKDN | |
| | REKIEKILTFRIPYYVGPLARGNSR | |
| | FAWMTRKSEETITPWNFEEVVDKGA | |
| | SAQSFIERMTNFDKNLPNEKVLPKH | |
| | SLLYEYFTVYNELTKVKYVTEGMRK | |
| | PAFLSGEQKKAIVDLLFKTNRKVTV | |
| | KQLKEDYFKKIECLSYETEILTVEY | |
| | GLLPIGKIVEKRIECTVYSVDNNGN | |
| | IYTQPVAQWHDRGEQEVFEYCLEDG | |
| | SLIRATKDHKFMTVDGQMLPIDEIF | |
| | ERELDLMRVDNLPNSGGSKRTADGS | |
| | EFEPKKKRKV | |
| Fusion Protein | MKRTADGSEFESPKKKRKVIKIATR | 70 |
| C-Terminus half | KYLGKQNVYDIGVERDHNFALKNGF | |
| NPU Fragment | IASNCFDSVEISGVEDRFNASLGTY | |
| spliced out upon | HDLLKIIKDKDFLDNEENEDILEDI | |
| recombination is | VLTLTLFEDREMIEERLKTYAHLFD | |
| underlined and | DKVMKQLKRRRYTGWGRLSRKLING | |
| bolded | IRDKQSGKTILDFLKSDGFANRNFM | |
| | QLIHDDSLTFKEDIQKAQVSGQGDS | |
| | LHEHIANLAGSPAIKKGILQTVKVV | |
| | DELVKVMGRHKPENIVIEMARENQT | |
| | TQKGQKNSRERMKRIEEGIKELGSQ | |
| | ILKEHPVENTQLQNEKLYLYYLQNG | |
| | RDMYVDQELDINRLSDYDVDHIVPQ | |
| | SFLKDDSIDNKVLTRSDKNRGKSDN | |
| | VPSEEVVKKMKNYWRQLLNAKLITQ | |
| | RKFDNLTKAERGGLSELDKAGFIKR | |
| | QLVETRQITKHVAQILDSRMNTKYD | |
| | ENDKLIREVKVITLKSKLVSDFRKD | |
| | FQFYKVREINNYHHAHDAYLNAVVG | |
| | TALIKKYPKLESEFVYGDYKVYDVR | |
| | KMIAKSEQEIGKATAKYFFYSNIMN | |
| | FFKTEITLANGEIRKRPLIETNGET | |
| | GEIVWDKGRDFATVRKVLSMPQVNI | |
| | VKKTEVQTGGFSKESILPKRNSDKL | |
| | IARKKDWDPKKYGGFVSPTVAYSVL | |
| | VVAKVEKGKSKKLKSVKELLGITIM | |
| | ERSSFEKNPIDFLEAKGYKEVKKDL | |
| | IIKLPKYSLFELENGRKRMLASARE | |
| | LQKGNELALPSKYVNFLYLASHYEK | |
| | LKGSPEDNEQKQLFVEQHKHYLDEI | |
| | IEQISEFSKRVILADANLDKVLSAY | |
| | NKHRDKPIREQAENIIHLFTLTNLG | |
| | APAAFKYFDTTIDRKQYRSTKEVLD | |
| | ATLIHQSITGLYETRIDLSQLGGDS | |
| | GGSKRTADGSEFEPKKKRKV | |

In some embodiments, AAV vectors disclosed herein may be packaged into virus particles which can be used to deliver the genome for transgene expression in target cells. In some embodiments, AAV vectors disclosed herein can be packaged into particles by transient transfection, use of producer cell lines, combining viral features into Ad-AAV hybrids, use of herpesvirus systems, or production in insect cells using baculoviruses.

In some embodiments, methods of generating a packaging cell herein involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

In some embodiments, a host cell is transiently or non-transiently transfected with one or more vectors, linear polynucleotides, polypeptides, nucleic acid-protein complexes, or any combination thereof as described herein. In some embodiments, a cell can be transfected in vitro, in culture, or ex vivo. In some embodiments, a cell can be transfected as it naturally occurs in a subject. In some embodiments, a cell that is transfected can be taken from a subject. In some embodiments, the cell is derived from cells taken from a subject, such as a cell line.

In some embodiments, a cell transfected with one or more vectors, linear polynucleotides, polypeptides, nucleic acid-protein complexes, or any combination thereof as described herein may be used to establish a new cell line can include one or more transfection-derived sequences. In some embodiments, a cell transiently transfected with the components of an engineered nucleic acid-guided nuclease system as described herein (such as by transient transfection of one or more vectors, or transfection with RNA), and modified through the activity of an engineered nuclease complex, may be used to establish a new cell line can include cells containing the modification but lacking any other exogenous sequence.

Some embodiments disclosed herein relate to use of CRISPR-Cas9 systems disclosed herein; for example, in order to target and knock out genes, amplify genes and/or repair particular mutations associated with DNA repeat instability and a medical disorder. In some embodiments, CRISPR-Cas9 systems herein can be used to harness and to correct these defects of genomic instability. In other embodiments, CRISPR-Cas9 systems disclosed herein can be used for correcting defects in the genes associated with a cardiomyopathy.

### C. Pharmaceutical Compositions

Any of the AAV viral particles, AAV vectors, polynucleotides, or vectors encoding polynucleotides disclosed herein may be formulated into a pharmaceutical composition. In some embodiments, pharmaceutical composition may further include one or more pharmaceutically acceptable carriers, diluents or excipients. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some embodiments, the pharmaceutical compositions or formulations can be for administration by subcutaneous, intramuscular, intravenous, intraperitoneal, intracardiac, intraarticular, or intracavernous injection. In some embodiments, the pharmaceutical compositions or formulations are for parenteral administration, such as intravenous, intracerebroventricular injection, intra-cisterna magna injection, intra-parenchymal injection, intraperitoneal, intracardiac, intraarticular, or intracavernous injection or a combination thereof. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating AAV particles in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

### D. Gene-Edited Organisms—Model Systems

Further aspects of the present disclosure are directed to gene edited organisms (e.g., mammalian organisms) that may be used to test the gene editing techniques and compositions provided herein. For example, in one aspect, the gene editing compositions herein generally comprise a gRNA and a fusion protein of a nickase and deaminase to perform base editing at a mutation site in a human gene in order to correct a gene mutation associated with cardiomyopathy. However, a suitable mouse model to test this strategy does not exist because the corresponding murine gene (MYH6) is different from the human gene (MYH7) and an equivalent mutation does not exist for murine MYH6 and human MYH7. This means that a CRISPR gene editing system optimized for the human MYH7 gene may not have any effect on the murine MYH6 gene.

Accordingly, in accordance with further aspects of the present disclosure, a gene edited mouse is provided, the mouse comprising a human nucleic acid comprising a MYH7 c.1208 G>A (p.R403Q) human missense mutation inserted within an endogenous murine Myh6 gene to form a humanized mutant Myh6 allele. In some aspects, the human nucleic acid further comprises a first polynucleotide adjacent to and upstream of the missense mutation and a second polynucleotide adjacent to and downstream of the missense mutation. For example, in some aspects, the first polynucleotide comprises about 30 to 75 nucleotides, about 35 to about 70 nucleotides, about 40 to about 65 nucleotides, or about 45 to about 60 nucleotides. For example, the first polynucleotide can comprise about 55 nucleotides. In other aspects, the second polynucleotide comprises about 10 to 30 nucleotides, about 15 to 25 nucleotides, or about 20 to 25 nucleotides. For example, the second polynucleotide may comprise or consists of 21 nucleotides. An exemplary human nucleic acid that may be inserted into the endogenous Myh6 gene is described in the Table below. Also provided is the native MyH6 allele. As is shown in Table 13, the humanized nucleic acid is identical to the equivalent portion of the MYH7 gene and includes substitutions relative to the murine MyH6 gene (underlined). The missense mutation is indicated in bold and underlined. SEQ ID NO: 158 (Table 14C) provides optional humanized alleles comprising the G>A mutation, wherein nucleotides N1 to N6 may be chosen from the native mouse nucleotide or a humanized nucleotide. In various aspects, the humanized mutant Myh6 allele comprises at least 1, at least 2, at least 3, at least 4, at least 5 or at least 6 mutations according to SEQ ID NO: 158 relative to a native Myh6 allele (SEQ ID NO: 99 or SEQ ID NO: 163). Tables 14A-14C further provide the full murine and human mutant and wildtype MYH6 and MYH7 protein sequences (Table 14A), full human and murine mutant and wildtype gene transcripts (cDNA sequences) (Table 14B) and additional sequences covering optional humanizing mutations in and around the Myh6 allele (Table 140).

In various aspects, at least one cell of the gene edited mouse expresses a mutant myosin protein comprising a R404Q substitution relative to a wildtype myosin protein comprising SEQ ID NO: 94. For ease of reference, Table 14 provides sequences ofthe native Myh6 protein (mouse), native human Myh7 protein, and the mutant Myh6 protein expressed by the humanized Myh6 allele described above. Accordingly, in various aspects, at least one cell of the gene edited mouse expresses a mutant myosin protein comprising SEQ ID NO: 96. In some aspects, the mouse is heterozygous for the mutant Myh6 allele and further comprises a wildtype Myh6 allele.

TABLE 13
Humanized and Wildtype Myh6 nucleic acids

| Sequence Name (SEQ ID NO) | Sequence |
|---|---|
| Humanized MyH6 nucleic acid | TGCCTACCTCATGGGGCTGAACTCAGCC |
| (SEQ ID NO: 98) | GACCTGCTCAAGGGGCTGTGCCACCCTC |
| | AGGTGAAAGTGGGCAATGAGTAC |
| Wildtype Myh6 nucleic acid (portion) | . . . AGCCTACCTTATGGGGCTGAACTCAGC |
| (SEQ ID NO: 99) | TGACCTGCTCAAGGGCCTGTGTCACCCT |
| | CGGGTGAAGGTGGGGAACGAGTAT . . . |

TABLE 14A
Mutant and WT MYH6 and MYH7 proteins

| Sequence Name (SEQ ID NO) | Sequence |
|---|---|
| Native Murine Myh6 Protein (SEQ ID NO: 95) | MTDAQMADFGAAAQYLRKSEKERLEAQTRPFDI |
| | RTECFVPDDKEEYVKAKVVSREGGKVTAETENGK |
| | TVTIKEDQVMQQNPPKFDKIEDMAMLTFLHEPA |
| | VLYNLKERYAAWMIYTYSGLFCVTVNPYKWLPVY |
| | NAEVVAAYRGKKRSEAPPHIFSISDNAYQYMLTD |
| | RENQSILITGESGAGKTVNTKRVIQYFASIAAIGDR |
| | SKKENPNANKGTLEDQIIQANPALEAFGNAKTVR |
| | NDNSSRFGKFIRIHFGATGKLASADIETYLLEKSRVI |
| | FQLKAERNYHIFYQILSNKKPELLDMLLVTNNPYD |
| | YAFVSQGEVSVASIDDSEELLATDSAFDVLSFTAEE |
| | KAGVYKLTGAIMHYGNMKFKQKQREEQAEPDG |
| | TEDADKSAYLMGLNSADLLKGLCHPRVKVGNEYV |
| | TKGQSVQQVYYSIGALAKSVYEKMFNWMVTRIN |
| | ATLETKQPRQYFIGVLDIAGFEIFDFNSFEQLCINFT |
| | NEKLQQFFNHHMFVLEQEEYKKEGIEWEFIDFG |
| | MDLQACIDLIEKPMGIMSILEEECMFPKASDMTF |
| | KAKLYDNHLGKSNNFQKPRNVKGKQEAHFSLVH |
| | YAGTVDYNIMGWLEKNKDPLNETVVGLYQKSSL |
| | KLMATLFSTYASADTGDSGKGKGGKKKGSSFQTV |
| | SALHRENLNKLMTNLKTTHPHFVRCIIPNERKAPG |
| | VMDNPLVMHQLRCNGVLEGIRICRKGFPNRILYG |
| | DFRQRYRILNPAAIPEGQFIDSRKGAEKLLGSLDID |
| | HNQYKFGHTKVFFKAGLLGLLEEMRDERLSRIITRI |
| | QAQARGQLMRIEFKKIVERRDALLVIQWNIRAFM |
| | GVKNWPWMKLYFKIKPLLKSAETEKEMANMKEE |
| | FGRVKDALEKSEARRKELEEKMVSLLQEKNDLQL |
| | QVQAEQDNLNDAEERCDQLIKNKIQLEAKVKEM |
| | TERLEDEEEMNAELTAKKRKLEDECSELKKDIDDL |
| | ELTLAKVEKEKHATENKVKNLTEEMAGLDEIIAKLT |
| | KEKKALQEAHQQALDDLQAEEDKVNTLTKSKVKL |
| | EQQVDDLEGSLEQEKKVRMDLERAKRKLEGDLKL |
| | TQESIMDLENDKLQLEEKLKKKEFDISQQNSKIED |
| | EQALALQLQKKLKENQARIEELEEELEAERTARAK |
| | VEKLRSDLSRELEEISERLEEAGGATSVQIEMNKKR |
| | EAEFQKMRRDLEEATLQHEATAAALRKKHADSV |
| | AELGEQIDNLQRVKQKLEKEKSEFKLELDDVTSN |
| | MEQIIKAKANLEKVSRTLEDQANEYRVKLEEAQRS |
| | LNDFTTQRAKLQTENGELARQLEEKEALISQLTRG |
| | KLSYTQQMEDLKRQLEEEGKAKNALAHALQSSRH |
| | DCDLLREQYEEEMEAKAELQRVLSKANSEVAQW |
| | RTKYETDAIQRTEELEEAKKKLAQRLQDAEEAVEA |
| | VNAKCSSLEKTKHRLQNEIEDLMVDVERSNAAAA |
| | ALDKKQRNFDKILAEWKQKYEESQSELESSQKEA |
| | RSLSTELFKLKNAYEESLEHLETFKRENKNLQEEISD |
| | LTEQLGEGGKNVHELEKIRKQLEVEKLELQSALEE |
| | AEASLEHEEGKILRAQLEFNQIKAEIERKLAEKDEE |
| | MEQAKRNHLRMVDSLQTSLDAETRSRNEALRVK |
| | KKMEGDLNEMEIQLSQANRIASEAQKHLKNSQA |
| | HLKDTQLQLDDAVHANDDLKENIAIVERRNNLLQ |
| | AELEELRAVVEQTERSRKLAEQELIETSERVQLLHS |
| | QNTSLINQKKKMESDLTQLQTEVEEAVQECRNAE |
| | EKAKKAITDAAMMAEELKKEQDTSAHLERMKKN |
| | MEQTIKDLQHRLDEAEQIALKGGKKQLQKLEARV |
| | RELENELEAEQKRNAESVKGMRKSERRIKELTYQT |
| | EEDKKNLMRLQDLVDKLQLKVKAYKRQAEEAEE |
| | QANTNLSKFRKVQHELDEAEERADIAESQVNKLR |
| | AKSRDIGAKKMHDEE |
| Humanized Murine Myh6 Protein (difference | MTDAQMADFGAAAQYLRKSEKERLEAQTRPFDI |
| between WT Myh6 is bolded and | RTECFVPDDKEEYVKAKVVSREGGKVTAETENGK |
| underlined) (SEQ ID NO: 96) | TVTIKEDQVMQQNPPKFDKIEDMAMLTFLHEPA |
| | VLYNLKERYAAWMIYTYSGLFCVTVNPYKWLPVY |
| | NAEVVAAYRGKKRSEAPPHIFSISDNAYQYMLTD |
| | RENQSILITGESGAGKTVNTKRVIQYFASIAAIGDR |
| | SKKENPNANKGTLEDQIIQANPALEAFGNAKTVR |
| | NDNSSRFGKFIRIHFGATGKLASADIETYLLEKSRVI |
| | FQLKAERNYHIFYQILSNKKPELLDMLLVTNNPYD |
| | YAFVSQGEVSVASIDDSEELLATDSAFDVLSFTAEE |
| | KAGVYKLTGAIMHYGNMKFKQKQREEQAEPDG |
| | TEDADKSAYLMGLNSADLLKGLCHPQVKVGNEY |
| | VTKGQSVQQVYYSIGALAKSVYEKMFNWMVTRI |
| | NATLETKQPRQYFIGVLDIAGFEIFDFNSFEQLCIN |
| | FTNEKLQQFFNHHMFVLEQEEYKKEGIEWEFIDF |
| | GMDLQACIDLIEKPMGIMSILEEECMFPKASDMT |
| | FKAKLYDNHLGKSNNFQKPRNVKGKQEAHFSLV |
| | HYAGTVDYNIMGWLEKNKDPLNETVVGLYQKSS |
| | LKLMATLFSTYASADTGDSGKGKGGKKKGSSFQT |
| | VSALHRENLNKLMTNLKTTHPHFVRCIIPNERKAP |
| | GVMDNPLVMHQLRCNGVLEGIRICRKGFPNRILY |
| | GDFRQRYRILNPAAIPEGQFIDSRKGAEKLLGSLDI |
| | DHNQYKFGHTKVFFKAGLLGLLEEMRDERLSRIIT |
| | RIQAQARGQLMRIEFKKIVERRDALLVIQWNIRAF |
| | MGVKNWPWMKLYFKIKPLLKSAETEKEMANMK |
| | EEFGRVKDALEKSEARRKELEEKMVSLLQEKNDL |
| | QLQVQAEQDNLNDAEERCDQLIKNKIQLEAKVKE |
| | MTERLEDEEEMNAELTAKKRKLEDECSELKKDIDD |
| | LELTLAKVEKEKHATENKVKNLTEEMAGLDEIIAKL |
| | TKEKKALQEAHQQALDDLQAEEDKVNTLTKSKVK |
| | LEQQVDDLEGSLEQEKKVRMDLERAKRKLEGDLK |
| | LTQESIMDLENDKLQLEEKLKKKEFDISQQNSKIED |
| | EQALALQLQKKLKENQARIEELEEELEAERTARAK |
| | VEKLRSDLSRELEEISERLEEAGGATSVQIEMNKKR |
| | EAEFQKMRRDLEEATLQHEATAAALRKKHADSV |
| | AELGEQIDNLQRVKQKLEKEKSEFKLELDDVTSN |
| | MEQIIKAKANLEKVSRTLEDQANEYRVKLEEAQRS |
| | LNDFTTQRAKLQTENGELARQLEEKEALISQLTRG |
| | KLSYTQQMEDLKRQLEEEGKAKNALAHALQSSRH |
| | DCDLLREQYEEEMEAKAELQRVLSKANSEVAQW |
| | RTKYETDAIQRTEELEEAKKKLAQRLQDAEEAVEA |
| | VNAKCSSLEKTKHRLQNEIEDLMVDVERSNAAAA |
| | ALDKKQRNFDKILAEWKQKYEESQSELESSQKEA |
| | RSLSTELFKLKNAYEESLEHLETFKRENKNLQEEISD |
| | LTEQLGEGGKNVHELEKIRKQLEVEKLELQSALEE |
| | AEASLEHEEGKILRAQLEFNQIKAEIERKLAEKDEE |
| | MEQAKRNHLRMVDSLQTSLDAETRSRNEALRVK |
| | KKMEGDLNEMEIQLSQANRIASEAQKHLKNSQA |
| | HLKDTQLQLDDAVHANDDLKENIAIVERRNNLLQ |
| | AELEELRAVVEQTERSRKLAEQELIETSERVQLLHS |
| | QNTSLINQKKKMESDLTQLQTEVEEAVQECRNAE |
| | EKAKKAITDAAMMAEELKKEQDTSAHLERMKKN |
| | MEQTIKDLQHRLDEAEQIALKGGKKQLQKLEARV |
| | RELENELEAEQKRNAESVKGMRKSERRIKELTYQT |
| | EEDKKNLMRLQDLVDKLQLKVKAYKRQAEEAEE |
| | QANTNLSKFRKVQHELDEAEERADIAESQVNKLR |
| | AKSRDIGAKKMHDEE |
| Native Human MYH7 protein (SEQ ID NO: 97) | MGDSEMAVFGAAAPYLRKSEKERLEAQTRPFDL |
| | KKDVFVPDDKQEFVKAKIVSREGGKVTAETEYGK |
| | TVTVKEDQVMQQNPPKFDKIEDMAMLTFLHEP |
| | AVLYNLKDRYGSWMIYTYSGLFCVTVNPYKWLPV |
| | YTPEVVAAYRGKKRSEAPPHIFSISDNAYQYMLTD |
| | RENQSILITGESGAGKTVNTKRVIQYFAVIAAIGDR |
| | SKKDQSPGKGTLEDQIIQANPALEAFGNAKTVRN |
| | DNSSRFGKFIRIHFGATGKLASADIETYLLEKSRVIF |
| | QLKAERDYHIFYQILSNKKPELLDMLLITNNPYDYA |
| | FISQGETTVASIDDAEELMATDNAFDVLGFTSEEK |
| | NSMYKLTGAIMHFGNMKFKLKQREEQAEPDGTE |
| | EADKSAYLMGLNSADLLKGLCHPRVKVGNEYVTK |
| | GQNVQQVIYATGALAKAVYERMFNWMVTRINA |
| | TLETKQPRQYFIGVLDIAGFEIFDFNSFEQLCINFT |
| | NEKLQQFFNHHMFVLEQEEYKKEGIEWTFIDFG |
| | MDLQACIDLIEKPMGIMSILEEECMFPKATDMTF |
| | KAKLFDNHLGKSANFQKPRNIKGKPEAHFSLIHYA |
| | GIVDYNIIGWLQKNKDPLNETVVGLYQKSSLKLLS |
| | TLFANYAGADAPIEKGKGKAKKGSSFQTVSALHR |
| | ENLNKLMTNLRSTHPHFVRCIIPNETKSPGVMDN |
| | PLVMHQLRCNGVLEGIRICRKGFPNRILYGDFRQ |
| | RYRILNPAAIPEGQFIDSRKGAEKLLSSLDIDHNQY |
| | KFGHTKVFFKAGLLGLLEEMRDERLSRIITRIQAQS |
| | RGVLARMEYKKLLERRDSLLVIQWNIRAFMGVKN |
| | WPWMKLYFKIKPLLKSAEREKEMASMKEEFTRLK |
| | EALEKSEARRKELEEKMVSLLQEKNDLQLQVQAE |
| | QDNLADAEERCDQLIKNKIQLEAKVKEMNERLED |
| | EEEMNAELTAKKRKLEDECSELKRDIDDLELTLAK |
| | VEKEKHATENKVKNLTEEMAGLDEIIAKLTKEKKA |
| | LQEAHQQALDDLQAEEDKVNTLTKAKVKLEQQV |
| | DDLEGSLEQEKKVRMDLERAKRKLEGDLKLTQESI |
| | MDLENDKQQLDERLKKKDFELNALNARIEDEQAL |
| | GSQLQKKLKELQARIEELEEELEAERTARAKVEKLR |
| | SDLSRELEEISERLEEAGGATSVQIEMNKKREAEF |
| | QKMRRDLEEATLQHEATAAALRKKHADSVAELG |
| | EQIDNLQRVKQKLEKEKSEFKLELDDVTSNMEQII |
| | KAKANLEKMCRTLEDQMNEHRSKAEETQRSVND |
| | LTSQRAKLQTENGELSRQLDEKEALISQLTRGKLTY |
| | TQQLEDLKRQLEEEVKAKNALAHALQSARHDCDL |
| | LREQYEEETEAKAELQRVLSKANSEVAQWRTKYE |
| | TDAIQRTEELEEAKKKLAQRLQEAEEAVEAVNAKC |
| | SSLEKTKHRLQNEIEDLMVDVERSNAAAAALDKK |
| | QRNFDKILAEWKQKYEESQSELESSQKEARSLSTE |
| | LFKLKNAYEESLEHLETFKRENKNLQEEISDLTEQL |
| | GSSGKTIHELEKVRKQLEAEKMELQSALEEAEASL |
| | EHEEGKILRAQLEFNQIKAEIERKLAEKDEEMEQA |
| | KRNHLRVVDSLQTSLDAETRSRNEALRVKKKMEG |
| | DLNEMEIQLSHANRMAAEAQKQVKSLQSLLKDT |
| | QIQLDDAVRANDDLKENIAIVERRNNLLQAELEEL |
| | RAVVEQTERSRKLAEQELIETSERVQLLHSQNTSLI |
| | NQKKKMDADLSQLQTEVEEAVQECRNAEEKAKK |
| | AITDAAMMAEELKKEQDTSAHLERMKKNMEQTI |
| | KDLQHRLDEAEQIALKGGKKQLQKLEARVRELEN |
| | ELEAEQKRNAESVKGMRKSERRIKELTYQTEEDRK |
| | NLLRLQDLVDKLQLKVKAYKRQAEEAEEQANTNL |
| | SKFRKVQHELDEAEERADIAESQVNKLRAKSRDIG |
| | TKGLNEE |
| Mutant Human MYH7 protein (SEQ ID NO: 155) | MGDSEMAVFGAAAPYLRKSEKERLEAQTRPFDL |
| (R403Q substitution underlined) | KKDVFVPDDKQEFVKAKIVSREGGKVTAETEYGK |
| | TVTVKEDQVMQQNPPKFDKIEDMAMLTFLHEP |
| | AVLYNLKDRYGSWMIYTYSGLFCVTVNPYKWLPV |
| | YTPEVVAAYRGKKRSEAPPHIFSISDNAYQYMLTD |
| | RENQSILITGESGAGKTVNTKRVIQYFAVIAAIGDR |
| | SKKDQSPGKGTLEDQIIQANPALEAFGNAKTVRN |
| | DNSSRFGKFIRIHFGATGKLASADIETYLLEKSRVIF |
| | QLKAERDYHIFYQILSNKKPELLDMLLITNNPYDYA |
| | FISQGETTVASIDDAEELMATDNAFDVLGFTSEEK |
| | NSMYKLTGAIMHFGNMKFKLKQREEQAEPDGTE |
| | EADKSAYLMGLNSADLLKGLCHPQVKVGNEYVT |
| | KGQNVQQVIYATGALAKAVYERMFNWMVTRIN |
| | ATLETKQPRQYFIGVLDIAGFEIFDFNSFEQLCINFT |
| | NEKLQQFFNHHMFVLEQEEYKKEGIEWTFIDFG |
| | MDLQACIDLIEKPMGIMSILEEECMFPKATDMTF |
| | KAKLFDNHLGKSANFQKPRNIKGKPEAHFSLIHYA |
| | GIVDYNIIGWLQKNKDPLNETVVGLYQKSSLKLLS |
| | TLFANYAGADAPIEKGKGKAKKGSSFQTVSALHR |
| | ENLNKLMTNLRSTHPHFVRCIIPNETKSPGVMDN |
| | PLVMHQLRCNGVLEGIRICRKGFPNRILYGDFRQ |
| | RYRILNPAAIPEGQFIDSRKGAEKLLSSLDIDHNQY |
| | KFGHTKVFFKAGLLGLLEEMRDERLSRIITRIQAQS |
| | RGVLARMEYKKLLERRDSLLVIQWNIRAFMGVKN |
| | WPWMKLYFKIKPLLKSAEREKEMASMKEEFTRLK |
| | EALEKSEARRKELEEKMVSLLQEKNDLQLQVQAE |
| | QDNLADAEERCDQLIKNKIQLEAKVKEMNERLED |
| | EEEMNAELTAKKRKLEDECSELKRDIDDLELTLAK |
| | VEKEKHATENKVKNLTEEMAGLDEIIAKLTKEKKA |
| | LQEAHQQALDDLQAEEDKVNTLTKAKVKLEQQV |
| | DDLEGSLEQEKKVRMDLERAKRKLEGDLKLTQESI |
| | MDLENDKQQLDERLKKKDFELNALNARIEDEQAL |
| | GSQLQKKLKELQARIEELEEELEAERTARAKVEKLR |
| | SDLSRELEEISERLEEAGGATSVQIEMNKKREAEF |
| | QKMRRDLEEATLQHEATAAALRKKHADSVAELG |
| | EQIDNLQRVKQKLEKEKSEFKLELDDVTSNMEQII |
| | KAKANLEKMCRTLEDQMNEHRSKAEETQRSVND |
| | LTSQRAKLQTENGELSRQLDEKEALISQLTRGKLTY |
| | TQQLEDLKRQLEEEVKAKNALAHALQSARHDCDL |
| | LREQYEEETEAKAELQRVLSKANSEVAQWRTKYE |
| | TDAIQRTEELEEAKKKLAQRLQEAEEAVEAVNAKC |
| | SSLEKTKHRLQNEIEDLMVDVERSNAAAAALDKK |
| | QRNFDKILAEWKQKYEESQSELESSQKEARSLSTE |
| | LFKLKNAYEESLEHLETFKRENKNLQEEISDLTEQL |
| | GSSGKTIHELEKVRKQLEAEKMELQSALEEAEASL |
| | EHEEGKILRAQLEFNQIKAEIERKLAEKDEEMEQA |
| | KRNHLRVVDSLQTSLDAETRSRNEALRVKKKMEG |
| | DLNEMEIQLSHANRMAAEAQKQVKSLQSLLKDT |
| | QIQLDDAVRANDDLKENIAIVERRNNLLQAELEEL |
| | RAVVEQTERSRKLAEQELIETSERVQLLHSQNTSLI |
| | NQKKKMDADLSQLQTEVEEAVQECRNAEEKAKK |
| | AITDAAMMAEELKKEQDTSAHLERMKKNMEQTI |
| | KDLQHRLDEAEQIALKGGKKQLQKLEARVRELEN |
| | ELEAEQKRNAESVKGMRKSERRIKELTYQTEEDRK |
| | NLLRLQDLVDKLQLKVKAYKRQAEEAEEQANTNL |
| | SKFRKVQHELDEAEERADIAESQVNKLRAKSRDIG |
| | TKGLNEE |

TABLE 14B
Mutant and WT Myh6 and Myh7 full transcripts

| Sequence Name (SEQ ID NO) | Sequence |
|---|---|
| Murine Myh6 gene with G > A mutation - no | ATATAAAGGGGCTGGAGCACTGAGAGCT |
| humanized nucleotides (SEQ ID NO: 156) | GTCAGACAGAGATTTCTCCAACCCAGGAT |
| | CTCTGGATTGGTCTCCCAGCCTCTGCTAC |
| | TCCTCTTCCTGCCTGTTCCTCTCTCCGTC |
| | CAGCTGCGCCACTGTGGTGCCTCGTTCC |
| | AGCTGTGGTCCACATTCTTCAGGATTCTC |
| | TGAAAAGTTAACCAGAGTTTGAGTGACAG |
| | AATGACGGACGCCCAGATGGCTGACTTC |
| | GGGGCAGCAGCCCAGTACCTCCGAAAGT |
| | CAGAGAAGGAACGCCTAGAGGCCCAGAC |
| | CCGGCCCTTTGACATCCGCACGGAGTGC |
| | TTCGTGCCTGATGACAAGGAGGAGTATGT |
| | TAAGGCCAAGGTCGTGTCCCGGGAAGGG |
| | GGCAAAGTCACTGCGGAAACTGAAAACG |
| | GAAAGACGGTGACCATAAAGGAGGACCA |
| | GGTGATGCAGCAGAACCCACCCAAGTTC |
| | GACAAGATCGAGGACATGGCCATGCTGA |
| | CCTTCCTGCACGAGCCGGCTGTGCTGTA |
| | CAACCTCAAGGAGCGCTACGCGGCCTGG |
| | ATGATCTATACCTACTCAGGCCTCTTCTG |
| | CGTCACCGTCAACCCCTATAAGTGGCTG |
| | CCTGTGTACAATGCGGAAGTGGTGGCCG |
| | CCTACCGGGGCAAGAAGAGGAGCGAGG |
| | CCCCTCCTCACATCTTCTCCATCTCTGAC |
| | AACGCCTATCAGTACATGCTGACAGATCG |
| | GGAGAATCAGTCCATCCTCATCACCGGA |
| | GAATCCGGAGCGGGGAAGACTGTGAACA |
| | CAAAACGTGTCATCCAGTACTTTGCCAGC |
| | ATTGCAGCCATAGGGGACCGTAGCAAGA |
| | AGGAAAATCCTAATGCAAACAAGGGCACC |
| | CTGGAGGACCAGATTATCCAGGCTAACC |
| | CCGCTCTGGAGGCCTTCGGCAACGCCAA |
| | GACTGTCCGGAATGACAACTCCTCCCGC |
| | TTTGGGAAATTCATCAGGATCCACTTTGG |
| | AGCTACTGGAAAGCTGGCTTCTGCAGAC |
| | ATAGAGACCTACCTTCTGGAGAAGTCCCG |
| | GGTGATCTTCCAGCTAAAGGCTGAGAGG |
| | AACTACCACATCTTCTACCAGATCCTGTC |
| | CAACAAGAAGCCGGAGCTGCTGGACATG |
| | CTGCTGGTCACCAACAACCCATACGACTA |
| | CGCCTTCGTCTCTCAGGGAGAGGTGTCC |
| | GTGGCCTCCATTGATGACTCTGAGGAGC |
| | TCTTGGCCACTGATAGTGCCTTTGATGTG |
| | CTGAGCTTCACGGCAGAGGAGAAGGCTG |
| | GTGTCTACAAGCTGACAGGGGCCATCAT |
| | GCACTACGGAAACATGAAGTTCAAGCAGA |
| | AGCAGCGGGAGGAGCAGGCGGAGCCTG |
| | ATGGCACAGAAGATGCTGACAAATCAGC |
| | CTACCTTATGGGGCTGAACTCAGCTGACC |
| | TGCTCAAGGGCCTGTGTCACCCTCAGGT |
| | GAAGGTGGGGAACGAGTATGTCACCAAG |
| | GGGCAGAGTGTACAGCAAGTGTACTATTC |
| | CATCGGGGCACTGGCCAAGTCAGTGTAC |
| | GAGAAGATGTTCAACTGGATGGTGACAC |
| | GCATCAACGCAACCCTGGAGACCAAGCA |
| | GCCGCGCCAGTACTTCATAGGTGTCCTG |
| | GACATTGCCGGCTTTGAGATCTTCGATTT |
| | CAACAGCTTTGAGCAGCTGTGCATCAACT |
| | TCACCAATGAGAAGCTGCAGCAGTTCTTC |
| | AACCACCACATGTTCGTGCTGGAGCAGG |
| | AGGAGTACAAGAAGGAGGGCATTGAGTG |
| | GGAGTTTATCGACTTCGGCATGGACCTG |
| | CAGGCCTGCATCGACCTCATCGAGAAGC |
| | CCATGGGCATCATGTCCATCCTCGAGGA |
| | GGAGTGCATGTTCCCCAAGGCCTCAGAC |
| | ATGACCTTCAAGGCCAAGCTGTATGACAA |
| | CCACCTGGGCAAATCCAACAACTTCCAGA |
| | AGCCTCGCAATGTCAAGGGGAAGCAGGA |
| | AGCCCACTTCTCCTTGGTCCACTATGCTG |
| | GCACCGTGGACTACAACATTATGGGCTG |
| | GCTGGAAAAGAACAAGGACCCACTCAAT |
| | GAGACGGTGGTGGGTTTGTACCAGAAGT |
| | CCTCCCTCAAGCTCATGGCTACACTCTTC |
| | TCTACCTATGCTTCTGCTGATACCGGTGA |
| | CAGTGGTAAAGGCAAAGGAGGCAAGAAG |
| | AAAGGCTCATCCTTCCAAACAGTGTCTGC |
| | TCTCCACCGGGAAAATCTGAACAAGCTGA |
| | TGACAAACCTGAAGACCACCCACCCTCAC |
| | TTTGTGCGCTGCATCATTCCCAACGAGCG |
| | AAAGGCTCCAGGGGTGATGGACAACCCC |
| | CTGGTCATGCACCAGCTGCGATGCAATG |
| | GCGTGCTGGAGGGTATCCGCATCTGCAG |
| | GAAGGGCTTCCCCAACCGCATTCTCTATG |
| | GGGACTTCCGGCAGAGGTATCGCATCCT |
| | GAACCCAGCAGCCATCCCTGAGGGGCAA |
| | TTCATTGATAGCAGGAAAGGGGCTGAGA |
| | AACTGCTGGGCTCCCTGGACATTGACCA |
| | CAACCAATACAAGTTTGGCCACACCAAGG |
| | TGTTCTTCAAGGCGGGCCTGCTGGGGCT |
| | GCTCGAGGAGATGCGAGATGAGAGGCTG |
| | AGCCGTATCATCACCAGAATCCAGGCCC |
| | AGGCCCGAGGGCAGCTCATGCGCATTGA |
| | GTTCAAGAAGATAGTGGAACGCAGGGAT |
| | GCCCTGCTGGTTATCCAGTGGAACATTCG |
| | GGCCTTCATGGGGGTCAAGAATTGGCCA |
| | TGGATGAAGCTCTACTTCAAGATCAAACC |
| | GCTGCTGAAGAGCGCAGAGACGGAGAAG |
| | GAGATGGCCAACATGAAGGAGGAGTTTG |
| | GGCGAGTCAAAGATGCACTGGAGAAGTC |
| | TGAGGCTCGCCGCAAGGAGCTGGAGGA |
| | GAAGATGGTGTCCCTGCTGCAGGAGAAG |
| | AATGACCTACAGCTCCAAGTGCAGGCGG |
| | AACAAGACAACCTCAATGATGCAGAGGA |
| | GCGCTGTGACCAGCTGATCAAGAACAAG |
| | ATCCAGCTGGAGGCCAAGGTGAAGGAGA |
| | TGACCGAGAGGCTGGAGGACGAGGAGG |
| | AGATGAACGCCGAGCTCACTGCCAAGAA |
| | GCGCAAGCTGGAAGATGAGTGCTCAGAG |
| | CTCAAGAAGGATATTGATGACCTGGAGCT |
| | GACGCTGGCCAAGGTGGAAAAGGAAAAG |
| | CATGCAACAGAGAACAAGGTTAAAAACCT |
| | AACAGAGGAGATGGCTGGGCTGGATGAA |
| | ATCATTGCCAAGCTGACCAAAGAGAAGAA |
| | AGCTCTGCAAGAAGCCCACCAGCAAGCC |
| | CTCGATGACCTGCAGGCTGAAGAAGACA |
| | AGGTCAACACGCTGACCAAGTCCAAAGT |
| | CAAGCTGGAGCAGCAGGTGGATGATCTG |
| | GAGGGATCCCTGGAGCAGGAGAAGAAAG |
| | TGCGCATGGACCTAGAGCGAGCCAAGCG |
| | GAAGCTGGAGGGAGACCTGAAGCTGACC |
| | CAGGAGAGCATCATGGACCTGGAGAATG |
| | ACAAGCTTCAGCTGGAAGAAAAGCTCAAG |
| | AAGAAAGAGTTCGACATCAGTCAGCAGAA |
| | CAGTAAAATTGAGGACGAGCAGGCCCTG |
| | GCTCTTCAGCTGCAGAAGAAACTGAAGG |
| | AAAACCAGGCACGCATCGAGGAGCTGGA |
| | GGAGGAGCTGGAGGCAGAGCGCACAGC |
| | CCGGGCTAAGGTGGAGAAGCTGCGCTCT |
| | GACCTGTCCCGGGAGCTGGAGGAGATCA |
| | GTGAGAGGCTGGAGGAGGCAGGCGGGG |
| | CCACATCCGTGCAGATAGAGATGAATAAG |
| | AAGCGCGAGGCCGAGTTCCAGAAGATGC |
| | GGCGGGACCTGGAGGAGGCCACGCTGC |
| | AGCACGAGGCCACGGCGGCGGCCCTGC |
| | GCAAGAAGCATGCTGACAGCGTGGCGGA |
| | GCTGGGCGAGCAGATCGACAACCTCCAG |
| | CGGGTGAAGCAGAAGCTGGAGAAAGAGA |
| | AGAGCGAGTTCAAGCTGGAGCTGGATGA |
| | CGTCACCTCCAACATGGAGCAGATCATCA |
| | AGGCCAAGGCCAACCTGGAGAAAGTGTC |
| | CCGGACACTGGAGGACCAGGCCAATGAG |
| | TACCGCGTGAAGCTGGAAGAAGCCCAGC |
| | GCTCCCTCAATGACTTCACCACACAGCGA |
| | GCCAAGCTGCAGACAGAGAACGGGGAGT |
| | TGGCTAGGCAACTGGAAGAAAAGGAGGC |
| | ATTGATTTCCCAGCTGACCCGAGGCAAG |
| | CTCTCCTACACCCAGCAGATGGAGGACC |
| | TCAAGAGGCAACTGGAGGAGGAAGGCAA |
| | GGCCAAGAACGCCCTGGCCCACGCACTG |
| | CAATCATCCCGGCATGACTGTGACCTGCT |
| | GAGGGAACAGTATGAAGAAGAAATGGAG |
| | GCCAAGGCTGAGCTACAGCGTGTCCTGT |
| | CCAAGGCCAACTCAGAGGTGGCCCAGTG |
| | GAGGACCAAGTATGAGACGGATGCCATA |
| | CAGAGGACGGAGGAGCTGGAGGAAGCC |
| | AAGAAGAAGCTGGCTCAGAGGCTGCAGG |
| | ATGCAGAGGAGGCAGTGGAGGCCGTCAA |
| | CGCCAAGTGTTCCTCCCTGGAGAAGACC |
| | AAGCACAGGCTGCAGAATGAGATCGAGG |
| | ACCTGATGGTGGACGTGGAGCGCTCCAA |
| | TGCCGCCGCCGCAGCCCTGGACAAGAAG |
| | CAGAGGAACTTTGACAAGATCCTGGCTGA |
| | GTGGAAGCAGAAGTATGAGGAGTCGCAG |
| | TCAGAGCTGGAGTCTTCCCAGAAGGAGG |
| | CGCGCTCCCTGAGCACAGAGCTCTTCAA |
| | GCTCAAGAACGCCTATGAGGAGTCTCTG |
| | GAGCACCTGGAGACCTTCAAGCGGGAGA |
| | ACAAGAACCTCCAGGAGGAGATCTCAGA |
| | CCTGACTGAACAGCTGGGAGAAGGGGGG |
| | AAAAACGTGCACGAGCTGGAGAAGATCC |
| | GCAAACAGCTGGAGGTGGAGAAGCTGGA |
| | GCTGCAGTCAGCCCTGGAGGAGGCTGAG |
| | GCCTCCCTGGAGCACGAGGAGGGCAAGA |
| | TCCTCCGTGCCCAGCTGGAGTTCAACCA |
| | GATCAAGGCAGAGATCGAAAGGAAGCTG |
| | GCAGAGAAGGATGAGGAGATGGAGCAGG |
| | CCAAGCGCAACCACCTGCGGATGGTGGA |
| | CTCCCTGCAGACCTCCCTGGATGCGGAG |
| | ACACGCAGCCGCAATGAGGCCCTGCGGG |
| | TGAAGAAGAAGATGGAGGGCGACCTCAA |
| | CGAGATGGAGATCCAGCTCAGCCAGGCC |
| | AATAGAATAGCCTCAGAGGCACAGAAACA |
| | CCTGAAGAATTCTCAAGCTCACTTGAAGG |
| | ACACCCAGCTCCAGCTGGATGATGCTGT |
| | CCATGCCAATGACGACCTGAAGGAGAAC |
| | ATCGCCATCGTGGAACGGCGCAACAACC |
| | TGCTGCAGGCGGAGCTGGAGGAGCTGC |
| | GGGCTGTGGTGGAGCAGACGGAGCGGT |
| | CTCGGAAGCTGGCAGAGCAGGAGCTGAT |
| | TGAGACCAGCGAGCGGGTGCAGCTGCTG |
| | CACTCGCAGAACACCAGCCTCATCAACCA |
| | GAAGAAGAAGATGGAGTCAGACCTGACC |
| | CAACTCCAGACAGAAGTAGAGGAGGCAG |
| | TGCAGGAGTGTAGGAACGCAGAGGAGAA |
| | GGCCAAGAAGGCCATCACAGATGCCGCA |
| | ATGATGGCTGAGGAGCTGAAGAAGGAGC |
| | AGGACACCAGCGCCCACCTGGAGCGCAT |
| | GAAGAAGAACATGGAGCAGACCATCAAG |
| | GACTTGCAGCACCGTCTGGACGAGGCAG |
| | AGCAGATCGCCCTCAAGGGCGGCAAGAA |
| | GCAGCTGCAGAAGCTGGAGGCCCGGGT |
| | CCGGGAGCTGGAGAATGAGCTGGAGGCT |
| | GAGCAGAAGCGCAATGCAGAGTCGGTGA |
| | AGGGCATGAGGAAGAGCGAGCGGCGCA |
| | TCAAGGAGCTCACCTACCAGACAGAGGA |
| | AGACAAGAAGAACTTAATGCGGCTGCAG |
| | GACCTGGTGGACAAGCTACAGTTGAAGG |
| | TGAAGGCCTACAAGCGCCAGGCTGAGGA |
| | GGCGGAGGAGCAGGCCAACACCAACCTG |
| | TCCAAGTTCCGCAAGGTGCAGCACGAGC |
| | TGGATGAGGCGGAGGAGAGGGCGGACA |
| | TCGCCGAGTCCCAGGTCAACAAGCTGCG |
| | GGCCAAGAGCCGGGACATTGGTGCCAAG |
| | AAGATGCACGACGAGGAATAACCTCTCCA |
| | GCAGACCCTCGCTGTGGCCAATCCACAA |
| | TAAACATAAACGTTCGACTCTGCC |
| Human Myh7 gene with G > A mutation | GGGGGTGGGGGTGCCCTGCTGCCCCAT |
| (SEQ ID NO: 157) | ATATACAGCCCCTGAGACCAGGTCTGGC |
| | TCCACAGCTCTGTCCTGCTCTGTGTCTTT |
| | CCCTGCTGCTCTCAGGTCCCCTGCAGGC |
| | CTTGGCCCCTTTCCTCATCTGTAGACACA |
| | CTTGAGTAGCCCAGGCACAGCCATGGGA |
| | GATTCGGAGATGGCAGTCTTTGGGGCTG |
| | CCGCCCCCTACCTGCGCAAGTCAGAGAA |
| | GGAGCGGCTAGAAGCGCAGACCAGGCCT |
| | TTTGACCTCAAGAAGGATGTCTTCGTGCC |
| | TGATGACAAACAGGAGTTTGTCAAGGCCA |
| | AGATCGTGTCTCGAGAGGGTGGCAAAGT |
| | CACTGCCGAGACCGAGTATGGCAAGACA |
| | GTGACCGTGAAGGAGGACCAGGTGATGC |
| | AGCAGAACCCACCCAAGTTCGACAAAATC |
| | GAGGACATGGCCATGCTGACCTTCCTGC |
| | ATGAGCCCGCGGTGCTCTACAACCTCAA |
| | GGATCGCTACGGCTCCTGGATGATCTAC |
| | ACCTACTCGGGCCTCTTCTGTGTCACCGT |
| | CAACCCTTACAAGTGGCTGCCGGTGTAC |
| | ACTCCTGAGGTGGTGGCTGCCTACCGGG |
| | GCAAGAAGAGGAGCGAGGCCCCGCCCC |
| | ACATCTTCTCCATCTCCGACAACGCCTAT |
| | CAGTACATGCTGACAGACAGAGAAAACCA |
| | GTCCATCCTGATCACCGGAGAATCCGGA |
| | GCAGGGAAGACAGTCAACACCAAGAGGG |
| | TCATCCAGTACTTTGCTGTTATTGCAGCC |
| | ATTGGGGACCGCAGCAAGAAGGACCAGA |
| | GCCCGGGCAAGGGCACCCTGGAGGACC |
| | AGATCATCCAGGCCAACCCTGCTCTGGA |
| | GGCCTTTGGCAATGCCAAGACCGTCCGG |
| | AACGACAACTCCTCCCGCTTCGGGAAATT |
| | CATTCGAATTCATTTTGGGGCAACAGGAA |
| | AGTTGGCATCTGCAGACATAGAGACCTAT |
| | CTTCTGGAAAAATCCAGAGTTATTTTCCA |
| | GCTGAAAGCAGAGAGAGATTATCACATTT |
| | TCTACCAAATCCTGTCTAACAAAAAGCCT |
| | GAGCTGCTGGACATGCTGCTGATCACCA |
| | ACAACCCCTACGATTATGCATTCATCTCC |
| | CAAGGAGAGACCACCGTGGCCTCCATTG |
| | ATGACGCTGAGGAGCTCATGGCCACTGA |
| | TAACGCTTTTGATGTGCTGGGCTTCACTT |
| | CAGAGGAGAAAAACTCCATGTATAAGCTG |
| | ACAGGCGCCATCATGCACTTTGGAAACAT |
| | GAAGTTCAAGCTGAAGCAGCGGGAGGAG |
| | CAGGCGGAGCCAGACGGCACTGAAGAG |
| | GCTGACAAGTCTGCCTACCTCATGGGGC |
| | TGAACTCAGCCGACCTGCTCAAGGGGCT |
| | GTGCCACCCTCAGGTGAAAGTGGGCAAT |
| | GAGTACGTCACCAAGGGGCAGAATGTCC |
| | AGCAGGTGATATATGCCACTGGGGCACT |
| | GGCCAAGGCAGTGTATGAGAGGATGTTC |
| | AACTGGATGGTGACGCGCATCAATGCCA |
| | CCCTGGAGACCAAGCAGCCACGCCAGTA |
| | CTTCATAGGAGTCCTGGACATCGCTGGCT |
| | TCGAGATCTTCGATTTCAACAGCTTTGAG |
| | CAGCTCTGCATCAACTTCACCAACGAGAA |
| | GCTGCAGCAGTTCTTCAACCACCACATGT |
| | TTGTGCTGGAGCAGGAGGAGTACAAGAA |
| | GGAGGGCATCGAGTGGACATTCATTGAC |
| | TTTGGCATGGACCTGCAGGCCTGCATTG |
| | ACCTCATCGAGAAGCCCATGGGCATCAT |
| | GTCCATCCTGGAAGAGGAGTGCATGTTC |
| | CCCAAGGCCACCGACATGACCTTCAAGG |
| | CCAAGCTGTTTGACAACCACCTGGGCAAA |
| | TCCGCCAACTTCCAGAAGCCACGCAATAT |
| | CAAGGGGAAGCCTGAAGCCCACTTCTCC |
| | CTGATCCACTATGCCGGCATCGTGGACTA |
| | CAACATCATTGGCTGGCTGCAGAAGAACA |
| | AGGATCCTCTCAATGAGACTGTCGTGGG |
| | CTTGTATCAGAAGTCTTCCCTCAAGCTGC |
| | TCAGCACCCTGTTTGCCAACTATGCTGGG |
| | GCTGATGCGCCTATTGAGAAGGGCAAAG |
| | GCAAGGCCAAGAAAGGCTCGTCCTTTCA |
| | GACTGTGTCAGCTCTGCACAGGGAAAAT |
| | CTGAACAAGCTGATGACCAACTTGCGCTC |
| | CACCCATCCCCACTTTGTACGTTGTATCA |
| | TCCCTAATGAGACAAAGTCTCCAGGGGT |
| | GATGGACAACCCCCTGGTCATGCACCAG |
| | CTGCGCTGCAATGGTGTGCTGGAGGGCA |
| | TCCGCATCTGCAGGAAAGGCTTCCCCAA |
| | CCGCATCCTCTACGGGGACTTCCGGCAG |
| | AGGTATCGCATCCTGAACCCAGCGGCCA |
| | TCCCTGAGGGACAGTTCATTGATAGCAG |
| | GAAGGGGGCAGAGAAGCTGCTCAGCTCC |
| | CTGGACATTGATCACAACCAGTACAAGTT |
| | TGGCCACACCAAGGTGTTCTTCAAGGCC |
| | GGGCTGCTGGGGCTGCTGGAGGAAATGA |
| | GGGACGAGAGGCTGAGCCGCATCATCAC |
| | GCGTATCCAGGCCCAGTCCCGAGGTGTG |
| | CTCGCCAGAATGGAGTACAAAAAGCTGCT |
| | GGAACGTAGAGACTCCCTGCTGGTAATC |
| | CAGTGGAACATTCGGGCCTTCATGGGGG |
| | TCAAGAATTGGCCCTGGATGAAGCTCTAC |
| | TTCAAGATCAAGCCGCTGCTGAAGAGTG |
| | CAGAAAGAGAGAAGGAGATGGCCTCCAT |
| | GAAGGAGGAGTTCACACGCCTCAAAGAG |
| | GCGCTAGAGAAGTCCGAGGCTCGCCGCA |
| | AGGAGCTGGAGGAGAAGATGGTGTCCCT |
| | GCTGCAGGAGAAGAATGACCTGCAGCTC |
| | CAAGTGCAGGCGGAACAAGACAACCTGG |
| | CAGATGCTGAGGAGCGCTGTGATCAGCT |
| | GATCAAAAACAAGATTCAGCTGGAGGCCA |
| | AGGTGAAGGAGATGAACGAGAGGCTGGA |
| | GGATGAGGAGGAGATGAATGCTGAGCTC |
| | ACTGCCAAGAAGCGCAAGCTGGAAGATG |
| | AGTGCTCAGAGCTCAAAAGGGACATCGA |
| | TGATCTGGAGCTGACACTGGCCAAAGTG |
| | GAGAAGGAGAAACACGCAACAGAGAACA |
| | AGGTGAAAAACCTGACAGAGGAGATGGC |
| | TGGGCTGGATGAGATCATTGCCAAGCTG |
| | ACCAAGGAGAAGAAAGCTCTGCAAGAGG |
| | CCCACCAACAGGCTCTGGATGACCTTCA |
| | GGCCGAGGAGGACAAGGTCAACACCCTG |
| | ACTAAGGCCAAAGTCAAGCTGGAGCAGC |
| | AAGTGGATGATCTGGAAGGATCCCTGGA |
| | GCAAGAGAAGAAGGTGCGCATGGACCTG |
| | GAGCGAGCGAAGCGGAAGCTGGAGGGC |
| | GACCTGAAGCTGACCCAGGAGAGCATCA |
| | TGGACCTGGAGAATGACAAGCAGCAGCT |
| | GGATGAGCGGCTGAAAAAAAAAGACTTTG |
| | AGCTGAATGCTCTCAACGCAAGGATTGAG |
| | GATGAACAGGCCCTCGGCAGCCAGCTGC |
| | AGAAGAAGCTCAAGGAGCTTCAGGCACG |
| | CATCGAGGAGCTGGAGGAGGAGCTGGA |
| | GGCCGAGCGCACCGCCAGGGCTAAGGT |
| | GGAGAAGCTGCGCTCAGACCTGTCTCGG |
| | GAGCTGGAGGAGATCAGCGAGCGGCTG |
| | GAAGAGGCCGGCGGGGCCACGTCCGTG |
| | CAGATCGAGATGAACAAGAAGCGCGAGG |
| | CCGAGTTCCAGAAGATGCGGCGGGACCT |
| | GGAGGAGGCCACGCTGCAGCACGAGGC |
| | CACTGCCGCGGCCCTGCGCAAGAAGCAC |
| | GCCGACAGCGTGGCCGAGCTGGGCGAG |
| | CAGATCGACAACCTGCAGCGGGTGAAGC |
| | AGAAGCTGGAGAAGGAGAAGAGCGAGTT |
| | CAAGCTGGAGCTGGATGACGTCACCTCC |
| | AACATGGAGCAGATCATCAAGGCCAAGG |
| | CTAACCTGGAGAAGATGTGCCGGACCTT |
| | GGAAGACCAGATGAATGAGCACCGGAGC |
| | AAGGCGGAGGAGACCCAGCGTTCTGTCA |
| | ACGACCTCACCAGCCAGCGGGCCAAGTT |
| | GCAAACCGAGAATGGTGAGCTGTCCCGG |
| | CAGCTGGATGAGAAGGAGGCACTGATCT |
| | CCCAGCTGACCCGAGGCAAGCTCACCTA |
| | CACCCAGCAGCTGGAGGACCTCAAGAGG |
| | CAGCTGGAGGAGGAGGTTAAGGCGAAGA |
| | ACGCCCTGGCCCACGCACTGCAGTCGGC |
| | CCGGCATGACTGCGACCTGCTGCGGGAG |
| | CAGTACGAGGAGGAGACGGAGGCCAAG |
| | GCCGAGCTGCAGCGCGTCCTTTCCAAGG |
| | CCAACTCGGAGGTGGCCCAGTGGAGGAC |
| | CAAGTATGAGACGGACGCCATTCAGCGG |
| | ACTGAGGAGCTCGAGGAGGCCAAGAAGA |
| | AGCTGGCCCAGCGGCTGCAGGAAGCTGA |
| | GGAGGCCGTGGAGGCTGTTAATGCCAAG |
| | TGCTCCTCGCTGGAGAAGACCAAGCACC |
| | GGCTACAGAATGAGATCGAGGACTTGAT |
| | GGTGGACGTAGAGCGCTCCAATGCTGCT |
| | GCTGCAGCCCTGGACAAGAAGCAGAGGA |
| | ACTTCGACAAGATCCTGGCCGAGTGGAA |
| | GCAGAAGTATGAGGAGTCGCAGTCGGAG |
| | CTGGAGTCCTCGCAGAAGGAGGCTCGCT |
| | CCCTCAGCACAGAGCTCTTCAAACTCAAG |
| | AACGCCTATGAGGAGTCCCTGGAACATCT |
| | GGAGACCTTCAAGCGGGAGAACAAAAAC |
| | CTGCAGGAGGAGATCTCCGACTTGACTG |
| | AGCAGTTGGGTTCCAGCGGAAAGACTAT |
| | CCATGAGCTGGAGAAGGTCCGAAAGCAG |
| | CTGGAGGCCGAGAAGATGGAGCTGCAGT |
| | CAGCCCTGGAGGAGGCCGAGGCCTCCCT |
| | GGAGCACGAGGAGGGCAAGATCCTCCG |
| | GGCCCAGCTGGAGTTCAACCAGATCAAG |
| | GCAGAGATCGAGCGGAAGCTGGCAGAGA |
| | AGGACGAGGAGATGGAACAGGCCAAGCG |
| | CAACCACCTGCGGGTGGTGGACTCGCTG |
| | CAGACCTCCCTGGACGCAGAGACACGCA |
| | GCCGCAACGAGGCCCTGAGGGTGAAGAA |
| | GAAGATGGAAGGAGACCTCAATGAGATG |
| | GAGATCCAGCTCAGCCACGCCAACCGCA |
| | TGGCCGCCGAGGCCCAGAAGCAAGTCAA |
| | GAGCCTCCAGAGCTTGTTGAAGGACACC |
| | CAGATTCAGCTGGACGATGCAGTCCGTG |
| | CCAACGACGACCTGAAGGAGAACATCGC |
| | CATCGTGGAGCGGCGCAACAACCTGCTG |
| | CAGGCTGAGCTGGAGGAGTTGCGTGCCG |
| | TGGTGGAGCAGACAGAGCGGTCCCGGAA |
| | GCTGGCGGAGCAGGAGCTGATTGAGACT |
| | AGTGAGCGGGTGCAGCTGCTGCATTCCC |
| | AGAACACCAGCCTCATCAACCAGAAGAA |
| | GAAGATGGATGCTGACCTGTCCCAGCTC |
| | CAGACTGAAGTGGAGGAGGCAGTGCAGG |
| | AGTGCAGGAATGCTGAGGAGAAGGCCAA |
| | GAAGGCCATCACGGATGCCGCCATGATG |
| | GCAGAGGAGCTGAAGAAGGAGCAGGACA |
| | CCAGCGCCCACCTGGAGCGCATGAAGAA |
| | GAACATGGAACAGACCATTAAGGACCTG |
| | CAGCACCGGCTGGACGAAGCCGAGCAGA |
| | TCGCCCTCAAGGGCGGCAAGAAGCAGCT |
| | GCAGAAGCTGGAAGCGCGGGTGCGGGA |
| | GCTGGAGAATGAGCTGGAGGCCGAGCAG |
| | AAGCGCAACGCAGAGTCGGTGAAGGGCA |
| | TGAGGAAGAGCGAGCGGCGCATCAAGGA |
| | GCTCACCTACCAGACGGAGGAGGACAGG |
| | AAAAACCTGCTGCGGCTGCAGGACCTGG |
| | TAGACAAGCTGCAGCTAAAGGTCAAGGC |
| | CTACAAGCGCCAGGCCGAGGAGGCGGA |
| | GGAGCAAGCCAACACCAACCTGTCCAAG |
| | TTCCGCAAGGTGCAGCACGAGCTGGATG |
| | AGGCAGAGGAGCGGGCGGACATCGCCG |
| | AGTCCCAGGTCAACAAGCTGCGGGCCAA |
| | GAGCCGTGACATTGGCACGAAGGGCTTG |
| | AATGAGGAGTAGCTTTGCCACATCTTGAT |
| | CTGCTCAGCCCTGGAGGTGCCAGCAAAG |
| | CCCCATGCTGGAGCCTGTGTAACAGCTC |
| | CTTGGGAGGAAGCAGAATAAAGCAATTTT |
| | CCTTGAAGCCGAGA |
| Murine Myh6 gene with G > A mutation - with | ATATAAAGGGGCTGGAGCACTGAGAGCT |
| humanized nucleotides (SEQ ID NO: 159) | GTCAGACAGAGATTTCTCCAACCCAGGAT |
| | CTCTGGATTGGTCTCCCAGCCTCTGCTAC |
| | TCCTCTTCCTGCCTGTTCCTCTCTCCGTC |
| | CAGCTGCGCCACTGTGGTGCCTCGTTCC |
| | AGCTGTGGTCCACATTCTTCAGGATTCTC |
| | TGAAAAGTTAACCAGAGTTTGAGTGACAG |
| | AATGACGGACGCCCAGATGGCTGACTTC |
| | GGGGCAGCAGCCCAGTACCTCCGAAAGT |
| | CAGAGAAGGAACGCCTAGAGGCCCAGAC |
| | CCGGCCCTTTGACATCCGCACGGAGTGC |
| | TTCGTGCCTGATGACAAGGAGGAGTATGT |
| | TAAGGCCAAGGTCGTGTCCCGGGAAGGG |
| | GGCAAAGTCACTGCGGAAACTGAAAACG |
| | GAAAGACGGTGACCATAAAGGAGGACCA |
| | GGTGATGCAGCAGAACCCACCCAAGTTC |
| | GACAAGATCGAGGACATGGCCATGCTGA |
| | CCTTCCTGCACGAGCCGGCTGTGCTGTA |
| | CAACCTCAAGGAGCGCTACGCGGCCTGG |
| | ATGATCTATACCTACTCAGGCCTCTTCTG |
| | CGTCACCGTCAACCCCTATAAGTGGCTG |
| | CCTGTGTACAATGCGGAAGTGGTGGCCG |
| | CCTACCGGGGCAAGAAGAGGAGCGAGG |
| | CCCCTCCTCACATCTTCTCCATCTCTGAC |
| | AACGCCTATCAGTACATGCTGACAGATCG |
| | GGAGAATCAGTCCATCCTCATCACCGGA |
| | GAATCCGGAGCGGGGAAGACTGTGAACA |
| | CAAAACGTGTCATCCAGTACTTTGCCAGC |
| | ATTGCAGCCATAGGGGACCGTAGCAAGA |
| | AGGAAAATCCTAATGCAAACAAGGGCACC |
| | CTGGAGGACCAGATTATCCAGGCTAACC |
| | CCGCTCTGGAGGCCTTCGGCAACGCCAA |
| | GACTGTCCGGAATGACAACTCCTCCCGC |
| | TTTGGGAAATTCATCAGGATCCACTTTGG |
| | AGCTACTGGAAAGCTGGCTTCTGCAGAC |
| | ATAGAGACCTACCTTCTGGAGAAGTCCCG |
| | GGTGATCTTCCAGCTAAAGGCTGAGAGG |
| | AACTACCACATCTTCTACCAGATCCTGTC |
| | CAACAAGAAGCCGGAGCTGCTGGACATG |
| | CTGCTGGTCACCAACAACCCATACGACTA |
| | CGCCTTCGTCTCTCAGGGAGAGGTGTCC |
| | GTGGCCTCCATTGATGACTCTGAGGAGC |
| | TCTTGGCCACTGATAGTGCCTTTGATGTG |
| | CTGAGCTTCACGGCAGAGGAGAAGGCTG |
| | GTGTCTACAAGCTGACAGGGGCCATCAT |
| | GCACTACGGAAACATGAAGTTCAAGCAGA |
| | AGCAGCGGGAGGAGCAGGCGGAGCCTG |
| | ATGGCACAGAAGATGCTGACAAATCAGC |
| | CTACCTCATGGGGCTGAACTCAGCCGAC |
| | CTGCTCAAGGGGCTGTGCCACCCTCAGG |
| | TGAAAGTGGGCAATGAGTATGTCACCAAG |
| | GGGCAGAGTGTACAGCAAGTGTACTATTC |
| | CATCGGGGCACTGGCCAAGTCAGTGTAC |
| | GAGAAGATGTTCAACTGGATGGTGACAC |
| | GCATCAACGCAACCCTGGAGACCAAGCA |
| | GCCGCGCCAGTACTTCATAGGTGTCCTG |
| | GACATTGCCGGCTTTGAGATCTTCGATTT |
| | CAACAGCTTTGAGCAGCTGTGCATCAACT |
| | TCACCAATGAGAAGCTGCAGCAGTTCTTC |
| | AACCACCACATGTTCGTGCTGGAGCAGG |
| | AGGAGTACAAGAAGGAGGGCATTGAGTG |
| | GGAGTTTATCGACTTCGGCATGGACCTG |
| | CAGGCCTGCATCGACCTCATCGAGAAGC |
| | CCATGGGCATCATGTCCATCCTCGAGGA |
| | GGAGTGCATGTTCCCCAAGGCCTCAGAC |
| | ATGACCTTCAAGGCCAAGCTGTATGACAA |
| | CCACCTGGGCAAATCCAACAACTTCCAGA |
| | AGCCTCGCAATGTCAAGGGGAAGCAGGA |
| | AGCCCACTTCTCCTTGGTCCACTATGCTG |
| | GCACCGTGGACTACAACATTATGGGCTG |
| | GCTGGAAAAGAACAAGGACCCACTCAAT |
| | GAGACGGTGGTGGGTTTGTACCAGAAGT |
| | CCTCCCTCAAGCTCATGGCTACACTCTTC |
| | TCTACCTATGCTTCTGCTGATACCGGTGA |
| | CAGTGGTAAAGGCAAAGGAGGCAAGAAG |
| | AAAGGCTCATCCTTCCAAACAGTGTCTGC |
| | TCTCCACCGGGAAAATCTGAACAAGCTGA |
| | TGACAAACCTGAAGACCACCCACCCTCAC |
| | TTTGTGCGCTGCATCATTCCCAACGAGCG |
| | AAAGGCTCCAGGGGTGATGGACAACCCC |
| | CTGGTCATGCACCAGCTGCGATGCAATG |
| | GCGTGCTGGAGGGTATCCGCATCTGCAG |
| | GAAGGGCTTCCCCAACCGCATTCTCTATG |
| | GGGACTTCCGGCAGAGGTATCGCATCCT |
| | GAACCCAGCAGCCATCCCTGAGGGGCAA |
| | TTCATTGATAGCAGGAAAGGGGCTGAGA |
| | AACTGCTGGGCTCCCTGGACATTGACCA |
| | CAACCAATACAAGTTTGGCCACACCAAGG |
| | TGTTCTTCAAGGCGGGCCTGCTGGGGCT |
| | GCTCGAGGAGATGCGAGATGAGAGGCTG |
| | AGCCGTATCATCACCAGAATCCAGGCCC |
| | AGGCCCGAGGGCAGCTCATGCGCATTGA |
| | GTTCAAGAAGATAGTGGAACGCAGGGAT |
| | GCCCTGCTGGTTATCCAGTGGAACATTCG |
| | GGCCTTCATGGGGGTCAAGAATTGGCCA |
| | TGGATGAAGCTCTACTTCAAGATCAAACC |
| | GCTGCTGAAGAGCGCAGAGACGGAGAAG |
| | GAGATGGCCAACATGAAGGAGGAGTTTG |
| | GGCGAGTCAAAGATGCACTGGAGAAGTC |
| | TGAGGCTCGCCGCAAGGAGCTGGAGGA |
| | GAAGATGGTGTCCCTGCTGCAGGAGAAG |
| | AATGACCTACAGCTCCAAGTGCAGGCGG |
| | AACAAGACAACCTCAATGATGCAGAGGA |
| | GCGCTGTGACCAGCTGATCAAGAACAAG |
| | ATCCAGCTGGAGGCCAAGGTGAAGGAGA |
| | TGACCGAGAGGCTGGAGGACGAGGAGG |
| | AGATGAACGCCGAGCTCACTGCCAAGAA |
| | GCGCAAGCTGGAAGATGAGTGCTCAGAG |
| | CTCAAGAAGGATATTGATGACCTGGAGCT |
| | GACGCTGGCCAAGGTGGAAAAGGAAAAG |
| | CATGCAACAGAGAACAAGGTTAAAAACCT |
| | AACAGAGGAGATGGCTGGGCTGGATGAA |
| | ATCATTGCCAAGCTGACCAAAGAGAAGAA |
| | AGCTCTGCAAGAAGCCCACCAGCAAGCC |
| | CTCGATGACCTGCAGGCTGAAGAAGACA |
| | AGGTCAACACGCTGACCAAGTCCAAAGT |
| | CAAGCTGGAGCAGCAGGTGGATGATCTG |
| | GAGGGATCCCTGGAGCAGGAGAAGAAAG |
| | TGCGCATGGACCTAGAGCGAGCCAAGCG |
| | GAAGCTGGAGGGAGACCTGAAGCTGACC |
| | CAGGAGAGCATCATGGACCTGGAGAATG |
| | ACAAGCTTCAGCTGGAAGAAAAGCTCAAG |
| | AAGAAAGAGTTCGACATCAGTCAGCAGAA |
| | CAGTAAAATTGAGGACGAGCAGGCCCTG |
| | GCTCTTCAGCTGCAGAAGAAACTGAAGG |
| | AAAACCAGGCACGCATCGAGGAGCTGGA |
| | GGAGGAGCTGGAGGCAGAGCGCACAGC |
| | CCGGGCTAAGGTGGAGAAGCTGCGCTCT |
| | GACCTGTCCCGGGAGCTGGAGGAGATCA |
| | GTGAGAGGCTGGAGGAGGCAGGCGGGG |
| | CCACATCCGTGCAGATAGAGATGAATAAG |
| | AAGCGCGAGGCCGAGTTCCAGAAGATGC |
| | GGCGGGACCTGGAGGAGGCCACGCTGC |
| | AGCACGAGGCCACGGCGGCGGCCCTGC |
| | GCAAGAAGCATGCTGACAGCGTGGCGGA |
| | GCTGGGCGAGCAGATCGACAACCTCCAG |
| | CGGGTGAAGCAGAAGCTGGAGAAAGAGA |
| | AGAGCGAGTTCAAGCTGGAGCTGGATGA |
| | CGTCACCTCCAACATGGAGCAGATCATCA |
| | AGGCCAAGGCCAACCTGGAGAAAGTGTC |
| | CCGGACACTGGAGGACCAGGCCAATGAG |
| | TACCGCGTGAAGCTGGAAGAAGCCCAGC |
| | GCTCCCTCAATGACTTCACCACACAGCGA |
| | GCCAAGCTGCAGACAGAGAACGGGGAGT |
| | TGGCTAGGCAACTGGAAGAAAAGGAGGC |
| | ATTGATTTCCCAGCTGACCCGAGGCAAG |
| | CTCTCCTACACCCAGCAGATGGAGGACC |
| | TCAAGAGGCAACTGGAGGAGGAAGGCAA |
| | GGCCAAGAACGCCCTGGCCCACGCACTG |
| | CAATCATCCCGGCATGACTGTGACCTGCT |
| | GAGGGAACAGTATGAAGAAGAAATGGAG |
| | GCCAAGGCTGAGCTACAGCGTGTCCTGT |
| | CCAAGGCCAACTCAGAGGTGGCCCAGTG |
| | GAGGACCAAGTATGAGACGGATGCCATA |
| | CAGAGGACGGAGGAGCTGGAGGAAGCC |
| | AAGAAGAAGCTGGCTCAGAGGCTGCAGG |
| | ATGCAGAGGAGGCAGTGGAGGCCGTCAA |
| | CGCCAAGTGTTCCTCCCTGGAGAAGACC |
| | AAGCACAGGCTGCAGAATGAGATCGAGG |
| | ACCTGATGGTGGACGTGGAGCGCTCCAA |
| | TGCCGCCGCCGCAGCCCTGGACAAGAAG |
| | CAGAGGAACTTTGACAAGATCCTGGCTGA |
| | GTGGAAGCAGAAGTATGAGGAGTCGCAG |
| | TCAGAGCTGGAGTCTTCCCAGAAGGAGG |
| | CGCGCTCCCTGAGCACAGAGCTCTTCAA |
| | GCTCAAGAACGCCTATGAGGAGTCTCTG |
| | GAGCACCTGGAGACCTTCAAGCGGGAGA |
| | ACAAGAACCTCCAGGAGGAGATCTCAGA |
| | CCTGACTGAACAGCTGGGAGAAGGGGGG |
| | AAAAACGTGCACGAGCTGGAGAAGATCC |
| | GCAAACAGCTGGAGGTGGAGAAGCTGGA |
| | GCTGCAGTCAGCCCTGGAGGAGGCTGAG |
| | GCCTCCCTGGAGCACGAGGAGGGCAAGA |
| | TCCTCCGTGCCCAGCTGGAGTTCAACCA |
| | GATCAAGGCAGAGATCGAAAGGAAGCTG |
| | GCAGAGAAGGATGAGGAGATGGAGCAGG |
| | CCAAGCGCAACCACCTGCGGATGGTGGA |
| | CTCCCTGCAGACCTCCCTGGATGCGGAG |
| | ACACGCAGCCGCAATGAGGCCCTGCGGG |
| | TGAAGAAGAAGATGGAGGGCGACCTCAA |
| | CGAGATGGAGATCCAGCTCAGCCAGGCC |
| | AATAGAATAGCCTCAGAGGCACAGAAACA |
| | CCTGAAGAATTCTCAAGCTCACTTGAAGG |
| | ACACCCAGCTCCAGCTGGATGATGCTGT |
| | CCATGCCAATGACGACCTGAAGGAGAAC |
| | ATCGCCATCGTGGAACGGCGCAACAACC |
| | TGCTGCAGGCGGAGCTGGAGGAGCTGC |
| | GGGCTGTGGTGGAGCAGACGGAGCGGT |
| | CTCGGAAGCTGGCAGAGCAGGAGCTGAT |
| | TGAGACCAGCGAGCGGGTGCAGCTGCTG |
| | CACTCGCAGAACACCAGCCTCATCAACCA |
| | GAAGAAGAAGATGGAGTCAGACCTGACC |
| | CAACTCCAGACAGAAGTAGAGGAGGCAG |
| | TGCAGGAGTGTAGGAACGCAGAGGAGAA |
| | GGCCAAGAAGGCCATCACAGATGCCGCA |
| | ATGATGGCTGAGGAGCTGAAGAAGGAGC |
| | AGGACACCAGCGCCCACCTGGAGCGCAT |
| | GAAGAAGAACATGGAGCAGACCATCAAG |
| | GACTTGCAGCACCGTCTGGACGAGGCAG |
| | AGCAGATCGCCCTCAAGGGCGGCAAGAA |
| | GCAGCTGCAGAAGCTGGAGGCCCGGGT |
| | CCGGGAGCTGGAGAATGAGCTGGAGGCT |
| | GAGCAGAAGCGCAATGCAGAGTCGGTGA |
| | AGGGCATGAGGAAGAGCGAGCGGCGCA |
| | TCAAGGAGCTCACCTACCAGACAGAGGA |
| | AGACAAGAAGAACTTAATGCGGCTGCAG |
| | GACCTGGTGGACAAGCTACAGTTGAAGG |
| | TGAAGGCCTACAAGCGCCAGGCTGAGGA |
| | GGCGGAGGAGCAGGCCAACACCAACCTG |
| | TCCAAGTTCCGCAAGGTGCAGCACGAGC |
| | TGGATGAGGCGGAGGAGAGGGCGGACA |
| | TCGCCGAGTCCCAGGTCAACAAGCTGCG |
| | GGCCAAGAGCCGGGACATTGGTGCCAAG |
| | AAGATGCACGACGAGGAATAACCTCTCCA |
| | GCAGACCCTCGCTGTGGCCAATCCACAA |
| | TAAACATAAACGTTCGACTCTGCC |
| WT Human Myh7 gene (SEQ ID NO: 162) | GGGGGTGGGGGTGCCCTGCTGCCCCAT |
| | ATATACAGCCCCTGAGACCAGGTCTGGC |
| | TCCACAGCTCTGTCCTGCTCTGTGTCTTT |
| | CCCTGCTGCTCTCAGGTCCCCTGCAGGC |
| | CTTGGCCCCTTTCCTCATCTGTAGACACA |
| | CTTGAGTAGCCCAGGCACAGCCATGGGA |
| | GATTCGGAGATGGCAGTCTTTGGGGCTG |
| | CCGCCCCCTACCTGCGCAAGTCAGAGAA |
| | GGAGCGGCTAGAAGCGCAGACCAGGCCT |
| | TTTGACCTCAAGAAGGATGTCTTCGTGCC |
| | TGATGACAAACAGGAGTTTGTCAAGGCCA |
| | AGATCGTGTCTCGAGAGGGTGGCAAAGT |
| | CACTGCCGAGACCGAGTATGGCAAGACA |
| | GTGACCGTGAAGGAGGACCAGGTGATGC |
| | AGCAGAACCCACCCAAGTTCGACAAAATC |
| | GAGGACATGGCCATGCTGACCTTCCTGC |
| | ATGAGCCCGCGGTGCTCTACAACCTCAA |
| | GGATCGCTACGGCTCCTGGATGATCTAC |
| | ACCTACTCGGGCCTCTTCTGTGTCACCGT |
| | CAACCCTTACAAGTGGCTGCCGGTGTAC |
| | ACTCCTGAGGTGGTGGCTGCCTACCGGG |
| | GCAAGAAGAGGAGCGAGGCCCCGCCCC |
| | ACATCTTCTCCATCTCCGACAACGCCTAT |
| | CAGTACATGCTGACAGACAGAGAAAACCA |
| | GTCCATCCTGATCACCGGAGAATCCGGA |
| | GCAGGGAAGACAGTCAACACCAAGAGGG |
| | TCATCCAGTACTTTGCTGTTATTGCAGCC |
| | ATTGGGGACCGCAGCAAGAAGGACCAGA |
| | GCCCGGGCAAGGGCACCCTGGAGGACC |
| | AGATCATCCAGGCCAACCCTGCTCTGGA |
| | GGCCTTTGGCAATGCCAAGACCGTCCGG |
| | AACGACAACTCCTCCCGCTTCGGGAAATT |
| | CATTCGAATTCATTTTGGGGCAACAGGAA |
| | AGTTGGCATCTGCAGACATAGAGACCTAT |
| | CTTCTGGAAAAATCCAGAGTTATTTTCCA |
| | GCTGAAAGCAGAGAGAGATTATCACATTT |
| | TCTACCAAATCCTGTCTAACAAAAAGCCT |
| | GAGCTGCTGGACATGCTGCTGATCACCA |
| | ACAACCCCTACGATTATGCATTCATCTCC |
| | CAAGGAGAGACCACCGTGGCCTCCATTG |
| | ATGACGCTGAGGAGCTCATGGCCACTGA |
| | TAACGCTTTTGATGTGCTGGGCTTCACTT |
| | CAGAGGAGAAAAACTCCATGTATAAGCTG |
| | ACAGGCGCCATCATGCACTTTGGAAACAT |
| | GAAGTTCAAGCTGAAGCAGCGGGAGGAG |
| | CAGGCGGAGCCAGACGGCACTGAAGAG |
| | GCTGACAAGTCTGCCTACCTCATGGGGC |
| | TGAACTCAGCCGACCTGCTCAAGGGGCT |
| | GTGCCACCCTCGGGTGAAAGTGGGCAAT |
| | GAGTACGTCACCAAGGGGCAGAATGTCC |
| | AGCAGGTGATATATGCCACTGGGGCACT |
| | GGCCAAGGCAGTGTATGAGAGGATGTTC |
| | AACTGGATGGTGACGCGCATCAATGCCA |
| | CCCTGGAGACCAAGCAGCCACGCCAGTA |
| | CTTCATAGGAGTCCTGGACATCGCTGGCT |
| | TCGAGATCTTCGATTTCAACAGCTTTGAG |
| | CAGCTCTGCATCAACTTCACCAACGAGAA |
| | GCTGCAGCAGTTCTTCAACCACCACATGT |
| | TTGTGCTGGAGCAGGAGGAGTACAAGAA |
| | GGAGGGCATCGAGTGGACATTCATTGAC |
| | TTTGGCATGGACCTGCAGGCCTGCATTG |
| | ACCTCATCGAGAAGCCCATGGGCATCAT |
| | GTCCATCCTGGAAGAGGAGTGCATGTTC |
| | CCCAAGGCCACCGACATGACCTTCAAGG |
| | CCAAGCTGTTTGACAACCACCTGGGCAAA |
| | TCCGCCAACTTCCAGAAGCCACGCAATAT |
| | CAAGGGGAAGCCTGAAGCCCACTTCTCC |
| | CTGATCCACTATGCCGGCATCGTGGACTA |
| | CAACATCATTGGCTGGCTGCAGAAGAACA |
| | AGGATCCTCTCAATGAGACTGTCGTGGG |
| | CTTGTATCAGAAGTCTTCCCTCAAGCTGC |
| | TCAGCACCCTGTTTGCCAACTATGCTGGG |
| | GCTGATGCGCCTATTGAGAAGGGCAAAG |
| | GCAAGGCCAAGAAAGGCTCGTCCTTTCA |
| | GACTGTGTCAGCTCTGCACAGGGAAAAT |
| | CTGAACAAGCTGATGACCAACTTGCGCTC |
| | CACCCATCCCCACTTTGTACGTTGTATCA |
| | TCCCTAATGAGACAAAGTCTCCAGGGGT |
| | GATGGACAACCCCCTGGTCATGCACCAG |
| | CTGCGCTGCAATGGTGTGCTGGAGGGCA |
| | TCCGCATCTGCAGGAAAGGCTTCCCCAA |
| | CCGCATCCTCTACGGGGACTTCCGGCAG |
| | AGGTATCGCATCCTGAACCCAGCGGCCA |
| | TCCCTGAGGGACAGTTCATTGATAGCAG |
| | GAAGGGGGCAGAGAAGCTGCTCAGCTCC |
| | CTGGACATTGATCACAACCAGTACAAGTT |
| | TGGCCACACCAAGGTGTTCTTCAAGGCC |
| | GGGCTGCTGGGGCTGCTGGAGGAAATGA |
| | GGGACGAGAGGCTGAGCCGCATCATCAC |
| | GCGTATCCAGGCCCAGTCCCGAGGTGTG |
| | CTCGCCAGAATGGAGTACAAAAAGCTGCT |
| | GGAACGTAGAGACTCCCTGCTGGTAATC |
| | CAGTGGAACATTCGGGCCTTCATGGGGG |
| | TCAAGAATTGGCCCTGGATGAAGCTCTAC |
| | TTCAAGATCAAGCCGCTGCTGAAGAGTG |
| | CAGAAAGAGAGAAGGAGATGGCCTCCAT |
| | GAAGGAGGAGTTCACACGCCTCAAAGAG |
| | GCGCTAGAGAAGTCCGAGGCTCGCCGCA |
| | AGGAGCTGGAGGAGAAGATGGTGTCCCT |
| | GCTGCAGGAGAAGAATGACCTGCAGCTC |
| | CAAGTGCAGGCGGAACAAGACAACCTGG |
| | CAGATGCTGAGGAGCGCTGTGATCAGCT |
| | GATCAAAAACAAGATTCAGCTGGAGGCCA |
| | AGGTGAAGGAGATGAACGAGAGGCTGGA |
| | GGATGAGGAGGAGATGAATGCTGAGCTC |
| | ACTGCCAAGAAGCGCAAGCTGGAAGATG |
| | AGTGCTCAGAGCTCAAAAGGGACATCGA |
| | TGATCTGGAGCTGACACTGGCCAAAGTG |
| | GAGAAGGAGAAACACGCAACAGAGAACA |
| | AGGTGAAAAACCTGACAGAGGAGATGGC |
| | TGGGCTGGATGAGATCATTGCCAAGCTG |
| | ACCAAGGAGAAGAAAGCTCTGCAAGAGG |
| | CCCACCAACAGGCTCTGGATGACCTTCA |
| | GGCCGAGGAGGACAAGGTCAACACCCTG |
| | ACTAAGGCCAAAGTCAAGCTGGAGCAGC |
| | AAGTGGATGATCTGGAAGGATCCCTGGA |
| | GCAAGAGAAGAAGGTGCGCATGGACCTG |
| | GAGCGAGCGAAGCGGAAGCTGGAGGGC |
| | GACCTGAAGCTGACCCAGGAGAGCATCA |
| | TGGACCTGGAGAATGACAAGCAGCAGCT |
| | GGATGAGCGGCTGAAAAAAAAAGACTTTG |
| | AGCTGAATGCTCTCAACGCAAGGATTGAG |
| | GATGAACAGGCCCTCGGCAGCCAGCTGC |
| | AGAAGAAGCTCAAGGAGCTTCAGGCACG |
| | CATCGAGGAGCTGGAGGAGGAGCTGGA |
| | GGCCGAGCGCACCGCCAGGGCTAAGGT |
| | GGAGAAGCTGCGCTCAGACCTGTCTCGG |
| | GAGCTGGAGGAGATCAGCGAGCGGCTG |
| | GAAGAGGCCGGCGGGGCCACGTCCGTG |
| | CAGATCGAGATGAACAAGAAGCGCGAGG |
| | CCGAGTTCCAGAAGATGCGGCGGGACCT |
| | GGAGGAGGCCACGCTGCAGCACGAGGC |
| | CACTGCCGCGGCCCTGCGCAAGAAGCAC |
| | GCCGACAGCGTGGCCGAGCTGGGCGAG |
| | CAGATCGACAACCTGCAGCGGGTGAAGC |
| | AGAAGCTGGAGAAGGAGAAGAGCGAGTT |
| | CAAGCTGGAGCTGGATGACGTCACCTCC |
| | AACATGGAGCAGATCATCAAGGCCAAGG |
| | CTAACCTGGAGAAGATGTGCCGGACCTT |
| | GGAAGACCAGATGAATGAGCACCGGAGC |
| | AAGGCGGAGGAGACCCAGCGTTCTGTCA |
| | ACGACCTCACCAGCCAGCGGGCCAAGTT |
| | GCAAACCGAGAATGGTGAGCTGTCCCGG |
| | CAGCTGGATGAGAAGGAGGCACTGATCT |
| | CCCAGCTGACCCGAGGCAAGCTCACCTA |
| | CACCCAGCAGCTGGAGGACCTCAAGAGG |
| | CAGCTGGAGGAGGAGGTTAAGGCGAAGA |
| | ACGCCCTGGCCCACGCACTGCAGTCGGC |
| | CCGGCATGACTGCGACCTGCTGCGGGAG |
| | CAGTACGAGGAGGAGACGGAGGCCAAG |
| | GCCGAGCTGCAGCGCGTCCTTTCCAAGG |
| | CCAACTCGGAGGTGGCCCAGTGGAGGAC |
| | CAAGTATGAGACGGACGCCATTCAGCGG |
| | ACTGAGGAGCTCGAGGAGGCCAAGAAGA |
| | AGCTGGCCCAGCGGCTGCAGGAAGCTGA |
| | GGAGGCCGTGGAGGCTGTTAATGCCAAG |
| | TGCTCCTCGCTGGAGAAGACCAAGCACC |
| | GGCTACAGAATGAGATCGAGGACTTGAT |
| | GGTGGACGTAGAGCGCTCCAATGCTGCT |
| | GCTGCAGCCCTGGACAAGAAGCAGAGGA |
| | ACTTCGACAAGATCCTGGCCGAGTGGAA |
| | GCAGAAGTATGAGGAGTCGCAGTCGGAG |
| | CTGGAGTCCTCGCAGAAGGAGGCTCGCT |
| | CCCTCAGCACAGAGCTCTTCAAACTCAAG |
| | AACGCCTATGAGGAGTCCCTGGAACATCT |
| | GGAGACCTTCAAGCGGGAGAACAAAAAC |
| | CTGCAGGAGGAGATCTCCGACTTGACTG |
| | AGCAGTTGGGTTCCAGCGGAAAGACTAT |
| | CCATGAGCTGGAGAAGGTCCGAAAGCAG |
| | CTGGAGGCCGAGAAGATGGAGCTGCAGT |
| | CAGCCCTGGAGGAGGCCGAGGCCTCCCT |
| | GGAGCACGAGGAGGGCAAGATCCTCCG |
| | GGCCCAGCTGGAGTTCAACCAGATCAAG |
| | GCAGAGATCGAGCGGAAGCTGGCAGAGA |
| | AGGACGAGGAGATGGAACAGGCCAAGCG |
| | CAACCACCTGCGGGTGGTGGACTCGCTG |
| | CAGACCTCCCTGGACGCAGAGACACGCA |
| | GCCGCAACGAGGCCCTGAGGGTGAAGAA |
| | GAAGATGGAAGGAGACCTCAATGAGATG |
| | GAGATCCAGCTCAGCCACGCCAACCGCA |
| | TGGCCGCCGAGGCCCAGAAGCAAGTCAA |
| | GAGCCTCCAGAGCTTGTTGAAGGACACC |
| | CAGATTCAGCTGGACGATGCAGTCCGTG |
| | CCAACGACGACCTGAAGGAGAACATCGC |
| | CATCGTGGAGCGGCGCAACAACCTGCTG |
| | CAGGCTGAGCTGGAGGAGTTGCGTGCCG |
| | TGGTGGAGCAGACAGAGCGGTCCCGGAA |
| | GCTGGCGGAGCAGGAGCTGATTGAGACT |
| | AGTGAGCGGGTGCAGCTGCTGCATTCCC |
| | AGAACACCAGCCTCATCAACCAGAAGAA |
| | GAAGATGGATGCTGACCTGTCCCAGCTC |
| | CAGACTGAAGTGGAGGAGGCAGTGCAGG |
| | AGTGCAGGAATGCTGAGGAGAAGGCCAA |
| | GAAGGCCATCACGGATGCCGCCATGATG |
| | GCAGAGGAGCTGAAGAAGGAGCAGGACA |
| | CCAGCGCCCACCTGGAGCGCATGAAGAA |
| | GAACATGGAACAGACCATTAAGGACCTG |
| | CAGCACCGGCTGGACGAAGCCGAGCAGA |
| | TCGCCCTCAAGGGCGGCAAGAAGCAGCT |
| | GCAGAAGCTGGAAGCGCGGGTGCGGGA |
| | GCTGGAGAATGAGCTGGAGGCCGAGCAG |
| | AAGCGCAACGCAGAGTCGGTGAAGGGCA |
| | TGAGGAAGAGCGAGCGGCGCATCAAGGA |
| | GCTCACCTACCAGACGGAGGAGGACAGG |
| | AAAAACCTGCTGCGGCTGCAGGACCTGG |
| | TAGACAAGCTGCAGCTAAAGGTCAAGGC |
| | CTACAAGCGCCAGGCCGAGGAGGCGGA |
| | GGAGCAAGCCAACACCAACCTGTCCAAG |
| | TTCCGCAAGGTGCAGCACGAGCTGGATG |
| | AGGCAGAGGAGCGGGCGGACATCGCCG |
| | AGTCCCAGGTCAACAAGCTGCGGGCCAA |
| | GAGCCGTGACATTGGCACGAAGGGCTTG |
| | AATGAGGAGTAGCTTTGCCACATCTTGAT |
| | CTGCTCAGCCCTGGAGGTGCCAGCAAAG |
| | CCCCATGCTGGAGCCTGTGTAACAGCTC |
| | CTTGGGAGGAAGCAGAATAAAGCAATTTT |
| | CCTTGAAGCCGAGA |
| WT Mouse Myh6 gene (SEQ ID NO: 163) | ATATAAAGGGGCTGGAGCACTGAGAGCT |
| | GTCAGACAGAGATTTCTCCAACCCAGGAT |
| | CTCTGGATTGGTCTCCCAGCCTCTGCTAC |
| | TCCTCTTCCTGCCTGTTCCTCTCTCCGTC |
| | CAGCTGCGCCACTGTGGTGCCTCGTTCC |
| | AGCTGTGGTCCACATTCTTCAGGATTCTC |
| | TGAAAAGTTAACCAGAGTTTGAGTGACAG |
| | AATGACGGACGCCCAGATGGCTGACTTC |
| | GGGGCAGCAGCCCAGTACCTCCGAAAGT |
| | CAGAGAAGGAACGCCTAGAGGCCCAGAC |
| | CCGGCCCTTTGACATCCGCACGGAGTGC |
| | TTCGTGCCTGATGACAAGGAGGAGTATGT |
| | TAAGGCCAAGGTCGTGTCCCGGGAAGGG |
| | GGCAAAGTCACTGCGGAAACTGAAAACG |
| | GAAAGACGGTGACCATAAAGGAGGACCA |
| | GGTGATGCAGCAGAACCCACCCAAGTTC |
| | GACAAGATCGAGGACATGGCCATGCTGA |
| | CCTTCCTGCACGAGCCGGCTGTGCTGTA |
| | CAACCTCAAGGAGCGCTACGCGGCCTGG |
| | ATGATCTATACCTACTCAGGCCTCTTCTG |
| | CGTCACCGTCAACCCCTATAAGTGGCTG |
| | CCTGTGTACAATGCGGAAGTGGTGGCCG |
| | CCTACCGGGGCAAGAAGAGGAGCGAGG |
| | CCCCTCCTCACATCTTCTCCATCTCTGAC |
| | AACGCCTATCAGTACATGCTGACAGATCG |
| | GGAGAATCAGTCCATCCTCATCACCGGA |
| | GAATCCGGAGCGGGGAAGACTGTGAACA |
| | CAAAACGTGTCATCCAGTACTTTGCCAGC |
| | ATTGCAGCCATAGGGGACCGTAGCAAGA |
| | AGGAAAATCCTAATGCAAACAAGGGCACC |
| | CTGGAGGACCAGATTATCCAGGCTAACC |
| | CCGCTCTGGAGGCCTTCGGCAACGCCAA |
| | GACTGTCCGGAATGACAACTCCTCCCGC |
| | TTTGGGAAATTCATCAGGATCCACTTTGG |
| | AGCTACTGGAAAGCTGGCTTCTGCAGAC |
| | ATAGAGACCTACCTTCTGGAGAAGTCCCG |
| | GGTGATCTTCCAGCTAAAGGCTGAGAGG |
| | AACTACCACATCTTCTACCAGATCCTGTC |
| | CAACAAGAAGCCGGAGCTGCTGGACATG |
| | CTGCTGGTCACCAACAACCCATACGACTA |
| | CGCCTTCGTCTCTCAGGGAGAGGTGTCC |
| | GTGGCCTCCATTGATGACTCTGAGGAGC |
| | TCTTGGCCACTGATAGTGCCTTTGATGTG |
| | CTGAGCTTCACGGCAGAGGAGAAGGCTG |
| | GTGTCTACAAGCTGACAGGGGCCATCAT |
| | GCACTACGGAAACATGAAGTTCAAGCAGA |
| | AGCAGCGGGAGGAGCAGGCGGAGCCTG |
| | ATGGCACAGAAGATGCTGACAAATCAGC |
| | CTACCTTATGGGGCTGAACTCAGCTGACC |
| | TGCTCAAGGGCCTGTGTCACCCTCGGGT |
| | GAAGGTGGGGAACGAGTATGTCACCAAG |
| | GGGCAGAGTGTACAGCAAGTGTACTATTC |
| | CATCGGGGCACTGGCCAAGTCAGTGTAC |
| | GAGAAGATGTTCAACTGGATGGTGACAC |
| | GCATCAACGCAACCCTGGAGACCAAGCA |
| | GCCGCGCCAGTACTTCATAGGTGTCCTG |
| | GACATTGCCGGCTTTGAGATCTTCGATTT |
| | CAACAGCTTTGAGCAGCTGTGCATCAACT |
| | TCACCAATGAGAAGCTGCAGCAGTTCTTC |
| | AACCACCACATGTTCGTGCTGGAGCAGG |
| | AGGAGTACAAGAAGGAGGGCATTGAGTG |
| | GGAGTTTATCGACTTCGGCATGGACCTG |
| | CAGGCCTGCATCGACCTCATCGAGAAGC |
| | CCATGGGCATCATGTCCATCCTCGAGGA |
| | GGAGTGCATGTTCCCCAAGGCCTCAGAC |
| | ATGACCTTCAAGGCCAAGCTGTATGACAA |
| | CCACCTGGGCAAATCCAACAACTTCCAGA |
| | AGCCTCGCAATGTCAAGGGGAAGCAGGA |
| | AGCCCACTTCTCCTTGGTCCACTATGCTG |
| | GCACCGTGGACTACAACATTATGGGCTG |
| | GCTGGAAAAGAACAAGGACCCACTCAAT |
| | GAGACGGTGGTGGGTTTGTACCAGAAGT |
| | CCTCCCTCAAGCTCATGGCTACACTCTTC |
| | TCTACCTATGCTTCTGCTGATACCGGTGA |
| | CAGTGGTAAAGGCAAAGGAGGCAAGAAG |
| | AAAGGCTCATCCTTCCAAACAGTGTCTGC |
| | TCTCCACCGGGAAAATCTGAACAAGCTGA |
| | TGACAAACCTGAAGACCACCCACCCTCAC |
| | TTTGTGCGCTGCATCATTCCCAACGAGCG |
| | AAAGGCTCCAGGGGTGATGGACAACCCC |
| | CTGGTCATGCACCAGCTGCGATGCAATG |
| | GCGTGCTGGAGGGTATCCGCATCTGCAG |
| | GAAGGGCTTCCCCAACCGCATTCTCTATG |
| | GGGACTTCCGGCAGAGGTATCGCATCCT |
| | GAACCCAGCAGCCATCCCTGAGGGGCAA |
| | TTCATTGATAGCAGGAAAGGGGCTGAGA |
| | AACTGCTGGGCTCCCTGGACATTGACCA |
| | CAACCAATACAAGTTTGGCCACACCAAGG |
| | TGTTCTTCAAGGCGGGCCTGCTGGGGCT |
| | GCTCGAGGAGATGCGAGATGAGAGGCTG |
| | AGCCGTATCATCACCAGAATCCAGGCCC |
| | AGGCCCGAGGGCAGCTCATGCGCATTGA |
| | GTTCAAGAAGATAGTGGAACGCAGGGAT |
| | GCCCTGCTGGTTATCCAGTGGAACATTCG |
| | GGCCTTCATGGGGGTCAAGAATTGGCCA |
| | TGGATGAAGCTCTACTTCAAGATCAAACC |
| | GCTGCTGAAGAGCGCAGAGACGGAGAAG |
| | GAGATGGCCAACATGAAGGAGGAGTTTG |
| | GGCGAGTCAAAGATGCACTGGAGAAGTC |
| | TGAGGCTCGCCGCAAGGAGCTGGAGGA |
| | GAAGATGGTGTCCCTGCTGCAGGAGAAG |
| | AATGACCTACAGCTCCAAGTGCAGGCGG |
| | AACAAGACAACCTCAATGATGCAGAGGA |
| | GCGCTGTGACCAGCTGATCAAGAACAAG |
| | ATCCAGCTGGAGGCCAAGGTGAAGGAGA |
| | TGACCGAGAGGCTGGAGGACGAGGAGG |
| | AGATGAACGCCGAGCTCACTGCCAAGAA |
| | GCGCAAGCTGGAAGATGAGTGCTCAGAG |
| | CTCAAGAAGGATATTGATGACCTGGAGCT |
| | GACGCTGGCCAAGGTGGAAAAGGAAAAG |
| | CATGCAACAGAGAACAAGGTTAAAAACCT |
| | AACAGAGGAGATGGCTGGGCTGGATGAA |
| | ATCATTGCCAAGCTGACCAAAGAGAAGAA |
| | AGCTCTGCAAGAAGCCCACCAGCAAGCC |
| | CTCGATGACCTGCAGGCTGAAGAAGACA |
| | AGGTCAACACGCTGACCAAGTCCAAAGT |
| | CAAGCTGGAGCAGCAGGTGGATGATCTG |
| | GAGGGATCCCTGGAGCAGGAGAAGAAAG |
| | TGCGCATGGACCTAGAGCGAGCCAAGCG |
| | GAAGCTGGAGGGAGACCTGAAGCTGACC |
| | CAGGAGAGCATCATGGACCTGGAGAATG |
| | ACAAGCTTCAGCTGGAAGAAAAGCTCAAG |
| | AAGAAAGAGTTCGACATCAGTCAGCAGAA |
| | CAGTAAAATTGAGGACGAGCAGGCCCTG |
| | GCTCTTCAGCTGCAGAAGAAACTGAAGG |
| | AAAACCAGGCACGCATCGAGGAGCTGGA |
| | GGAGGAGCTGGAGGCAGAGCGCACAGC |
| | CCGGGCTAAGGTGGAGAAGCTGCGCTCT |
| | GACCTGTCCCGGGAGCTGGAGGAGATCA |
| | GTGAGAGGCTGGAGGAGGCAGGGGGG |
| | CCACATCCGTGCAGATAGAGATGAATAAG |
| | AAGCGCGAGGCCGAGTTCCAGAAGATGC |
| | GGCGGGACCTGGAGGAGGCCACGCTGC |
| | AGCACGAGGCCACGGCGGCGGCCCTGC |
| | GCAAGAAGCATGCTGACAGCGTGGCGGA |
| | GCTGGGCGAGCAGATCGACAACCTCCAG |
| | CGGGTGAAGCAGAAGCTGGAGAAAGAGA |
| | AGAGCGAGTTCAAGCTGGAGCTGGATGA |
| | CGTCACCTCCAACATGGAGCAGATCATCA |
| | AGGCCAAGGCCAACCTGGAGAAAGTGTC |
| | CCGGACACTGGAGGACCAGGCCAATGAG |
| | TACCGCGTGAAGCTGGAAGAAGCCCAGC |
| | GCTCCCTCAATGACTTCACCACACAGCGA |
| | GCCAAGCTGCAGACAGAGAACGGGGAGT |
| | TGGCTAGGCAACTGGAAGAAAAGGAGGC |
| | ATTGATTTCCCAGCTGACCCGAGGCAAG |
| | CTCTCCTACACCCAGCAGATGGAGGACC |
| | TCAAGAGGCAACTGGAGGAGGAAGGCAA |
| | GGCCAAGAACGCCCTGGCCCACGCACTG |
| | CAATCATCCCGGCATGACTGTGACCTGCT |
| | GAGGGAACAGTATGAAGAAGAAATGGAG |
| | GCCAAGGCTGAGCTACAGCGTGTCCTGT |
| | CCAAGGCCAACTCAGAGGTGGCCCAGTG |
| | GAGGACCAAGTATGAGACGGATGCCATA |
| | CAGAGGACGGAGGAGCTGGAGGAAGCC |
| | AAGAAGAAGCTGGCTCAGAGGCTGCAGG |
| | ATGCAGAGGAGGCAGTGGAGGCCGTCAA |
| | CGCCAAGTGTTCCTCCCTGGAGAAGACC |
| | AAGCACAGGCTGCAGAATGAGATCGAGG |
| | ACCTGATGGTGGACGTGGAGCGCTCCAA |
| | TGCCGCCGCCGCAGCCCTGGACAAGAAG |
| | CAGAGGAACTTTGACAAGATCCTGGCTGA |
| | GTGGAAGCAGAAGTATGAGGAGTCGCAG |
| | TCAGAGCTGGAGTCTTCCCAGAAGGAGG |
| | CGCGCTCCCTGAGCACAGAGCTCTTCAA |
| | GCTCAAGAACGCCTATGAGGAGTCTCTG |
| | GAGCACCTGGAGACCTTCAAGCGGGAGA |
| | ACAAGAACCTCCAGGAGGAGATCTCAGA |
| | CCTGACTGAACAGCTGGGAGAAGGGGGG |
| | AAAAACGTGCACGAGCTGGAGAAGATCC |
| | GCAAACAGCTGGAGGTGGAGAAGCTGGA |
| | GCTGCAGTCAGCCCTGGAGGAGGCTGAG |
| | GCCTCCCTGGAGCACGAGGAGGGCAAGA |
| | TCCTCCGTGCCCAGCTGGAGTTCAACCA |
| | GATCAAGGCAGAGATCGAAAGGAAGCTG |
| | GCAGAGAAGGATGAGGAGATGGAGCAGG |
| | CCAAGCGCAACCACCTGCGGATGGTGGA |
| | CTCCCTGCAGACCTCCCTGGATGCGGAG |
| | ACACGCAGCCGCAATGAGGCCCTGCGGG |
| | TGAAGAAGAAGATGGAGGGCGACCTCAA |
| | CGAGATGGAGATCCAGCTCAGCCAGGCC |
| | AATAGAATAGCCTCAGAGGCACAGAAACA |
| | CCTGAAGAATTCTCAAGCTCACTTGAAGG |
| | ACACCCAGCTCCAGCTGGATGATGCTGT |
| | CCATGCCAATGACGACCTGAAGGAGAAC |
| | ATCGCCATCGTGGAACGGCGCAACAACC |
| | TGCTGCAGGCGGAGCTGGAGGAGCTGC |
| | GGGCTGTGGTGGAGCAGACGGAGCGGT |
| | CTCGGAAGCTGGCAGAGCAGGAGCTGAT |
| | TGAGACCAGCGAGCGGGTGCAGCTGCTG |
| | CACTCGCAGAACACCAGCCTCATCAACCA |
| | GAAGAAGAAGATGGAGTCAGACCTGACC |
| | CAACTCCAGACAGAAGTAGAGGAGGCAG |
| | TGCAGGAGTGTAGGAACGCAGAGGAGAA |
| | GGCCAAGAAGGCCATCACAGATGCCGCA |
| | ATGATGGCTGAGGAGCTGAAGAAGGAGC |
| | AGGACACCAGCGCCCACCTGGAGCGCAT |
| | GAAGAAGAACATGGAGCAGACCATCAAG |
| | GACTTGCAGCACCGTCTGGACGAGGCAG |
| | AGCAGATCGCCCTCAAGGGCGGCAAGAA |
| | GCAGCTGCAGAAGCTGGAGGCCCGGGT |
| | CCGGGAGCTGGAGAATGAGCTGGAGGCT |
| | GAGCAGAAGCGCAATGCAGAGTCGGTGA |
| | AGGGCATGAGGAAGAGCGAGCGGCGCA |
| | TCAAGGAGCTCACCTACCAGACAGAGGA |
| | AGACAAGAAGAACTTAATGCGGCTGCAG |
| | GACCTGGTGGACAAGCTACAGTTGAAGG |
| | TGAAGGCCTACAAGCGCCAGGCTGAGGA |
| | GGCGGAGGAGCAGGCCAACACCAACCTG |
| | TCCAAGTTCCGCAAGGTGCAGCACGAGC |
| | TGGATGAGGCGGAGGAGAGGGCGGACA |
| | TCGCCGAGTCCCAGGTCAACAAGCTGCG |
| | GGCCAAGAGCCGGGACATTGGTGCCAAG |
| | AAGATGCACGACGAGGAATAACCTCTCCA |
| | GCAGACCCTCGCTGTGGCCAATCCACAA |
| | TAAACATAAACGTTCGACTCTGCC |

TABLE 14C
Humanized Myh6 Sequences

| Sequence Name | |
|---|---|
| (SEQ ID NO) | Sequence |
| Myh6 403mut- | TGCCTACCTN1ATGGGGCTGAACTCAGCN |
| with optional | 2GACCTGCTCAAGGGN3CTGTGN4CACCC |
| humanized | TCAGGTGAAAGTGGGN5AAN6GAGTAC |
| alleles | N1 = C or T; N2 = C or T; |
| (SEQ ID NO: 158) | N3 = G or C; N4 is C or T; |
| | N5 is C or G; N6 is T or C |
| Myh6 403/+ | TGCCTACCTCATGGGGCTGAACTCAGCC |
| (wt and mut) | GACCTGCTCAAGGGGCTGTGCCACCCTC |
| with all | NGGTGAAAGTGGGCAATGAGTAC |
| humanized | N = A or G |
| alleles | |
| (SEQ ID NO: 160) | |
| Myh6 403/+ | TGCCTACCTN1ATGGGGCTGAACTCAGCN |
| (wt and mut) | 2GACCTGCTCAAGGGN3CTGTGN4CACCC |
| with optional | TCAGGTGAAN5GTGGGN6AAN7GAGTAC |
| humanized | N1 = C or T; N2 = C or T; |
| alleles | N3 = G or C; N4 is C or T; |
| (SEQ ID NO: 164) | N5 = A or G; N6 is C or G; |
| | N7 is T or C |

The gene edited mouse may be created according to methods known in the art. In some aspects, the gene edited mouse is created by microinjection of zygotes with Cas9 mRNA (50 ng/μL) (SEQ ID NO: 94, IDT), a sgRNA (20 ng/μL) (SEQ ID NO: 93, IDT), and a ssODN donor template (15 ng/μL) (SEQ ID NO: 92, IDT) following a protocols described in the art (e.g., H. Miura, R. M. Quadros, C. B. Gurumurthy, M. Ohtsuka, Easi-CRISPR for creating knock-in and conditional knockout mouse models using long ssDNA donors. Nat Protoc 13, 195-215 (2018, which is incorporated herein by reference in its entirety). Table 15, below provides, illustrative nucleic acids of the Cas9 mRNA, sgRNA and ssODN donor template that may be used in accordance with these methods to generate the gene edited mouse herein.

TABLE 15
Gene Editing Components for Gene-Edited Mouse Model

| Sequence Description | Sequence | SEQ ID NO: |
|---|---|---|
| ssODN donor sequence | TGGGACAAAGGAATGGAGGTACTGAAAA | 92 |
| | TGCTTCCCCTCTCCTTGTCTATCAGATGC | |
| | TGACAAATCAGCCTACCTCATGGGGCTG | |
| | AACTCAGCCGACCTGCTCAAGGGGCTGT | |
| | GCCACCCTCAGGTGAAAGTGGGCAATGA | |
| | GTACGTCACCAAGGGGCAGAGTGTACAG | |
| | CAAGTGTACTAT | |
| sgRNA | UCGUUCCCCACCUUCACCCGGUUUUAG | 93 |
| | AGCUAGAAAUAGCAAGUUAAAAUAAGGC | |
| | UAGUCCGUUAUCAACUUGAAAAAGUGG | |
| | CACCGAGUCGGUGCUUUU | |
| Cas9 mRNA | AUGGCCCCCAAGAAGAAGCGGAAGGUG | 94 |
| | GGCAUCCACGGCGUGCCCGCCGCCGAC | |
| | AAGAAGUACAGCAUCGGCCUGGACAUC | |
| | GGCACCAACAGCGUGGGCUGGGCCGUG | |
| | AUCACCGACGAGUACAAGGUGCCCAGC | |
| | AAGAAGUUCAAGGUGCUGGGCAACACC | |
| | GACCGGCACAGCAUCAAGAAGAACCUGA | |
| | UCGGCGCCCUGCUGUUCGACAGCGGCG | |
| | AGACCGCCGAGGCCACCCGGCUGAAGC | |
| | GGACCGCCCGGCGGCGGUACACCCGGC | |
| | GGAAGAACCGGAUCUGCUACCUGCAGG | |
| | AGAUCUUCAGCAACGAGAUGGCCAAGG | |
| | UGGACGACAGCUUCUUCCACCGGCUGG | |
| | AGGAGAGCUUCCUGGUGGAGGAGGACA | |
| | AGAAGCACGAGCGGCACCCCAUCUUCG | |
| | GCAACAUCGUGGACGAGGUGGCCUACC | |
| | ACGAGAAGUACCCCACCAUCUACCACCU | |
| | GCGGAAGAAGCUGGUGGACAGCACCGA | |
| | CAAGGCCGACCUGCGGCUGAUCUACCU | |
| | GGCCCUGGCCCACAUGAUCAAGUUCCG | |
| | GGGCCACUUCCUGAUCGAGGGCGACCU | |
| | GAACCCCGACAACAGCGACGUGGACAA | |
| | GCUGUUCAUCCAGCUGGUGCAGACCUA | |
| | CAACCAGCUGUUCGAGGAGAACCCCAU | |
| | CAACGCCAGCGGCGUGGACGCCAAGGC | |
| | CAUCCUGAGCGCCCGGCUGAGCAAGAG | |
| | CCGGCGGCUGGAGAACCUGAUCGCCCA | |
| | GCUGCCCGGCGAGAAGAAGAACGGCCU | |
| | GUUCGGCAACCUGAUCGCCCUGAGCCU | |
| | GGGCCUGACCCCCAACUUCAAGAGCAA | |
| | CUUCGACCUGGCCGAGGACGCCAAGCU | |
| | GCAGCUGAGCAAGGACACCUACGACGA | |
| | CGACCUGGACAACCUGCUGGCCCAGAU | |
| | CGGCGACCAGUACGCCGACCUGUUCCU | |
| | GGCCGCCAAGAACCUGAGCGACGCCAU | |
| | CCUGCUGAGCGACAUCCUGCGGGUGAA | |
| | CACCGAGAUCACCAAGGCCCCCCUGAG | |
| | CGCCAGCAUGAUCAAGCGGUACGACGA | |
| | GCACCACCAGGACCUGACCCUGCUGAA | |
| | GGCCCUGGUGCGGCAGCAGCUGCCCGA | |
| | GAAGUACAAGGAGAUCUUCUUCGACCA | |
| | GAGCAAGAACGGCUACGCCGGCUACAU | |
| | CGACGGCGGCGCCAGCCAGGAGGAGUU | |
| | CUACAAGUUCAUCAAGCCCAUCCUGGA | |
| | GAAGAUGGACGGCACCGAGGAGCUGCU | |
| | GGUGAAGCUGAACCGGGAGGACCUGCU | |
| | GCGGAAGCAGCGGACCUUCGACAACGG | |
| | CAGCAUCCCCCACCAGAUCCACCUGGG | |
| | CGAGCUGCACGCCAUCCUGCGGCGGCA | |
| | GGAGGACUUCUACCCCUUCCUGAAGGA | |
| | CAACCGGGAGAAGAUCGAGAAGAUCCU | |
| | GACCUUCCGGAUCCCCUACUACGUGGG | |
| | CCCCCUGGCCCGGGGCAACAGCCGGUU | |
| | CGCCUGGAUGACCCGGAAGAGCGAGGA | |
| | GACCAUCACCCCCUGGAACUUCGAGGA | |
| | GGUGGUGGACAAGGGCGCCAGCGCCCA | |
| | GAGCUUCAUCGAGCGGAUGACCAACUU | |
| | CGACAAGAACCUGCCCAACGAGAAGGU | |
| | GCUGCCCAAGCACAGCCUGCUGUACGA | |
| | GUACUUCACCGUGUACAACGAGCUGAC | |
| | CAAGGUGAAGUACGUGACCGAGGGCAU | |
| | GCGGAAGCCCGCCUUCCUGAGCGGCGA | |
| | GCAGAAGAAGGCCAUCGUGGACCUGCU | |
| | GUUCAAGACCAACCGGAAGGUGACCGU | |
| | GAAGCAGCUGAAGGAGGACUACUUCAA | |
| | GAAGAUCGAGUGCUUCGACAGCGUGGA | |
| | GAUCAGCGGCGUGGAGGACCGGUUCAA | |
| | CGCCAGCCUGGGCACCUACCACGACCU | |
| | GCUGAAGAUCAUCAAGGACAAGGACUU | |
| | CCUGGACAACGAGGAGAACGAGGACAU | |
| | CCUGGAGGACAUCGUGCUGACCCUGAC | |
| | CCUGUUCGAGGACCGGGAGAUGAUCGA | |
| | GGAGCGGCUGAAGACCUACGCCCACCU | |
| | GUUCGACGACAAGGUGAUGAAGCAGCU | |
| | GAAGCGGCGGCGGUACACCGGCUGGG | |
| | GCCGGCUGAGCCGGAAGCUGAUCAACG | |
| | GCAUCCGGGACAAGCAGAGCGGCAAGA | |
| | CCAUCCUGGACUUCCUGAAGAGCGACG | |
| | GCUUCGCCAACCGGAACUUCAUGCAGC | |
| | UGAUCCACGACGACAGCCUGACCUUCA | |
| | AGGAGGACAUCCAGAAGGCCCAGGUGA | |
| | GCGGCCAGGGCGACAGCCUGCACGAGC | |
| | ACAUCGCCAACCUGGCCGGCAGCCCCG | |
| | CCAUCAAGAAGGGCAUCCUGCAGACCG | |
| | UGAAGGUGGUGGACGAGCUGGUGAAGG | |
| | UGAUGGGCCGGCACAAGCCCGAGAACA | |
| | UCGUGAUCGAGAUGGCCCGGGAGAACC | |
| | AGACCACCCAGAAGGGCCAGAAGAACAG | |
| | CCGGGAGCGGAUGAAGCGGAUCGAGGA | |
| | GGGCAUCAAGGAGCUGGGCAGCCAGAU | |
| | CCUGAAGGAGCACCCCGUGGAGAACAC | |
| | CCAGCUGCAGAACGAGAAGCUGUACCU | |
| | GUACUACCUGCAGAACGGCCGGGACAU | |
| | GUACGUGGACCAGGAGCUGGACAUCAA | |
| | CCGGCUGAGCGACUACGACGUGGACCA | |
| | CAUCGUGCCCCAGAGCUUCCUGAAGGA | |
| | CGACAGCAUCGACAACAAGGUGCUGAC | |
| | CCGGAGCGACAAGAACCGGGGCAAGAG | |
| | CGACAACGUGCCCAGCGAGGAGGUGGU | |
| | GAAGAAGAUGAAGAACUACUGGCGGCA | |
| | GCUGCUGAACGCCAAGCUGAUCACCCA | |
| | GCGGAAGUUCGACAACCUGACCAAGGC | |
| | CGAGCGGGGCGGCCUGAGCGAGCUGG | |
| | ACAAGGCCGGCUUCAUCAAGCGGCAGC | |
| | UGGUGGAGACCCGGCAGAUCACCAAGC | |
| | ACGUGGCCCAGAUCCUGGACAGCCGGA | |
| | UGAACACCAAGUACGACGAGAACGACAA | |
| | GCUGAUCCGGGAGGUGAAGGUGAUCAC | |
| | CCUGAAGAGCAAGCUGGUGAGCGACUU | |
| | CCGGAAGGACUUCCAGUUCUACAAGGU | |
| | GCGGGAGAUCAACAACUACCACCACGC | |
| | CCACGACGCCUACCUGAACGCCGUGGU | |
| | GGGCACCGCCCUGAUCAAGAAGUACCC | |
| | CAAGCUGGAGAGCGAGUUCGUGUACGG | |
| | CGACUACAAGGUGUACGACGUGCGGAA | |
| | GAUGAUCGCCAAGAGCGAGCAGGAGAU | |
| | CGGCAAGGCCACCGCCAAGUACUUCUU | |
| | CUACAGCAACAUCAUGAACUUCUUCAAG | |
| | ACCGAGAUCACCCUGGCCAACGGCGAG | |
| | AUCCGGAAGCGGCCCCUGAUCGAGACC | |
| | AACGGCGAGACCGGCGAGAUCGUGUGG | |
| | GACAAGGGCCGGGACUUCGCCACCGUG | |
| | CGGAAGGUGCUGAGCAUGCCCCAGGUG | |
| | AACAUCGUGAAGAAGACCGAGGUGCAG | |
| | ACCGGCGGCUUCAGCAAGGAGAGCAUC | |
| | CUGCCCAAGCGGAACAGCGACAAGCUG | |
| | AUCGCCCGGAAGAAGGACUGGGACCCC | |
| | AAGAAGUACGGCGGCUUCGACAGCCCC | |
| | ACCGUGGCCUACAGCGUGCUGGUGGUG | |
| | GCCAAGGUGGAGAAGGGCAAGAGCAAG | |
| | AAGCUGAAGAGCGUGAAGGAGCUGCUG | |
| | GGCAUCACCAUCAUGGAGCGGAGCAGC | |
| | UUCGAGAAGAACCCCAUCGACUUCCUG | |
| | GAGGCCAAGGGCUACAAGGAGGUGAAG | |
| | AAGGACCUGAUCAUCAAGCUGCCCAAG | |
| | UACAGCCUGUUCGAGCUGGAGAACGGC | |
| | CGGAAGCGGAUGCUGGCCAGCGCCGGC | |
| | GAGCUGCAGAAGGGCAACGAGCUGGCC | |
| | CUGCCCAGCAAGUACGUGAACUUCCUG | |
| | UACCUGGCCAGCCACUACGAGAAGCUG | |
| | AAGGGCAGCCCCGAGGACAACGAGCAG | |
| | AAGCAGCUGUUCGUGGAGCAGCACAAG | |
| | CACUACCUGGACGAGAUCAUCGAGCAG | |
| | AUCAGCGAGUUCAGCAAGCGGGUGAUC | |
| | CUGGCCGACGCCAACCUGGACAAGGUG | |
| | CUGAGCGCCUACAACAAGCACCGGGAC | |
| | AAGCCCAUCCGGGAGCAGGCCGAGAAC | |
| | AUCAUCCACCUGUUCACCCUGACCAACC | |
| | UGGGCGCCCCCGCCGCCUUCAAGUACU | |
| | UCGACACCACCAUCGACCGGAAGCGGU | |
| | ACACCAGCACCAAGGAGGUGCUGGACG | |
| | CCACCCUGAUCCACCAGAGCAUCACCG | |
| | GCCUGUACGAGACCCGGAUCGACCUGA | |
| | GCCAGCUGGGCGGCGACAGCGGCGGCA | |
| | AGCGGCCCGCCGCCACCAAGAAGGCCG | |
| | GCCAGGCCAAGAAGAAGAAGGGCAGCU | |
| | ACCCCUACGACGUGCCCGACUACGCCU | |
| | GA | |

### III. Methods

In various aspects, a method correcting a mutation in an MYH7 gene in a cell is provided, the method comprising delivering to the cell: an Cas9 nickase or deactivated Cas9 endonuclease, a deaminase, and a gRNA targeting a DNA nucleotide sequence selected from any one of SEQ ID NOs. 1 or 2, or one or more nucleic acids encoding Cas9 nickase or deactivated Cas9 endonuclease, deaminase and/or gRNA, a to effect one or more single-strand breaks (SSBs) within or near the MYH7 gene that results in one or more mutations of at least one nucleotide within or near the MYH7 gene, thereby correcting the mutation in the MYH7 gene. In various aspects, the method may comprise delivering to the cell a nucleic acid encoding a gRNA and/or the fusion proteins described herein. The nucleic acid may be delivered in a viral vector. In some aspect, the nucleic acid may be delivered in two viral vectors (e.g., vectors described in Tables 12 and 13 above).

In further aspects, a method is provided of treating a cardiomyopathy caused by a mutation in an MYH7 gene in a subject in need thereof, the method comprising delivering to at least one cell in the subject expressing the MYH7 gene: a Cas9 nickase or deactivated Cas9 endonuclease, a deaminase, and a gRNA targeting a DNA nucleotide sequence selected from any one of SEQ ID NOs. 1 or 2, or one or more nucleic acids encoding the RNA guided nickase, deaminase and/or gRNA, a to effect one or more single-strand breaks (SSBs) within or near the MYH7 gene that results in one or more mutations of at least one nucleotide within or near the MYH7 gene, thereby correcting the mutation in the MYH7 gene in at least one cell of the subject. In various aspects, the RNA guided nickase, deaminase, and gRNA may be delivered in any pharmaceutical composition described herein. In some aspects, the Cas9 nickase/deactivated Cas9 endonuclease and deaminase are delivered as a fusion protein (e.g., any fusion protein described herein). in various aspects, the method comprises administering to the subject one or more viral vector encoding for the fusion protein and/or gRNA.

In various aspects, the mutation in the MYH7 gene corrected by any of these methods comprises one or more single nucleotide polymorphisms that result in a single amino acid substitution in a protein product encoded by the mutated MYH7 gene. In some instances, the protein product is a myosin protein or peptide and the single amino substitution comprises R403Q according to SEQ ID NO: 96.

In various embodiments, compositions disclosed herein may be effective for treating heart disease following administration to a subject in need. In other embodiments, compositions disclosed herein may be effective for treating one or more cardiomyopathies following administration to a subject in need. In still other embodiments, compositions disclosed herein may be effective for treating HCM following administration to a subject in need. In other embodiments, compositions disclosed herein may be effective for improving at least one symptom of HCM following administration to a subject in need.

A suitable subject herein includes a human, a livestock animal, a companion animal, a lab animal, or a zoological animal. In some embodiments, the subject may be a rodent, e.g., a mouse, a rat, a guinea pig, etc. In some embodiments, the subject may be a livestock animal. Non-limiting examples of suitable livestock animals may include pigs, cows, horses, goats, sheep, llamas and alpacas. In some embodiments, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In various embodiments, a subject in need may have been diagnosed with at least one heart disease. In some aspects, the subject may have one or more cardiomyopathies. In some embodiments, the subject may have HCM. In some embodiments, a subject may at least one symptom of HCM. In some aspects, a symptom of HCM can be fatigue. In some embodiments, a symptom of HCM can be dyspnea. In some embodiments, a symptom of HCM can be edema. In some embodiments, a symptom of HCM can be ascites. In some embodiments, a symptom of HCM can be chest pain. In still other aspects, a symptom of HCM can be a heart murmur.

In some embodiments, methods of administering compositions disclosed herein may decrease and/or reverse cardiomyopathy-induced cardiac fibrosis compared to cardiomyopathy-induced cardiac fibrosis in an untreated subject with identical disease condition and predicted outcome. In some embodiments, methods of administering compositions disclosed herein may decrease and/or reverse cardiomyopathy-induced left ventricle dilation compared to cardiomyopathy-induced left ventricle dilation in an untreated subject with identical disease condition and predicted outcome.

Other embodiments of the present disclosure are methods of administering compositions disclosed herein to a subject in need wherein administration treats cardiomyopathy (e.g., HCM). Still other embodiments of the present disclosure are methods of administering compositions disclosed herein to a subject in need wherein at least one symptom of cardiomyopathy (e.g., HCM) is improved by at least 25% within one month after administration.

In various embodiments, compositions disclosed herein may be administered by parenteral administration. As used herein, “by parenteral administration” refers to administration of the compositions disclosed herein via a route other than through the digestive tract. In some embodiments, compositions disclosed herein may be administered by parenteral injection. In some aspects, administration of the disclosed compositions by parenteral injection may be by subcutaneous, intramuscular, intravenous, intraperitoneal, intracardiac, intraarticular, or intracavernous injection. In some embodiments, administration of the disclosed compositions by parenteral injection may be by slow or bolus methods as known in the field. In some embodiments, the route of administration by parenteral injection can be determined by the target location. In some embodiments, compositions disclosed herein may be formulated for parenteral administration by intracardiac injection. In some embodiments, compositions disclosed herein may be formulated for parenteral administration by catheter-based intracoronary infusion. In some embodiments, compositions disclosed herein may formulated for parenteral administration by pericardial injection.

In various embodiments, the dose of compositions disclosed herein to be administered are not particularly limited and may be appropriately chosen depending on conditions such as a purpose of preventive and/or therapeutic treatment, a type of a disease, the body weight or age of a subject, severity of a disease and the like. In some embodiments, administration of a dose of a composition disclosed herein may comprise a therapeutically effective amount of the composition disclosed herein. As used herein, the term “therapeutically effective” refers to an amount of administered composition that treats heart disease, reduces presentation of at least one symptom associated with heart disease, reverses/prevents cardio fibrosis, reverse/prevent dilation of at least one heart ventricle, reduces total heart weight, improved heart function, increases survivability, or a combination thereof.

In some embodiments, a composition disclosed herein may be administered to a subject in need thereof once. In some embodiments, a composition disclosed herein may be administered to a subject in need thereof more than once. In some embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

The number of times a composition may be administered to a subject in need thereof can depend on the discretion of a medical professional, the severity of the heart disease, and the subject's response to the formulation. In some embodiments, a composition disclosed herein may be administered continuously; alternatively, the dose of composition being administered may be temporarily reduced or temporarily suspended for a certain length of time (i.e., a “composition holiday”). In some aspects, the length of the composition holiday can vary between 2 days and 1 year, including by way of example only, 2 days, 1 week, 1 month, 6 months, and 1 year. In another aspect, dose reduction during a composition holiday may be from 10%-100%, including by way of example only 10%, 25%, 50%, 75%, and 100%.

In various embodiments, the desired daily dose of compositions disclosed herein may be presented in a single dose or as divided doses administered simultaneously (or over a short period of time) or at appropriate intervals. In other embodiments, administration of a composition disclosed herein may be administered to a subject about once a day, about twice a day, about three times a day. In still other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of a composition disclosed herein may be administered to a subject once about 4 weeks.

In some embodiments, a composition as disclosed may be initially administered followed by a subsequent administration of one for more different compositions or treatment regimens. In other embodiments, a composition as disclosed may be administered after administration of one for more different compositions or treatment regimens.

### IV. Kits

Some embodiments of the present disclosure include kits for packaging and transporting CRISPR-Cas9 systems and/or novel gRNAs disclosed herein or known gRNAs disclosed herein and further include at least one container.

In some embodiments, the kit can additionally comprise instructions for use of CRISPR-Cas9 systems, gRNAs, and or AAV particles in any of the methods described herein. The included instructions may comprise a description of administration of pharmaceutical compositions as disclosed herein to a subject to achieve the intended activity in a subject. The kit may further comprise a description of selecting a subject suitable for treatment based on identifying whether the subject is in need of the treatment. In some embodiments, the instructions may comprise a description of administering pharmaceutical compositions disclosed herein to a subject who has or is suspected of having a cardiomyopathy.

As will be apparent, it is envisaged that the present system can be used to target any polynucleotide sequence of interest. Some examples of conditions or diseases that might be use fully treated using the present system are included in the figures and tables herein and examples of genes currently associated with those conditions are also provided there. However, the genes exemplified are not exhaustive. Additional objects, advantages, and novel features of this disclosure will become apparent to those skilled in the art upon review of the following examples in light of this disclosure. The following examples are not intended to be limiting.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1

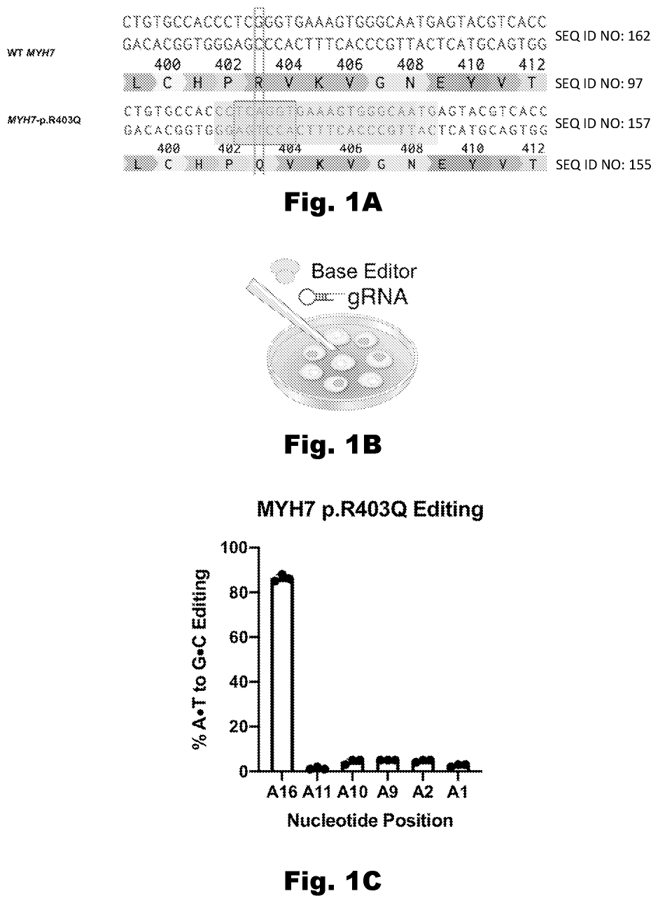

In an exemplary method, CRISPR-Cas9 was used for correction of a MYH7 mutation in human cell. In brief, patient-derived induced pluripotent stem cells (iPSCs) containing an MYH7 c.1208G>A (p.R403Q) mutation (Mut) were used in these exemplary studies. The MYH7 p.R403Q mutation occurs in one-third of all HCM-causing mutations and results in a mutation in coding nucleotide 1208 from a guanine to an adenine, resulting in conversion of amino acid 403 from an arginine to a glutamine in the final protein FIG. 1A shows a gRNA with the sequence 5′-CCT CAG GTG AAA GTG GGC AA-3′ (SEQ ID NO: 1) with the protospacer adjacent motif (PAM) 5′-TGAG-3′. Following nucleofection of a plasmid encoding the gRNA with the sequence 5′-CCT CAG GTG AAA GTG GGC AA-3′ (SEQ ID NO: 1) with the protospacer adjacent motif (PAM) 5′-TGAG-3′ and a plasmid encoding ABEmax-SpCas9-NG (FIG. 1B), a robust editing of the mutant adenine nucleotide back to the wildtype guanine nucleotide with no significant bystander editing of neighboring adenine nucleotides (FIG. 1C).

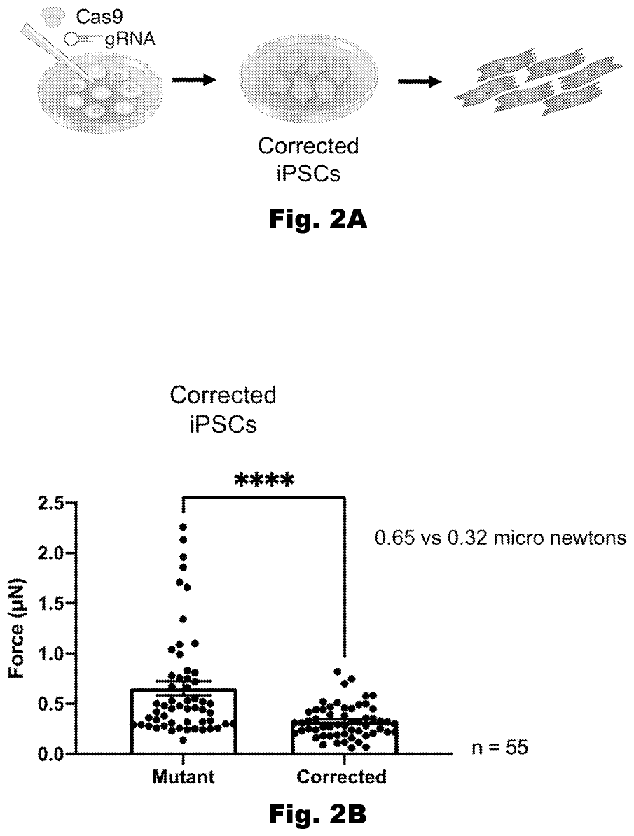

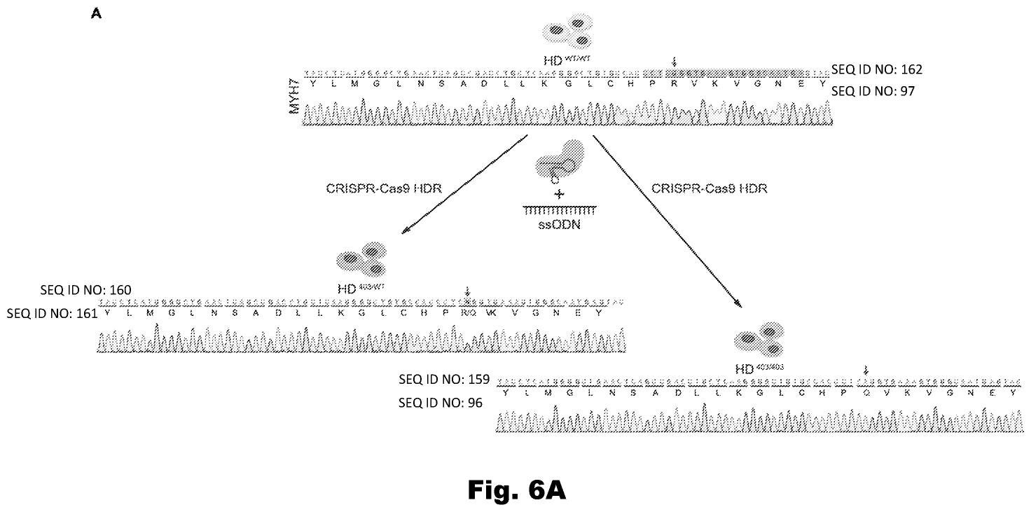

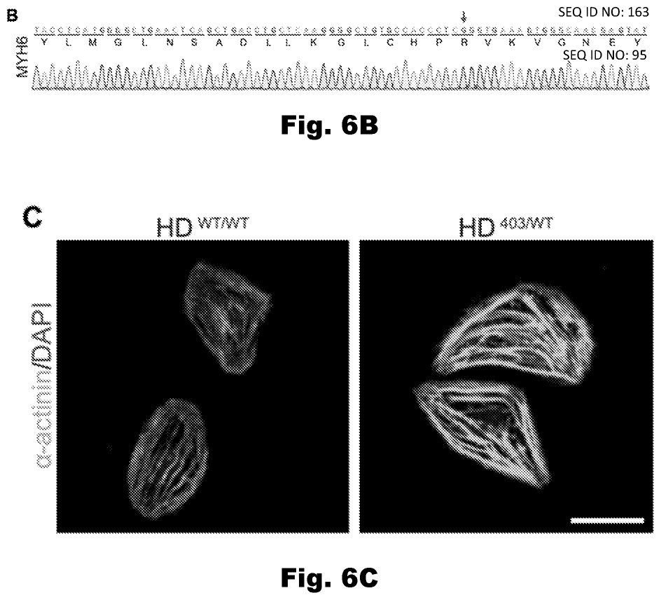

Next patient-derived induced pluripotent stem cells (iPSCs) containing the MYH7 c.1208G>A (p.R403Q) mutation (Mut) or iPSCs corrected using the CRISPR-Cas9 method described above (Cor) were isolated and differentiated into cardiomyocytes (iPSC-CMs) (FIG. 2A, FIG. 6C). Analysis of force generation by Mut iPSC-CMs and Cor iPSC-CMs showed a significant reduction in the Cor line, demonstrating that correction of the MYH7 p.R403Q mutation decreased the hypercontractility phenotype (FIG. 2B). These data suggested that CRISPR-Cas9 can be used for amelioration of the hypercontractile phenotype found in patients.

### Example 2

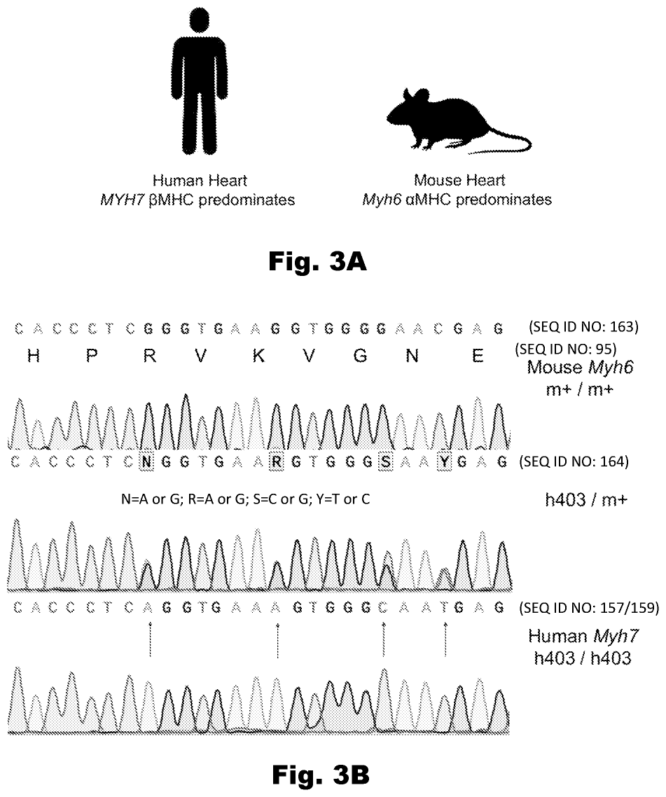

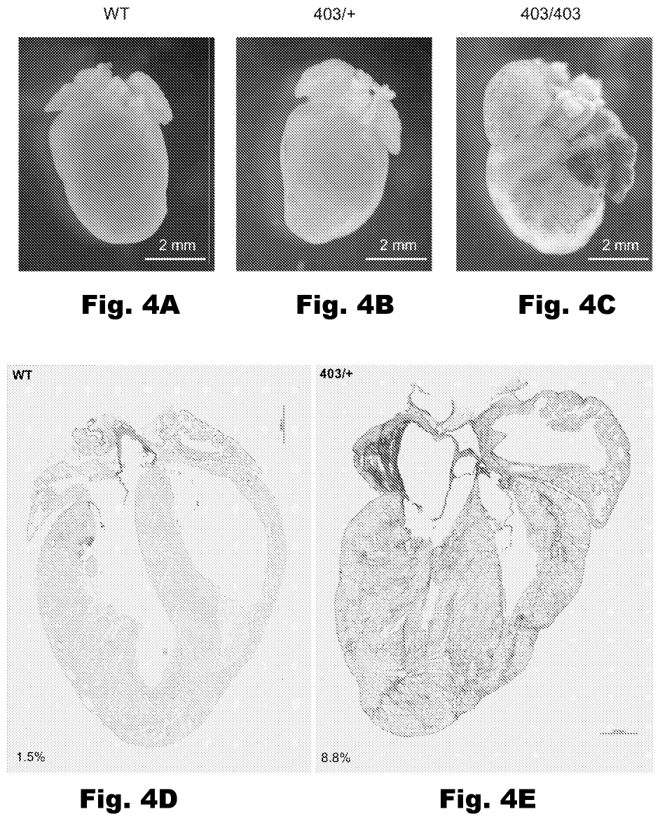

In another exemplary method, a genetically modified mouse line was generated to model the human MYH7 p.R403Q mutation (FIG. 3A). Specifically, the mouse line contained the same human disease-causing mutation within the mouse myosin heavy chain 6 (Myh6) gene, the dominantly expressed myosin isoform in mice (FIG. 3B). Mice that carried the missense mutation on one allele (403/+) and mice that were carried the missense mutation on both alleles (403/403) were monitored for cardiac phenotypes from development in a head to head manner with a mouse contain not missense mutation (wild type, or “WT”). 403/403 mice begin showing enlarged hearts at P8 (FIGS. 4A-4C). Marked cardiac fibrosis was observed in 403/+ mice 6 months after birth (FIGS. 4D and 4E).

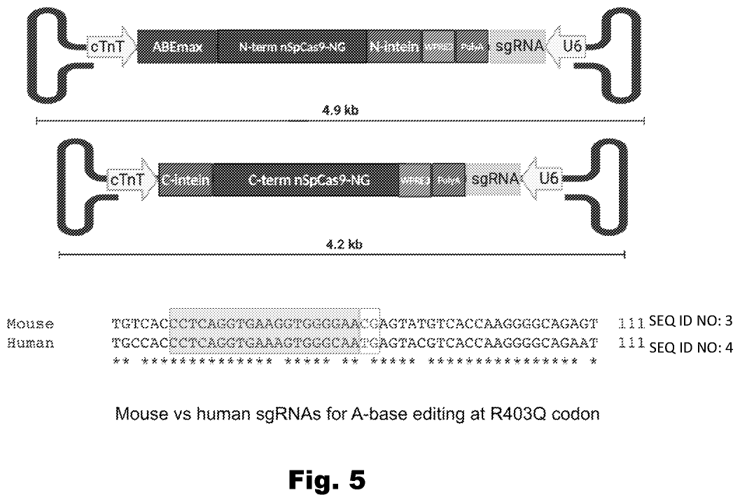

To correct the Myh6.R403Q mutation in the mouse model of the human MYH7 p.R403Q mutation, a sgRNA was designed with the sequence 5′-CCT CAG GTG AAG GTG GGG AA-3′ (SEQ ID NO: 2) with the PAM 5′-CGAG-3′ (SEQ ID NO: 4) for adeno-associated virus (AAV)-based correction in the mouse line (FIG. 5). On-target and off-target editing efficiency in the mice is determined using AAV delivery and/or A-base editor. After administering the sgRNA via AAV into the mouse model of the human MYH7 p.R403Q mutation, cardiac function will be assessed and compared to cardiac function prior to administration of sgRNA to measure phenotypic rescue in the mice.

### Example 3 Identification of an ABE to Correct the R403Q Mutation in Human iPSCs

Base editors are fusion proteins of Cas9 nickase or deactivated Cas9 and a deaminase protein, which allow base pair edits without double-strand breaks within a defined editing window in relation to the protospacer adjacent motif (PAM) site of a single-guide RNA (sgRNA). Adenine base editors (ABEs) use deoxyadenosine deaminase to convert DNA A•T base pairs to G•C base pairs via an inosine intermediate. To screen various adenine base editors (ABEs) for their efficiencies, a MYH7 c.1208 G>A (p.R403Q) pathogenic missense mutation was inserted using CRISPR-Cas9-based homology-directed repair in a human induced pluripotent stem cell (iPSC) line derived from a healthy donor (HD^{WT}). An isogenic heterozygous mutation clone (HD^{403/+}) was isolated that mirrors the heterozygous genotype found in patients, as well as an isogenic homozygous mutation clone (HD^{401/403}) that had not been previously described in patients. Sequencing confirmed no mutations on the highly homologous MYH6 gene during generation of these clones (FIG. 6A-6B).

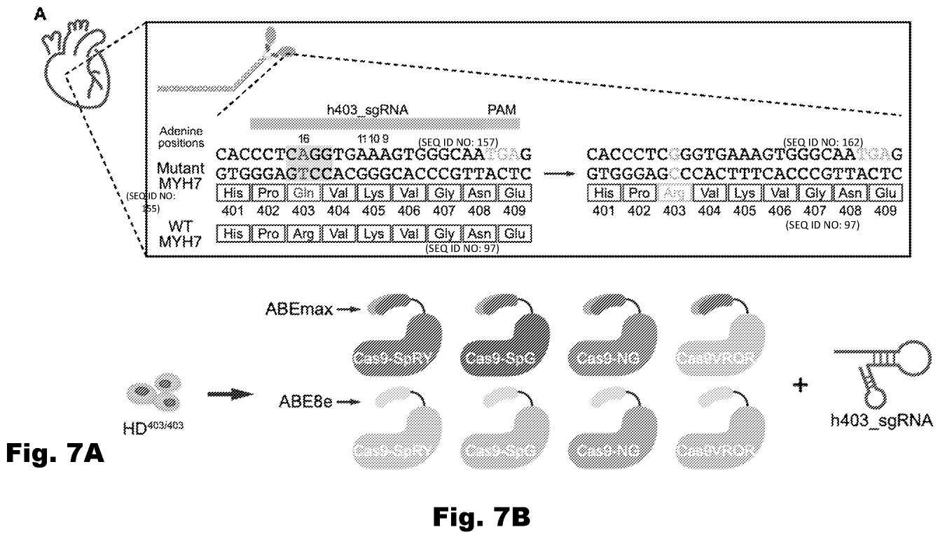

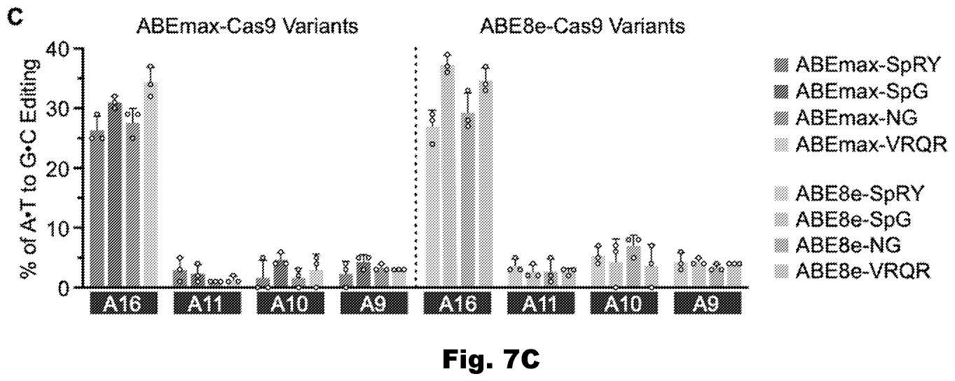

As ABEs have an optimal activity window in protospacer positions 14-17 (counting the first nucleotide immediately 5′ of the PAM sequence as protospacer position 1), an sgRNA was chosen with an NGA PAM that places the MYH7 c.1208 G>A mutation in protospacer position 16 (h403_sgRNA) (FIG. 7A). To identify an optimal ABE capable of efficiently correcting the pathogenic nucleotide back to the wildtype nucleotide without introducing any bystander edits, various engineered deaminases were tested including either ABEmax (SEQ ID NO: 7), which is an optimized, narrow-windowed ABE7.10 variant (SEQ ID NO: 11), or ABE8e, (SEQ ID NO: 9) which is a highly processive, wide-windowed, evolved ABE7.10 variant. Amino acid and nucleic acid sequence for each deaminase variant are provided in Tables 1 and 2 above. Each engineered deaminase variant was fused to engineered SpCas9 variants including SpRY (SEQ ID NO: 17), which targets NRN PAMs; SpG (SEQ ID NO: 19), which targets NGN PAMs; SpCas9-NG (SEQ ID NO: 21), which targets NG PAMs; or SpCas9-VRQR (SEQ ID NO: 15), which targets NGA PAMs. Amino acid and nucleic acid sequences for each SpCas9 variant are provided in Tables 3 and 4 above. These ABEs were then screened for their efficiency of correction in our HD^{403/403} iPSC line via transient transfection with h403_sgRNA (SEQ ID NO: 1, FIG. 7B). Similar editing efficiency of the pathogenic adenine was achieved with all ABEmax-SpCas9 variants tested, ranging from 26±2.3% with ABEmax-SpRY to 34±2.5% with ABEmax-VRQR, with minimal bystander editing of neighboring adenines (the average across three bystanders was 2.6±1.7%). ABE8e-SpCas9 variants achieved higher editing efficiencies, ranging from 27±2.6% with ABE8e-SpRY (SEQ ID NO: 57) to 37±1.5% with ABE8e-SpG (SEQ ID NO: 59) with slightly increased bystander editing of neighboring adenines (the average across three bystanders was 4.0±2.0%) (FIG. 7C). These bystander edits are predicted to result in K405E, K405R, or K405G mutations in β-myosin heavy chain depending on the combination of edits, although the consequences of these mutations on β-myosin heavy chain function have not been described. For subsequent experiments, the more narrow-windowed ABEmax was used to reduce potential bystander edits, and the SpCas9-VRQR variant with its more stringent PAM requirements was used to reduce potential Cas-dependent off-target editing. The resulting fusion protein (ABEmax-VRQR) had an amino acid sequence of SEQ ID NO: 45. The same fusion protein further comprising nuclear localization sequences, which was used in the following examples, has an amino acid sequence of SEQ ID NO: 46. Amino acid sequences and encoding nucleic acids for all deaminase-nickase proteins described in these examples are provided in Tables 7 and 8 above.

### Example 4—Correction Efficiency and Off-Target DNA Editing Analysis in HCM Patient-Derived iPSCs

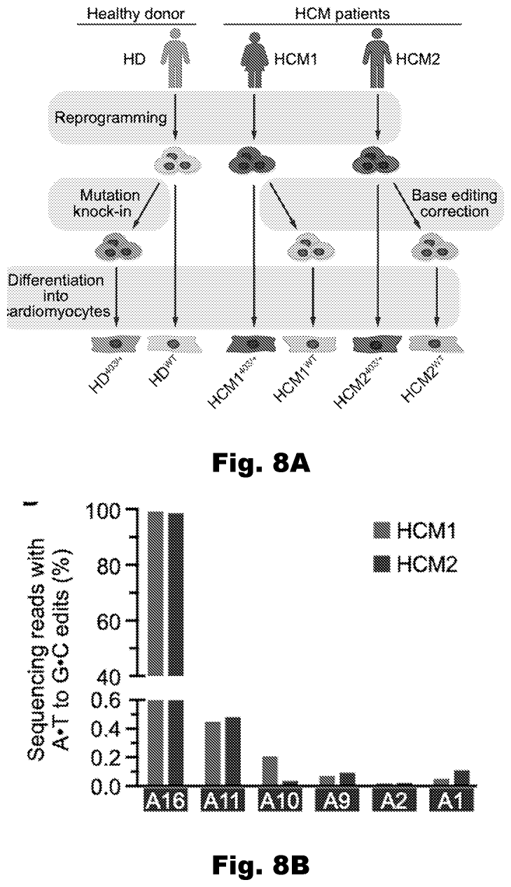

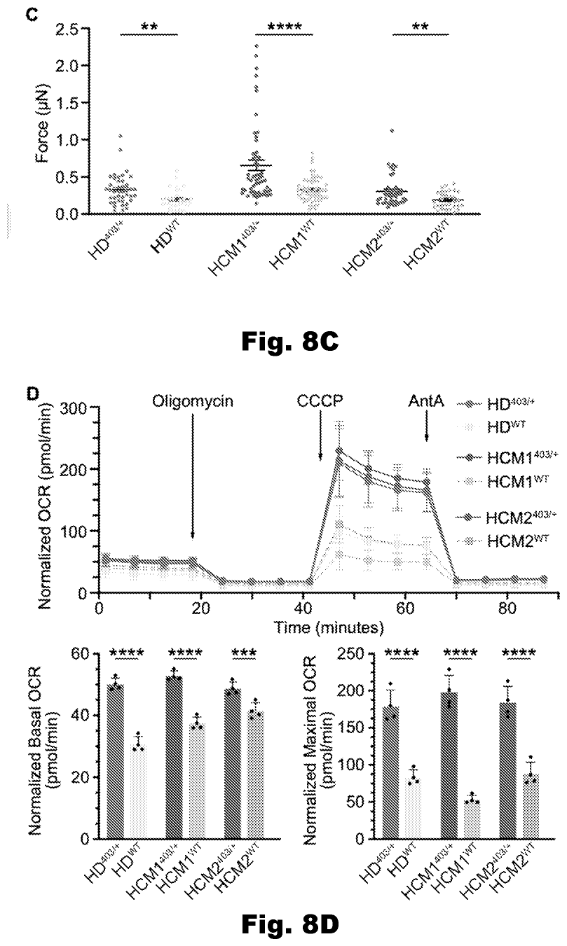

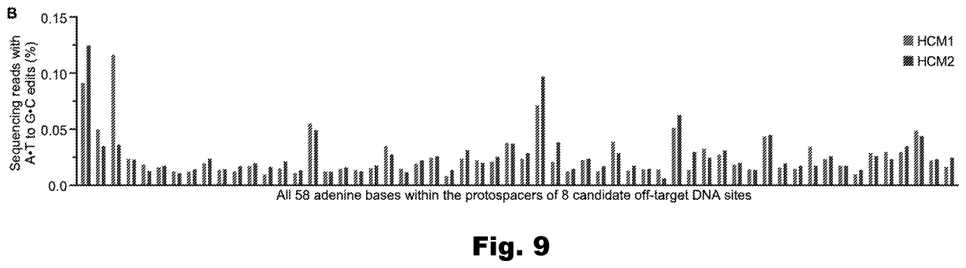

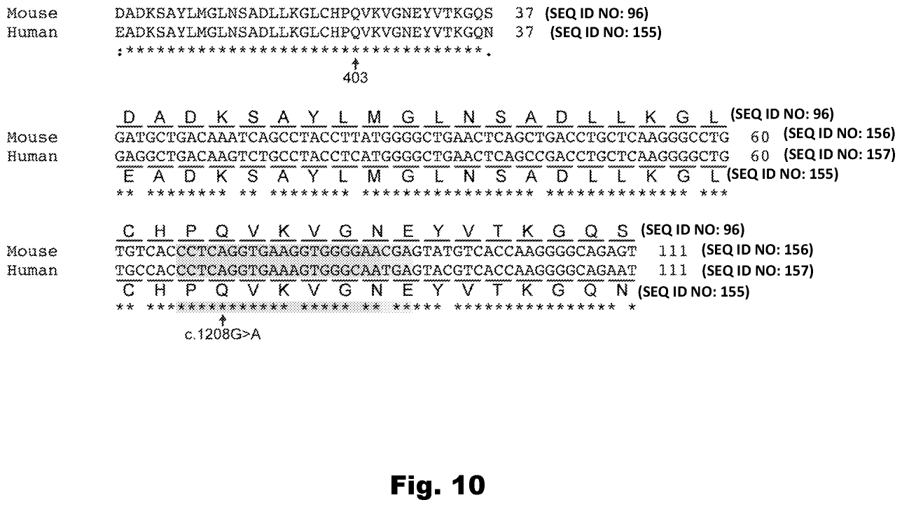

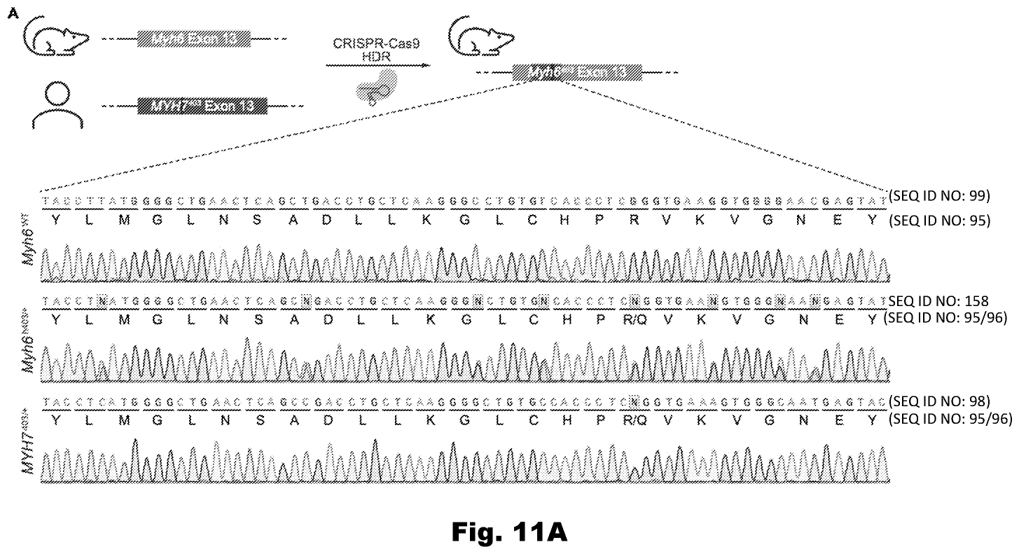

To apply the ABEmax-VRQR and h403_sgRNA system to a disease model, human induced pluripotent stem cells (iPSCs) were derived from two HCM patients with the MYH7^{403/+} mutation (HCM1^{403/+} and HCM2^{403/+}) the MYH7^{403/+} mutation was corrected via plasmid nucleofection of ABEmax-VRQR-P2a-EGFP and h403_sgRNA (SEQ ID NO: 1), and fluorescence-activated cell sorting of GFP⁺ cells (FIG. 8A). High throughput sequencing (HTS), revealed that, despite 98-99% on-target editing, minimal to no off-target DNA editing (0.12% or less) occurred at all 58 adenine bases for 8 tested candidate off-target loci, which were identified using the bioinformatic tool CRISPOR (FIG. 8B, and FIG. 9 and Table 16 below). A low frequency (0.03-0.48%) of bystander editing was observed at the three bystander adenines for amino acid 505 (K505) of β-myosin. For subsequent characterization, corrected clonal lines of the HCM patient-derived iPSCs (HCM1^{WT} and HCM2^{WT}) were isolated that contained no bystander edits or editing of the highly homologous MYH6 gene. These results suggest that h403_sgRNA with ABEmax-VRQR can efficiently and specifically correct the target pathogenic missense mutation with minimal bystander editing and little to no DNA off-target editing.

TABLE 16

| | | PAM | SEQ ID | |
|---|---|---|---|---|
| Target | gRNA Sequence | | NO: | Gene |
| On | CCTCAGGTGAAAGTGGGCAA | TGA | 1 | MYH7 |
| Target | | | | |
| OT1 | CCTCGGGTGAAAGTGGGCAA | CGA | 105 | MYH6 |
| OT2 | CCTAAAGAGAAAATGGGCAA | AGA | 106 | Intron; CEP57 |
| OT3 | TCTCAGATGAAAGTGAGCTA | AGA | 107 | FRYL |
| OT4 | CATCAAGTGAAAGTGGACAG | GGA | 108 | Intron; |
| | | | | SMPDL3B/RP11- |
| | | | | 460113.2 |
| OT5 | CCTCAGGAGAAGATGGACAA | AGA | 109 | Intergenic; |
| | | | | RP11-27814.2- |
| | | | | COLEC10 |
| OT6 | TATCAGGTGAAGGTAGGCAA | TGA | 110 | STAU2 |
| OT7 | GCTCAGGAGAAGGTGGACAA | TGA | 111 | RP6-127F18.2 |
| OT8 | TCTCAAGGGAGAGTGGGCAA | GGA | 112 | Intron; FERMT1- |
| | | | | TARDBPP1 |

### Example 5—Functional Analyses of ABE-Corrected Patient iPSC-Derived CMs

To determine the functional consequences of base editing correction in human cardiomyocytes (CMs), both MYH7^{403/+} mutant and MYH7^{WT} healthy clonal lines were differentiated for all three patient-derived lines (HD, HCM1, and HCM2) into CMs to investigate the effects of gene editing correction on CM function (FIG. 8A).

A hallmark feature of CMs is the generation of contractile force. HCM results in hypercontractility, which can lead to increased force generation. To investigate whether gene editing correction could reduce hypercontractile force generation in our HCM patient-derived lines, iPSC-CMs were plated at single-cell density on soft polydimethylsiloxane surfaces, recorded high frame-rate videos of contracting CMs, and calculated peak systolic force. The HD^{403/+} iPSC-CMs showed a 1.7-fold increase in peak systolic force compared to HD^{WT} iPSC-CMs originally derived from a healthy donor. On the other hand, corrected HCM1^{WT} and HCM2^{WT} CMs showed a 2.0-fold and 1.6-fold decrease in peak systolic force, respectively, compared to their isogenic HCM1^{403/+} and HCM2^{403/+} counterparts. (FIG. 8C).

As previous studies have shown that HCM mutations lead to increased ATP consumption and altered cellular metabolism, changes in cellular energetics were assessed via metabolic flux assays following gene editing correction. Basal oxygen consumption rates (OCR) were increased 1.6-fold in HD^{403/+} iPSC-CMs compared to HD^{WT} iPSC-CMs, and HD^{403/+} iPSC-CMs had a 2.1-fold increase in maximum OCR compared to HD^{WT} iPSC-CMs. Corrected HCM1^{WT} and HCM2^{WT} CMs showed a 1.4-fold and 1.2-fold reduction in basal OCR, respectively, and a 3.7-fold and 2.1-fold reduction in maximum OCR, respectively, compared to isogenic HCM1^{403/+} and HCM2^{403/+} CMs (FIG. 8D). These data demonstrate that correction of the pathogenic mutation in human HCM CMs is sufficient to reduce the hypercontractility phenotype and restore normal cellular energetics.

### Example 6—Development of a Humanized Mouse Model of HCM

The methods of base editing described above were applied to a mouse model of HCM. While β-myosin heavy chain is the dominant myosin isoform found in adult human hearts, the highly homologous α-myosin heavy chain is the dominant myosin isoform expressed in adult mouse hearts and is encoded by the Myh6 gene. Consequently, previously described mouse models for HCM have placed the corresponding human MYH7 mutation on the mouse Myh6 gene to account for these expression differences. While the 30 amino acids around R403 are 100% identical between human MYH7 and mouse Myh6, the DNA sequence encoding this region of the protein is not identical (FIG. 10). Thus, sgRNAs and editing strategies developed for the human genome might not be directly applicable to a mouse model.

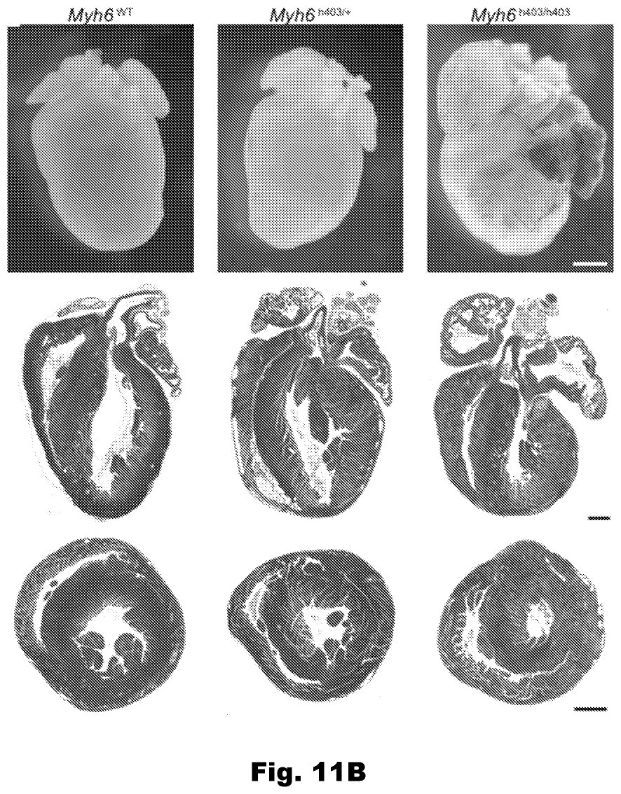

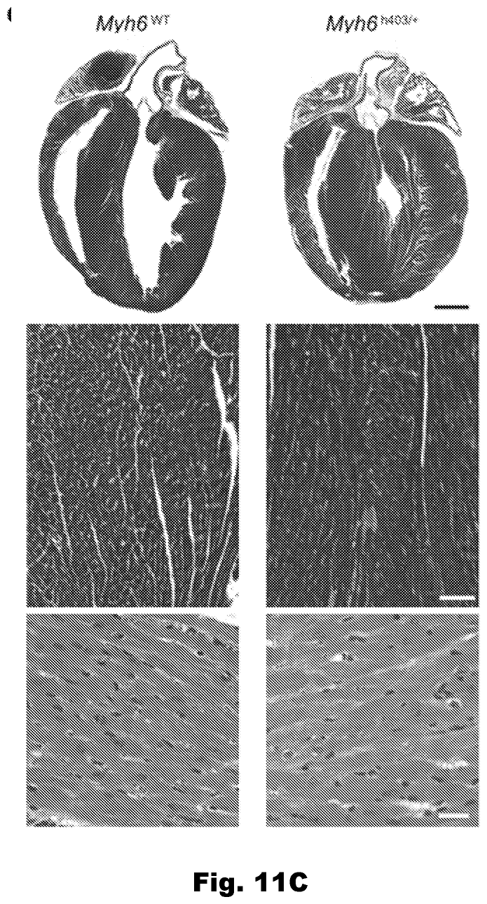

To perform preclinical studies using our human sequence-specific base editing strategy, a humanized mouse model was generated that contained the MYH7 c.1208 G>A (p.R403Q) human missense mutation within the mouse Myh6 gene that also has human DNA sequence identity of at least 22 nucleotides upstream and downstream from the mutation to allow testing of human genome specific CRISPR strategies (FIG. 11A). The other Myh6 allele contained the unmodified mouse genomic sequence. This humanized mouse model (Myh6^{h403/+}) mirrors the phenotype of previously described Myh6 p.R403Q mouse models. Most notably, homozygous mice (Myh6^{h403/h403}) have enlarged atria, extensive interstitial fibrosis, and die within the first week of life (FIG. 11B). At 9 months of age, Myh6^{h403/+} mice have developed cardiomyopathy with significant ventricular hypertrophy, myocyte disarray, and fibrosis (FIG. 11C).

### Example 7—In Vivo ABE Treatment of a Mouse Model of Human HCM

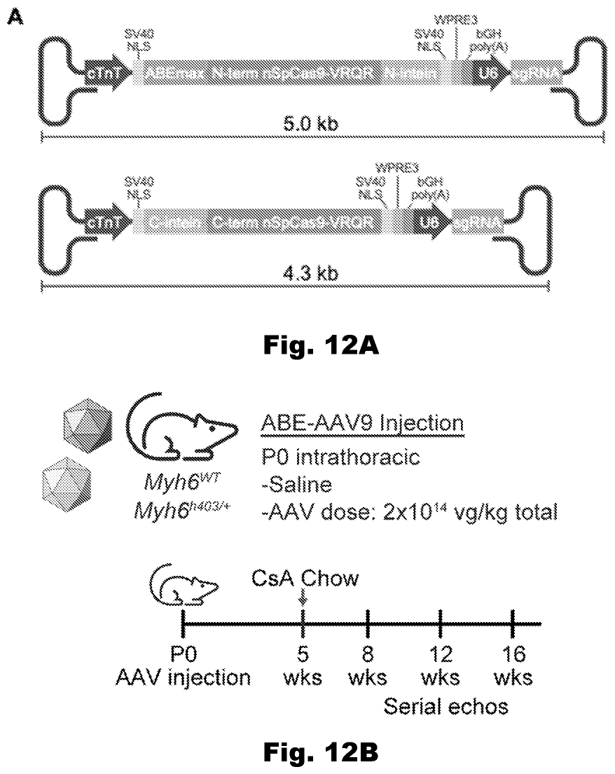

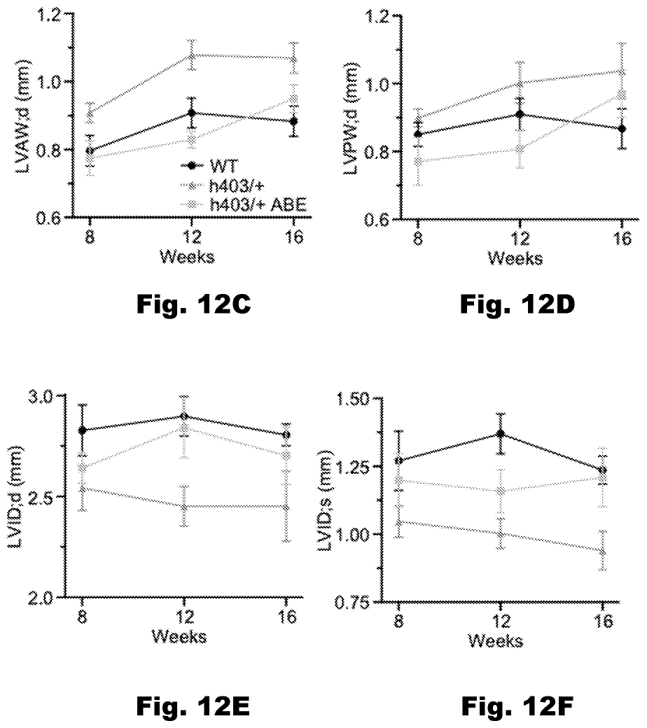

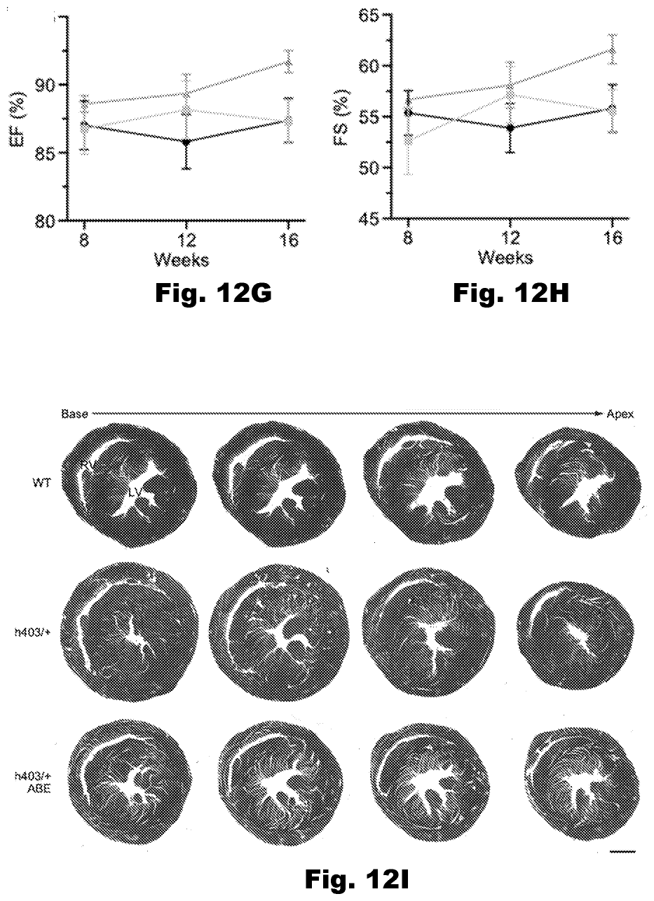

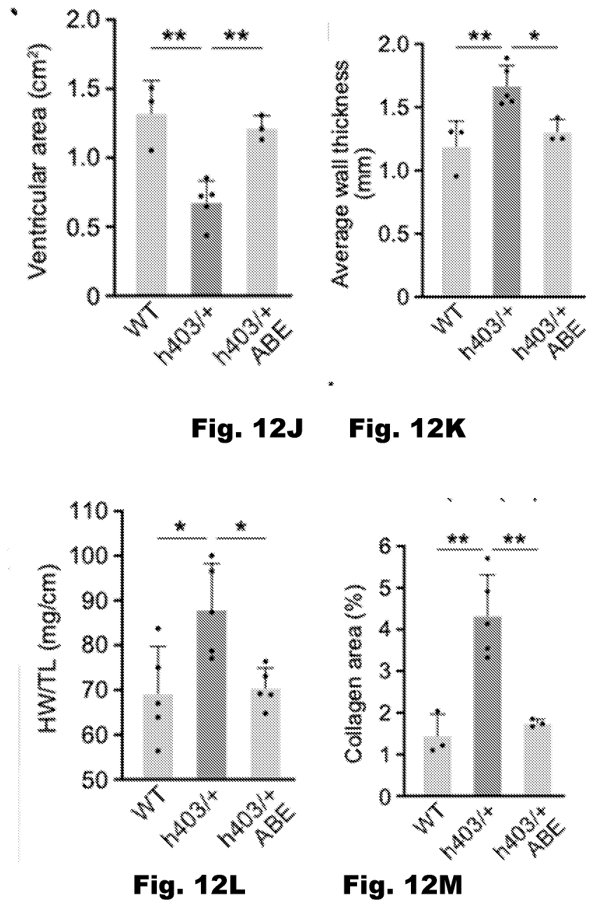

The ABEmax-VRQR and h403_sgRNA were packaged within adeno-associated virus (AAV). As the full-length base editor (˜5.6 kb) exceeded the packaging limit of a single AAV9 (˜4.7 kb), the base editor was split across two AAV9s (SEQ ID NOs: 86 and 91) and used trans-splicing inteins to reconstitute the full-length base editor in cells upon protein expression. As AAV9 contains broad tissue tropism, a cardiac troponin T promoter was used to limit expression of the base editor to CMs. For this dual AAV9 system, each AAV9 also contained a single copy of an expression cassette encoding h403_sgRNA (FIG. 12A). The two vectors are described in Tables 9 and 10 above, along with their constituent components.

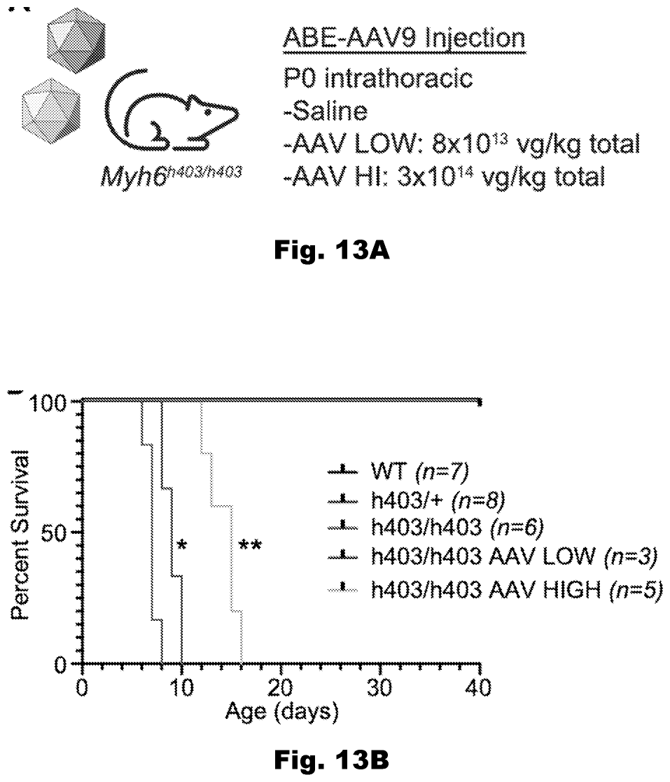

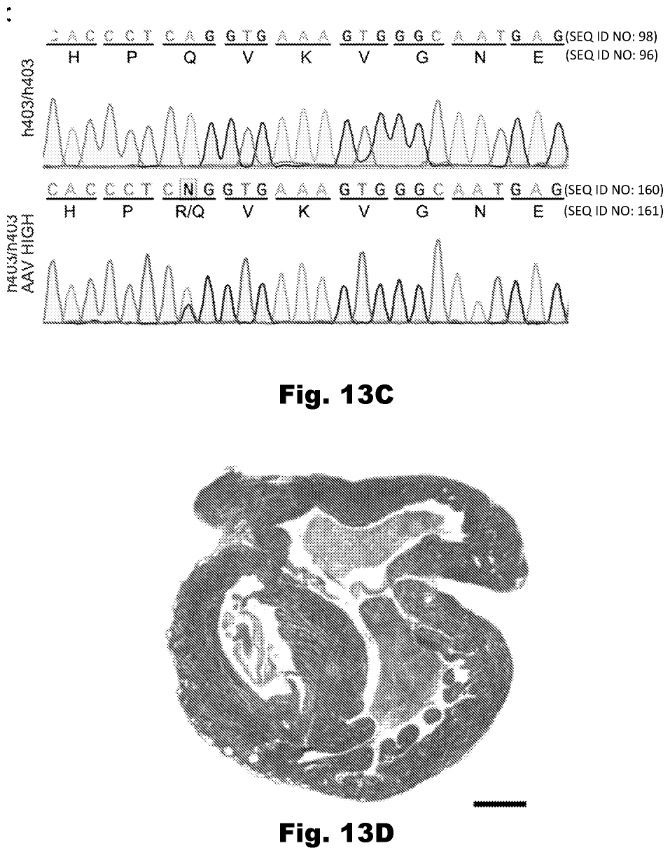

The efficiency of our dual AAV9 ABE system was validated by trying to rescue Myh6^{h403/h1403} mice, which die within the first week of life. Notably, no human patients have been reported to have the homozygous genotype. P0 (postnatal day 0) Myh6^{h403/h1403} pups were injected intrathoracically with either saline, a low dose (4×10¹³ vg/kg), or a high dose (1.5×10¹⁴ vg/kg) of each AAV9 (total of 8×10¹³ vg/kg for low, and 3×10¹⁴ vg/kg for high) and their development was monitored (FIG. 13A). The 3×10¹⁴ vg/kg high dose is the highest dose administered in clinical trials. The Myh6^{403/+} and Myh6^{WT} mice survived past weaning and well into adulthood. The median survival of saline-injected mice was 7.0 days, whereas that of low-dose ABE-treated mice was increased to 9.0 days (1.3-fold longer, P<0.05 by Mantel-Cox test). The median survival of high-dose ABE-treated mice was increased to 15.0 days (2.1-fold longer, P<0.01 by Mantel-Cox test) (FIG. 13B). Sanger sequencing of cDNA of the heart from a high-dose mouse indicated 35% correction of the pathogenic mutant nucleotide at the transcript level suggesting that our dual AAV9 ABE system enabled editing in the heart (FIGS. 13A-13D).

As the MYH7 p.R403Q mutation only exists in a heterozygous form in human patients, the AAV9 ABE system was deployed to prevent HCM disease onset in Myh6^{h403/+} mice. Myh6^{h403/+} P0 pups were injected intrathoracically with either saline or 1×10¹⁴ vg/kg of each AAV9 (2×10¹⁴ vg/kg total) and their littermate Myh6^{WT} control pups with saline (FIG. 12B). At 5 weeks of age, the mice were put on a chow diet of 0.1% cyclosporine A, which has previously been shown to accelerate the onset of HCM in mouse models of sarcomere mutations. Serial echocardiograms were conducted at 8, 12, and 16 weeks of age to monitor disease progression. Myh6^{h403/+} mice had increased features of HCM compared to Myh6^{WT} controls, including increased left ventricular anterior wall thickness at diastole (LVAW;d) (1.07±0.0443 mm vs. 0.883±0.0441 mm, P=0.017) and increased left ventricular posterior wall thickness (LVPW;d) (1.04±0.0809 mm vs. 0.867±0.0590 mm, P=0.128). These mice also had decreased left ventricular internal diameter at diastole (LVID;d) (2.34±0.142 mm vs. 2.81±0.0540 mm, P=0.015) and systole (LVID;s) (0.940±0.0713 mm vs. 1.24±0.0520, P=0.010), with slightly increased ejection fraction (EF) and fractional shortening (FS). The increased ventricular wall thickness and a concomitant decrease in ventricular diameter of Myh6^{h403/+} mice is consistent with the clinical progression in human patients.

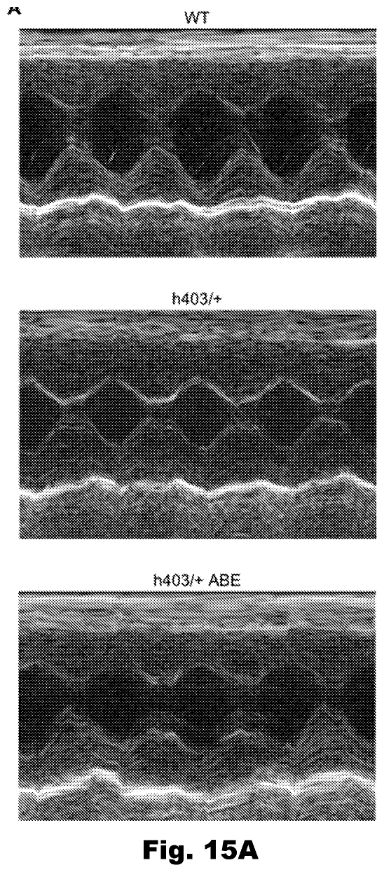

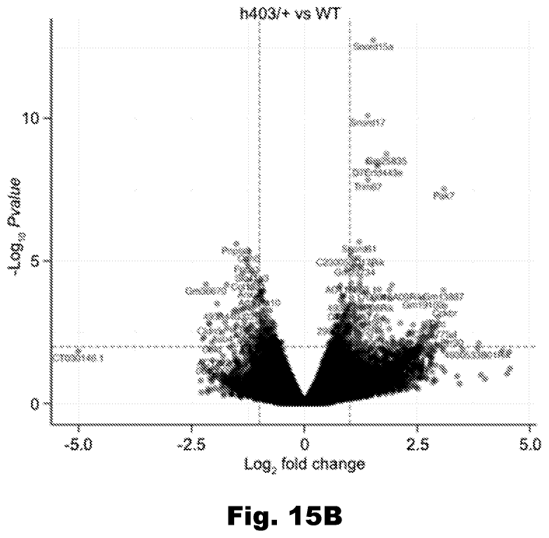

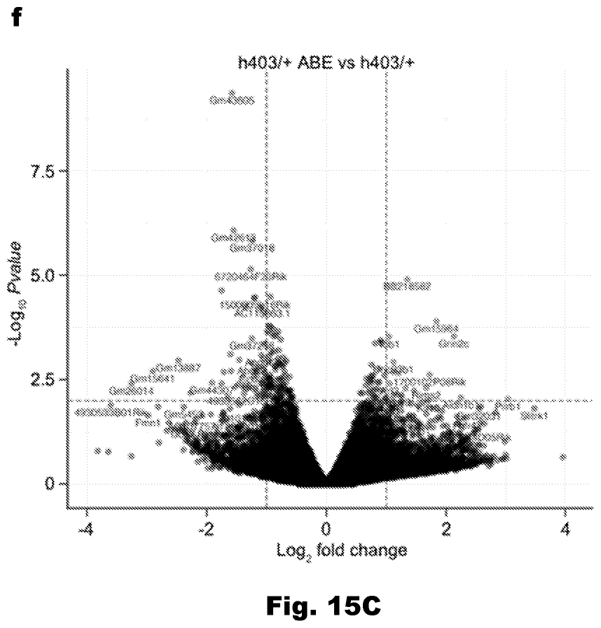

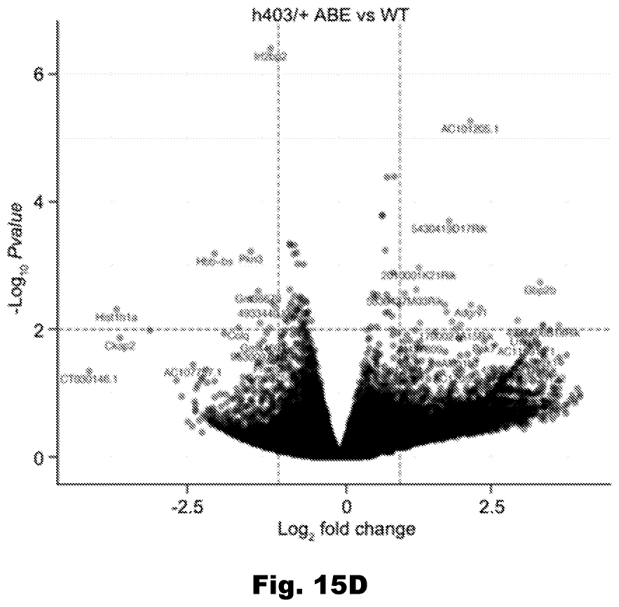

In contrast, ABE-treated Myh6^{h403/+} mice, had comparable echocardiographic measurements to Myh6^{WT} control mice, suggesting that gene correction of the pathogenic nucleotide was sufficient to prevent the onset of HCM (FIGS. 12C-12H, Table 1, FIG. 15A). Histological analysis also revealed increased cardiac wall thickness and decreased ventricular diameter in Myh6^{h403/+} mice compared to Myh6^{WT} control mice, while ABE-treated Myh6^{403/+} mice had similar cardiac dimensions to Myh6^{WT} control mice (FIGS. 12I-12K). When normalized to tibia length, Myh6^{h403/+} mice had 1.3-fold larger hearts by heart weight compared to Myh6^{WT} control mice, while ABE-treated Myh6^{h403/+} mice had no significant difference in heart weight compared to Myh6^{WT} mice (FIG. 12L). As a measure of fibrosis, hearts from Myh6^{403/+} mice had 3.0-fold more collagen area compared to Myh6^{WT} control mice, while ABE-treated Myh6^{h403/+} mice had no significant difference in collagen area compared to Myh6^{WT} mice (FIG. 12M). These data suggest that dual AAV9 ABE treatment was sufficient to prevent the onset of HCM-mediated pathological remodeling of the heart.

### Example 8—Genomic and Transcriptomic Analyses of ABE-Treated Mice

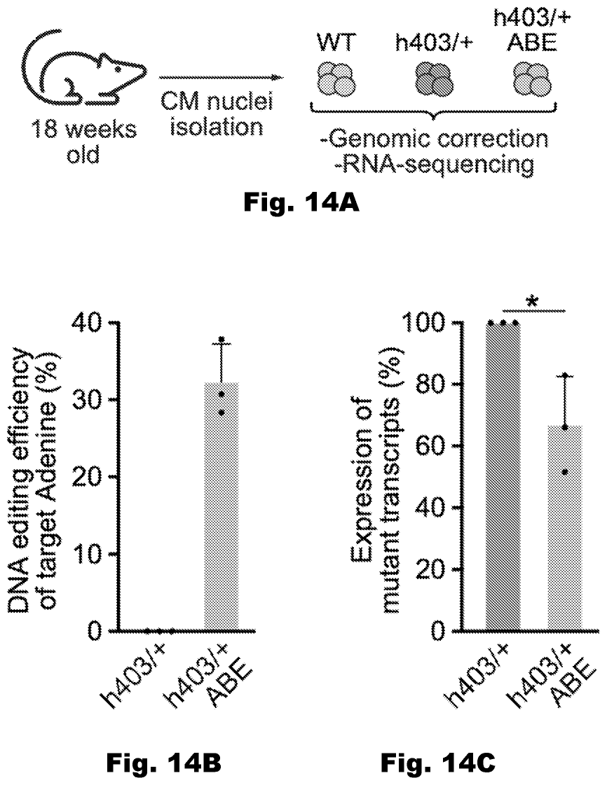

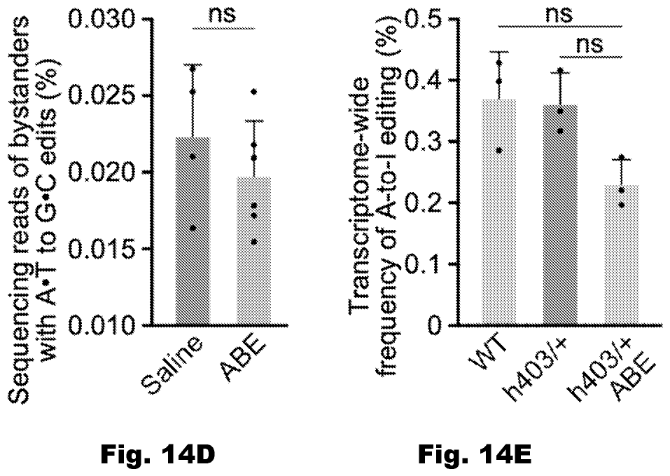

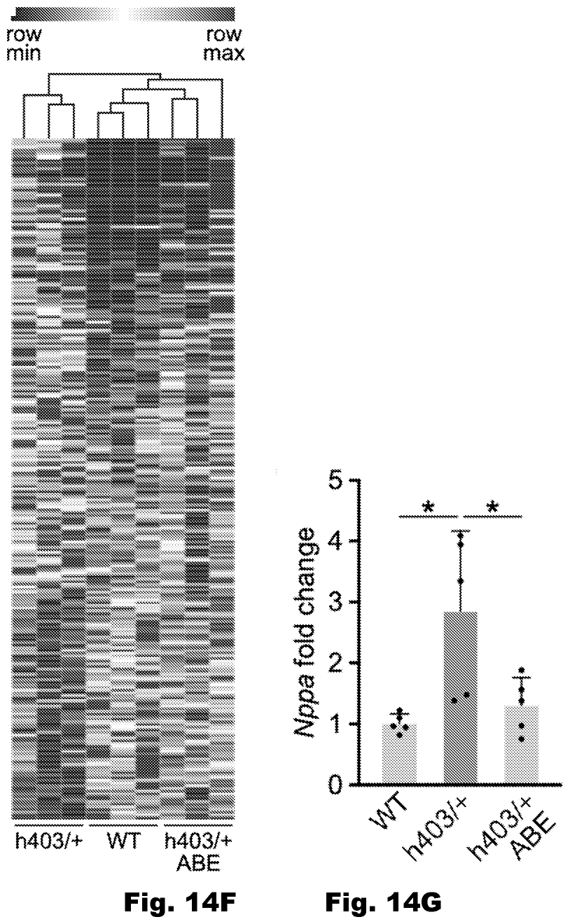

To identify genomic and transcriptomic changes following base editing, CM nuclei were isolated from saline-treated Myh6^{WT} control mice, saline-treated Myh6^{403/+} mice, and ABE-treated Myh6^{h403/+} mice (FIG. 14A). On-target editing efficiencies following dual AAV9 ABE treatment was evaluated first. In ABE-treated Myh6^{h403/+} mice, DNA editing efficiency of the target pathogenic adenine was 32.3±2.87%, resulting in a 33.1±9.08% reduction in mutant transcripts compared to Myh6^{h403/+} mice (FIGS. 14B-C), which is comparable to other in vivo studies using base editing or RNAi-based knockdown of mutant transcripts. Furthermore, there was no detectable bystander editing in ABE-treated Myh6^{h403/+} mice (FIG. 14D). Potential off-target RNA editing was then evaluated using transcriptome-wide RNA sequencing (RNA-seq), as ABEmax contains deaminase activity. RNA-seq analysis revealed no significant change in the average frequency of A-to-I editing in the transcriptome of ABE-treated mice compared to that of saline-treated mice (FIG. 14E). This finding suggests that in vivo treatment with our dual AAV9 ABE system does not increase RNA deamination above background levels of endogenous cellular deaminase activity.

Transcriptome-wide changes were evaluated in ABE-treated Myh6^{h403/+} mice via RNA-seq. 257 differentially regulated genes were identified between Myh6^{WT} mice and Myh6^{h403/+} mice. Heat maps showed that ABE-treated Myh6^{h403/+} mice had transcriptome profiles more similar to Myh6^{WT} mice than to Myh6^{h403/+} mice (FIG. 14F, FIGS. 15B-15D). Gene ontology analyses of differentially regulated genes between Myh6^{h403/+} mice and Myh6^{WT} mice indicate dysregulation of intercellular signaling and angiogenesis, while intercellular signaling was dysregulated between Myh6^{h403/+} mice and ABE-treated Myh6^{h403/+} mice (Table 17, below). Additionally, expression of the prototypic hypertrophic marker Nppa was 2.8-fold higher in Myh6^{h403/+} mice compared to Myh6^{WT} mice, while expression of Nppa in the ABE-treated Myh6^{h403/+} mice was not significantly different from Myh^{WT} mice (FIG. 14G). Taken together, these data suggest that the dual AAV9 ABE system can efficiently correct the pathogenic mutant nucleotide in genomic DNA and prevent transcriptomic dysregulation.

TABLE 17
h403/+ vs WT

| GO Terms (h403/+ up) | Log P value |
|---|---|
| Regulation of synaptic transmission, | −4.9469 |
| GABAergic | |
| Negative regulation of synaptic transmission | −3.9054 |
| Positive regulation of cell junction assembly | −3.0722 |
| Regulation of morphogenesis of an epithelium | −2.7041 |

| GO Terms (h403/+ down) | Log P value |
|---|---|
| regulation of angiogenesis | −3.9387 |
| Vasculature development | −3.6032 |
| Regulation of epithelial cell differentiation | −3.5925 |
| Enzyme linked receptor protein signaling | −3.5706 |
| pathway | |

h403/+ ABE vs h403/+

| GO Terms (h403/+ ABE up) | LogP value |
|---|---|
| Regulation of synaptic plasticity | −3.6564 |
| Regulation of membrane potential | −2.2081 |
| Response to inorganic substance | −2.1142 |

| GO Terms (h403/+ down) | Log P value |
|---|---|
| Transmembrane receptor protein tyrosine | −3.2181 |
| kinase signaling pathway | |

### Example 9—Materials and Methods

Study design and approval. The objective of this study was to determine whether base editing correction of a pathogenic HCM-causing mutation could prevent the onset of HCM pathological features in human CMs and a humanized mouse model. In human CMs, this was done by base editing correction of HCM patient-derived iPSCs and measuring changes in characteristic CM function. In a humanized mouse model, a dual AAV9 system was used to deliver the base editing components to CMs and changes in heart function, dimensions, and transcriptomics were measured. For all experiments, the number of replicates, type of replicates, and statistical test used is reported in the figure legends. For in vitro CM experiments, data are collected from three separate differentiations, and no outliers or other data points were excluded. For in vivo experiments, male mice were assigned to treatment based on genotype. Echocardiographic measurements were conducted in a blinded fashion. Runt mice with reduced body weights more than 2 standard deviations from the mean were excluded. Endpoints were guided by changes in echocardiographic measurements. Animal work described in this manuscript has been approved and conducted under the oversight of the UT Southwestern Institutional Animal Care and Use Committee.

Plasmids and vector construction The pSpCas9(BB)-2A-GFP (PX458) plasmid was a gift from Feng Zhang (Addgene plasmid #48138), and was used as the primary scaffold to clone in the following base editors and SpCas9 nickases: ABE8e, a gift from David Liu (Addgene plasmid #138489); VRQR-ABEmax, a gift from David Liu (Addgene plasmid #119811; NG-ABEmax, a gift from David Liu (Addgene plasmid #124163); pCMV-T7-SpG-HF1-P2A-EGFP (RTW5000), a gift from Benjamin Kleinstiver (Addgene plasmid #139996); and pCMV-T7-SpRY-HF1-P2A-EGFP (RTW5008), a gift from Benjamin Kleinstiver (Addgene plasmid #139997). The N-terminal ABE and C-terminal ABE constructs were adapted from Cbh_v5 AAV-ABE N terminus (Addgene plasmid #137177) and Cbh_v5 AAV-ABE C terminus (Addgene plasmid #137178) and synthesized by Twist Bioscience. PCR amplification of select plasmids was done using PrimeStar GXL Polymerase (Takara), and cloning was done using NEBuilder HiFi DNA Assembly (NEB) into restriction enzyme-digested destination vectors.

Generation of patient-derived iPSCs and isogenic mutant lines Peripheral blood mononuclear cells (PBMCs) from two patients with the MYH7 c.1208 G>A (p.R403Q) mutation were reprogrammed to iPSCs (HCM1 and HCM1) using Sendai virus. The HCM1 line was derived from a 56-year-old female with extensive family history of HCM, and nonobstructive HCM with a history of reduced left ventricular ejection fraction and low maximal oxygen uptake (VO₂ max). A biventricular pacemaker was placed for a complete heart block. The HCM2 line was derived from a 32-year-old male with a history of HCM, an implantable cardioverter-defibrillator, and a strong family history of HCM. He has a dilated left atrium but has improved VO₂ max, metabolic equivalent (METs), and no evidence of atrial fibrillation by cardiopulmonary exercise testing. PBMCs from a healthy male donor (HD) were reprogrammed to iPSCs at the UT Southwestern Wellstone Myoediting Core using Sendai virus (CytoTune 2.0 Sendai Reprogramming Kit, ThermoFisher Scientific). To generate isogenic iPSCs containing the MYH7 c.1208 G>A (p.R403Q) mutation via homology-directed repair, HD iPSCs were nucleofected using the P3 Primary Cell 4D-NucleofectorX Kit (Lonza) with a single-stranded oligodeoxynucleotide (ssODN) template (Integrated DNA Technologies, IDT) encoding for the mutation, and the PX458 plasmid encoding SpCas9-P2a-EGFP and a sgRNA targeting MYH7. For base editing correction of HCM1 and HCM2 patient derived lines, iPSCS were nucleofected with plasmid encoding for ABEmax-VRQR-P2a-EGFP and h403_sgRNA. After 48 hours, GFP+ iPSCs were collected by fluorescence-activated cell sorting, clonally expanded, and genotyped by Sanger sequencing (see Table 18 for primers used).

iPSC maintenance and differentiation iPSC culture and differentiation were performed as previously described (F. Chemello, A. C. Chai, H. Li, C. Rodriguez-Caycedo, E. Sanchez-Ortiz, A. Atmanli, A. A. Mireault, N. Liu, R. Bassel-Duby, E. N. Olson, Precise correction of Duchenne muscular dystrophy exon deletion mutations by base and prime editing. Sci Adv 7, (2021). Briefly, iPSCs were cultured on Matrigel (Corning)-coated tissue culture polystyrene plates and maintained in mTeSR1 media (STEMCELL) and passaged at 70-80% confluency using Versene. iPSCs were differentiated into CMs at 70-80% confluency by treatment with CHIR99021 (Selleckchem) in RPMI supplemented with ascorbic acid (50 μg/mL) and B27 without insulin (RPMI/B27−) for 24 hrs (from day (d) 0 to d1). At d1, media was replaced with RPMI/B27−. At d3, cells were treated with RPMI/B27− supplemented with WNT-C59 (Selleckchem). At d5, media was refreshed with RPMI/B27−. From d7 onwards, iPSC-CMs were maintained in RPMI supplemented with ascorbic acid (50 μg/mL) and B27 (RPMI/B27) with media refreshed every 3-4 days. Metabolic selection of CMs was performed for 6 days starting d10 by culturing cells in RPMI without glucose and supplemented with 5 mM sodium DL-lactate and CDM3 supplement (500 μg/mL Oryza sativa-derived recombinant human albumin, A0237, Sigma-Aldrich; and 213 μg/mL L-ascorbic acid 2-phosphate, Sigma-Aldrich). To induce their maturation, iPSC-CMs were maintained in RPMI without glucose supplemented with B27, 50 μmol palmitic acid, 100 μmol oleic acid, 10 mmol galactose, and 1 mmol glutamine (Sigma-Aldrich). All CM functional studies were done at d40-50.

Plasmid transfection and editing efficiency analysis iPSCs were seeded on a 48-well plate 24 h before transfection. At ˜20% confluency, cells were transiently transfected with 0.5 μg of plasmid encoding for a base editor and the h403_sgRNA using 1 μL of Lipofectamine Stem Transfection Reagent (Thermo Fisher) per well. Following 48 h post-transfection, cells were lysed in Direct PCR Lysis Reagent (Cell) (Viagen). PCR amplification of target sites was done using PrimeStar GXL Polymerase (Takara), and PCR cleanup was done using ExoSap-IT Express (ThermoFisher) before Sanger sequencing. Chromatograms were analyzed using EditR to determine base editing efficiencies.

Contractility analyses of iPSC-CMs iPSC-CMs were plated at single-cell density on flexible polydimethylsiloxane (PDMS) 527 substrates (Young's modulus=5 kPa) prepared according to a previously established protocol (A. Atmanli, A. C. Chai, M. Cui, Z. Wang, T. Nishiyama, R. Bassel-Duby, E. N. Olson, Cardiac Myoediting Attenuates Cardiac Abnormalities in Human and Mouse Models of Duchenne Muscular Dystrophy. Circ Res 129, 602-616 (2021)). Recordings of contracting iPSC-CMs were captured at 37° C. using a Nikon A1R+ confocal system at 59 frames per second in resonance scanning mode. Contractile force generation of iPSC-CMs was quantified using a previously established method. In brief, recordings were analyzed using Fiji to measure maximum and minimum cell lengths, and cell widths during contraction. A previously published customized Matlab code was used to calculate peak systolic forces (J. D. Kijlstra, D. Hu, N. Mittal, E. Kausel, P. van der Meer, A. Garakani, I. J. Domian, Integrated Analysis of Contractile Kinetics, Force Generation, and Electrical Activity in Single Human Stem Cell-Derived Cardiomyocytes. Stem Cell Reports 5, 1226-1238 (2015)).

Extracellular flux analyses of iPSC-CMs iPSC-CMs were plated at 40,000 cells per well in Seahorse XFe96 V3 PS Cell Culture Microplates (Agilent) coated with Matrigel. One-week post-plating, cells were washed three times with prewarmed assay media (pyruvate-free DMEM (Sigma D5030) supplemented with 2 mM L-glutamine, 1 mM sodium pyruvate, and 10 mM glucose, pH 7.4) and incubated at 37° C. for 60 min in a non-CO₂ incubator. Oxygen consumption rate (OCR) was measured in a Seahorse XFe96 instrument using consecutive cycles of 2 mins of measurement, 10 seconds of waiting, and 3 minutes of mixing. Mitochondrial stress testing was performed by injecting oligomycin (final concentration 2 μM), CCCP (final concentration 1 μM), and antimycin A (final concentration 1 μM) at indicated time intervals. Data were analyzed using the WAVE software (Agilent).

Immunofluorescence staining. iPSC-CMs were plated on glass surfaces and fixed with 4% paraformaldehyde for 10 min, followed by blocking with 5% goat serum/0.1% Tween-20 (Sigma-Aldrich) for 1 hr. Primary and secondary antibodies were diluted in blocking buffer and added to cells for 2 hr and 1 hr, respectively. Nuclei were counterstained using DAPI. Antibodies used included sarcomeric α-actinin (clone EA-53, A7811, Sigma-Aldrich, 1:600 dilution), and goat anti-mouse IgG1 Alexa 488 (A21121, Thermo-Fisher, 1:600 dilution).

Off-target analyses. Candidate off-target sites were identified with CRISPOR, and the top 8 sites by cutting frequency determination (CFD) score, for which PCR products were successfully obtained, were selected. Genomic DNA was isolated using a DNeasy Blood & Tissue Kit (Qiagen) from HCM1, HCM2 and HD cell lines that had been nucleofected with plasmids encoding for ABEmax-VRQR-P2a-EGFP and h403_sgRNA and sorted for GFP+ cells. Target sites were PCR amplified using PrimeStar GXL Polymerase (Takara), and a second round of PCR was used to add Illumina flow cell binding sequences and barcodes. PCR products were purified with AMPure XP Beads (Beckman Coulter), analyzed for integrity on a 2200 TapeStation System (Agilent), and quantified by QuBit dsDNA high-sensitivity assay (Invitrogen) before pooling and loading onto an Illumina MiSeq. Following demultiplexing, resulting reads were analyzed with CRISPResso2 for editing frequency (K. Clement, H. Rees, M. C. Canver, J. M. Gehrke, R. Farouni, J. Y. Hsu, M. A. Cole, D. R. Liu, J. K. Joung, D. E. Bauer, L. Pinello, CRISPResso2 provides accurate and rapid genome editing sequence analysis. Nat Biotechnol 37, 224-226 (2019).

Generation of adeno-associated viruses. Recombinant AAV9 (rAAV9) viruses were made at the University of Michigan Vector Core using ultracentrifugation through an iodixanol gradient. rAAV9s were washed 3 times with PBS using Amicon Ultra Centrifugal Filter Units (Millipore) and resuspended in PBS+0.001% Pluronic F68. Titers were assessed by qPCR. rAAV9 was stored in 25 μL aliquots at −80° C.

Mice. Mice were housed in a barrier facility with a 12-hour:12-hour light:dark cycle and maintained on standard chow (2916 Teklad Global). The humanized Myh6^{403/+} mutation was introduced via microinjection of zygotes with Cas9 mRNA (50 ng/μL) (TriLink Biotechnologies), a sgRNA (20 ng/μL) (IDT), and a ssODN donor template (15 ng/μL) (IDT) following a modified protocol (H. Miura, R. M. Quadros, C. B. Gurumurthy, M. Ohtsuka, Easi-CRISPR for creating knock-in and conditional knockout mouse models using long ssDNA donors. Nat Protoc 13, 195-215 (2018). Genotyping was performed using a custom TaqMan SNP Genotyping Assay (ThermoFisher). To accelerate the onset of HCM, mice were treated with a custom chow (2916 Teklad Global base) containing Cyclosporine A (Alfa Aesar) at 1 g/kg and blue food dye at 0.2 g/kg. For injections, mice were genotyped at P0 and received either saline or a AAV9 dose via a single 40 μL bolus using a 31G insulin syringe through the diaphragm by a subxiphoid approach into the inferior mediastinum, avoiding the heart and the lung.

Transthoracic echocardiography. Cardiac function on conscious mice was evaluated by two-dimensional transthoracic echocardiography using a VisualSonics Vevo2100 imaging system. M-mode tracings were used to measure LV anterior wall thickness at diastole (LVAW;d), LV posterior wall thickness at diastole (LVPW;d), and LV internal diameter at end diastole (LVIDd) and end systole (LVIDs). FS was calculated according to the following formula: FS (%)=[(LVIDd−LVIDs)/LVIDd]×100. EF was calculated according to the following formula: EF (%)=[(LVEDV−LVESV)/LVEDV]×100. All measurements were performed by an experienced operator blinded to the study.

Histology. Mouse hearts were dissected out and submerged in PBS with cardioplegic 0.2M KCl for 5 minutes before fixation in 4% paraformaldehyde in PBS overnight, followed by dehydration in 70% ethanol and paraffin embedding. Serial transverse cross-sections at 500 m intervals were cut and mounted on slides, followed by H&E staining or Masson's Trichrome staining. Images were captured on a BZ-X all-in-one microscope (Keyence) at 10× or 40× magnification.

CM nuclei isolation. For each nuclear sample, ventricular heart tissue was isolated. CM nuclei were isolated as previously described (M. Cui, E. N. Olson, Protocol for Single-Nucleus Transcriptomics of Diploid and Tetraploid Cardiomyocytes in Murine Hearts. STAR Protoc 1, 100049 (2020). Isolated nuclei were immediately used for downstream processing, or stored in Nuclei PURE Storage Buffer (Sigma Aldrich) at −80° C. For RNA-seq and qPCR, RNA was isolated from nuclei using the RNeasy Micro Kit (Qiagen). For DNA sequencing, nuclei were lysed in Direct PCR Lysis Reagent (Cell) (Viagen).

RNA-seq library preparation, sequencing, and analysis. RNA-seq libraries were generated using the SMARTer Stranded Total RNA-Seq Kit v2-Pico Input Mammalian kit (Takara), containing Illumina sequencing adapters. Libraries were visualized on a 2200 TapeStation System (Agilent) and quantified by QuBit dsDNA high-sensitivity assay (Invitrogen) before pooling and loading onto an Illumina NextSeq 500. FastQC tool (Version 0.11.8) was used for quality control of RNA-seq data to determine low quality or adaptor portions of the reads for trimming. Read trimming was performed using Trimmomatic (Version 0.39) and strandness was determined using RSeQC (Version 4.0.0) and then reads were aligned to the mm10 reference genome using HiSAT2 (Version 2.1.0) with default settings and -rna-strandness R. Aligned reads were counted using featureCounts (Version 1.6.2). Differential gene expression analysis was performed using R package DESeq (Version 1.38.0). Genes with fold-change >2 and p-value <0.01 were designated as DEGs between sample group comparisons. To calculate the average percentage of A-to-I editing amongst adenosines sequenced in transcriptome-wide sequencing analysis, we adopted a previous strategy (L. W. Koblan, M. R. Erdos, C. Wilson, W. A. Cabral, J. M. Levy, Z. M. Xiong, U. L. Tavarez, L. M. Davison, Y. G. Gete, X. Mao, G. A. Newby, S. P. Doherty, N. Narisu, Q. Sheng, C. Krilow, C. Y. Lin, L. B. Gordon, K. Cao, F. S. Collins, J. D. Brown, D. R. Liu, In vivo base editing rescues Hutchinson-Gilford progeria syndrome in mice. Nature 589, 608-614 (2021). In brief, REDItools2 was used to quantify the percentage editing in each sample. Nucleotides except adenosines were removed and remaining adenosines with read coverage less than 10 or read quality score below 25 were also filtered to avoid errors due to low sampling or low sequencing quality. We then calculated the number of A-to-I conversion in each sample and divided this by the total number of adenosines in our dataset after filtering to get the percentage of A-to-I editing in the transcriptome.

Quantitative real-time PCR analysis. Quantitative Polymerase Chain Reaction (qPCR) reactions were assembled using Applied Biosystems TaqMan Fast Advanced Master Mix (Applied Biosystems). Assays were performed using Applied Biosystems QuantStudio 5 Real-Time PCR System (Applied Biosystems). Expression values were normalized to 18S mRNA and represented as fold change.

Statistics. All data are presented as means±s.e.m. or means±s.d. as indicated. Unpaired two-tailed Student's t tests were performed for comparison between the respective two groups as indicated in the figures. Kaplan-Meier analysis and Log-rank (Mantel-Cox) test were used to evaluate the difference in survival between different genotypes. Data analyses were performed with statistical software (GraphPad Prism Software). P values less than 0.05 were considered statistically significant.

Oligos/primers and other nucleic acids used in the methods above are provided in Table 18 below.

TABLE 18
Summary of Oligos

| Oligo | | SEQ ID |
|---|---|---|
| Name | Oligo Sequence | NO: |
| sgRNA for | TCATTGCCCACTTTCACCCG | 113 |
| HDR | | |
| Knock-In | | |
| of MYH7 | | |
| R403Q | | |
| ssODN for | TGCTACTTGCCTTTTCCTTCCAGAGGCTGACAAGTCT | 114 |
| HDR | GCCTACCTCATGGGGCTGAACTCAGCCGACCTGCTC | |
| Knock-In | AAGGGGCTGTGCCACCCTCAGGTGAAAGTGGGCAAT | |
| of MYH7 | GAGTACGTCACCAAGGGGCAG | |
| R403Q | | |
| Sequencing | ACCTCCACATCCTGGGTTCAA | 115 |
| for | | |
| hMYH7 F | | |
| Sequencing | GTGGAGGAGAGACCCATATT | 116 |
| for | | |
| hMYH7 R | | |
| Sequencing | ggaggctgtagtgagccaag | 117 |
| for | | |
| hMYH6 F | | |
| Sequencing | aggaGCAAGCGAGTGATTGT | 118 |
| for | | |
| hMYH6 R | | |
| h403_ | CCGCAGGTGAAAGTGGGCAA | 119 |
| sgRNA | | |
| HTS ON- | TCGTCGGCAGCGTCAGATGTGTATAAGAGACAGTCCT | 120 |
| Target F | CTCATACACTGCCTTGG | |
| HTS ON- | GTCTCGTGGGCTCGGAGATGTGTATAAGAGACAGCA | 121 |
| Target R | CCATGCCTGGCTAATTTT | |
| HTS | TCGTCGGCAGCGTCAGATGTGTATAAGAGACAGGGG | 122 |
| OFF1 F | ACAATGACTGCCTCTGT | |
| HTS | GTCTCGTGGGCTCGGAGATGTGTATAAGAGACAGTAC | 123 |
| OFF1 R | CTCATGGGGCTGAACTC | |
| HTS | TCGTCGGCAGCGTCAGATGTGTATAAGAGACAGCAG | 124 |
| OFF2 F | GTCTCGATTCCAAGGAG | |
| HTS | GTCTCGTGGGCTCGGAGATGTGTATAAGAGACAGGC | 125 |
| OFF2 R | ACAACCCACAAGTTTGTTT | |
| HTS | TCGTCGGCAGCGTCAGATGTGTATAAGAGACAGTTTT | 126 |
| OFF3 F | CAAAATATTCCTGCTCACT | |
| HTS | GTCTCGTGGGCTCGGAGATGTGTATAAGAGACAGAG | 127 |
| OFF3 R | GCACCTTTCTGTGTGCTT | |
| HTS | TCGTCGGCAGCGTCAGATGTGTATAAGAGACAGATTC | 128 |
| OFF4 F | TGGATGCAGGATTTGC | |
| HTS | GTCTCGTGGGCTCGGAGATGTGTATAAGAGACAGGT | 129 |
| OFF4 R | GGACAACAGGCCACTCTT | |
| HTS | TCGTCGGCAGCGTCAGATGTGTATAAGAGACAGGGA | 130 |
| OFF5 F | CAATTTGTATTTTAGCTTATTTTC | |
| HTS | GTCTCGTGGGCTCGGAGATGTGTATAAGAGACAGTCC | 131 |
| OFF5 R | CCTGCTTTTCTCTGTGT | |
| HTS | TCGTCGGCAGCGTCAGATGTGTATAAGAGACAGTGAT | 132 |
| OFF6 F | CCTGAAGATTAGTGGATGC | |
| HTS | GTCTCGTGGGCTCGGAGATGTGTATAAGAGACAGCC | 133 |
| OFF6 R | ATCCTGAGATAATCCTCCA | |
| HTS | TCGTCGGCAGCGTCAGATGTGTATAAGAGACAGACCT | 134 |
| OFF7 F | AGGAGGCTGGGATTGT | |
| HTS | GTCTCGTGGGCTCGGAGATGTGTATAAGAGACAGCAT | 135 |
| OFF7 R | GACAAGGAGTCCGAGGT | |
| HTS | TCGTCGGCAGCGTCAGATGTGTATAAGAGACAGGCC | 136 |
| OFF8 F | CCTGGTTACAGCATAAG | |
| HTS | GTCTCGTGGGCTCGGAGATGTGTATAAGAGACAGCC | 137 |
| OFF8 R | ACAACCACTGACTGACTGA | |
| sgRNA for | TCGTTCCCCACCTTCACCCG | 138 |
| Knock-In | | |
| of MYH7 | | |
| R403Q | | |
| into | | |
| murine | | |
| Myh6 | | |
| ssODN for | TGGGACAAAGGAATGGAGGTACTGAAAATGCTTCCCC | 92 |
| Knock-In | TCTCCTTGTCTATCAGATGCTGACAAATCAGCCTACCT | |
| of MYH7 | CATGGGGCTGAACTCAGCCGACCTGCTCAAGGGGCT | |
| R403Q | GTGCCACCCTCAGGTGAAAGTGGGCAATGAGTACGT | |
| into | CACCAAGGGGCAGAGTGTACAGCAAGTGTACTAT | |
| murine | | |
| Myh6 | | |
| Genotyping | GAGAAGCAGTGGTCATCATC | 139 |
| for Myh6 | | |
| F | | |
| Genotyping | GTGAGAAACACGTGGTGTCC | 140 |
| for Myh6 | | |
| R | | |
| HTS Myh6 | TCGTCGGCAGCGTCAGATGTGTATAAGAGACAGGGAT | 141 |
| On-Target | CAAGGACATGGCAAAT | |
| F | | |
| HTS Myh6 | GTCTCGTGGGCTCGGAGATGTGTATAAGAGACAGGC | 142 |
| On-Target | TTGGTCTCCAGGGTTG | |
| R | | |
| HTS Myh6 | TCGTCGGCAGCGTCAGATGTGTATAAGAGACAGGATG | 143 |
| cDNA On- | GCACAGAAGATGCTGA | |
| Target F | | |
| HTS Myh6 | GTCTCGTGGGCTCGGAGATGTGTATAAGAGACAGCG | 144 |
| cDNA On- | AACATGTGGTGGTTGAAG | |
| Target R | | |
| Sanger | GCTCTTGGCCACTGATAGTGC | 145 |
| Myh6 | | |
| cDNA On- | | |
| Target F | | |
| Sanger | GCTCAAAGCTGTTGAAATCG | 146 |
| Myh6 | | |
| cDNA On- | | |
| Target R | | |

1. A gRNA comprising a spacer sequence corresponding to a DNA nucleotide sequence of SEQ ID NO: 1 or 2.

2. The gRNA of claim 1, wherein the gRNA comprises a spacer sequence having at least 85%, at least 90%, at least 95%, at least 96%, at least 97%, at least 98%, at least 99% or 100% sequence identity to SEQ ID NO: 5 or 6.

3. (canceled)

4. A fusion protein comprising a deaminase covalently linked to a Cas9 nickase or deactivated Cas9 endonuclease.

5. The fusion protein of claim 4 wherein the deaminase is selected from the group consisting of ABEmax, ABE8e, ABE7.10 and any functional variant thereof.

6. The fusion protein of claim 5, wherein the deaminase comprises an amino acid sequence having at least 85%, at least 90%, at least 95%, at least 96%, at least 97%, at least 98%, at least 99% sequence homology to any one of SEQ ID NOS 7, 9 or 11.

7-8. (canceled)

9. The fusion protein of claim 1, wherein the Cas9 nickase or deactivated Cas9 endonuclease is selected from SpRY, SpG, SpCas9-NG, SpCas9-VRQR or a variant thereof.

10. The fusion protein of claim 9, wherein the Cas9 nickase or deactivated Cas9 endonuclease comprises an amino acid sequence having at least 85%, at least 90%, at least 95%, at least 96%, at least 97%, at least 98%, at least 99% sequence homology with any one of SEQ ID NO 15, 17, 19, or 21.

11-14. (canceled)

15. The fusion protein of claim 4, wherein the deaminase and/or the Cas9 nickase or deactivated Cas9 endonuclease further comprises a nuclear localization signal (NLS) peptide.

16. The fusion protein of claim 15, wherein the nuclear localization signal (NLS) peptide is selected from any one of SEQ ID NOS: 31-42.

17. (canceled)

18. The fusion protein of claim 4, wherein the fusion protein comprises an amino acid sequence having at least 85%, at least 90%, at least 95%, at least 96%, at least 97%, at least 98%, or at least 99% sequence homology to any one of SEQ ID NOS: 45-60.

19-20. (canceled)

21. An isolated nucleic acid encoding the gRNA of claim 1.

22. An isolated nucleic acid encoding the fusion protein of claim 4 or a fragment thereof.

23. A viral vector comprising the nucleic acid of claim 21.

24. A pair of viral vectors, comprising:
(a) a first viral vector comprising a nucleic acid encoding a first fragment of the fusion protein of claim 4; and
(b) a second viral vector encoding a second fragment of the fusion protein of claim 4, wherein the first fragment of the fusion protein of claim 4 and the second fragment of the fusion protein of claim 4 can undergo protein trans-splicing to form the fusion protein of claim 4.

25. The pair of viral vectors of claim 24, wherein the first and/or second viral vector further comprise a nucleic acid encoding a gRNA comprising a spacer sequence corresponding to a DNA nucleotide sequence of SEQ ID NO:1 or 2.

26. A pharmaceutical composition comprising a nucleic acid of claim 21, and a pharmaceutically acceptable carrier, diluent and/or excipient.

27. (canceled)

28. A method of correcting a mutation in an MYH7 gene in a cell, the method comprising delivering to the cell: a Cas9 nickase or deactivated Cas9 endonuclease, a deaminase, and a gRNA targeting a DNA nucleotide sequence selected from any one of SEQ ID NOS: 1 or 2, or one or more nucleic acids encoding the Cas9 nickase or deactivated Cas9 endonuclease, deaminase and/or gRNA, to effect one or more single-strand breaks (SSBs) within or near the MYH7 gene that results in one or more mutations of at least one nucleotide within or near the MYH7 gene, thereby correcting the mutation in the MYH7 gene.

29-31. (canceled)

32. A method of treating a cardiomyopathy caused by a mutation in an MYH7 gene in a subject in need thereof, the method comprising delivering to at least one cell in the subject expressing the MYH7 gene: an RNA guided nickase, a deaminase, and a gRNA targeting a DNA nucleotide sequence selected from any one of SEQ ID NOS: 1 or 2, or one or more nucleic acids encoding the RNA guided nickase, deaminase and/or gRNA, a to effect one or more single-strand breaks (SSBs) within or near the MYH7 gene that results in one or more mutations of at least one nucleotide within or near the MYH7 gene, thereby correcting the mutation in the MYH7 gene in at least one cell of the subject.

33. (canceled)

34. The method of claim 32, wherein the mutation in the MYH7 gene comprises one or more single nucleotide polymorphisms that result in a single amino acid substitution in a protein product encoded by the mutated MYH7 gene.

35. The method of claim 34, wherein the protein product is a myosin protein or peptide and the single amino substitution comprises R403Q according to SEQ ID NO: 96.

36. A gene edited mouse comprising a human nucleic acid comprising a MYH7 c.1208 G>A (p.R403Q) human missense mutation inserted within an endogenous murine Myh6 gene to form a humanized mutant Myh6 allele.

37. The gene edited mouse of claim 36, wherein the human nucleic acid further comprises a first polynucleotide adjacent to and upstream of the missense mutation and a second polynucleotide adjacent to and downstream of the missense mutation.

38. The gene edited mouse of claim 37, wherein the first polynucleotide comprises about 30 to 75 nucleotides, about 35 to about 70 nucleotides, about 40 to about 65 nucleotides, or about 45 to about 60 nucleotides, or about 55 nucleotides, or the second polynucleotide comprises about 10 to 30 nucleotides, about 15 to 25 nucleotides, or about 20 to 25 nucleotides, or about 21 nucleotides.

39-41. (canceled)

42. The gene edited mouse of claim 36, wherein the human nucleic acid comprises a nucleotide sequence of SEQ ID NO: 97.

43. The gene edited mouse of claim 36, wherein at least one cell of the mouse expresses a mutant myosin protein comprising a R404Q substitution relative to a wildtype myosin protein comprising SEQ ID NO: 94.

44. The gene edited mouse of claim 36, wherein the mouse further comprises a wildtype Myh6 allele and the mouse is heterozygous for the humanized mutant Myh6 allele.


SEQUENCE LISTING (166 sequences)

SEQ ID NO: 1 - DNA, 20 bp, synthetic construct
  source 1..20: mol_type = other DNA
cctcaggtgaaagtgggcaa

SEQ ID NO: 2 - DNA, 20 bp, synthetic construct
  source 1..20: mol_type = other DNA
cctcaggtgaaggtggggaa

SEQ ID NO: 3 - RNA, 51 nt, synthetic construct
  source 1..51: mol_type = other RNA
tgtcaccctcaggtgaaggtggggaacgagtatgtcaccaaggggcagagt

SEQ ID NO: 4 - RNA, 51 nt, synthetic construct
  source 1..51: mol_type = other RNA
tgccaccctcaggtgaaagtgggcaatgagtacgtcaccaaggggcagaat

SEQ ID NO: 5 - RNA, 20 nt, synthetic construct
  source 1..20: mol_type = other RNA
cctcaggtgaaagtgggcaa

SEQ ID NO: 6 - RNA, 20 nt, synthetic construct
  source 1..20: mol_type = other RNA
cctcaggtgaaggtggggaa

SEQ ID NO: 7 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 8 - protein, 383 aa, synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 9 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 10 - protein, 185 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 11 - protein, 365 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 12 - DNA, 1149 bp, synthetic construct
  source 1..1149: mol_type = other DNA
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgac

SEQ ID NO: 13 - DNA, 555 bp, synthetic construct
  source 1..555: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 14 - DNA, 1095 bp, synthetic construct
  source 1..1095: mol_type = other DNA
atgtccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttctggtggttcttctggtggttcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgac

SEQ ID NO: 15 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 16 - protein, 1389 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSKRTADGSEFEPKKKRKV

SEQ ID NO: 17 - protein, 1368 aa, synthetic construct
MDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 18 - protein, 1389 aa, synthetic construct
MDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 19 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 20 - protein, 1389 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSKRTADGSEFEPKKKRKV

SEQ ID NO: 21 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 22 - protein, 1389 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKV

SEQ ID NO: 23 - DNA, 4167 bp, synthetic construct
  source 1..4167: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 24 - DNA, 4167 bp, synthetic construct
  source 1..4167: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 25 - DNA, 4167 bp, synthetic construct
  source 1..4167: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 26 - DNA, 4167 bp, synthetic construct
  source 1..4167: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 27 - protein, 32 aa, synthetic construct
SGGSSGGSSGSETPGTSESATPESSGGSSGGS

SEQ ID NO: 28 - DNA, 1149 bp, synthetic construct
  source 1..1149: mol_type = other DNA
atgaaacggacagccgacggaagcgagttcgagtcaccaaagaagaagcggaaagtctccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttctggtggttcttctggtggttcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgac

SEQ ID NO: 29 - DNA, 96 bp, synthetic construct
  source 1..96: mol_type = other DNA
tccggaggatctagcggaggctcctctggctctgagacacctggcacaagcgagagcgcaacacctgaaagcagcgggggcagcagcggggggtca

SEQ ID NO: 30 - DNA, 96 bp, synthetic construct
  source 1..96: mol_type = other DNA
tctggtggttcttctggtggttctagcggcagcgagactcccgggacctcagagtccgccacacccgaaagttctggtggttcttctggtggttct

SEQ ID NO: 31 - protein, 19 aa, synthetic construct
MKRTADGSEFESPKKKRKV

SEQ ID NO: 32 - protein, 17 aa, synthetic construct
KRTADGSEFEPKKKRKV

SEQ ID NO: 33 - protein, 41 aa, synthetic construct
RMRKFKNKGKDTAELRRRRVEVSVELRKAKKDEQILKRRNV

SEQ ID NO: 34 - protein, 8 aa, synthetic construct
VSRKRPRP

SEQ ID NO: 35 - protein, 8 aa, synthetic construct
PPKKARED

SEQ ID NO: 36 - protein, 12 aa, synthetic construct
SALIKKKKKMAP

SEQ ID NO: 37 - protein, 5 aa, synthetic construct
DRLRR

SEQ ID NO: 38 - protein, 7 aa, synthetic construct
PKQKKRK

SEQ ID NO: 39 - protein, 10 aa, synthetic construct
RKLKKKIKKL

SEQ ID NO: 40 - protein, 10 aa, synthetic construct
REKKKFLKRR

SEQ ID NO: 41 - protein, 20 aa, synthetic construct
KRKGDEVDGVDEVAKKKSKK

SEQ ID NO: 42 - protein, 17 aa, synthetic construct
RKCLQAGMNLEARKTKK

SEQ ID NO: 43 - DNA, 57 bp, synthetic construct
  source 1..57: mol_type = other DNA
atgaaacggacagccgacggaagcgagttcgagtcaccaaagaagaagcggaaagtc

SEQ ID NO: 44 - DNA, 53 bp, synthetic construct
  source 1..53: mol_type = other DNA
aaagaacagcagacgggagtgagtttgagcccaagaaaaagagaaaggtgtaa

SEQ ID NO: 45 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 46 - protein, 1803 aa, synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSKRTADGSEFEPKKKRKV

SEQ ID NO: 47 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 48 - protein, 1803 aa, synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKV

SEQ ID NO: 49 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 50 - protein, 1803 aa, synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 51 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 52 - protein, 1803 aa, synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSKRTADGSEFEPKKKRKV

SEQ ID NO: 53 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 54 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSKRTADGSEFEPKKKRKV

SEQ ID NO: 55 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 56 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKV

SEQ ID NO: 57 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 58 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 59 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 60 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSKRTADGSEFEPKKKRKV

SEQ ID NO: 61 - DNA, 5409 bp, synthetic construct
  source 1..5409: mol_type = other DNA
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 62 - DNA, 5409 bp, synthetic construct
  source 1..5409: mol_type = other DNA
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 63 - DNA, 5412 bp, synthetic construct
  source 1..5412: mol_type = other DNA
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 64 - DNA, 5409 bp, synthetic construct
  source 1..5409: mol_type = other DNA
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 65 - DNA, 4815 bp, synthetic construct
  source 1..4815: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 66 - DNA, 4815 bp, synthetic construct
  source 1..4815: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 67 - DNA, 4815 bp, synthetic construct
  source 1..4815: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 68 - DNA, 4815 bp, synthetic construct
  source 1..4815: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 69 - protein, 1110 aa, synthetic construct
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNSGGSKRTADGSEFEPKKKRKV

SEQ ID NO: 70 - protein, 870 aa, synthetic construct
MKRTADGSEFESPKKKRKVIKIATRKYLGKQNVYDIGVERDHNFALKNGFIASNCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSKRTADGSEFEPKKKRKV

SEQ ID NO: 71 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 72 - DNA, 414 bp, synthetic construct
  source 1..414: mol_type = other DNA
cgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 73 - DNA, 5289 bp, synthetic construct
  source 1..5289: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 74 - DNA, 1092 bp, synthetic construct
  source 1..1092: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgac

SEQ ID NO: 75 - DNA, 498 bp, synthetic construct
  source 1..498: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 76 - DNA, 1716 bp, synthetic construct
  source 1..1716: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgag

SEQ ID NO: 77 - DNA, 255 bp, synthetic construct
  source 1..255: mol_type = other DNA
tgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgac

SEQ ID NO: 78 - DNA, 64 bp, synthetic construct
  source 1..64: mol_type = other DNA
gagattttcgagcgggagctggacctgatgagagtggataacctgcctaatagcggaggcagta

SEQ ID NO: 79 - DNA, 5289 bp, synthetic construct
  source 1..5289: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgac

SEQ ID NO: 80 - DNA, 249 bp, synthetic construct
  source 1..249: mol_type = other DNA
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtg

SEQ ID NO: 81 - DNA, 225 bp, unidentified
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 82 - DNA, 250 bp, synthetic construct
  source 1..250: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 83 - DNA, 4104 bp, synthetic construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 84 - DNA, 86 bp, synthetic construct
  source 1..86: mol_type = other DNA
gcttaagagctatgctggaaacagcatagcaagtttaagtaaggctagtccgttatcaacttgaaaaagtggcaccgagtcggtgc

SEQ ID NO: 85 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 86 - DNA, 4997 bp, synthetic construct
  source 1..4997: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagattaattaagaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggaaccggtgccaccatgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgacgagattttcgagcgggagctggacctgatgagagtggataacctgcctaatagcggaggcagtaaaagaacagcagacgggagtgagtttgagcccaagaaaaagagaaaggtgtaagatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctcaggtgaaagtgggcaagcttaagagctatgctggaaacagcatagcaagtttaagtaaggctagtccgttatcaacttgaaaaagtggcaccgagtcggtgctttttttgcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 87 - DNA, 105 bp, synthetic construct
  source 1..105: mol_type = other DNA
atcaagattgctacacggaaatacctgggaaagcagaacgtgtacgacatcggcgtggagcgggatcacaacttcgccctgaagaatggctttatcgccagcaat

SEQ ID NO: 88 - DNA, 2385 bp, synthetic construct
  source 1..2385: mol_type = other DNA
tgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgataacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 89 - DNA, 12 bp, synthetic construct
  source 1..12: mol_type = other DNA
tctggcggctca

SEQ ID NO: 90 - DNA, 52 bp, synthetic construct
  source 1..52: mol_type = other DNA
aaaagaaccgccgacggcagcgaattcgagcccaagaagaagaggaaagtct

SEQ ID NO: 91 - DNA, 4276 bp, synthetic construct
  source 1..4276: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagattaattaagaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggaaccggtgccaccatgaaacggacagccgacggaagcgagttcgagtcaccaaagaagaagcggaaagtcatcaagattgctacacggaaatacctgggaaagcagaacgtgtacgacatcggcgtggagcgggatcacaacttcgccctgaagaatggctttatcgccagcaattgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgataacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaagatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctcaggtgaaagtgggcaagcttaagagctatgctggaaacagcatagcaagtttaagtaaggctagtccgttatcaacttgaaaaagtggcaccgagtcggtgctttttttgcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 92 - DNA, 181 bp, synthetic construct
  source 1..181: mol_type = other DNA
tgggacaaaggaatggaggtactgaaaatgcttcccctctccttgtctatcagatgctgacaaatcagcctacctcatggggctgaactcagccgacctgctcaaggggctgtgccaccctcaggtgaaagtgggcaatgagtacgtcaccaaggggcagagtgtacagcaagtgtactat

SEQ ID NO: 93 - DNA, 100 bp, synthetic construct
  source 1..100: mol_type = other DNA
  misc_RNA 1: note = phosphorothioated 2'-O-methyl RNA bases
  misc_RNA 2: note = phosphorothioated 2'-O-methyl RNA bases
  misc_RNA 3: note = phosphorothioated 2'-O-methyl RNA bases
  misc_RNA 99: note = phosphorothioated 2'-O-methyl RNA bases
  misc_RNA 98: note = phosphorothioated 2'-O-methyl RNA bases
  misc_RNA 100: note = phosphorothioated 2'-O-methyl RNA bases
tcgttccccaccttcacccggttttagagctagaaatagcaagttaaaataaggctagtccgttatcaacttgaaaaagtggcaccgagtcggtgctttt

SEQ ID NO: 94 - DNA, 4245 bp, Streptococcus pyogenes
  source 1..4245: mol_type = other DNA
atggcccccaagaagaagcggaaggtgggcatccacggcgtgcccgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctga

SEQ ID NO: 95 - protein, 1938 aa, Mus musculus
MTDAQMADFGAAAQYLRKSEKERLEAQTRPFDIRTECFVPDDKEEYVKAKVVSREGGKVTAETENGKTVTIKEDQVMQQNPPKFDKIEDMAMLTFLHEPAVLYNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRSKKENPNANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELLATDSAFDVLSFTAEEKAGVYKLTGAIMHYGNMKFKQKQREEQAEPDGTEDADKSAYLMGLNSADLLKGLCHPRVKVGNEYVTKGQSVQQVYYSIGALAKSVYEKMFNWMVTRINATLETKQPRQYFIGVLDIAGFEIFDFNSFEQLCINFTNEKLQQFFNHHMFVLEQEEYKKEGIEWEFIDFGMDLQACIDLIEKPMGIMSILEEECMFPKASDMTFKAKLYDNHLGKSNNFQKPRNVKGKQEAHFSLVHYAGTVDYNIMGWLEKNKDPLNETVVGLYQKSSLKLMATLFSTYASADTGDSGKGKGGKKKGSSFQTVSALHRENLNKLMTNLKTTHPHFVRCIIPNERKAPGVMDNPLVMHQLRCNGVLEGIRICRKGFPNRILYGDFRQRYRILNPAAIPEGQFIDSRKGAEKLLGSLDIDHNQYKFGHTKVFFKAGLLGLLEEMRDERLSRIITRIQAQARGQLMRIEFKKIVERRDALLVIQWNIRAFMGVKNWPWMKLYFKIKPLLKSAETEKEMANMKEEFGRVKDALEKSEARRKELEEKMVSLLQEKNDLQLQVQAEQDNLNDAEERCDQLIKNKIQLEAKVKEMTERLEDEEEMNAELTAKKRKLEDECSELKKDIDDLELTLAKVEKEKHATENKVKNLTEEMAGLDEIIAKLTKEKKALQEAHQQALDDLQAEEDKVNTLTKSKVKLEQQVDDLEGSLEQEKKVRMDLERAKRKLEGDLKLTQESIMDLENDKLQLEEKLKKKEFDISQQNSKIEDEQALALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSSRHDCDLLREQYEEEMEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKIRKQLEVEKLELQSALEEAEASLEHEEGKILRAQLEFNQIKAEIERKLAEKDEEMEQAKRNHLRMVDSLQTSLDAETRSRNEALRVKKKMEGDLNEMEIQLSQANRIASEAQKHLKNSQAHLKDTQLQLDDAVHANDDLKENIAIVERRNNLLQAELEELRAVVEQTERSRKLAEQELIETSERVQLLHSQNTSLINQKKKMESDLTQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDKKNLMRLQDLVDKLQLKVKAYKRQAEEAEEQANTNLSKFRKVQHELDEAEERADIAESQVNKLRAKSRDIGAKKMHDEE

SEQ ID NO: 96 - protein, 1938 aa, synthetic construct
MTDAQMADFGAAAQYLRKSEKERLEAQTRPFDIRTECFVPDDKEEYVKAKVVSREGGKVTAETENGKTVTIKEDQVMQQNPPKFDKIEDMAMLTFLHEPAVLYNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRSKKENPNANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELLATDSAFDVLSFTAEEKAGVYKLTGAIMHYGNMKFKQKQREEQAEPDGTEDADKSAYLMGLNSADLLKGLCHPQVKVGNEYVTKGQSVQQVYYSIGALAKSVYEKMFNWMVTRINATLETKQPRQYFIGVLDIAGFEIFDFNSFEQLCINFTNEKLQQFFNHHMFVLEQEEYKKEGIEWEFIDFGMDLQACIDLIEKPMGIMSILEEECMFPKASDMTFKAKLYDNHLGKSNNFQKPRNVKGKQEAHFSLVHYAGTVDYNIMGWLEKNKDPLNETVVGLYQKSSLKLMATLFSTYASADTGDSGKGKGGKKKGSSFQTVSALHRENLNKLMTNLKTTHPHFVRCIIPNERKAPGVMDNPLVMHQLRCNGVLEGIRICRKGFPNRILYGDFRQRYRILNPAAIPEGQFIDSRKGAEKLLGSLDIDHNQYKFGHTKVFFKAGLLGLLEEMRDERLSRIITRIQAQARGQLMRIEFKKIVERRDALLVIQWNIRAFMGVKNWPWMKLYFKIKPLLKSAETEKEMANMKEEFGRVKDALEKSEARRKELEEKMVSLLQEKNDLQLQVQAEQDNLNDAEERCDQLIKNKIQLEAKVKEMTERLEDEEEMNAELTAKKRKLEDECSELKKDIDDLELTLAKVEKEKHATENKVKNLTEEMAGLDEIIAKLTKEKKALQEAHQQALDDLQAEEDKVNTLTKSKVKLEQQVDDLEGSLEQEKKVRMDLERAKRKLEGDLKLTQESIMDLENDKLQLEEKLKKKEFDISQQNSKIEDEQALALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSSRHDCDLLREQYEEEMEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKIRKQLEVEKLELQSALEEAEASLEHEEGKILRAQLEFNQIKAEIERKLAEKDEEMEQAKRNHLRMVDSLQTSLDAETRSRNEALRVKKKMEGDLNEMEIQLSQANRIASEAQKHLKNSQAHLKDTQLQLDDAVHANDDLKENIAIVERRNNLLQAELEELRAVVEQTERSRKLAEQELIETSERVQLLHSQNTSLINQKKKMESDLTQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDKKNLMRLQDLVDKLQLKVKAYKRQAEEAEEQANTNLSKFRKVQHELDEAEERADIAESQVNKLRAKSRDIGAKKMHDEE

SEQ ID NO: 97 - protein, 1935 aa, Homo sapiens
MGDSEMAVFGAAAPYLRKSEKERLEAQTRPFDLKKDVFVPDDKQEFVKAKIVSREGGKVTAETEYGKTVTVKEDQVMQQNPPKFDKIEDMAMLTFLHEPAVLYNLKDRYGSWMIYTYSGLFCVTVNPYKWLPVYTPEVVAAYRGKKRSEAPPHIFSISDNAYQYMLTDRENQSILITGESGAGKTVNTKRVIQYFAVIAAIGDRSKKDQSPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPRVKVGNEYVTKGQNVQQVIYATGALAKAVYERMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLFDNHLGKSANFQKPRNIKGKPEAHFSLIHYAGIVDYNIIGWLQKNKDPLNETVVGLYQKSSLKLLSTLFANYAGADAPIEKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLSSLDIDHNQYKFGHTKVFFKAGLLGLLEEMRDERLSRIITRIQAQSRGVLARMEYKKLLERRDSLLVIQWNIRAFMGVKNWPWMKLYFKIKPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQEAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKMELQSALEEAEASLEHEEGKILRAQLEFNQIKAEIERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

SEQ ID NO: 98 - DNA, 79 bp, synthetic construct
  source 1..79: mol_type = other DNA
tgcctacctcatggggctgaactcagccgacctgctcaaggggctgtgccaccctcaggtgaaagtgggcaatgagtac

SEQ ID NO: 99 - DNA, 79 bp, Mus musculus
  source 1..79: mol_type = other DNA
agcctaccttatggggctgaactcagctgacctgctcaagggcctgtgtcaccctcgggtgaaggtggggaacgagtat

SEQ ID NO: 100 - DNA, 4104 bp, synthetic construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 101 - DNA, 4104 bp, synthetic construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 102 - DNA, 4105 bp, synthetic construct
  source 1..4105: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgact

SEQ ID NO: 103 - protein, 4 aa, synthetic construct
SGGS

SEQ ID NO: 104 - protein, 7 aa, synthetic construct
PQPKKKP

SEQ ID NO: 105 - DNA, 20 bp, synthetic construct
  source 1..20: mol_type = other DNA
cctcgggtgaaagtgggcaa

SEQ ID NO: 106 - DNA, 20 bp, synthetic construct
  source 1..20: mol_type = other DNA
cctaaagagaaaatgggcaa

SEQ ID NO: 107 - DNA, 20 bp, synthetic construct
  source 1..20: mol_type = other DNA
cctaaagagaaaatgggcaa

SEQ ID NO: 108 - DNA, 20 bp, synthetic construct
  source 1..20: mol_type = other DNA
catcaagtgaaagtggacag

SEQ ID NO: 109 - DNA, 20 bp, synthetic construct
  source 1..20: mol_type = other DNA
cctcaggagaagatggacaa

SEQ ID NO: 110 - DNA, 20 bp, synthetic construct
  source 1..20: mol_type = other DNA
tatcaggtgaaggtaggcaa

SEQ ID NO: 111 - DNA, 20 bp, synthetic construct
  source 1..20: mol_type = other DNA
gctcaggagaaggtggacaa

SEQ ID NO: 112 - DNA, 20 bp, synthetic construct
  source 1..20: mol_type = other DNA
tctcaagggagagtgggcaa

SEQ ID NO: 113 - DNA, 20 bp, synthetic construct
  source 1..20: mol_type = other DNA
tcattgcccactttcacccg

SEQ ID NO: 114 - DNA, 130 bp, synthetic construct
  source 1..130: mol_type = other DNA
tgctacttgccttttccttccagaggctgacaagtctgcctacctcatggggctgaactcagccgacctgctcaaggggctgtgccaccctcaggtgaaagtgggcaatgagtacgtcaccaaggggcag

SEQ ID NO: 115 - DNA, 21 bp, synthetic construct
  source 1..21: mol_type = other DNA
acctccacatcctgggttcaa

SEQ ID NO: 116 - DNA, 20 bp, synthetic construct
  source 1..20: mol_type = other DNA
gtggaggagagacccatatt

SEQ ID NO: 117 - DNA, 20 bp, synthetic construct
  source 1..20: mol_type = other DNA
ggaggctgtagtgagccaag

SEQ ID NO: 118 - DNA, 20 bp, synthetic construct
  source 1..20: mol_type = other DNA
aggagcaagcgagtgattgt

SEQ ID NO: 119 - DNA, 20 bp, synthetic construct
  source 1..20: mol_type = other DNA
ccgcaggtgaaagtgggcaa

SEQ ID NO: 120 - DNA, 54 bp, synthetic construct
  source 1..54: mol_type = other DNA
tcgtcggcagcgtcagatgtgtataagagacagtcctctcatacactgccttgg

SEQ ID NO: 121 - DNA, 54 bp, synthetic construct
  source 1..54: mol_type = other DNA
gtctcgtgggctcggagatgtgtataagagacagcaccatgcctggctaatttt

SEQ ID NO: 122 - DNA, 53 bp, synthetic construct
  source 1..53: mol_type = other DNA
tcgtcggcagcgtcagatgtgtataagagacaggggacaatgactgcctctgt

SEQ ID NO: 123 - DNA, 54 bp, synthetic construct
  source 1..54: mol_type = other DNA
gtctcgtgggctcggagatgtgtataagagacagtacctcatggggctgaactc

SEQ ID NO: 124 - DNA, 53 bp, synthetic construct
  source 1..53: mol_type = other DNA
tcgtcggcagcgtcagatgtgtataagagacagcaggtctcgattccaaggag

SEQ ID NO: 125 - DNA, 55 bp, synthetic construct
  source 1..55: mol_type = other DNA
gtctcgtgggctcggagatgtgtataagagacaggcacaacccacaagtttgttt

SEQ ID NO: 126 - DNA, 56 bp, synthetic construct
  source 1..56: mol_type = other DNA
tcgtcggcagcgtcagatgtgtataagagacagttttcaaaatattcctgctcact

SEQ ID NO: 127 - DNA, 54 bp, synthetic construct
  source 1..54: mol_type = other DNA
gtctcgtgggctcggagatgtgtataagagacagaggcacctttctgtgtgctt

SEQ ID NO: 128 - DNA, 53 bp, synthetic construct
  source 1..53: mol_type = other DNA
tcgtcggcagcgtcagatgtgtataagagacagattctggatgcaggatttgc

SEQ ID NO: 129 - DNA, 54 bp, synthetic construct
  source 1..54: mol_type = other DNA
gtctcgtgggctcggagatgtgtataagagacaggtggacaacaggccactctt

SEQ ID NO: 130 - DNA, 60 bp, synthetic construct
  source 1..60: mol_type = other DNA
tcgtcggcagcgtcagatgtgtataagagacagggacaatttgtattttagcttattttc

SEQ ID NO: 131 - DNA, 54 bp, synthetic construct
  source 1..54: mol_type = other DNA
gtctcgtgggctcggagatgtgtataagagacagtcccctgcttttctctgtgt

SEQ ID NO: 132 - DNA, 56 bp, synthetic construct
  source 1..56: mol_type = other DNA
tcgtcggcagcgtcagatgtgtataagagacagtgatcctgaagattagtggatgc

SEQ ID NO: 133 - DNA, 55 bp, synthetic construct
  source 1..55: mol_type = other DNA
gtctcgtgggctcggagatgtgtataagagacagccatcctgagataatcctcca

SEQ ID NO: 134 - DNA, 53 bp, synthetic construct
  source 1..53: mol_type = other DNA
tcgtcggcagcgtcagatgtgtataagagacagacctaggaggctgggattgt

SEQ ID NO: 135 - DNA, 54 bp, synthetic construct
  source 1..54: mol_type = other DNA
gtctcgtgggctcggagatgtgtataagagacagcatgacaaggagtccgaggt

SEQ ID NO: 136 - DNA, 53 bp, synthetic construct
  source 1..53: mol_type = other DNA
tcgtcggcagcgtcagatgtgtataagagacaggcccctggttacagcataag

SEQ ID NO: 137 - DNA, 55 bp, synthetic construct
  source 1..55: mol_type = other DNA
gtctcgtgggctcggagatgtgtataagagacagccacaaccactgactgactga

SEQ ID NO: 138 - DNA, 20 bp, synthetic construct
  source 1..20: mol_type = other DNA
tcgttccccaccttcacccg

SEQ ID NO: 139 - DNA, 20 bp, synthetic construct
  source 1..20: mol_type = other DNA
gagaagcagtggtcatcatc

SEQ ID NO: 140 - DNA, 20 bp, synthetic construct
  source 1..20: mol_type = other DNA
gtgagaaacacgtggtgtcc

SEQ ID NO: 141 - DNA, 53 bp, synthetic construct
  source 1..53: mol_type = other DNA
tcgtcggcagcgtcagatgtgtataagagacagggatcaaggacatggcaaat

SEQ ID NO: 142 - DNA, 52 bp, synthetic construct
  source 1..52: mol_type = other DNA
gtctcgtgggctcggagatgtgtataagagacaggcttggtctccagggttg

SEQ ID NO: 143 - DNA, 53 bp, synthetic construct
  source 1..53: mol_type = other DNA
tcgtcggcagcgtcagatgtgtataagagacaggatggcacagaagatgctga

SEQ ID NO: 144 - DNA, 54 bp, synthetic construct
  source 1..54: mol_type = other DNA
gtctcgtgggctcggagatgtgtataagagacagcgaacatgtggtggttgaag

SEQ ID NO: 145 - DNA, 21 bp, synthetic construct
  source 1..21: mol_type = other DNA
gctcttggccactgatagtgc

SEQ ID NO: 146 - DNA, 20 bp, synthetic construct
  source 1..20: mol_type = other DNA
gctcaaagctgttgaaatcg

SEQ ID NO: 147 - DNA, 5292 bp, synthetic construct
  source 1..5292: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 148 - DNA, 5289 bp, synthetic construct
  source 1..5289: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 149 - DNA, 4695 bp, synthetic construct
  source 1..4695: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctcagccagagaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 150 - DNA, 4695 bp, synthetic construct
  source 1..4695: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgac

SEQ ID NO: 151 - DNA, 4695 bp, synthetic construct
  source 1..4695: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 152 - DNA, 4695 bp, synthetic construct
  source 1..4695: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 153 - protein, 85 aa, synthetic construct
CLSYETEILTVEYGLLPIGKIVEKRIECTVYSVDNNGNIYTQPVAQWHDRGEQEVFEYCLEDGSLIRATKDHKFMTVDGQMLPID

SEQ ID NO: 154 - protein, 35 aa, synthetic construct
IKIATRKYLGKQNVYDIGVERDHNFALKNGFIASN

SEQ ID NO: 155 - protein, 1935 aa, Homo sapiens
MGDSEMAVFGAAAPYLRKSEKERLEAQTRPFDLKKDVFVPDDKQEFVKAKIVSREGGKVTAETEYGKTVTVKEDQVMQQNPPKFDKIEDMAMLTFLHEPAVLYNLKDRYGSWMIYTYSGLFCVTVNPYKWLPVYTPEVVAAYRGKKRSEAPPHIFSISDNAYQYMLTDRENQSILITGESGAGKTVNTKRVIQYFAVIAAIGDRSKKDQSPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPQVKVGNEYVTKGQNVQQVIYATGALAKAVYERMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLFDNHLGKSANFQKPRNIKGKPEAHFSLIHYAGIVDYNIIGWLQKNKDPLNETVVGLYQKSSLKLLSTLFANYAGADAPIEKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLSSLDIDHNQYKFGHTKVFFKAGLLGLLEEMRDERLSRIITRIQAQSRGVLARMEYKKLLERRDSLLVIQWNIRAFMGVKNWPWMKLYFKIKPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQEAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKMELQSALEEAEASLEHEEGKILRAQLEFNQIKAEIERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

SEQ ID NO: 156 - DNA, 6079 bp, Mus musculus
  source 1..6079: mol_type = other DNA
atataaaggggctggagcactgagagctgtcagacagagatttctccaacccaggatctctggattggtctcccagcctctgctactcctcttcctgcctgttcctctctccgtccagctgcgccactgtggtgcctcgttccagctgtggtccacattcttcaggattctctgaaaagttaaccagagtttgagtgacagaatgacggacgcccagatggctgacttcggggcagcagcccagtacctccgaaagtcagagaaggaacgcctagaggcccagacccggccctttgacatccgcacggagtgcttcgtgcctgatgacaaggaggagtatgttaaggccaaggtcgtgtcccgggaagggggcaaagtcactgcggaaactgaaaacggaaagacggtgaccataaaggaggaccaggtgatgcagcagaacccacccaagttcgacaagatcgaggacatggccatgctgaccttcctgcacgagccggctgtgctgtacaacctcaaggagcgctacgcggcctggatgatctatacctactcaggcctcttctgcgtcaccgtcaacccctataagtggctgcctgtgtacaatgcggaagtggtggccgcctaccggggcaagaagaggagcgaggcccctcctcacatcttctccatctctgacaacgcctatcagtacatgctgacagatcgggagaatcagtccatcctcatcaccggagaatccggagcggggaagactgtgaacacaaaacgtgtcatccagtactttgccagcattgcagccataggggaccgtagcaagaaggaaaatcctaatgcaaacaagggcaccctggaggaccagattatccaggctaaccccgctctggaggccttcggcaacgccaagactgtccggaatgacaactcctcccgctttgggaaattcatcaggatccactttggagctactggaaagctggcttctgcagacatagagacctaccttctggagaagtcccgggtgatcttccagctaaaggctgagaggaactaccacatcttctaccagatcctgtccaacaagaagccggagctgctggacatgctgctggtcaccaacaacccatacgactacgccttcgtctctcagggagaggtgtccgtggcctccattgatgactctgaggagctcttggccactgatagtgcctttgatgtgctgagcttcacggcagaggagaaggctggtgtctacaagctgacaggggccatcatgcactacggaaacatgaagttcaagcagaagcagcgggaggagcaggcggagcctgatggcacagaagatgctgacaaatcagcctaccttatggggctgaactcagctgacctgctcaagggcctgtgtcaccctcaggtgaaggtggggaacgagtatgtcaccaaggggcagagtgtacagcaagtgtactattccatcggggcactggccaagtcagtgtacgagaagatgttcaactggatggtgacacgcatcaacgcaaccctggagaccaagcagccgcgccagtacttcataggtgtcctggacattgccggctttgagatcttcgatttcaacagctttgagcagctgtgcatcaacttcaccaatgagaagctgcagcagttcttcaaccaccacatgttcgtgctggagcaggaggagtacaagaaggagggcattgagtgggagtttatcgacttcggcatggacctgcaggcctgcatcgacctcatcgagaagcccatgggcatcatgtccatcctcgaggaggagtgcatgttccccaaggcctcagacatgaccttcaaggccaagctgtatgacaaccacctgggcaaatccaacaacttccagaagcctcgcaatgtcaaggggaagcaggaagcccacttctccttggtccactatgctggcaccgtggactacaacattatgggctggctggaaaagaacaaggacccactcaatgagacggtggtgggtttgtaccagaagtcctccctcaagctcatggctacactcttctctacctatgcttctgctgataccggtgacagtggtaaaggcaaaggaggcaagaagaaaggctcatccttccaaacagtgtctgctctccaccgggaaaatctgaacaagctgatgacaaacctgaagaccacccaccctcactttgtgcgctgcatcattcccaacgagcgaaaggctccaggggtgatggacaaccccctggtcatgcaccagctgcgatgcaatggcgtgctggagggtatccgcatctgcaggaagggcttccccaaccgcattctctatggggacttccggcagaggtatcgcatcctgaacccagcagccatccctgaggggcaattcattgatagcaggaaaggggctgagaaactgctgggctccctggacattgaccacaaccaatacaagtttggccacaccaaggtgttcttcaaggcgggcctgctggggctgctcgaggagatgcgagatgagaggctgagccgtatcatcaccagaatccaggcccaggcccgagggcagctcatgcgcattgagttcaagaagatagtggaacgcagggatgccctgctggttatccagtggaacattcgggccttcatgggggtcaagaattggccatggatgaagctctacttcaagatcaaaccgctgctgaagagcgcagagacggagaaggagatggccaacatgaaggaggagtttgggcgagtcaaagatgcactggagaagtctgaggctcgccgcaaggagctggaggagaagatggtgtccctgctgcaggagaagaatgacctacagctccaagtgcaggcggaacaagacaacctcaatgatgcagaggagcgctgtgaccagctgatcaagaacaagatccagctggaggccaaggtgaaggagatgaccgagaggctggaggacgaggaggagatgaacgccgagctcactgccaagaagcgcaagctggaagatgagtgctcagagctcaagaaggatattgatgacctggagctgacgctggccaaggtggaaaaggaaaagcatgcaacagagaacaaggttaaaaacctaacagaggagatggctgggctggatgaaatcattgccaagctgaccaaagagaagaaagctctgcaagaagcccaccagcaagccctcgatgacctgcaggctgaagaagacaaggtcaacacgctgaccaagtccaaagtcaagctggagcagcaggtggatgatctggagggatccctggagcaggagaagaaagtgcgcatggacctagagcgagccaagcggaagctggagggagacctgaagctgacccaggagagcatcatggacctggagaatgacaagcttcagctggaagaaaagctcaagaagaaagagttcgacatcagtcagcagaacagtaaaattgaggacgagcaggccctggctcttcagctgcagaagaaactgaaggaaaaccaggcacgcatcgaggagctggaggaggagctggaggcagagcgcacagcccgggctaaggtggagaagctgcgctctgacctgtcccgggagctggaggagatcagtgagaggctggaggaggcaggcggggccacatccgtgcagatagagatgaataagaagcgcgaggccgagttccagaagatgcggcgggacctggaggaggccacgctgcagcacgaggccacggcggcggccctgcgcaagaagcatgctgacagcgtggcggagctgggcgagcagatcgacaacctccagcgggtgaagcagaagctggagaaagagaagagcgagttcaagctggagctggatgacgtcacctccaacatggagcagatcatcaaggccaaggccaacctggagaaagtgtcccggacactggaggaccaggccaatgagtaccgcgtgaagctggaagaagcccagcgctccctcaatgacttcaccacacagcgagccaagctgcagacagagaacggggagttggctaggcaactggaagaaaaggaggcattgatttcccagctgacccgaggcaagctctcctacacccagcagatggaggacctcaagaggcaactggaggaggaaggcaaggccaagaacgccctggcccacgcactgcaatcatcccggcatgactgtgacctgctgagggaacagtatgaagaagaaatggaggccaaggctgagctacagcgtgtcctgtccaaggccaactcagaggtggcccagtggaggaccaagtatgagacggatgccatacagaggacggaggagctggaggaagccaagaagaagctggctcagaggctgcaggatgcagaggaggcagtggaggccgtcaacgccaagtgttcctccctggagaagaccaagcacaggctgcagaatgagatcgaggacctgatggtggacgtggagcgctccaatgccgccgccgcagccctggacaagaagcagaggaactttgacaagatcctggctgagtggaagcagaagtatgaggagtcgcagtcagagctggagtcttcccagaaggaggcgcgctccctgagcacagagctcttcaagctcaagaacgcctatgaggagtctctggagcacctggagaccttcaagcgggagaacaagaacctccaggaggagatctcagacctgactgaacagctgggagaaggggggaaaaacgtgcacgagctggagaagatccgcaaacagctggaggtggagaagctggagctgcagtcagccctggaggaggctgaggcctccctggagcacgaggagggcaagatcctccgtgcccagctggagttcaaccagatcaaggcagagatcgaaaggaagctggcagagaaggatgaggagatggagcaggccaagcgcaaccacctgcggatggtggactccctgcagacctccctggatgcggagacacgcagccgcaatgaggccctgcgggtgaagaagaagatggagggcgacctcaacgagatggagatccagctcagccaggccaatagaatagcctcagaggcacagaaacacctgaagaattctcaagctcacttgaaggacacccagctccagctggatgatgctgtccatgccaatgacgacctgaaggagaacatcgccatcgtggaacggcgcaacaacctgctgcaggcggagctggaggagctgcgggctgtggtggagcagacggagcggtctcggaagctggcagagcaggagctgattgagaccagcgagcgggtgcagctgctgcactcgcagaacaccagcctcatcaaccagaagaagaagatggagtcagacctgacccaactccagacagaagtagaggaggcagtgcaggagtgtaggaacgcagaggagaaggccaagaaggccatcacagatgccgcaatgatggctgaggagctgaagaaggagcaggacaccagcgcccacctggagcgcatgaagaagaacatggagcagaccatcaaggacttgcagcaccgtctggacgaggcagagcagatcgccctcaagggcggcaagaagcagctgcagaagctggaggcccgggtccgggagctggagaatgagctggaggctgagcagaagcgcaatgcagagtcggtgaagggcatgaggaagagcgagcggcgcatcaaggagctcacctaccagacagaggaagacaagaagaacttaatgcggctgcaggacctggtggacaagctacagttgaaggtgaaggcctacaagcgccaggctgaggaggcggaggagcaggccaacaccaacctgtccaagttccgcaaggtgcagcacgagctggatgaggcggaggagagggcggacatcgccgagtcccaggtcaacaagctgcgggccaagagccgggacattggtgccaagaagatgcacgacgaggaataacctctccagcagaccctcgctgtggccaatccacaataaacataaacgttcgactctgcc

SEQ ID NO: 157 - DNA, 6087 bp, Homo sapiens
  source 1..6087: mol_type = other DNA
gggggtgggggtgccctgctgccccatatatacagcccctgagaccaggtctggctccacagctctgtcctgctctgtgtctttccctgctgctctcaggtcccctgcaggccttggcccctttcctcatctgtagacacacttgagtagcccaggcacagccatgggagattcggagatggcagtctttggggctgccgccccctacctgcgcaagtcagagaaggagcggctagaagcgcagaccaggccttttgacctcaagaaggatgtcttcgtgcctgatgacaaacaggagtttgtcaaggccaagatcgtgtctcgagagggtggcaaagtcactgccgagaccgagtatggcaagacagtgaccgtgaaggaggaccaggtgatgcagcagaacccacccaagttcgacaaaatcgaggacatggccatgctgaccttcctgcatgagcccgcggtgctctacaacctcaaggatcgctacggctcctggatgatctacacctactcgggcctcttctgtgtcaccgtcaacccttacaagtggctgccggtgtacactcctgaggtggtggctgcctaccggggcaagaagaggagcgaggccccgccccacatcttctccatctccgacaacgcctatcagtacatgctgacagacagagaaaaccagtccatcctgatcaccggagaatccggagcagggaagacagtcaacaccaagagggtcatccagtactttgctgttattgcagccattggggaccgcagcaagaaggaccagagcccgggcaagggcaccctggaggaccagatcatccaggccaaccctgctctggaggcctttggcaatgccaagaccgtccggaacgacaactcctcccgcttcgggaaattcattcgaattcattttggggcaacaggaaagttggcatctgcagacatagagacctatcttctggaaaaatccagagttattttccagctgaaagcagagagagattatcacattttctaccaaatcctgtctaacaaaaagcctgagctgctggacatgctgctgatcaccaacaacccctacgattatgcattcatctcccaaggagagaccaccgtggcctccattgatgacgctgaggagctcatggccactgataacgcttttgatgtgctgggcttcacttcagaggagaaaaactccatgtataagctgacaggcgccatcatgcactttggaaacatgaagttcaagctgaagcagcgggaggagcaggcggagccagacggcactgaagaggctgacaagtctgcctacctcatggggctgaactcagccgacctgctcaaggggctgtgccaccctcaggtgaaagtgggcaatgagtacgtcaccaaggggcagaatgtccagcaggtgatatatgccactggggcactggccaaggcagtgtatgagaggatgttcaactggatggtgacgcgcatcaatgccaccctggagaccaagcagccacgccagtacttcataggagtcctggacatcgctggcttcgagatcttcgatttcaacagctttgagcagctctgcatcaacttcaccaacgagaagctgcagcagttcttcaaccaccacatgtttgtgctggagcaggaggagtacaagaaggagggcatcgagtggacattcattgactttggcatggacctgcaggcctgcattgacctcatcgagaagcccatgggcatcatgtccatcctggaagaggagtgcatgttccccaaggccaccgacatgaccttcaaggccaagctgtttgacaaccacctgggcaaatccgccaacttccagaagccacgcaatatcaaggggaagcctgaagcccacttctccctgatccactatgccggcatcgtggactacaacatcattggctggctgcagaagaacaaggatcctctcaatgagactgtcgtgggcttgtatcagaagtcttccctcaagctgctcagcaccctgtttgccaactatgctggggctgatgcgcctattgagaagggcaaaggcaaggccaagaaaggctcgtcctttcagactgtgtcagctctgcacagggaaaatctgaacaagctgatgaccaacttgcgctccacccatccccactttgtacgttgtatcatccctaatgagacaaagtctccaggggtgatggacaaccccctggtcatgcaccagctgcgctgcaatggtgtgctggagggcatccgcatctgcaggaaaggcttccccaaccgcatcctctacggggacttccggcagaggtatcgcatcctgaacccagcggccatccctgagggacagttcattgatagcaggaagggggcagagaagctgctcagctccctggacattgatcacaaccagtacaagtttggccacaccaaggtgttcttcaaggccgggctgctggggctgctggaggaaatgagggacgagaggctgagccgcatcatcacgcgtatccaggcccagtcccgaggtgtgctcgccagaatggagtacaaaaagctgctggaacgtagagactccctgctggtaatccagtggaacattcgggccttcatgggggtcaagaattggccctggatgaagctctacttcaagatcaagccgctgctgaagagtgcagaaagagagaaggagatggcctccatgaaggaggagttcacacgcctcaaagaggcgctagagaagtccgaggctcgccgcaaggagctggaggagaagatggtgtccctgctgcaggagaagaatgacctgcagctccaagtgcaggcggaacaagacaacctggcagatgctgaggagcgctgtgatcagctgatcaaaaacaagattcagctggaggccaaggtgaaggagatgaacgagaggctggaggatgaggaggagatgaatgctgagctcactgccaagaagcgcaagctggaagatgagtgctcagagctcaaaagggacatcgatgatctggagctgacactggccaaagtggagaaggagaaacacgcaacagagaacaaggtgaaaaacctgacagaggagatggctgggctggatgagatcattgccaagctgaccaaggagaagaaagctctgcaagaggcccaccaacaggctctggatgaccttcaggccgaggaggacaaggtcaacaccctgactaaggccaaagtcaagctggagcagcaagtggatgatctggaaggatccctggagcaagagaagaaggtgcgcatggacctggagcgagcgaagcggaagctggagggcgacctgaagctgacccaggagagcatcatggacctggagaatgacaagcagcagctggatgagcggctgaaaaaaaaagactttgagctgaatgctctcaacgcaaggattgaggatgaacaggccctcggcagccagctgcagaagaagctcaaggagcttcaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggctaacctggagaagatgtgccggaccttggaagaccagatgaatgagcaccggagcaaggcggaggagacccagcgttctgtcaacgacctcaccagccagcgggccaagttgcaaaccgagaatggtgagctgtcccggcagctggatgagaaggaggcactgatctcccagctgacccgaggcaagctcacctacacccagcagctggaggacctcaagaggcagctggaggaggaggttaaggcgaagaacgccctggcccacgcactgcagtcggcccggcatgactgcgacctgctgcgggagcagtacgaggaggagacggaggccaaggccgagctgcagcgcgtcctttccaaggccaactcggaggtggcccagtggaggaccaagtatgagacggacgccattcagcggactgaggagctcgaggaggccaagaagaagctggcccagcggctgcaggaagctgaggaggccgtggaggctgttaatgccaagtgctcctcgctggagaagaccaagcaccggctacagaatgagatcgaggacttgatggtggacgtagagcgctccaatgctgctgctgcagccctggacaagaagcagaggaacttcgacaagatcctggccgagtggaagcagaagtatgaggagtcgcagtcggagctggagtcctcgcagaaggaggctcgctccctcagcacagagctcttcaaactcaagaacgcctatgaggagtccctggaacatctggagaccttcaagcgggagaacaaaaacctgcaggaggagatctccgacttgactgagcagttgggttccagcggaaagactatccatgagctggagaaggtccgaaagcagctggaggccgagaagatggagctgcagtcagccctggaggaggccgaggcctccctggagcacgaggagggcaagatcctccgggcccagctggagttcaaccagatcaaggcagagatcgagcggaagctggcagagaaggacgaggagatggaacaggccaagcgcaaccacctgcgggtggtggactcgctgcagacctccctggacgcagagacacgcagccgcaacgaggccctgagggtgaagaagaagatggaaggagacctcaatgagatggagatccagctcagccacgccaaccgcatggccgccgaggcccagaagcaagtcaagagcctccagagcttgttgaaggacacccagattcagctggacgatgcagtccgtgccaacgacgacctgaaggagaacatcgccatcgtggagcggcgcaacaacctgctgcaggctgagctggaggagttgcgtgccgtggtggagcagacagagcggtcccggaagctggcggagcaggagctgattgagactagtgagcgggtgcagctgctgcattcccagaacaccagcctcatcaaccagaagaagaagatggatgctgacctgtcccagctccagactgaagtggaggaggcagtgcaggagtgcaggaatgctgaggagaaggccaagaaggccatcacggatgccgccatgatggcagaggagctgaagaaggagcaggacaccagcgcccacctggagcgcatgaagaagaacatggaacagaccattaaggacctgcagcaccggctggacgaagccgagcagatcgccctcaagggcggcaagaagcagctgcagaagctggaagcgcgggtgcgggagctggagaatgagctggaggccgagcagaagcgcaacgcagagtcggtgaagggcatgaggaagagcgagcggcgcatcaaggagctcacctaccagacggaggaggacaggaaaaacctgctgcggctgcaggacctggtagacaagctgcagctaaaggtcaaggcctacaagcgccaggccgaggaggcggaggagcaagccaacaccaacctgtccaagttccgcaaggtgcagcacgagctggatgaggcagaggagcgggcggacatcgccgagtcccaggtcaacaagctgcgggccaagagccgtgacattggcacgaagggcttgaatgaggagtagctttgccacatcttgatctgctcagccctggaggtgccagcaaagccccatgctggagcctgtgtaacagctccttgggaggaagcagaataaagcaattttccttgaagccgaga

SEQ ID NO: 158 - DNA, 79 bp, synthetic construct
  source 1..79: mol_type = other DNA
  variation 10: note = N = C or T
  variation 28: note = N = C or T
  variation 49: note = N = C or T
  variation 70: note = N = C or G
  variation 73: note = N = T or C
  variation 43: note = N = G or C
tgcctacctnatggggctgaactcagcngacctgctcaagggnctgtgncaccctcaggtgaaagtgggnaangagtac

SEQ ID NO: 159 - DNA, 6079 bp, Mus musculus
  source 1..6079: mol_type = other DNA
atataaaggggctggagcactgagagctgtcagacagagatttctccaacccaggatctctggattggtctcccagcctctgctactcctcttcctgcctgttcctctctccgtccagctgcgccactgtggtgcctcgttccagctgtggtccacattcttcaggattctctgaaaagttaaccagagtttgagtgacagaatgacggacgcccagatggctgacttcggggcagcagcccagtacctccgaaagtcagagaaggaacgcctagaggcccagacccggccctttgacatccgcacggagtgcttcgtgcctgatgacaaggaggagtatgttaaggccaaggtcgtgtcccgggaagggggcaaagtcactgcggaaactgaaaacggaaagacggtgaccataaaggaggaccaggtgatgcagcagaacccacccaagttcgacaagatcgaggacatggccatgctgaccttcctgcacgagccggctgtgctgtacaacctcaaggagcgctacgcggcctggatgatctatacctactcaggcctcttctgcgtcaccgtcaacccctataagtggctgcctgtgtacaatgcggaagtggtggccgcctaccggggcaagaagaggagcgaggcccctcctcacatcttctccatctctgacaacgcctatcagtacatgctgacagatcgggagaatcagtccatcctcatcaccggagaatccggagcggggaagactgtgaacacaaaacgtgtcatccagtactttgccagcattgcagccataggggaccgtagcaagaaggaaaatcctaatgcaaacaagggcaccctggaggaccagattatccaggctaaccccgctctggaggccttcggcaacgccaagactgtccggaatgacaactcctcccgctttgggaaattcatcaggatccactttggagctactggaaagctggcttctgcagacatagagacctaccttctggagaagtcccgggtgatcttccagctaaaggctgagaggaactaccacatcttctaccagatcctgtccaacaagaagccggagctgctggacatgctgctggtcaccaacaacccatacgactacgccttcgtctctcagggagaggtgtccgtggcctccattgatgactctgaggagctcttggccactgatagtgcctttgatgtgctgagcttcacggcagaggagaaggctggtgtctacaagctgacaggggccatcatgcactacggaaacatgaagttcaagcagaagcagcgggaggagcaggcggagcctgatggcacagaagatgctgacaaatcagcctacctcatggggctgaactcagccgacctgctcaaggggctgtgccaccctcaggtgaaagtgggcaatgagtatgtcaccaaggggcagagtgtacagcaagtgtactattccatcggggcactggccaagtcagtgtacgagaagatgttcaactggatggtgacacgcatcaacgcaaccctggagaccaagcagccgcgccagtacttcataggtgtcctggacattgccggctttgagatcttcgatttcaacagctttgagcagctgtgcatcaacttcaccaatgagaagctgcagcagttcttcaaccaccacatgttcgtgctggagcaggaggagtacaagaaggagggcattgagtgggagtttatcgacttcggcatggacctgcaggcctgcatcgacctcatcgagaagcccatgggcatcatgtccatcctcgaggaggagtgcatgttccccaaggcctcagacatgaccttcaaggccaagctgtatgacaaccacctgggcaaatccaacaacttccagaagcctcgcaatgtcaaggggaagcaggaagcccacttctccttggtccactatgctggcaccgtggactacaacattatgggctggctggaaaagaacaaggacccactcaatgagacggtggtgggtttgtaccagaagtcctccctcaagctcatggctacactcttctctacctatgcttctgctgataccggtgacagtggtaaaggcaaaggaggcaagaagaaaggctcatccttccaaacagtgtctgctctccaccgggaaaatctgaacaagctgatgacaaacctgaagaccacccaccctcactttgtgcgctgcatcattcccaacgagcgaaaggctccaggggtgatggacaaccccctggtcatgcaccagctgcgatgcaatggcgtgctggagggtatccgcatctgcaggaagggcttccccaaccgcattctctatggggacttccggcagaggtatcgcatcctgaacccagcagccatccctgaggggcaattcattgatagcaggaaaggggctgagaaactgctgggctccctggacattgaccacaaccaatacaagtttggccacaccaaggtgttcttcaaggcgggcctgctggggctgctcgaggagatgcgagatgagaggctgagccgtatcatcaccagaatccaggcccaggcccgagggcagctcatgcgcattgagttcaagaagatagtggaacgcagggatgccctgctggttatccagtggaacattcgggccttcatgggggtcaagaattggccatggatgaagctctacttcaagatcaaaccgctgctgaagagcgcagagacggagaaggagatggccaacatgaaggaggagtttgggcgagtcaaagatgcactggagaagtctgaggctcgccgcaaggagctggaggagaagatggtgtccctgctgcaggagaagaatgacctacagctccaagtgcaggcggaacaagacaacctcaatgatgcagaggagcgctgtgaccagctgatcaagaacaagatccagctggaggccaaggtgaaggagatgaccgagaggctggaggacgaggaggagatgaacgccgagctcactgccaagaagcgcaagctggaagatgagtgctcagagctcaagaaggatattgatgacctggagctgacgctggccaaggtggaaaaggaaaagcatgcaacagagaacaaggttaaaaacctaacagaggagatggctgggctggatgaaatcattgccaagctgaccaaagagaagaaagctctgcaagaagcccaccagcaagccctcgatgacctgcaggctgaagaagacaaggtcaacacgctgaccaagtccaaagtcaagctggagcagcaggtggatgatctggagggatccctggagcaggagaagaaagtgcgcatggacctagagcgagccaagcggaagctggagggagacctgaagctgacccaggagagcatcatggacctggagaatgacaagcttcagctggaagaaaagctcaagaagaaagagttcgacatcagtcagcagaacagtaaaattgaggacgagcaggccctggctcttcagctgcagaagaaactgaaggaaaaccaggcacgcatcgaggagctggaggaggagctggaggcagagcgcacagcccgggctaaggtggagaagctgcgctctgacctgtcccgggagctggaggagatcagtgagaggctggaggaggcaggcggggccacatccgtgcagatagagatgaataagaagcgcgaggccgagttccagaagatgcggcgggacctggaggaggccacgctgcagcacgaggccacggcggcggccctgcgcaagaagcatgctgacagcgtggcggagctgggcgagcagatcgacaacctccagcgggtgaagcagaagctggagaaagagaagagcgagttcaagctggagctggatgacgtcacctccaacatggagcagatcatcaaggccaaggccaacctggagaaagtgtcccggacactggaggaccaggccaatgagtaccgcgtgaagctggaagaagcccagcgctccctcaatgacttcaccacacagcgagccaagctgcagacagagaacggggagttggctaggcaactggaagaaaaggaggcattgatttcccagctgacccgaggcaagctctcctacacccagcagatggaggacctcaagaggcaactggaggaggaaggcaaggccaagaacgccctggcccacgcactgcaatcatcccggcatgactgtgacctgctgagggaacagtatgaagaagaaatggaggccaaggctgagctacagcgtgtcctgtccaaggccaactcagaggtggcccagtggaggaccaagtatgagacggatgccatacagaggacggaggagctggaggaagccaagaagaagctggctcagaggctgcaggatgcagaggaggcagtggaggccgtcaacgccaagtgttcctccctggagaagaccaagcacaggctgcagaatgagatcgaggacctgatggtggacgtggagcgctccaatgccgccgccgcagccctggacaagaagcagaggaactttgacaagatcctggctgagtggaagcagaagtatgaggagtcgcagtcagagctggagtcttcccagaaggaggcgcgctccctgagcacagagctcttcaagctcaagaacgcctatgaggagtctctggagcacctggagaccttcaagcgggagaacaagaacctccaggaggagatctcagacctgactgaacagctgggagaaggggggaaaaacgtgcacgagctggagaagatccgcaaacagctggaggtggagaagctggagctgcagtcagccctggaggaggctgaggcctccctggagcacgaggagggcaagatcctccgtgcccagctggagttcaaccagatcaaggcagagatcgaaaggaagctggcagagaaggatgaggagatggagcaggccaagcgcaaccacctgcggatggtggactccctgcagacctccctggatgcggagacacgcagccgcaatgaggccctgcgggtgaagaagaagatggagggcgacctcaacgagatggagatccagctcagccaggccaatagaatagcctcagaggcacagaaacacctgaagaattctcaagctcacttgaaggacacccagctccagctggatgatgctgtccatgccaatgacgacctgaaggagaacatcgccatcgtggaacggcgcaacaacctgctgcaggcggagctggaggagctgcgggctgtggtggagcagacggagcggtctcggaagctggcagagcaggagctgattgagaccagcgagcgggtgcagctgctgcactcgcagaacaccagcctcatcaaccagaagaagaagatggagtcagacctgacccaactccagacagaagtagaggaggcagtgcaggagtgtaggaacgcagaggagaaggccaagaaggccatcacagatgccgcaatgatggctgaggagctgaagaaggagcaggacaccagcgcccacctggagcgcatgaagaagaacatggagcagaccatcaaggacttgcagcaccgtctggacgaggcagagcagatcgccctcaagggcggcaagaagcagctgcagaagctggaggcccgggtccgggagctggagaatgagctggaggctgagcagaagcgcaatgcagagtcggtgaagggcatgaggaagagcgagcggcgcatcaaggagctcacctaccagacagaggaagacaagaagaacttaatgcggctgcaggacctggtggacaagctacagttgaaggtgaaggcctacaagcgccaggctgaggaggcggaggagcaggccaacaccaacctgtccaagttccgcaaggtgcagcacgagctggatgaggcggaggagagggcggacatcgccgagtcccaggtcaacaagctgcgggccaagagccgggacattggtgccaagaagatgcacgacgaggaataacctctccagcagaccctcgctgtggccaatccacaataaacataaacgttcgactctgcc

SEQ ID NO: 160 - DNA, 79 bp, synthetic construct
  source 1..79: mol_type = other DNA
  variation 57: note = N = A or G
tgcctacctcatggggctgaactcagccgacctgctcaaggggctgtgccaccctcnggtgaaagtgggcaatgagtac

SEQ ID NO: 161 - protein, 9 aa, Homo sapiens
  VARIANT 3: note = X = R or Q
HPXVKVGNE

SEQ ID NO: 162 - DNA, 6087 bp, Homo sapiens
  source 1..6087: mol_type = other DNA
gggggtgggggtgccctgctgccccatatatacagcccctgagaccaggtctggctccacagctctgtcctgctctgtgtctttccctgctgctctcaggtcccctgcaggccttggcccctttcctcatctgtagacacacttgagtagcccaggcacagccatgggagattcggagatggcagtctttggggctgccgccccctacctgcgcaagtcagagaaggagcggctagaagcgcagaccaggccttttgacctcaagaaggatgtcttcgtgcctgatgacaaacaggagtttgtcaaggccaagatcgtgtctcgagagggtggcaaagtcactgccgagaccgagtatggcaagacagtgaccgtgaaggaggaccaggtgatgcagcagaacccacccaagttcgacaaaatcgaggacatggccatgctgaccttcctgcatgagcccgcggtgctctacaacctcaaggatcgctacggctcctggatgatctacacctactcgggcctcttctgtgtcaccgtcaacccttacaagtggctgccggtgtacactcctgaggtggtggctgcctaccggggcaagaagaggagcgaggccccgccccacatcttctccatctccgacaacgcctatcagtacatgctgacagacagagaaaaccagtccatcctgatcaccggagaatccggagcagggaagacagtcaacaccaagagggtcatccagtactttgctgttattgcagccattggggaccgcagcaagaaggaccagagcccgggcaagggcaccctggaggaccagatcatccaggccaaccctgctctggaggcctttggcaatgccaagaccgtccggaacgacaactcctcccgcttcgggaaattcattcgaattcattttggggcaacaggaaagttggcatctgcagacatagagacctatcttctggaaaaatccagagttattttccagctgaaagcagagagagattatcacattttctaccaaatcctgtctaacaaaaagcctgagctgctggacatgctgctgatcaccaacaacccctacgattatgcattcatctcccaaggagagaccaccgtggcctccattgatgacgctgaggagctcatggccactgataacgcttttgatgtgctgggcttcacttcagaggagaaaaactccatgtataagctgacaggcgccatcatgcactttggaaacatgaagttcaagctgaagcagcgggaggagcaggcggagccagacggcactgaagaggctgacaagtctgcctacctcatggggctgaactcagccgacctgctcaaggggctgtgccaccctcgggtgaaagtgggcaatgagtacgtcaccaaggggcagaatgtccagcaggtgatatatgccactggggcactggccaaggcagtgtatgagaggatgttcaactggatggtgacgcgcatcaatgccaccctggagaccaagcagccacgccagtacttcataggagtcctggacatcgctggcttcgagatcttcgatttcaacagctttgagcagctctgcatcaacttcaccaacgagaagctgcagcagttcttcaaccaccacatgtttgtgctggagcaggaggagtacaagaaggagggcatcgagtggacattcattgactttggcatggacctgcaggcctgcattgacctcatcgagaagcccatgggcatcatgtccatcctggaagaggagtgcatgttccccaaggccaccgacatgaccttcaaggccaagctgtttgacaaccacctgggcaaatccgccaacttccagaagccacgcaatatcaaggggaagcctgaagcccacttctccctgatccactatgccggcatcgtggactacaacatcattggctggctgcagaagaacaaggatcctctcaatgagactgtcgtgggcttgtatcagaagtcttccctcaagctgctcagcaccctgtttgccaactatgctggggctgatgcgcctattgagaagggcaaaggcaaggccaagaaaggctcgtcctttcagactgtgtcagctctgcacagggaaaatctgaacaagctgatgaccaacttgcgctccacccatccccactttgtacgttgtatcatccctaatgagacaaagtctccaggggtgatggacaaccccctggtcatgcaccagctgcgctgcaatggtgtgctggagggcatccgcatctgcaggaaaggcttccccaaccgcatcctctacggggacttccggcagaggtatcgcatcctgaacccagcggccatccctgagggacagttcattgatagcaggaagggggcagagaagctgctcagctccctggacattgatcacaaccagtacaagtttggccacaccaaggtgttcttcaaggccgggctgctggggctgctggaggaaatgagggacgagaggctgagccgcatcatcacgcgtatccaggcccagtcccgaggtgtgctcgccagaatggagtacaaaaagctgctggaacgtagagactccctgctggtaatccagtggaacattcgggccttcatgggggtcaagaattggccctggatgaagctctacttcaagatcaagccgctgctgaagagtgcagaaagagagaaggagatggcctccatgaaggaggagttcacacgcctcaaagaggcgctagagaagtccgaggctcgccgcaaggagctggaggagaagatggtgtccctgctgcaggagaagaatgacctgcagctccaagtgcaggcggaacaagacaacctggcagatgctgaggagcgctgtgatcagctgatcaaaaacaagattcagctggaggccaaggtgaaggagatgaacgagaggctggaggatgaggaggagatgaatgctgagctcactgccaagaagcgcaagctggaagatgagtgctcagagctcaaaagggacatcgatgatctggagctgacactggccaaagtggagaaggagaaacacgcaacagagaacaaggtgaaaaacctgacagaggagatggctgggctggatgagatcattgccaagctgaccaaggagaagaaagctctgcaagaggcccaccaacaggctctggatgaccttcaggccgaggaggacaaggtcaacaccctgactaaggccaaagtcaagctggagcagcaagtggatgatctggaaggatccctggagcaagagaagaaggtgcgcatggacctggagcgagcgaagcggaagctggagggcgacctgaagctgacccaggagagcatcatggacctggagaatgacaagcagcagctggatgagcggctgaaaaaaaaagactttgagctgaatgctctcaacgcaaggattgaggatgaacaggccctcggcagccagctgcagaagaagctcaaggagcttcaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggctaacctggagaagatgtgccggaccttggaagaccagatgaatgagcaccggagcaaggcggaggagacccagcgttctgtcaacgacctcaccagccagcgggccaagttgcaaaccgagaatggtgagctgtcccggcagctggatgagaaggaggcactgatctcccagctgacccgaggcaagctcacctacacccagcagctggaggacctcaagaggcagctggaggaggaggttaaggcgaagaacgccctggcccacgcactgcagtcggcccggcatgactgcgacctgctgcgggagcagtacgaggaggagacggaggccaaggccgagctgcagcgcgtcctttccaaggccaactcggaggtggcccagtggaggaccaagtatgagacggacgccattcagcggactgaggagctcgaggaggccaagaagaagctggcccagcggctgcaggaagctgaggaggccgtggaggctgttaatgccaagtgctcctcgctggagaagaccaagcaccggctacagaatgagatcgaggacttgatggtggacgtagagcgctccaatgctgctgctgcagccctggacaagaagcagaggaacttcgacaagatcctggccgagtggaagcagaagtatgaggagtcgcagtcggagctggagtcctcgcagaaggaggctcgctccctcagcacagagctcttcaaactcaagaacgcctatgaggagtccctggaacatctggagaccttcaagcgggagaacaaaaacctgcaggaggagatctccgacttgactgagcagttgggttccagcggaaagactatccatgagctggagaaggtccgaaagcagctggaggccgagaagatggagctgcagtcagccctggaggaggccgaggcctccctggagcacgaggagggcaagatcctccgggcccagctggagttcaaccagatcaaggcagagatcgagcggaagctggcagagaaggacgaggagatggaacaggccaagcgcaaccacctgcgggtggtggactcgctgcagacctccctggacgcagagacacgcagccgcaacgaggccctgagggtgaagaagaagatggaaggagacctcaatgagatggagatccagctcagccacgccaaccgcatggccgccgaggcccagaagcaagtcaagagcctccagagcttgttgaaggacacccagattcagctggacgatgcagtccgtgccaacgacgacctgaaggagaacatcgccatcgtggagcggcgcaacaacctgctgcaggctgagctggaggagttgcgtgccgtggtggagcagacagagcggtcccggaagctggcggagcaggagctgattgagactagtgagcgggtgcagctgctgcattcccagaacaccagcctcatcaaccagaagaagaagatggatgctgacctgtcccagctccagactgaagtggaggaggcagtgcaggagtgcaggaatgctgaggagaaggccaagaaggccatcacggatgccgccatgatggcagaggagctgaagaaggagcaggacaccagcgcccacctggagcgcatgaagaagaacatggaacagaccattaaggacctgcagcaccggctggacgaagccgagcagatcgccctcaagggcggcaagaagcagctgcagaagctggaagcgcgggtgcgggagctggagaatgagctggaggccgagcagaagcgcaacgcagagtcggtgaagggcatgaggaagagcgagcggcgcatcaaggagctcacctaccagacggaggaggacaggaaaaacctgctgcggctgcaggacctggtagacaagctgcagctaaaggtcaaggcctacaagcgccaggccgaggaggcggaggagcaagccaacaccaacctgtccaagttccgcaaggtgcagcacgagctggatgaggcagaggagcgggcggacatcgccgagtcccaggtcaacaagctgcgggccaagagccgtgacattggcacgaagggcttgaatgaggagtagctttgccacatcttgatctgctcagccctggaggtgccagcaaagccccatgctggagcctgtgtaacagctccttgggaggaagcagaataaagcaattttccttgaagccgaga

SEQ ID NO: 163 - DNA, 6079 bp, Mus musculus
  source 1..6079: mol_type = other DNA
atataaaggggctggagcactgagagctgtcagacagagatttctccaacccaggatctctggattggtctcccagcctctgctactcctcttcctgcctgttcctctctccgtccagctgcgccactgtggtgcctcgttccagctgtggtccacattcttcaggattctctgaaaagttaaccagagtttgagtgacagaatgacggacgcccagatggctgacttcggggcagcagcccagtacctccgaaagtcagagaaggaacgcctagaggcccagacccggccctttgacatccgcacggagtgcttcgtgcctgatgacaaggaggagtatgttaaggccaaggtcgtgtcccgggaagggggcaaagtcactgcggaaactgaaaacggaaagacggtgaccataaaggaggaccaggtgatgcagcagaacccacccaagttcgacaagatcgaggacatggccatgctgaccttcctgcacgagccggctgtgctgtacaacctcaaggagcgctacgcggcctggatgatctatacctactcaggcctcttctgcgtcaccgtcaacccctataagtggctgcctgtgtacaatgcggaagtggtggccgcctaccggggcaagaagaggagcgaggcccctcctcacatcttctccatctctgacaacgcctatcagtacatgctgacagatcgggagaatcagtccatcctcatcaccggagaatccggagcggggaagactgtgaacacaaaacgtgtcatccagtactttgccagcattgcagccataggggaccgtagcaagaaggaaaatcctaatgcaaacaagggcaccctggaggaccagattatccaggctaaccccgctctggaggccttcggcaacgccaagactgtccggaatgacaactcctcccgctttgggaaattcatcaggatccactttggagctactggaaagctggcttctgcagacatagagacctaccttctggagaagtcccgggtgatcttccagctaaaggctgagaggaactaccacatcttctaccagatcctgtccaacaagaagccggagctgctggacatgctgctggtcaccaacaacccatacgactacgccttcgtctctcagggagaggtgtccgtggcctccattgatgactctgaggagctcttggccactgatagtgcctttgatgtgctgagcttcacggcagaggagaaggctggtgtctacaagctgacaggggccatcatgcactacggaaacatgaagttcaagcagaagcagcgggaggagcaggcggagcctgatggcacagaagatgctgacaaatcagcctaccttatggggctgaactcagctgacctgctcaagggcctgtgtcaccctcgggtgaaggtggggaacgagtatgtcaccaaggggcagagtgtacagcaagtgtactattccatcggggcactggccaagtcagtgtacgagaagatgttcaactggatggtgacacgcatcaacgcaaccctggagaccaagcagccgcgccagtacttcataggtgtcctggacattgccggctttgagatcttcgatttcaacagctttgagcagctgtgcatcaacttcaccaatgagaagctgcagcagttcttcaaccaccacatgttcgtgctggagcaggaggagtacaagaaggagggcattgagtgggagtttatcgacttcggcatggacctgcaggcctgcatcgacctcatcgagaagcccatgggcatcatgtccatcctcgaggaggagtgcatgttccccaaggcctcagacatgaccttcaaggccaagctgtatgacaaccacctgggcaaatccaacaacttccagaagcctcgcaatgtcaaggggaagcaggaagcccacttctccttggtccactatgctggcaccgtggactacaacattatgggctggctggaaaagaacaaggacccactcaatgagacggtggtgggtttgtaccagaagtcctccctcaagctcatggctacactcttctctacctatgcttctgctgataccggtgacagtggtaaaggcaaaggaggcaagaagaaaggctcatccttccaaacagtgtctgctctccaccgggaaaatctgaacaagctgatgacaaacctgaagaccacccaccctcactttgtgcgctgcatcattcccaacgagcgaaaggctccaggggtgatggacaaccccctggtcatgcaccagctgcgatgcaatggcgtgctggagggtatccgcatctgcaggaagggcttccccaaccgcattctctatggggacttccggcagaggtatcgcatcctgaacccagcagccatccctgaggggcaattcattgatagcaggaaaggggctgagaaactgctgggctccctggacattgaccacaaccaatacaagtttggccacaccaaggtgttcttcaaggcgggcctgctggggctgctcgaggagatgcgagatgagaggctgagccgtatcatcaccagaatccaggcccaggcccgagggcagctcatgcgcattgagttcaagaagatagtggaacgcagggatgccctgctggttatccagtggaacattcgggccttcatgggggtcaagaattggccatggatgaagctctacttcaagatcaaaccgctgctgaagagcgcagagacggagaaggagatggccaacatgaaggaggagtttgggcgagtcaaagatgcactggagaagtctgaggctcgccgcaaggagctggaggagaagatggtgtccctgctgcaggagaagaatgacctacagctccaagtgcaggcggaacaagacaacctcaatgatgcagaggagcgctgtgaccagctgatcaagaacaagatccagctggaggccaaggtgaaggagatgaccgagaggctggaggacgaggaggagatgaacgccgagctcactgccaagaagcgcaagctggaagatgagtgctcagagctcaagaaggatattgatgacctggagctgacgctggccaaggtggaaaaggaaaagcatgcaacagagaacaaggttaaaaacctaacagaggagatggctgggctggatgaaatcattgccaagctgaccaaagagaagaaagctctgcaagaagcccaccagcaagccctcgatgacctgcaggctgaagaagacaaggtcaacacgctgaccaagtccaaagtcaagctggagcagcaggtggatgatctggagggatccctggagcaggagaagaaagtgcgcatggacctagagcgagccaagcggaagctggagggagacctgaagctgacccaggagagcatcatggacctggagaatgacaagcttcagctggaagaaaagctcaagaagaaagagttcgacatcagtcagcagaacagtaaaattgaggacgagcaggccctggctcttcagctgcagaagaaactgaaggaaaaccaggcacgcatcgaggagctggaggaggagctggaggcagagcgcacagcccgggctaaggtggagaagctgcgctctgacctgtcccgggagctggaggagatcagtgagaggctggaggaggcaggcggggccacatccgtgcagatagagatgaataagaagcgcgaggccgagttccagaagatgcggcgggacctggaggaggccacgctgcagcacgaggccacggcggcggccctgcgcaagaagcatgctgacagcgtggcggagctgggcgagcagatcgacaacctccagcgggtgaagcagaagctggagaaagagaagagcgagttcaagctggagctggatgacgtcacctccaacatggagcagatcatcaaggccaaggccaacctggagaaagtgtcccggacactggaggaccaggccaatgagtaccgcgtgaagctggaagaagcccagcgctccctcaatgacttcaccacacagcgagccaagctgcagacagagaacggggagttggctaggcaactggaagaaaaggaggcattgatttcccagctgacccgaggcaagctctcctacacccagcagatggaggacctcaagaggcaactggaggaggaaggcaaggccaagaacgccctggcccacgcactgcaatcatcccggcatgactgtgacctgctgagggaacagtatgaagaagaaatggaggccaaggctgagctacagcgtgtcctgtccaaggccaactcagaggtggcccagtggaggaccaagtatgagacggatgccatacagaggacggaggagctggaggaagccaagaagaagctggctcagaggctgcaggatgcagaggaggcagtggaggccgtcaacgccaagtgttcctccctggagaagaccaagcacaggctgcagaatgagatcgaggacctgatggtggacgtggagcgctccaatgccgccgccgcagccctggacaagaagcagaggaactttgacaagatcctggctgagtggaagcagaagtatgaggagtcgcagtcagagctggagtcttcccagaaggaggcgcgctccctgagcacagagctcttcaagctcaagaacgcctatgaggagtctctggagcacctggagaccttcaagcgggagaacaagaacctccaggaggagatctcagacctgactgaacagctgggagaaggggggaaaaacgtgcacgagctggagaagatccgcaaacagctggaggtggagaagctggagctgcagtcagccctggaggaggctgaggcctccctggagcacgaggagggcaagatcctccgtgcccagctggagttcaaccagatcaaggcagagatcgaaaggaagctggcagagaaggatgaggagatggagcaggccaagcgcaaccacctgcggatggtggactccctgcagacctccctggatgcggagacacgcagccgcaatgaggccctgcgggtgaagaagaagatggagggcgacctcaacgagatggagatccagctcagccaggccaatagaatagcctcagaggcacagaaacacctgaagaattctcaagctcacttgaaggacacccagctccagctggatgatgctgtccatgccaatgacgacctgaaggagaacatcgccatcgtggaacggcgcaacaacctgctgcaggcggagctggaggagctgcgggctgtggtggagcagacggagcggtctcggaagctggcagagcaggagctgattgagaccagcgagcgggtgcagctgctgcactcgcagaacaccagcctcatcaaccagaagaagaagatggagtcagacctgacccaactccagacagaagtagaggaggcagtgcaggagtgtaggaacgcagaggagaaggccaagaaggccatcacagatgccgcaatgatggctgaggagctgaagaaggagcaggacaccagcgcccacctggagcgcatgaagaagaacatggagcagaccatcaaggacttgcagcaccgtctggacgaggcagagcagatcgccctcaagggcggcaagaagcagctgcagaagctggaggcccgggtccgggagctggagaatgagctggaggctgagcagaagcgcaatgcagagtcggtgaagggcatgaggaagagcgagcggcgcatcaaggagctcacctaccagacagaggaagacaagaagaacttaatgcggctgcaggacctggtggacaagctacagttgaaggtgaaggcctacaagcgccaggctgaggaggcggaggagcaggccaacaccaacctgtccaagttccgcaaggtgcagcacgagctggatgaggcggaggagagggcggacatcgccgagtcccaggtcaacaagctgcgggccaagagccgggacattggtgccaagaagatgcacgacgaggaataacctctccagcagaccctcgctgtggccaatccacaataaacataaacgttcgactctgcc

SEQ ID NO: 164 - DNA, 79 bp, synthetic construct
  source 1..79: mol_type = other DNA
  variation 10: note = N = C or T
  variation 28: note = N = C or T
  variation 43: note = N = G or C
  variation 48: note = N = C or T
  variation 64: note = N = A or G
  variation 70: note = N = C or G
  variation 73: note = N = T or C
tgcctacctnatggggctgaactcagcngacctgctcaagggnctgtgncaccctcaggtgaangtgggnaangagtac

SEQ ID NO: 165 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 166 - DNA, 90 bp, synthetic construct
  source 1..90: mol_type = other DNA
gtttaagagctatgctggaaacagcatagcaagtttaaataaggctagtccgttatcaacttgaaaaagtggcaccgagtcggtgctttt